(19) United States
(12) Patent Application Publication
(10) Pub. No.: US 2024/0293525 A1
(43) Pub. Date: Sep. 5, 2024

(54) VACCINE AGAINST AFRICAN SWINE FEVER VIRUS INFECTION

(71) Applicants: The Pirbright Institute, Woking (GB); Kumamoto University, Kumamoto (GB); The Chancellor Masters and Scholars of the University of Oxford, Oxford (GB)

(72) Inventors: Linda Dixon, Woking (GB); Ana Reis, Woking (GB); Anusyah Rathakrishnan, Woking (GB); Simon Davis, Oxford (GB); Yuan Jenq Lui, Oxford (GB); Shinji Ikemizu, Kumamoto (JP)

(21) Appl. No.: 17/905,490

(22) PCT Filed: Mar. 5, 2021
(86) PCT No.: PCT/GB2021/050562

§ 371 Date: Sep. 1, 2022

(30) Foreign Application Priority Data
Mar. 6, 2020 (GB) 2003289.2
Mar. 6, 2020 (GB) 2003292.6
Apr. 22, 2020 (GB) 2005878.0
Apr. 22, 2020 (GB) 2005880.6
Aug. 28, 2020 (GB) 2013541.4

(51) Int. Cl. A61K 39/12; A61K 39/00; A61P 37/04; C12N 7/00

(52) U.S. Cl. CPC A61K 39/12; A61P 37/04; C12N 7/00; A61K 2039/5254; A61K 2039/552; A61K 2039/575; C12N 2710/12034; C12N 2710/12061

(57) ABSTRACT

The present invention relates to attenuated African Swine Fever viruses. The attenuated viruses protect pigs against subsequent challenge with virulent virus. The present invention also relates to the use of such attenuated viruses to treat and/or prevent African Swine Fever. The invention also relates to EP402R proteins of African Swine Fever virus comprising particular amino acid substitutions, as well as polynucleotides encoding such proteins and African Swine Fever viruses comprising such proteins.

### FIELD OF THE INVENTION

The present invention relates to attenuated African Swine Fever viruses. The attenuated viruses protect pigs against subsequent challenge with virulent virus. The present invention also relates to the use of such attenuated viruses to treat and/or prevent African Swine Fever. The invention also relates to EP402R proteins of African Swine Fever virus comprising particular amino acid substitutions, as well as polynucleotides encoding such proteins and African Swine Fever viruses comprising such proteins.

### BACKGROUND TO THE INVENTION

### African Swine Fever (ASF)

African swine fever is a devastating haemorrhagic disease of domestic pigs caused by a double-stranded DNA virus, African swine fever virus (ASFV). ASFV is the only member of the Asfarviridae family and replicates predominantly in the cytoplasm of cells. Virulent strains of ASFV can kill domestic pigs within about 5-14 days of infection with a mortality rate approaching 100%.

ASFV can infect and replicate in warthogs (Phacochoerus sp.), bushpigs (Potamocherus sp.) and soft ticks of the Ornithodoros species (which are thought to be a vector), but in these species few if any clinical signs are observed and long term persistent infections can be established. ASFV was first described after European settlers brought pigs into areas endemic with ASFV and, as such, is an example of an “emerging infection”. The disease is currently endemic in many sub-Saharan countries and in Europe in Sardinia. Following its introduction to Georgia in the Trans Caucasus region in 2007, ASFV has spread extensively through neighbouring countries including the Russian Federation. In 2012 the first outbreak was reported in Ukraine and in 2013 the first outbreaks in Belarus. In 2014 further outbreaks were reported in pigs in Ukraine and detection in wild boar in Lithuania and Poland. In 2018 ASFV spread to China and has since spread extensively in China and a number of other Asian countries (Mongolia, Vietnam, Cambodia, Myanmar, N and S Korea, Indonesia, Philippines, Papua New Guinea and Timor-Leste).

There is currently no treatment for ASF. Prevention in countries outside Africa has been attempted on a national basis by restrictions on incoming pigs and pork products, compulsory boiling of waste animal products under license before feeding to pigs and the application of a slaughter policy when the disease is diagnosed. Prevention in Africa is based on measures to keep warthogs and materials contaminated by warthogs away from the herd.

There is thus a need for improved measures to control ASFV infection and prevent spread of the disease.

### African Swine Fever Virus (ASFV)

The complete genome sequences of ASFV isolate Benin 97/1 (a highly pathogenic virus from West Africa, Group1), isolate OURT88/3 (non-pathogenic, attenuated virus from Portugal, Group 1) and isolate BA71V (Vero cell tissue culture adapted non-pathogenic virus, Group 1) have been compared (Chapman et al. 2008 J. Gen. Virol. 89: 397-408). The complete genome of genotype II isolate Georgia 2007/1 has also been sequenced (Chapman et al. 2011 Emerg Infect Dis 17(4): 599-605)

In the OURT88/3 genome, the multigene family (MGF) 360 18R (DP148R) gene, EP153R gene and EP402R gene are each interrupted by frameshift mutations. Additionally, the following MGF genes are absent from the OURT88/3 genome: MGF 110 3L, 6L, 7L, 8L, 10L, 11L and 12L, MGF 300 3L, MGF 360 5L, 6L, 7L, 10L, 11L, 12L, 13L, 14L, 20R, 21R and 22R, and MGF 505 1R, 2R and 6R. The MGF 505 3R gene is also truncated.

The sequences of the high-virulence Lisboa60 strain and the low-virulence NH/P68 strain have also been compared (Portugal et al. 2015 J. Gen. Virol. 96: 408-419).

In the NH/P68 genome, the MGF 360 18R (DP148R) gene, EP153R gene and EP402R gene are each interrupted by a premature stop codon. Additionally, the following MGF genes are absent from the NH/P68 genome: MGF 110 3L, 6L, 7L, 8L, 10L, 11L and 12L, MGF 360 5L, 6L, 7L, 10L, 11L, 12L, 13L, 14L, 20R, 21R and 22R, and MGF 505 1R, 2R and 6R. The MGF 360 9L and MGF 505 3R genes are also truncated.

The sequences of the high-virulence BA71 strain and the low-virulence BA71V strain have also been compared (Rodríguez et al. 2015 PLOS ONE 10(11): e0142889).

Deletion of the DP148R gene from the virulent Benin 97/1 isolate reduced virulence and induced protection against challenge with the parental virus (Reis et al. 2017 J. Virol. 91, 24: e01428-17).

### Differentiation of Infected and Vaccinated Animals (DIVA) Vaccines

A DIVA vaccine (also referred to as a marker vaccine) allows differentiation of animals that have been infected with a wild type pathogen from animals that have been immunised with the vaccine. DIVA vaccines lack at least one immunogenic antigen (a DIVA marker) which is present in the wild type pathogen. Animals infected with the wild type pathogen produce antibodies against the DIVA marker, whereas vaccinated animals do not. Antibodies to the DIVA marker may be detected using a serological assay. Infected animals (which have antibodies to the DIVA marker) may thus be differentiated from vaccinated animals (which do not have antibodies to the DIVA marker), despite both groups of animals having antibodies to other immunogens of the pathogen.

A DIVA marker should be immunogenic, but deletion of the gene should not affect the vaccine's protective capacity.

### SUMMARY OF THE INVENTION

Generally, the invention relates to an attenuated African Swine Fever virus in which expression and/or activity of the genes EP153R and EP402R is disrupted, whilst expression and/or activity of particular MGF genes is not disrupted.

The invention also relates to the determination that disruption of expression and/or activity of the EP153R and EP402R genes in combination with a Differentiation of Infected from Vaccinated Animals (DIVA) mutation can attenuate African Swine Fever virus. The invention concerns particularly a DIVA mutation in the K145R gene.

The invention also relates to the determination of particular amino acid changes in the EP402R protein of African Swine Fever virus which disrupt haemadsorption. Accordingly, the invention includes EP402R proteins comprising such amino acid changes, polynucleotides encoding such proteins and African Swine Fever virus comprising such EP402R proteins and polynucleotides.

Furthermore, the invention concerns the combination of the foregoing, in that the amino acid changes in EP402R may be combined with disruption of the activity and/or expression of the EP153R gene and/or a DIVA mutation to attenuate African Swine Fever virus.

The attenuated African Swine Fever viruses of the invention are of particular benefit as when used in a vaccine they provide protection against infection by wild type African Swine Fever virus strains, as demonstrated in the Examples herein.

In one aspect the invention provides an attenuated African Swine Fever (ASF) virus in which the expression and/or activity of the genes EP153R and EP402R is disrupted; and which comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

The invention also provides an attenuated ASF virus in which the expression and/or activity of the genes EP153R and EP402R is disrupted;
and which comprises a functional version of one or more of the following genes:
   multigene family (MGF) 110 5L, 6L, 8L and 12L,
   MGF 360 6L, 10L, 11L, 12L, 13L, 14L and 21R, and
   MGF 505 1R and 2R.

The invention also provides an attenuated ASF virus in which the expression and/or activity of the genes EP153R and EP402R is disrupted;
and which comprises a functional version of one or more of the following genes:
   multigene family (MGF) 110 11L and 12L,
   MGF 360 6L, 10L, 11L, 12L, 13L, and 14L, and
   MGF 505 1R and 2R.

In another aspect the invention provides an attenuated ASF virus in which the expression and/or activity of the genes EP153R and EP402R is disrupted and which comprises a DIVA mutation. In some embodiments the DIVA mutation disrupts expression of the K145R gene.

In another aspect the invention provides an EP402R protein comprising one or more amino acid changes in the ligand-binding domain wherein the amino acid changes disrupt ligand-binding of the EP402R protein.

In another aspect the invention provides an EP402R protein comprising an amino acid change at a position which corresponds to Q96 and/or W99 of the Georgia 2007/1 EP402R protein (SEQ ID No. 24).

The invention also provides a polynucleotide encoding an EP402R protein of the invention.

The invention also provides a vector comprising a polynucleotide of the invention.

In another aspect the invention provides an ASF virus comprising the EP402R protein of the invention. The invention also provides an ASF virus comprising the polynucleotide of the invention.

The invention also provides the ASF virus of the invention for use in treating and/or preventing a disease in a subject, and the use of an ASF virus of the invention for manufacture of a medicament for treating and/or preventing disease in a subject.

The invention also provides a pharmaceutical composition comprising an ASF virus of the invention, and such a pharmaceutical composition for use in treating and/or preventing a disease in a subject.

The invention also provides a vaccine comprising an ASF virus of the invention, and such a vaccine for use in treating and/or preventing African Swine Fever in a subject.

The invention also provides a method for treating and/or preventing African Swine Fever in a subject which comprises the step of administering to the subject an effective amount of a pharmaceutical composition according to the invention or a vaccine according to the invention.

In another aspect the invention provides a method of producing an ASF virus of the invention, the method comprising changing one or more amino acid(s) in the ligand-binding domain of the EP402R protein wherein the amino acid change disrupts ligand-binding of the EP402R protein.

In another aspect the invention provides a method of producing an ASF virus of the invention, the method comprising changing one or more amino acid(s) in the EP402R protein at a position which corresponds to Q96 and/or W99 of the Georgia 2007/1 EP402R protein (SEQ ID No. 24).

In another aspect the invention provides a method of reducing the ability of an ASF virus to induce haemadsorption, the method comprising changing one or more amino acid(s) in the ligand-binding domain of the EP402R protein wherein the amino acid changes disrupt ligand-binding of the EP402R protein.

In another aspect the invention provides a method of reducing the ability of an ASF virus to induce haemadsorption, the method comprising changing one or more amino acid(s) in the EP402R protein at a position which corresponds to Q96 and/or W99 of the Georgia 2007/1 EP402R protein (SEQ ID No. 24).

In another aspect the invention provides a method of attenuating an ASF virus which comprises disrupting the expression and/or activity of the EP153R and EP402R genes.

### DESCRIPTION OF THE FIGURES

FIG. 1 shows confocal microscopy images of non-permeabilised cells expressing wild-type (A) or mutant (B) CD2v stained with sera from pigs immunised with attenuated ASFV containing a wild-type CD2v gene to detect surface expression.

FIG. 2 shows exemplary images from a HAD (haemadsorption) assay. Vero cells were infected with modified vaccinia virus Ankara expressing T7RNA polymerase and transfected with plasmids (pcDNA3) expressing wild-type or mutant CD2v full-length proteins with a C-terminal HA epitope tag. Pig red blood cells were added and cells observed for attachment of red blood cells to the surface. HAD of red blood cells is observed around three cells transfected with a plasmid expressing wild-type Benin CD2v (A). Partial HAD is observed around one cell expressing CD2v with the Y102 residue mutated to D (B). No HAD is observed for cells expressing CD2v with residue E99 mutated to R (C).

FIG. 3 depicts an alignment of the amino acid sequence of CD2v ligand-binding domain from different ASFV isolates of varying genotypes. E99 and corresponding residues in other isolates are highlighted in yellow.

FIG. 4 shows exemplary images from a HAD assay. Vero cells were infected with modified vaccinia virus Ankara expressing T7RNA polymerase and transfected with plasmids (pcDNA3) expressing wild-type or mutant CD2v full-length proteins from Benin or Georgia strains with a C-terminal HA epitope tag. Pig red blood cells were added and cells observed for attachment of red blood cells to the surface. HAD of red blood cells is observed around four cells transfected with a plasmid expressing wild-type Benin CD2v (A) and around two cells transfected with a plasmid expressing wild-type Georgia CD2v (B). No HAD is observed for cells expressing Georgia CD2v with residue Q96 mutated to R (C) or for untransfected Vero cells (D).

FIG. 5 shows Vero cells transfected with plasmids expressing K145R (A) or B125R (B). Green staining shows the expressed proteins and blue DAPI stain detects DNA.

FIG. 6 shows K145R (A) and B125R (B) expressed in Vero cells and detected by antisera from pigs immunised with ASFV. Cells were fixed, permeabilised and stained with anti-HA (red) to detect the expressed proteins and with sera from pigs immunised with an attenuated genotype|Benin97/1 gene deleted ASFV strain (green). Images are shown of cells stained with sera collected before immunisation and at day 38 post-immunisation. DNA is stained in blue.

FIG. 7 shows exemplary images from HAD assay. Porcine bone marrow cells were infected with wild type Georgia 2007/1 ASFV (A) as control or GeorgiaΔK145RΔEP153RCD2vQ96R ASFV (B) and pig red blood cells added. HAD is observed in cells infected with wild type Georgia 2007/1 at 1 day post-infection (A) but not in cells infected with GeorgiaΔK145RΔEP153RCD2vQ96R at 1 day post-infection (B).

FIG. 8 depicts the experimental protocol used to immunise, boost and challenge pigs with GeorgiaΔK145RΔEP153RCD2vQ96R ASFV (Group K).

FIG. 9 shows rectal temperatures of pigs in control, non-immunised Group M (A) and Group K immunised with GeorgiaΔK145RΔEP153RCD2vQ96R (B) during immunisation and challenge.

FIG. 10 shows clinical scores of pigs in control, non-immunised Group M (A) and Group K immunised with GeorgiaΔK145RΔEP153RCD2vQ96R (B) during immunisation and challenge.

FIG. 11 shows macroscopic lesions in different organs scored at necropsy in pigs from Group K (immunised with GeorgiaΔK145RΔEP153RCD2vQ96R prior to challenge) and Group M (non-immunised control).

FIG. 12 shows the antibody response of pigs in Group K during immunisation and challenge (red dashed line) measured using a commercially available competitive ELISA based on the VP72/B646L major capsid protein.

FIG. 13 shows the cell-mediated immune response in Group K during immunisation and challenge. Peripheral blood mononuclear cells were collected pre-immunization, boost and challenge and stimulated with ASFV genotype|Benin 97/1 (red bars) or ASFV genotype II Georgia 2007/1 (green bars). Numbers of IFN gamma producing cells were measured by Elispot assay.

FIG. 14 shows the number of IFN gamma producing cells for different pigs following stimulation with Georgia 2007/1 isolate over time.

FIG. 15 shows levels of infectious virus detected in whole blood at different days post-immunization and challenge (x-axis). Virus titres were measured by limiting dilution in porcine bone marrow cells and infected cells were detected by fluorescence and are given as TCID₅₀ per ml on the y-axis. Values for different pigs are shown in different colours as indicated on the figure.

### DETAILED DESCRIPTION

### African Swine Fever Virus

African swine fever virus (ASFV) is the causative agent of African swine fever (ASF). The genome structure of ASFV is known in the art, as detailed in Chapman et al. 2008 J. Gen. Virol. 89: 397-408. ASFV is a large, icosahedral, double-stranded DNA virus with a linear genome containing at least 150 genes. The number of genes differs slightly between different isolates of the virus. ASFV has similarities to the other large DNA viruses, e.g., poxvirus, iridovirus and mimivirus. In common with other viral haemorrhagic fevers, the main target cells for replication are those of monocyte, macrophage lineage.

Based on sequence variation in the C-terminal region of the B646L gene encoding the major capsid protein p72, 22 ASFV genotypes (I-XXII) have been identified. All ASFV p72 genotypes have been circulating in eastern and southern Africa. Genotype I has been circulating in Europe, South America, the Caribbean and western Africa. Genotype II is circulating in a number of countries in Europe and Asia. Genotype IX is confined to several East African countries.

Examples of strains from some of the genotypes are given below:
Genotype I: OURT88/3; Brazil/79; Lisbon/60; BA715; Pret; Benin 97/1; IC/1/96; IC/576; CAM/82; Madrid/62; Malta/78; ZAR85; Katange63; Togo; Dakar59; Ourt88/1; BEN/1/97; Dom_Rep; VAL/76; IC/2/96; Awoshie/99; NIG/1/99; NIG/1/98; ANG/70; BEL/85; SPEC120; Lisbon/57; ASFV-Warm; GHA/1/00; GAM/1/00; Ghana; HOL/86; NAM/1/80; NUR/90/1; CAM/4/85; ASFV-Teng; Tegani; ASFV-E75.
Genotype II: Georgia 2007/1; POL/2015/Podlaskie (Polish strain); Belgium/Etalle/wb/2018; ASFV/Kyiv/2016/131; China/2018/AnhuiXCGQ
Genotype III: BOT 1/99
Genotype IV: ASFV-War; RSA/1/99/W
Genotype VI: MOZ 94/1
Genotype VII: VICT/90/1; ASFV-Mku; RSA/1/98
Genotype VIII: NDA/1/90; KAL88/1; ZAM/2/84; JON89/13; KAV89/1; DEZda; AFSV-Mal; Malawi LIL 20/1
Genotype IX: UGA/1/95
Genotype X: BUR/1/84; BUR/2/84; BUR/90/1; UGA/3/95; TAN/Kwh12; Hindell; ASFV-Ken; Virulent Uganda 65.

In an embodiment, the ASF virus of the invention may be attenuated. The attenuated ASF virus of the invention may comprise any of the modifications/mutations described herein, in any combination. The modifications/mutations described herein may attenuate the ASF virus.

### ASFV Isolates

The attenuated ASF virus of the present invention may be derivable or be derived from a wild-type ASF virus isolate, by including mutations in its genome such that the expression and/or activity of the genes EP153R and EP402R is disrupted. The virus may also include a DIVA mutation, such as a DIVA mutation that disrupts expression of the K145R gene.

The term “wild-type” indicates that the virus existed (at some point) in the field, and was isolated from a natural host, such as a domestic pig, tick or warthog. ASFV isolates described to date are summarised in Table 1 below, together with their Genbank Accession numbers.

TABLE 1

| Isolate | Country | Host | Year | Virulence | GenBank accession no. |
|---|---|---|---|---|---|
| BA71qqV | Spain | Pig | 1971 | Tissue culture adapted | U18466 |
| Benin 97/1 | Spain | Pig | 1997 | High | AM712239 |
| Georgia 2007/1 | Georgia | Pig | 2007 | High | FR682468 |
| Kenya | Kenya | Pig | 1950 | High | AY261360 |
| Malawi Lil20/1 | Malawi | Tick | 1983 | High | AY261361 |
| Mkuzi | Zululand | Tick | 1978 | Unknown | AY261362 |
| OURT88/3 | Portugal | Tick | 1988 | Low | AM712240 |
| Pretorisuskop/96/4 | South Africa | Tick | 1996 | High | AY261363 |
| Tangani 62 | Malawi | Pig | 1962 | High | AY261364 |
| Warmbaths | South Africa | Tick | 1987 | Unknown | AY261365 |
| Warthog | Namibia | Warthog | 1980 | Unknown | AY261366 |
| Pol16_20186_07 | Poland | Pig | 2018 | High | MG939583 |
| Pig/HLJ/2018 | China | Pig | 2018 | Virulent | MK333180 |
| DB/LN/2018 | China | Dried blood pig feed | 2018 | Unknown | MK333181 |
| Belgium 2018/1 | Belgium | Wild boar | 2018 | Virulent | LR536725 |
| China/2018/AnhuiXCGQ | China | Pig | 2018 | Virulent | MK128995 |
| Ken05/TK1, Ken06 Bus | Kenya | | 2005/06 | Unknown | NC_044945 KM111295 |
| R35, R25, R7, R8, N10 | Uganda | | 2018 | Unknown | MH025920, MH025918, MH025917, |
| | | | | | MH025916, MH025919 |
| Pol_17_03029_C201 | Poland | Pig | 2017 | Unknown | MG939587 |
| 26544/OG10 | Sardinia/Italy | Pig | 2017 | Unknown | KM102979 |
| 47/Ss?2008 | Sardinia/Italy | Pig | 2008 | Unknown | KX354450 |
| Belgium/Etalle/wb/2018 | Belgium | Wild boar | 2018 | Virulent | MK543947 |
| ASFV/Kyiv/2016/131 | Ukraine | Pig | 2016 | Unknown | MN194591 |
| ASFV-SY18 | China | Pig | 2018 | Unknown | MH766894 |
| ASFV_HU_2018 | Hungary | Wild boar | 2019 | Unknown | MN715134 |
| ASFV-wbBS01 | China | Wild boar | 2019 | Unknown | MK645909 |
| ASFV Georgia 2007/1 (new version) | Georgia | Pig | 2007 | Virulent | LR743116 |
| ASFV/pig/China/CAS19-01/2019 | China | Pig | 2019 | Unknown | MN172368 |
| ASFV/LT14/1490 | Latvia | Wild boar | 2019 | Unknown | MK628478 |
| Odintsovo_02/14 | Russia | Pig | 2014 | | NC_044948 |
| ASFV CzechRepublic 2017/1 | Czech Republic | Wild boar | 2017 | Unknown | LR722600 |
| ASFV Moldova 2017/1 | Moldova | Pig | 2019 | Unknown | LR722599 |
| RSA_2_2008 | South Africa | Pig | 2008 | Unknown | MN336500 |
| LIV_5_40 | Zambia | Tick | | Unknown | MN318203 |
| ASFV/POL/2015/Podlaskie | Poland | Wild boar | 2015 | Virulent | MH681419 |

The genome of the attenuated ASFV of the invention may correspond to any ASFV genotype. The genome of the attenuated ASFV of the invention may essentially correspond to any ASFV genotype.

The term “corresponds to” means that the remainder of the genome of the attenuated ASFV of the invention is the same as a wild-type strain (i.e. a virus that existed at some point in the field). “The remainder of the genome” may refer to all genes other than the genes EP153R and EP402R. “The remainder of the genome” may refer to all genes other than the genes EP153R, EP402R and K145R. In other words, the genes of the attenuated ASFV of the invention may be the same as the genes of the wild-type strain except the genes that are disrupted according to the invention. The genes of the attenuated ASFV of the invention may be the same as the genes of the wild-type strain, except for the genes EP153R and EP402R. The genes of the attenuated ASFV of the invention may be the same as the genes of the wild-type strain, except for the genes EP153R, EP402R and K145R. In an embodiment the genes of the attenuated ASFV of the invention are the same as the genes of the wild-type strain, except for EP153R and EP402R. In an embodiment the genes of the attenuated ASFV of the invention are the same as the genes of the wild-type strain, except for EP153R, EP402R and K145R.

The disrupted genes may also correspond to the wild-type strain. In an embodiment the genes EP153R and EP402R correspond to the wild-type strain. In such an embodiment (i.e. where EP153R and EP402R correspond to the wild-type strain) expression and/or activity of EP153R and EP402R may be disrupted by one or more mutation in an intergenic region and/or non-coding sequence such as a promoter. In other words, the EP153R and EP402R genes are the same as in the wild-type genome but their expression or activity is altered by mutation of a non-genic sequence. Thus all of the genes of the attenuated ASFV of the invention may be the same as the genes of the wild-type strain. In an embodiment all genes of the attenuated ASFV of the invention are the same as the genes of the wild-type strain. In an embodiment all genes of the attenuated ASFV of the invention are the same as the genes of the wild-type strain, except for the K145R gene.

The term “essentially corresponds to” means the same as “corresponds to” with the additional exception that the remainder of the genome may comprise one or more mutations. The one or more mutations may be in other genes (i.e. not in the genes EP153R and EP402R, or not in the genes EP153R, EP402R and K145R).

The genome of the attenuated ASFV may correspond or essentially correspond to genotype I. The genome of the attenuated ASFV may correspond or essentially correspond to genotype II. The genome of the attenuated ASFV may correspond or essentially correspond to genotype III. The genome of the attenuated ASFV may correspond or essentially correspond to genotype IV. The genome of the attenuated ASFV may correspond or essentially correspond to genotype V. The genome of the attenuated ASFV may correspond or essentially correspond to genotype VI. The genome of the attenuated ASFV may correspond or essentially correspond to genotype VII. The genome of the attenuated ASFV may correspond or essentially correspond to genotype VIII. The genome of the attenuated ASFV may correspond or essentially correspond to genotype IX. The genome of the attenuated ASFV may correspond or essentially correspond to genotype X. The genome of the attenuated ASFV may correspond or essentially correspond to genotype XIV.

The genome of the attenuated ASFV may correspond or essentially correspond to genotype I. The genome of the attenuated ASFV may correspond or essentially correspond to genotype II.

Preferably, the genome of the attenuated ASFV may correspond or essentially correspond to genotype II.

The genome of the attenuated ASFV of the invention may correspond or essentially correspond to that of a virulent ASFV strain. Known virulent ASF virus strains include: Georgia 2007/1, Benin 97/1, Kenyan, Malawi Lil20/1, Pretorisuskop/96/4 and Tengani 62. The genome of the attenuated ASFV may correspond or essentially correspond to that of the Benin 97/1 strain.

The genome of the attenuated ASFV may correspond or essentially correspond to that of the Georgia 2007/1 strain.

The genome of the attenuated ASFV of the invention may correspond or essentially correspond to that of an ASFV strain whose virulence is currently unknown, for example: Mkuzi, Warmbaths and Warthog.

In an embodiment the genome of the attenuated ASFV of the invention does not correspond to that of OURT88/3. In an embodiment the genome of the attenuated ASFV of the invention does not correspond to that of NH/P68. In an embodiment the attenuated ASFV of the invention is not OURT88/3. In an embodiment the attenuated ASFV of the invention is not NH/P68. In an embodiment the attenuated ASFV of the invention is neither OURT88/3 nor NH/P68.

### EP402R

In certain aspects, the invention provides an ASF virus in which expression and/or activity of the EP402R gene has been disrupted.

In other aspects, the invention provides an EP402R protein comprising particular amino acid changes.

The EP402R gene encodes a protein which is incorporated in the external layer of the virus and is partly similar to the mammalian T-lymphocyte surface adhesion receptor CD2. In particular, the N-terminal extracellular region of the EP402R protein consists of two immunoglobulin-like (Ig-like) domains similar to the extracellular ligand-binding region of CD2. The EP402R protein may be referred to as CD2v due to this similarity. Accordingly the terms “EP402R” and “CD2v” may be used interchangeably herein. The N-terminal extracellular domain of the EP402R protein may be referred to as the “ligand-binding domain”. The cytoplasmic domain of EP402R protein is dissimilar to CD2.

EP402R is immunogenic (i.e. evokes an immune response) (Netherton et al. 2019 Front. Immunol. 10, 1318). EP402R is required for and directly involved in haemadsorption (Sereda et al. 2018 Slov. Vet. Res, 55(3) 141-150) and may have a role in virus entry or spread. Antibodies from ASFV infected pigs that inhibit haemadsorption can correlate with protection induced against diverse strains supporting a role for antibodies against EP402R in protection of pigs (Malogolovkin et al. 2015 J. Gen. Virol. 96(4) 866-873, Burmakina et al. 2016 J. Gen. Virol. 97(7) 1670-1675). EP402R can bind the host protein AP-1. The functions of EP402R may be mediated by its extracellular, N-terminal, Ig-like domain binding to ligands in the same manner that mammalian CD2 binds extracellular adhesion molecules.

### EP402R Gene Sequences

The gene (i.e. nucleotide) sequences and positions in the genome of EP402R genes from different ASFV strains are presented below.

Georgia 2007/1 EP402R
(NC_044959.1: 73369-74451)
SEQ ID No. 229
ATGATAATACTTATTTTTTTAATATTTTCTAACATAGTTTTAAGT
ATTGATTATTGGGTTAGTTTTAATAAAACAATAATTTTAGATAGT
AATATTACTAATGATAATAATGATATAAATGGAGTATCATGGAAT
TTTTTTAATAATTCTTTTAATACACTAGCTACATGTGGAAAAGCA
GGTAACTTTTGTGAATGTTCTAATTATAGTACATCAATATATAAT
ATAACAAATAATTGTAGCTTAACTATTTTTCCTCATAATGATGTA
TTTGATACAACATATCAAGTAGTATGGAATCAAATAATTAATTAT
ACAATAAAATTATTAACACCTGCTACTCCCCCAAATATCACATAT
AATTGTACTAATTTTTTAATAACATGTAAAAAAAATAATGGAACA
AACACTAATATATATTTAAATATAAATGATACTTTTGTTAAATAT
ACTAATGAAAGTATACTTGAATATAACTGGAATAATAGTAACATT
AACAATTTTACAGCTACATGTATAATTAATAATACAATTAGTACA
TCTAATGAAACAACACTTATAAATTGTACTTATTTAACATTGTCA
TCTAACTATTTTTATACTTTTTTTAAATTATATTATATTCCATTA
AGCATCATAATTGGGATAACAATAAGTATTCTTCTTATATCCATC
ATAACTTTTTTATCTTTACGAAAAAGAAAAAAACATGTTGAAGAA
ATAGAAAGTCCACCACCTGAATCTAATGAAGAAGAACAATGTCAG
CATGATGACACCACTTCCATACATGAACCATCTCCCAGAGAACCA
TTACTTCCTAAGCCTTACAGTCGTTATCAGTATAATACACCTATT
TACTACATGCGTCCCTCAACACAACCACTCAACCCATTTCCCTTA
CCTAAACCGTGTCCTCCACCCAAACCATGTCCGCCACCCAAACCA
TGTCCTCCACCTAAACCATGTCCTTCAGCTGAATCCTATTCTCCA
CCCAAACCACTACCTAGTATCCCGCTACTACCCAATATCCCGCCA
TTATCTACCCAAAATATTTCGCTTATTCACGTAGATAGAATTATT
TAA
China/2018/AnhuiXCGQ EP402R
(MK128995.1: 73383-74465)
SEQ ID No. 230
ATGATAATACTTATTTTTTTAATATTTTCTAACATAGTTTTAAGT
ATTGATTATTGGGTTAGTTTTAATAAAACAATAATTTTAGATAGT
AATATTACTAATGATAATAATGATATAAATGGAGTATCATGGAAT
TTTTTTAATAATTCTTTTAATACACTAGCTACATGTGGAAAAGCA
GGTAACTTTTGTGAATGTTCTAATTATAGTACATCAATATATAAT
ATAACAAATAATTGTAGCTTAACTATTTTTCCTCATAATGATGTA
TTTGATACAACATATCAAGTAGTATGGAATCAAATAATTAATTAT
ACAATAAAATTATTAACACCTGCTACTCCCCCAAATATCACATAT
AATTGTACTAATTTTTTAATAACATGTAAAAAAAATAATGGAACA
AACACTAATATATATTTAAATATAAATGATACTTTTGTTAAATAT
ACTAATGAAAGTATACTTGAATATAACTGGAATAATAGTAACATT
AACAATTTTACAGCTACATGTATAATTAATAATACAATTAGTACA
TCTAATGAAACAACACTTATAAATTGTACTTATTTAACATTGTCA
TCTAACTATTTTTATACTTTTTTTAAATTATATTATATTCCATTA
AGCATCATAATTGGGATAACAATAAGTATTCTTCTTATATCCATC
ATAACTTTTTTATCTTTACGAAAAAGAAAAAAACATGTTGAAGAA
ATAGAAAGTCCACCACCTGAATCTAATGAAGAAGAACAATGTCAG
CATGATGACACCACTTCCATACATGAACCATCTCCCAGAGAACCA
TTACTTCCTAAGCCTTACAGTCGTTATCAGTATAATACACCTATT
TACTACATGCGTCCCTCAACACAACCACTCAACCCATTTCCCTTA
CCTAAACCGTGTCCTCCACCCAAACCATGTCCGCCACCCAAACCA
TGTCCTCCACCTAAACCATGTCCTTCAGCTGAATCCTATTCTCCA
CCCAAACCACTACCTAGTATCCCGCTACTACCCAATATCCCGCCA
TTATCTACCCAAAATATTTCGCTTATTCACGTAGATAGAATTATT
TAA
Warthog EP402R (AY261365.1: 73281-74450)
SEQ ID No. 231
ATGATAATAATCCTTATTTTTTTAATATATTTAAAACTGGTCTTA
AATAATAATATTATTATATGGCATCCTACTTTAAATGAAACAATT
TTTTTAAATGTTACAACTATAGGGAATATTAGTGGTCTATTTTGG
AATACATATTATAATAATAATCGTAGTGAACTTACTAAGTGCGGA
ATATATAATAATTATTGTTCATGTGATAGTCTTAATACATCATTG
TATAATATAAATAATTGTAGTTTAGTTATTTTTCCAAATAATACA
AAAATATTTAATACAATATATGAATTAATATATTCAGGTAATCAA
ACTAATTATACAATAAAATTGCTAGAATCTACTACTCCTCCAAAT
ATTACATATAATTGTACTACATCTGTAATAACATGTAAAAAAAAT
AATGGGACAAATACTAATATATTTTTAACTATAAATAATACGATT
GTTAATTCTACTAATGAAGATGTTATTTACTACTATTGGAATAGA
AGTGAATTTAATAACTTTACAGCTACATGTATGATTAATAATACA
ATTAATTCATCAAATATATCAGAAATTATAGACTGTACTAATACA
TTATTATTAAACAGTTATTTAGACTTTTTTCAAGTAGCCGGTTAT
ATGTTTTATATGATAATTTTTATTGTAACTGGAATAACAGTAAGT
ATTTTTATTTCAATCATAACTTTTTTATCTTTACGAAAAAGAAAA
AAACATGTTGAAGAAATAGAAAGTCTACCACCTGAATCTAATGAA
GAAGAATGTCAGCATGATGATGACACCACTTCCATACATGAACCA
TCCCCCAGAGAACCATTACTTCCTAAACCTTACAGTCGTTATCAG
TATAATACACCTATTTACTACATGCGTCCCTCAACACAACCACTC
AACCCATTTCCCCTACCTAAACCATGCCCACCACCTAAACCGTGT
CCGCCACCCAAACCATGTCCTCCACCCAAACCATGCCCGCCACCC
AAACCATGCCCGCCACCTAAACCATGTCCTCCACCTGAATCATGT
TCTTCACCTGAATCATGTTCTTCACCTGAATCATATTCTCCACCC
AAACCACTACCTAGTATCCCGCTGCTACCCAATATCCCGCCATTA
TCTACACAAAATATTTCGCTTATTCATGTAGATAGAATTATTTAA
Pretorisuskop/96/4 EP402R
(AY261363.1: 74089-75309)
SEQ ID No. 232
ATGATAATGTTCATAACACTTATTTTTTTAAGTTATATTAATATA
GTTTTAAGTAATAATTATTGGGCTAGACTTAACGAAACAATAACT
TTAAATAGTAACATTACTAATGATACTAATAATGAATTAGGTATA
TTTTGGAATTCTTATAATAATACTTATTATAATAATACATTTAAT
AATATTGCTATATGTGGAAAAAAAGGTATTTTTTGTGAATGTAAT
ATTAATTATAATACATCTATATCTAATACATCTATATCTAATACA
TCAATATATAATGTAACAAATAATTGTAGTTTAACGATTTTTCTT
TATGATGATAATATATTTAAAACATATCAATTAGTATATCAGAAT
TACAAAATTAATTATACAATAAACTTATTATTACCTGTTACTTCT
CCAAATATTACATATAATTGTACTAACTCTTTAATAACATGTGAA
AAAAATGATGGGACAAACACTAATATGTTTTTATCTATAAATAAT
ATAACCATTAATCATACTAATCAAGATATTCTTACTTACTATTGG
AATAATAGTGAGTTTAATAATTTTACAGCTACATGTATGATTAAT
AATACACTTAATTCAGCAAATACCACAAAAGTTATCAATTGCACT
AATCCATTATTAAATTCTTACCAAAATTATTTTCTTGAAAATATT
CATACATTATTTTATATCATAATTTTTATTGTGAGTGGATTAATA
GCAAGTATTTTTATTTCAATTATAACTTTTTTATCTTTACGAAAA
AGAAAAAAACATGTTGAAGAAATAGAAAGTCCACCACCTGAATCT
AATGAAGAAGAACAATGTCAGCATGATGACACCACTTCCATACAT
GAACCATCTCCCAGAGAACCATTACTTCCTAAACCTTACAGTCGT
TATCAATATAATACACCTATTTACTACATGCGTCCCTCAACACAA
CCACTCAACCCATTTCCCCTACCTAACCCATGTCCTCCACCTAAA
CCGTGTCCTCCACCCAAACCATGCCCGCCACCTAAACCATGTCCC
CCACCCAAACCATGCCCGCCACCTAAACCGTGTCCTCCACCCAAA
CCATGTCCACCACCTAAACCATGTTCTTCACCTGAATCATATTCT
CCACCCAAACCACTACCTAGTATCCCGCTACTACCCAATATCCCA
CCATTATCTACACAAAATATTTCACTTATTCATGTAGATAGAATT
ATTTAA
L60 EP402R (NC_044941.1: 67916-69037)
SEQ ID No. 233
ATGATAATAATAGTTATTTTTTTAATGTGTTTAAAAATAGTTTTA
AACAATATTATAATATGGAGTACTTTAAATCAAACTGTATTTTTA
AATAATATTTTTACAATTAATGATACATATGGTGGTCTATTTTGG
AATACATATTATGATAATAATCGTAGTAATTTTACTTATTGTGGA
ATAGCAGGAAATTATTGTTCATGTTGTGGTCATAACATATCATTG
TATAATACAACAAATAATTGTAGTTTAATTATTTTTCCTAACAAT
ACAGAAATATTTAATAGAACATATGAATTAGTATATTTGGACAAA
AAAATTAATTATACAGTAAAACTATTAAAATCTGTTGATTCCCCA
ACTATTACATATAATTGTACTAATTCTTTAATAACATGTAAAAAT
AATAATGGGACAAATGTTAATATATATTTAATTATTAATAATACA
ATTAGTTCATTGAATGAAACAGAAAATATAAATTGTACTAATCCA
ATATTAAAATATCAAAATTATTTATCCACATTATTTTATATCATA
ATTTTTATTGTGAGTGGATTAATAATAGGTATTTTTATTTCAATC
ATATCTGTATTATCTATACGAAGAAAAAGAAAAAAACATGTTGAA
GAAATAGAAAGTCCACCACCCTCTGAATCTAATGAAGAAGATATT
TCTCACGATGACACCACTTCCATACATGAACCATCTCCCAGAGAA
CCATTACTTCCTAAGCCTTACAGTCGTTATCAGTATAATACACCT
ATTTACTACATGCGTCCCTCAACACAACCACTCAACCCATTTCCC
CTACCTAAACCATGCCCGCCACCTAAACCATGTCCTCCACCCAAG
CCATGCCCGCCACCCAAACCATGTCCTCCACCTAAACCGTGTTCT
CCACCCAAACCGTGTCGTCCACCTAAACCATGTCCTCCACCTAAA
CCATGTCCTCCACCTAAACCATGTCCTCCACCTAAACCATGTCCT
CCATCCAAACCATGTCCTTCACCTGAATCCTATTCTCCACCCAAA
CCACTACCTAGTATCCCGTTACTACCCAATATCCCGCCATTATCT
ACACAAAATATTTCGCTTATTCATGTAGATAGAATTATTTAA
Benin 97/1 EP402R (NC_044956.1: 67567-68775)
SEQ ID No. 234
ATGATAATAATAGTTATTTTTTTAATGTGTTTAAAAATAGTTTTA
AACAATATTATAATATGGAGTACTTTAAATCAAACTGTATTTTTA
AATAATATTTTTACAATTAATGATACATATGGTGGTCTATTTTGG
AATACATATTATGATAATAATCGTAGTAATTTTACTTATTGTGGA
ATAGCAGGAAATTATTGTTCATGTTGTGGTCATAACATATCATTG
TATAATACAACAAATAATTGTAGTTTAATTATTTTTCCTAACAAT
ACAGAAATATTTAATAGAACATATGAATTAGTATATTTGGACAAA
AAAATTAATTATACAGTAAAACTATTAAAATCTGTTGATTCCCCA
ACTATTACATATAATTGTACTAATTCTTTAATAACATGTAAAAAT
AATAATGGGACAAATGTTAATATATATTTAATTATTAACAATACA
ATTGTTAATGATACTAATGGAGATATCCTTAATTATTATTGGAAT
GGTAATAATAATTTTACAGCTACATGTATGATTAATAATACAATT
AGTTCATTGAATGAAACAGAAAATATAAATTGTACTAATCCAATA
TTAAAATATCAAAATTATTTATCCACATTATTTTATATCATAATT
TTTATTGTGAGTGGATTAATAATAGGTATTTTTATTTCAATCATA
TCTGTATTATCTATACGAAGAAAAAGAAAAAAACATGTTGAAGAA
ATAGAAAGTCCACCACCCTCTGAATCTAATGAAGAAGATATTTCT
CACGATGACACCACTTCCATACATGAACCATCTCCCAGAGAACCA
TTACTTCCTAAGCCTTACAGTCGTTATCAGTATAATACACCTATT
TACTACATGCGTCCCTCAACACAACCACTCAACCCATTTCCCCTA
CCTAAACCATGCCCGCCACCTAAACCATGTCCTCCACCCAAGCCA
TGCCCGCCACCCAAACCATGTCCTCCACCTAAACCGTGTTCTCCA
CCCAAACCGTGTCGTCCACCTAAACCATGTCCTCCACCTAAACCA
TGTCCTCCACCTAAACCATGTCCTCCACCTAAACCATGTCCTCCA
TCCAAACCATGTCCTTCACCTGAATCCTATTCTCCACCCAAACCA
CTACCTAGTATCCCGTTACTACCCAATATCCCGCCATTATCTACA
CAAAATATTTCGCTTATTCATGTAGATAGAATTATTTAA
Tengani 62 EP402R (AY261364.1: 68693-69841)
SEQ ID No. 235
ATGATAATAAAGGTTATTTTTTTAGTTTTCTTTAAAATGGTTTTA
AGTAATATTATAATATGGAGTACTTTAAATAACACAATATATTTA
AATATTAATTATTCAGATAGTAATTTATATACTGGTCTATTTTGG
AATAAATATTATAATAATACTCGTAATAATTTTACTACTTGTGTA
ACATCAAATAGTAGCTATTGTACATGTAAAGGCCATAACACATCA
TTGTATAATATAACAAATAATTGTAGTTTAATCATTTTTCCAAAT
AATACAAAAATATTTAATACAACATATGAATTAGTATATTTAAAT
AATAAAATTAATTATACAATACAAATGGTACAACCTGTTGATCCT
CCAATTATTATATATAATGATACTAATAATTCTTTAATAACATGT
GAAAAAAATAATGGAACGAATACTGAAATCTATTTATATTTAAAT
GATACATTTATTAATAATACTAATGAAAATAGTATTAAATACTAT
TGGAATTGTAGCGAATTAAACCATAATATTACAACTACATGTATT
ATTAATAATACACTTAATTCGGCAAATACCACAAAAGTTATAAAT
TGCACTAATCTATTGTTAAAATCTGACCAAAATTATTTTCTTAAA
AATATTCATACATTATTTTATATCATAATTTTTATTGTGGGTGGA
ACATTAATAAGCATTATTATATCAATCATAACTTTTTTATCTTTA
CGAAAAAGAAAAAAACATGTTGAAGAAATAGAAAGTCCACCACCC
TCTGAATCTAATGAAGAAGAACATTGTCAGCATGATGATACCACT
TCCATACATGAACCATCTCCCAGAGAACCATTACTTCCTAAGCCT
TACAGTCGTTATCAGTATAATACACCTATTTACTACATGCGTCCC
TCAACACAACCACTCAACCCATTTCCCCTACCTAAACCATGTCCT
CCACCTAAACCGTGTCCTCCACCCAAACCATGTCCTCCACCTAAA
CCGTGTCCTCCACCTAAACCGTGTCCCCCACCTAAACCATGTCCT
TCACCTGAATCCCATTCTCCACCCAAACCACTACCTAGTATCCCG
CTGCTACCCAATATTCCCCCATTATCTACACAAAATATTTCGCTT
ATTCATGTAGATAGAATTATTTAA
Malawi Lil-20/1 (1983) EP402R
(AY261361.1: 72275-72458)
SEQ ID No. 236
ATGATAATAATACTTATTTTTTTAATAATTCCTAACATAGTTTTA
AGTATTGATTATTGGGTTAGTTTTAATAAAACAATAATTTTAGAT
AGTAATATTACTAATGATAATAATGATATAAATGGAGTATCATGG
AATTTCCTTAATAATTCTCTTAATACACTAGCTACATGTGGAAAA
GCAGGTAACTTTTGTGAATGCTCTAATTATAGTACATCACTATAT
AATATAGCACATAATTGTAGCTTAACTATTTTCCCTCATAATGAT
GTATTTGGTACACCATATCAAGTAGTATGGAATCAAATAATTAAT
TATACAATAAAATTATTAACACCTGTTACTCCCCCAAATATTACA
TATAATTGTACTAATTTTTTAATAACATGTAAAAAAAATAATGGA
ACAAATACTATTATATATTTCAATATAAATGATACTAATGTTAAA
TATACTAATGAAAGTATACTTGAATATAACTGGAATAATAGTAAC
TTTAACAATTTTACAGCTACATGTATAATTAATAATACAATTAAT
TCATCAAATGATACACAAACTATAGACTGCATCAATACATTATTA
AGCAGTTATTTAGACTTTTTTCAAGTAGCCAGTTATATGTTTTAT
ATGATAATTTTTATTGCAACTGGAATAATAGCAAGTATTTTTATA
TCAATCATAACTTTTTTATCTTTACGAAAAAGAAAAAAACATGTT
GAAGAAATAGAAAGTCCATCGCCATCTGAATCTAATGAAGAAGAA
CAATGTCAGCATGATGACACCACTTCCATACATGAACCGTCTCCC
AGAGAACCATTACTTCCTAAGCCTTACAGTCGATATCAGTATAAT
ACACCTATTTACTATATGCGTCCCTTAACACAACCACTCAATCCA
TCTCCCCTACCCAAACTGTGTCCTCCACCCAAACCGTGTCCTCCA
CCCAAACCGTGTCCTCCACCCAAACCGTGTCCTCCACCCAAACCG
TGTCCTTCATCTGAATCATGTTCTCCACCTGAATCATATTCTCTA
CCCAAACCACTACCTAATATCCCGCTACTACCCAATATCCCGCCA
TTATCTACACAAAATATCTCGCTTATTCACGTAGATAGAATTATT
TAA
Ken06.Bus EP402R (NC_044946.1: 69827-70984)
SEQ ID No. 237
ATGATAATAAAGCTTATTTTTTTAATATCATTTAAAATGATTTTA
GGTATTGATTATTGGGTTAGTCTTAATAATACAATAATTTTAGAT
AGTAACATTACTATTAATAATATTACTAATACTACTAATAATCCT
ACATTAAATGGTATATTTTGGAATATTTATAATAATAGTTATAAT
AATACTTTTAATTTACTTACCACATGTGGGAATACATATAATATA
TGTTCTTGTTCTAATAATTATAATACAATATTATTTAATTATAAT
ACCACAAATAATTGTAGTTTAATTATTTCTCCTCATGATGAAAAA
ATTTTTGATACGATGTTTCAGATAATATATTTAACTAATAAAATT
AATTATACAATACGATGGTTACAACCTGTTGATCCCCCAAATATT
TCATTTAATGATAGTAATTCTTTAATAAAATGTGAAAAAAATAAT
GGAACAAATACTGAAATCTATTTATATTTAAATGATACATTTATT
AATAATACTAATGAAAATGATCTTAAATATTATTGGAATTGTAGT
GAGTTAAACTATAATATTACAGCTACATGTATTATTAATAATACA
CTTAATTCGGCAAATACCACAAAAGTTATAAATTGCACTAATCTA
TTATTAAAATCTGACCAAAATTATTTTCTTAAAAACATTCATACA
TTATTTTATATCATAATTTTTATTGTAACTGGGATAATAATAAGT
ATTTTTATAGCAATCATAACTTTTTTATCTTTACGAAAAAGAAAA
AAACATGTTGAAGAAATAGAAAGTCCATCGCCTGAATCTAATGAA
GAGGAAGAACAACAACATCATCATGACACTACTTCCATACATGAA
CCGTCTCCCAGAGAACCATTACTTCCTAAGCCTTACAGTCGTTAT
CAGTATAATACACCTATTTATTACATGCGTCCCTCAACACAACAA
CTATTTAAATCATATTCTTTACCCAAACCATGTCCCCCACCTAAA
CCATGTCCTCCACCTAAACCATGTCCCCCACCCAAACCATGTCCC
TCACCAGAATCATATCCCTCACCTGAACCATATCCTCTACTACCT
AATATCCCACTACCACCCAATATCCCGCCATTATCTACACAAAAT
ATTTCGCTTATTCACGTAGATAGAATTATTTAA
Ken05/Tk1 EP402R (NC_044945.1: 74107-75249)
SEQ ID No. 238
ATGATAATAAAACTTATTTTTTTAATTTGTTTTAAAATAGTTTTA
AGTATTGATAATAAAACAAAATTTAATGAAACATTAATTTTAGAT
AATATTGATTATTGGGTAAAGTTTAATGATACAATAACTTTAGAT
AGTAATATTACTAGTGAAATAGAAGCTGTATCTTGGAATTTTTAT
AATAATACTTTTAATCTACTTGCTACATGTGGAAAAGCAAGTAAC
TTTTGTAGTTGTTCTAATTATAGTACATCATTTTTTAATATTACA
AATAATTGTAGTTTAACTATTTTTTTAATAGACGAGACGTTATTT
AATACAACATATCAAATAGTATATTCAACTAATATAATTAATTAT
AAAATAAACTTATTAATACCTGTTACTCCTCCAATTATTTCATAT
AATTGTACTAATTGTTCTATAAATTGTAAAAAAAGTAATGGTACA
AATACTAATATTTTTTTATCTATAAATGATACAATTGTTACATAT
ACTAATGAAAGTATTCTTAATTATGATTATAACTGTAGTATTTTA
AATAATAATTTTACAGTTACATGTATAATTAATAATACAATTAGT
ACATCAAATACTACAGAATTTATAAACTGCACTAATATATTATTA
AGTAGTTATCTAGATTTTTTTCAAGTAACTAGTTATATATTTTAT
ATGATAATTTTTATTGTAACTGGAATAACAGTAAGTATTCTTATA
TCCATCATAACTTTCTTATTTATACGAAAAAGAAAACATGTTGAA
GAAATAGAAAGTCCACCGCCTGAATCTAATGAAGAAGAACAACAA
TGTCATCATGACACCACTTCTATACATGAACCGTCTCCCAGAGAA
CCATTACTTCCTAAGCCTTACAGTCGTTATCAATATAATACACCT
ATTTACTACATGCGTCCCTCAACACAACAACTATTTAAATCATAT
TCTTTACCCAAACCATGTCCCCCACCTAAACCATGTCCTCCACCC
AAACCATGTCCCCCACCCAAACCATGTCCTCCACCCAAACCATGT
CCTCCATCCAAACCATATCCTTCACTACCTAGTATCCCGCTACCA
CCCGATATCCCGCCATTATCTACACAAAATATTTCGCTTATTCAC
GTAGATAGAATTATTTAA
Kenya 1950 EP402R (AY261360.1: 76800-77978)
SEQ ID No. 239
ATGATAATAAAACTTATTTTTTTAATTTGTTTTAAAATAGTTTTA
AGTATTGATAATAAAACAAAATTTAATGAAACATTAATTTTAGAT
AATATTGATTATTGGGTAAAGTTTAATGATACAATAACTTTAGAT
AGTAATATTACTAGTGAAATAGAAGCTGTATCTTGGAATTTTTAT
AATAATACTTTTAATCTACTTGCTATATGTGGAAAAGCAAGTAAC
TTTTGTAGTTGTTCTAATTATAGTACATCATTTTTTAATATTACA
AATAATTGTAGTTTAACTATTTTTTTAATAGACGAGACGTTATTT
AATACAACATATCAAATAGTATATTCAACTAATATAATTAATTAT
AAAATAAACTTATTAATACCTGTTACTCCTCCAATTATTTCATAT
AATTGTGCTAATTGTTCTATAAATTGTAAAAAAAGTAATGGTACA
AATACTAATATTTTTTTATCTATAAATGATACAATTGTTACATAT
ACTAATGAAAGTATTCTTAATTATGATTATAACTGTAGTATTTTA
AATAATAATTTTACAGTTACATGTATAATTAATAATACAATTAGC
ACATCAAATACTACAGAATTTATAAACTGCACTAATATATTATTA
AGTAGTTATCTAGATTTTTTTCAAGTAACTAGTTATATATTTTAT
ATGATAATTTTTATTGTAACTGGAATAACAGTAAGTATTCTTATA
TCCATCATAACTTTCTTATTTATACGAAAAAGAAAACATGTTGAA
GAAATAGAAAGTCCACCGCCTGAATCTAATGAAGAAGAACAACAA
TGTCATCATGACACCACTTCTATACATGAACCGTCTCCCAGAGAA
CCATTACTTCCTAAGCCTTACAGTCGTTATCAATATAATACACCT
ATTTACTACATGCGTCCCTCAACACAACAACTATTTAAATCATAT
TCTTTACCCAAACCATGTCCCCCACCTAAACCATGTCCTCCACCT
AAACCATGTCCCCCACCTAAACCATGTCCTCCATCCAAACCATGT
CCTCCACCTGAACCATATTCTCCACCCAAACCATGTCCTCCACCC
AAACCATATCCTTCACTACCTAGTATCCCGCTACCACCCGATATC
CCGCCATTATCTACACAAAATATTTCGCTTATTCACGTAGATAGA
ATTATTTAA
Warthog EP402R (AY261366.1: 70011-71156)
SEQ ID No. 240
ATGATAATTAAACTTATTTTTTTAATTTGTTTCAAAATAGTTTTA
AGTATTAATTATTGGGTTAGGTATAATGATACAGTAACTTTAAAT
AGTAATATCAATAGCGAGACAGAAGGTATATTTTGGAATTTTTAT
AATAATACTTTTAATACTATTGCCACATGTGGAAAAAAAAATAAT
GTTTGTGAATGTTCTAATTATGATAAATCCTTATATAATATAACA
AATAATTGTAGTTTAACAATTTTTCCAAATAATACAAAAATATTT
AATACAACATATCAATTAGTATATTCACGCAATAGAATTAATTAC
ACAATAAATTTGTTATTACCTGTTACATCTCCAATTATTACATAT
AATTGTACTCAATCTTTAATAACATGTGAAAAAACTAATGGAACA
AATATTCATTTATTCTTAAATTTAAATGATACAATTAATGAATAT
ACTAACAAAAGTTTTCTCAATTACTATTGGAATAGTAGTGAGTTA
AATAATATTTTTCTTGCTACATGTATTATTAATAATACACTTAAT
TCAGCAAATACTACAAAAGTTATCAATTGCACTAACCCATTATTA
AAATCTTACCAAAATTATTTTCTTGAAAATATTCATACATTATTT
TATATGATAATTTTTATTGTATCTGGAATAACAATAAGTATTTTT
ATATCAATCATAACTTTTTTATCTTTACGAAAAAGAAAAAAACAT
GTTGAAGAAATAGAGAGTCCACCACCTGAATCTAATGAAGAAGAA
CAATGTCAGCATGATGACACCACTTCTATACATGAACCATCTCCC
AGAGAACCATTACTTCCTAAACCTTACAGTCGTTATCAGTATAAT
ACACCTATTTACTACATGCGTCCCTCAACACAACCACTCAACCCA
TTTCCCCTACCTAAACCATGCCCGCCACCTAAACCGTGTCCTCCA
CCCAAACCATGCCCGCCACCCAAACCATGCCCGCCACCTAAACCA
TGTCCTCCACCCAAACCATGCCCGCCACCTAAACCATGTCCTCCA
CCTGAATCATATTCTCCACCCAAACCACTACCTAGTATCCCGCTG
CTACCCAATATCCCGCCATTATCTACACAAAATATTTCGCTTATT
CATGTAGATAGAATTATTTAA
Mkuzi 1979 EP402R (AY261362.1: 75106-76287)
SEQ ID No. 241
ATGATGATAATTATACTTATTTTTTTAATATGTTTAAAAATAGTT
TTAAATAATAATATTATAATATGGCATACTTTAAATGAAACAATT
TTTTTAAATAATAGTATTACATTTACGGATAATCTTGGTGGTCTA
TTTTGGAATACATATTATAATAATAATCATAGTATCCTTACTAGT
TGCGGAATATATCAAAATTATTGTTCATGCACTAATAATTATAGC
ACATCATTATATAATATAACAAATAATTGTGGTTTAATAATTTTT
CCAAATAATACAAAAAGATTTAACACAACGTATGAATTAGTATAT
TTAAATAATAGTACTAAATATACAATAAACTTATTAGTACCTGTT
ACTCCTCCAAATATTACATATAATTGTACTACATCTGTAATAACA
TGTAAAAAAAATGATGGCACAAATACTAATATGTTTTTAAAAATA
AATGATACAATCGTTAAAAGTAGTAATGAAAGTGTTTTTGAATAC
TCTTGGAATAGTACTGATTTAAATAACAATTTTACGGCTATATGT
ATAATTAATAATACAATTAATTCATCAAATGACACACAAACTATA
GAATGTCCCAATATATTATTAAACAATTATTTAGACTTTTTTCAA
GTAACTAGTTACATGTTTTATATGATAATTTTTATTGTAACTGGA
ATAATAGCAAGTATTTTTATTTCAATCATAACTTTTTTATCTTTA
CGAAAAAGAAAAAAACATGTTGAAGAAATAGAAAGTCCACCACCC
TCTGAATCTAATGAAGAAGATATTTCTCACGATGACACCACTTCC
ATACATGAACCATCTCCCAGAGAACCATTACTTCCTAAGCCTTAC
AGTCGTTATCAGTATAATACACCTATTTACTACATGCGTCCCTCA
ACACAACCACTCAACCCATTTCCCCTACCTAAACCATGCCCGCCA
CCTAAACCATGCCCGCCACCCAAACCATGTCCTCCACCTAAACCG
TGTCCTCCACCCAAACCGTGTCGTCCACCTAAACCATGTCCTCCA
CCTAAACCATGCCCACCACCTAAACCATGTCCTTCACCTGAAACA
TATTCTCCACCCAAACCACTACCTAATATCCCGCTGCTACCCAAT
ATCCCGCCATTATCTACACAAAATATTTCGCTTATTCACGTAGAT
AGGATTATTTAA

In an embodiment the invention provides an ASF virus in which the expression and/or activity of the EP402R gene is disrupted. Suitably, the EP402R gene comprises the sequence of SEQ ID No. 229, 230, 231, 232, 233, 234, 235, 236, 237, 238, 239, 240 or 241. Suitably, the EP402R gene comprises a sequence having at least 70%, at least 80%, at least 90% or at least 95% identity with SEQ ID No. 229, 230, 231, 232, 233, 234, 235, 236, 237, 238, 239, 240 or 241. Suitably, the EP402R gene consists of the sequence of SEQ ID No. 229, 230, 231, 232, 233, 234, 235, 236, 237, 238, 239, 240 or 241.

In an embodiment of the ASFV of the invention the EP402R gene may be partially or completely deleted. Suitably part or all of the sequence of SEQ ID No. 229, 230, 231, 232, 233, 234, 235, 236, 237, 238, 239, 240 or 241 is removed from the ASFV genome. Suitably the ASFV genome lacks any of these sequences.

### EP402R Protein Sequences

The amino acid sequences of EP402R proteins from different ASFV strains are presented below as SEQ ID Nos 21 to 30 and SEQ ID Nos 242 to 246.

Benin 97/1 EP402R protein
SEQ ID No. 21
MIIIVIFLMCLKIVLNNIIIWSTLNQTVFLNNIFTINDTYGGLFW
NTYYDNNRSNFTYCGIAGNYCSCCGHNISLYNTTNNCSLIIFPNN
TEIFNRTYELVYLDKKINYTVKLLKSVDSPTITYNCTNSLITCKN
NNGTNVNIYLIINNTIVNDINGDILNYYWNGNNNFTATCMINNTI
SSLNETENINCTNPILKYQNYLSTLFYIIIFIVSGLIIGIFISII
SVLSIRRKRKKHVEEIESPPPSESNEEDISHDDTTSIHEPSPREP
LLPKPYSRYQYNTPIYYMRPSTQPLNPFPLPKPCPPPKPCPPPKP
CPPPKPCPPPKPCSPPKPCRPPKPCPPPKPCPPPKPCPPPKPCPP
SKPCPSPESYSPPKPLPSIPLLPNIPPLSTQNISLIHVDRII
Mkuzi EP402R protein
SEQ ID No. 22
MIIILIFLICLKIVLNNNIIIWHTLNETIFLNNSITFTDNLGGLF
WNTYYNNNHSILTSCGIYQNYCSCINNYSTSLYNITNNCGLIIFP
NNTKRFNTTYELVYLNNSTKYTINLLVPVTPPNITYNCTTSVITC
KKNDGTNTNMFLKINDTIVKSSNESVFEYSWNSTDLNNNFTAICI
INNTINSSNDTQTIECPNILLNNYLDFFQVTSYMFYMIIFIVTGI
IASIFISIITFLSLRKRKKHVEEIESPPPSESNEEDISHDDTTSI
HEPSPREPLLPKPYSRYQYNTPIYYMRPSTQPLNPFPLPKPCPPP
KPCPPPKPCPPPKPCPPPKPCRPPKPCPPPKPCPPPKPCPSPETY
SPPKPLPNIPLLPNIPPLSTQNISLIHVDRII
Warmbaths EP402R protein
SEQ ID No. 23
MIIILIFLIYLKLVLNNNIIIWHPTLNETIFLNVTTIGNISGLFW
NTYYNNNRSELTKCGIYNNYCSCDSLNTSLYNINNCSLVIFPNNT
KIFNTIYELIYSGNQTNYTIKLLESTTPPNITYNCTTSVITCKKN
NGTNTNIFLTINNTIVNSTNEDVIYYYWNRSEFNNFTATCMINNT
INSSNISEIIDCTNTLLLNSYLDFFQVAGYMFYMIIFIVTGITVS
IFISIITFLSLRKRKKHVEEIESLPPESNEEECQHDDDTTSIHEP
SPREPLLPKPYSRYQYNTPIYYMRPSTQPLNPFPLPKPCPPPKPC
PPPKPCPPPKPCPPPKPCPPPKPCPPPESCSSPESCSSPESYSPP
KPLPSIPLLPNIPPLSTQNISLIHVDRII
Georgia 2007/1 EP402R protein
SEQ ID No. 24
MIILIFLIFSNIVLSIDYWVSFNKTIILDSNITNDNNDINGVSWN
FENNSENTLATCGKAGNFCECSNYSTSIYNITNNCSLTIFPHNDV
FDTTYQVVWNQIINYTIKLLTPATPPNITYNCTNFLITCKKNNGT
NTNIYLNINDTFVKYTNESILEYNWNNSNINNFTATCIINNTIST
SNETTLINCTYLTLSSNYFYTFFKLYYIPLSIIIGITISILLISI
ITFLSLRKRKKHVEEIESPPPESNEEEQCQHDDTTSIHEPSPREP
LLPKPYSRYQYNTPIYYMRPSTQPLNPFPLPKPCPPPKPCPPPKP
CPPPKPCPSAESYSPPKPLPSIPLLPNIPPLSTQNISLIHVDRII
Malawi Lil-20/1 (1983) EP402R protein
SEQ ID No. 25
MIIILIFLIIPNIVLSIDYWVSENKTIILDSNITNDNNDINGVSW
NFLNNSLNTLATCGKAGNFCECSNYSTSLYNIAHNCSLTIFPHND
VFGTPYQVVWNQIINYTIKLLTPVTPPNITYNCTNFLITCKKNNG
TNTIIYENINDTNVKYTNESILEYNWNNSNFNNFTATCIINNTIN
SSNDTQTIDCINTLLSSYLDFFQVASYMFYMIIFIATGIIASIFI
SIITFLSLRKRKKHVEEIESPSPSESNEEEQCQHDDTTSIHEPSP
REPLLPKPYSRYQYNTPIYYMRPLTQPLNPSPLPKLCPPPKPCPP
PKPCPPPKPCPPPKPCPSSESCSPPESYSLPKPLPNIPLLPNIPP
LSTQNISLIHVDRII
Kenya 1950 EP402R protein
SEQ ID No. 26
MIIKLIFLICFKIVLSIDNKTKFNETLILDNIDYWVKENDTITLD
SNITSEIEAVSWNFYNNTENLLAICGKASNFCSCSNYSTSFFNIT
NNCSLTIFLIDETLENTTYQIVYSTNIINYKINLLIPVTPPIISY
NCANCSINCKKSNGTNTNIFLSINDTIVTYTNESILNYDYNCSIL
NNNFTVTCIINNTISTSNTTEFINCTNILLSSYLDFFQVTSYIFY
MIIFIVTGITVSILISIITFLFIRKRKHVEEIESPPPESNEEEQQ
CHHDTTSIHEPSPREPLLPKPYSRYQYNTPIYYMRPSTQQLFKSY
SLPKPCPPPKPCPPPKPCPPPKPCPPSKPCPPPEPYSPPKPCPPP
KPYPSLPSIPLPPDIPPLSTQNISLIHVDRII
N10 EP402R protein
SEQ ID No. 27
MIIKLIFLISFKMILGIDYWVSLNNTIILDSNITINNITNTTNNP
TLNGIFWNIYNNSYNNTENLLTTCGNTYNICSCSNNYNTILFNYN
TTNNCSLIISPHDEKIFDTMFQIIYLTNKINYTIRWLQPVDPPNI
SENDSNSLIKCEKNNGTNTEIYLYLNDTFINNTNENDLKYYWNCS
ELNYNITATCIINNTLNSANTTKVINCTNLLLKSDQNYFLKNIHT
LFYIIIFIVTGIIISIFIAIITFLSLRKRKKHVEEIESPSPESNE
EEEQQHHHDTTSIHEPSPREPLLPKPYSRYQYNTPIYYMRPSTQQ
LFKSYSLPKPCPPPKPCPPPKPCPPPKPCPSPESYPSPEPYPLLP
NIPLPPNIPPLSTQNISLIHVDRII
Tengani 62 EP402R protein
SEQ ID No. 28
MIIKVIFLVFFKMVLSNIIIWSTLNNTIYLNINYSDSNLYTGLFW
NKYYNNTRNNFTTCVTSNSSYCTCKGHNTSLYNITNNCSLIIFPN
NTKIFNTTYELVYLNNKINYTIQMVQPVDPPIIIYNDTNNSLITC
EKNNGTNTEIYLYLNDTFINNTNENSIKYYWNCSELNHNITTTCI
INNTLNSANTTKVINCTNLLLKSDQNYFLKNIHTLFYIIIFIVGG
TLISIIISIITFLSLRKRKKHVEEIESPPPSESNEEEHCQHDDTT
SIHEPSPREPLLPKPYSRYQYNTPIYYMRPSTQPLNPFPLPKPCP
PPKPCPPPKPCPPPKPCPPPKPCPPPKPCPSPESHSPPKPLPSIP
LLPNIPPLSTQNISLIHVDRII
Pr4 EP402R protein
SEQ ID No. 29
MFITLIFLSYINIVLSNNYWARLNETITLNSNITNDTNNELGIFW
NSYNNTYYNNTENNIAICGKKGIFCECNINYNTSISNTSISNTSI
YNVTNNCSLTIFLYDDNIFKTYQLVYQNYKINYTINLLLPVTSPN
ITYNCTNSLITCEKNDGTNTNMFLSINNITINHTNQDILTYYWNN
SEFNNFTATCMINNTLNSANTTKVINCTNPLLNSYQNYFLENIHT
LFYIIIFIVSGLIASIFISIITFLSLRKRKKHVEEIESPPPESNE
EEQCQHDDTTSIHEPSPREPLLPKPYSRYQYNTPIYYMRPSTQPL
NPFPLPNPCPPPKPCPPPKPCPPPKPCPPPKPCPPPKPCPPPKPC
PPPKPCSSPESYSPPKPLPSIPLLPNIPPLSTQNISLIHVDRII
Warthog EP402R protein
SEQ ID No. 30
MIIKLIFLICFKIVLSINYWVRYNDTVTLNSNINSETEGIFWNFY
NNTENTIATCGKKNNVCECSNYDKSLYNITNNCSLTIFPNNTKIF
NTTYQLVYSRNRINYTINLLLPVTSPIITYNCTQSLITCEKTNGT
NIHLFLNLNDTINEYTNKSFLNYYWNSSELNNIFLATCIINNTLN
SANTTKVINCTNPLLKSYQNYFLENIHTLFYMIIFIVSGITISIF
ISIITFLSLRKRKKHVEEIESPPPESNEEEQCQHDDTTSIHEPSP
REPLLPKPYSRYQYNTPIYYMRPSTQPLNPFPLPKPCPPPKPCPP
PKPCPPPKPCPPPKPCPPPKPCPPPKPCPPPESYSPPKPLPSIPL
LPNIPPLSTQNISLIHVDRII
SEQ ID No. 242
China/2018/AnhuiXCGQ EP402R protein
MIILIFLIFSNIVLSIDYWVSENKTIILDSNITNDNNDINGVSWN
FENNSENTLATCGKAGNFCECSNYSTSIYNITNNCSLTIFPHNDV
FDTTYQVVWNQIINYTIKLLTPATPPNITYNCTNFLITCKKNNGT
NTNIYLNINDTFVKYTNESILEYNWNNSNINNFTATCIINNTIST
SNETTLINCTYLTLSSNYFYTFFKLYYIPLSIIIGITISILLISI
ITFLSLRKRKKHVEEIESPPPESNEEEQCQHDDTTSIHEPSPREP
LLPKPYSRYQYNTPIYYMRPSTQPLNPFPLPKPCPPPKPCPPPKP
CPPPKPCPSAESYSPPKPLPSIPLLPNIPPLSTQNISLIHVDRII
Pretorisuskop/96/4 EP402R protein
SEQ ID No. 243
MIMFITLIFLSYINIVLSNNYWARLNETITLNSNITNDTNNELGI
FWNSYNNTYYNNTENNIAICGKKGIFCECNINYNTSISNTSISNT
SIYNVTNNCSLTIFLYDDNIFKTYQLVYQNYKINYTINLLLPVTS
PNITYNCTNSLITCEKNDGTNTNMFLSINNITINHTNQDILTYYW
NNSEFNNFTATCMINNTLNSANTTKVINCTNPLLNSYQNYFLENI
HTLFYIIIFIVSGLIASIFISIITFLSLRKRKKHVEEIESPPPES
NEEEQCQHDDTTSIHEPSPREPLLPKPYSRYQYNTPIYYMRPSTQ
PLNPFPLPNPCPPPKPCPPPKPCPPPKPCPPPKPCPPPKPCPPPK
PCPPPKPCSSPESYSPPKPLPSIPLLPNIPPLSTQNISLIHVDRI
I
L60 EP402R protein
SEQ ID No. 244
MIIIVIFLMCLKIVLNNIIIWSTLNQTVFLNNIFTINDTYGGLFW
NTYYDNNRSNFTYCGIAGNYCSCCGHNISLYNTTNNCSLIIFPNN
TEIFNRTYELVYLDKKINYTVKLLKSVDSPTITYNCTNSLITCKN
NNGTNVNIYLIINNTISSLNETENINCTNPILKYQNYLSTLFYII
IFIVSGLIIGIFISIISVLSIRRKRKKHVEEIESPPPSESNEEDI
SHDDTTSIHEPSPREPLLPKPYSRYQYNTPIYYMRPSTQPLNPFP
LPKPCPPPKPCPPPKPCPPPKPCPPPKPCSPPKPCRPPKPCPPPK
PCPPPKPCPPPKPCPPSKPCPSPESYSPPKPLPSIPLLPNIPPLS
TQNISLIHVDRII
Ken06.Bus EP402R protein
SEQ ID No. 245
MIIKLIFLISFKMILGIDYWVSLNNTIILDSNITINNITNTTNNP
TLNGIFWNIYNNSYNNTFNLLTTCGNTYNICSCSNNYNTILFNYN
TTNNCSLIISPHDEKIFDTMFQIIYLTNKINYTIRWLQPVDPPNI
SFNDSNSLIKCEKNNGTNTEIYLYLNDTFINNTNENDLKYYWNCS
ELNYNITATCIINNTLNSANTTKVINCTNLLLKSDQNYFLKNIHT
LFYIIIFIVTGIIISIFIAIITFLSLRKRKKHVEEIESPSPESNE
EEEQQHHHDTTSIHEPSPREPLLPKPYSRYQYNTPIYYMRPSTQQ
LFKSYSLPKPCPPPKPCPPPKPCPPPKPCPSPESYPSPEPYPLLP
NIPLPPNIPPLSTQNISLIHVDRII
Ken05/Tk1 EP402R protein
SEQ ID No. 246
MIIKLIFLICFKIVLSIDNKTKFNETLILDNIDYWVKENDTITLD
SNITSEIEAVSWNFYNNTENLLATCGKASNFCSCSNYSTSFFNIT
NNCSLTIFLIDETLENTTYQIVYSTNIINYKINLLIPVTPPIISY
NCTNCSINCKKSNGTNTNIFLSINDTIVTYTNESILNYDYNCSIL
NNNFTVTCIINNTISTSNTTEFINCTNILLSSYLDFFQVTSYIFY
MIIFIVTGITVSILISIITFLFIRKRKHVEEIESPPPESNEEEQQ
CHHDTTSIHEPSPREPLLPKPYSRYQYNTPIYYMRPSTQQLFKSY
SLPKPCPPPKPCPPPKPCPPPKPCPPPKPCPPSKPYPSLPSIPLP
PDIPPLSTQNISLIHVDRII

In an embodiment the invention provides an attenuated ASF virus in which the expression and/or activity of the EP402R gene is disrupted. Suitably, the EP402R gene encodes a protein comprising a sequence having at least 70%, 75%, 80%, 85%, 90% or 95% identity with any of SEQ ID Nos 21, 22, 23, 24, 25, 26, 27, 28, 29, 30, 242, 243, 244, 245 or 246. Suitably, the EP402R gene encodes a protein comprising the sequence of any of SEQ ID Nos 21, 22, 23, 24, 25, 26, 27, 28, 29, 30, 242, 243, 244, 245 or 246.

In an embodiment the ASFV of the invention does not express EP402R protein. In other words, the ASFV of the invention does not express any proteins with sequences of SEQ ID Nos 21, 22, 23, 24, 25, 26, 27, 28, 29, 30, 242, 243, 244, 245 or 246.

### EP402R Mutant Proteins

In one aspect the invention provides an EP402R protein comprising one or more amino acid changes in the ligand-binding domain wherein the amino acid changes disrupt ligand-binding of the EP402R protein.

The ligand-binding domain of EP402R is formed by amino acids starting at position 1 (N-terminus) and running to roughly position 200. For example, the ligand-binding domain of Benin 97/1 EP402R is formed by amino acids 1-198 of SEQ ID No. 21 which are presented below as SEQ ID No. 34.

Benin 97/1 EP402R protein
ligand-binding domain
SEQ ID No. 34
MIIIVIFLMCLKIVLNNIIIWSTLNQTVFLNNIFTINDTYGGLFW
NTYYDNNRSNFTYCGIAGNYCSCCGHNISLYNTTNNCSLIIFPNN
TEIFNRTYELVYLDKKINYTVKLLKSVDSPTITYNCTNSLITCKN
NNGTNVNIYLIINNTIVNDINGDILNYYWNGNNNFTATCMINNTI
SSLNETENINCTNPILKY

The ligand-binding domain of Georgia 2007/1 EP402R is formed by amino acids 1-203 of SEQ ID No. 24 which are presented below as SEQ ID No. 380.

Georgia 2007/1 EP402R protein
ligand-binding domain
SEQ ID No. 380
MIILIFLIFSNIVLSIDYWVSENKTIILDSNITNDNNDINGVSWN
FENNSENTLATCGKAGNFCECSNYSTSIYNITNNCSLTIFPHNDV
FDTTYQVVWNQIINYTIKLLTPATPPNITYNCTNFLITCKKNNGT
NTNIYLNINDTFVKYTNESILEYNWNNSNINNFTATCIINNTIST
SNETTLINCTYLTLSSNYFYTFF

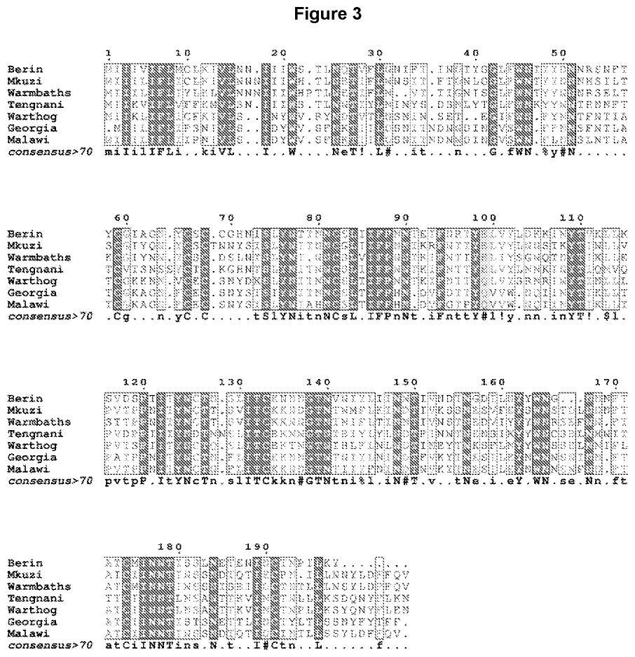

Suitably, the ligand-binding domain of the EP402R protein comprises the sequence of SEQ ID No. 34 or 380. Suitably, the ligand-binding domain of the EP402R protein comprises a sequence having at least 70%, 80%, 90% or at least 95% identity with SEQ ID No. 34 or 380. The ligand-binding domain of the EP402R protein from other strains can be readily identified by alignment with the sequence of Benin 97/1 EP402R protein and/or Georgia 2007/1 EP402R protein, such as shown in FIG. 3.

Suitably one or more amino acids (such as two or more, three or more, four or more or five or more amino acids) in the ligand-binding domain of the EP402R protein are changed compared to the wild type EP402R protein. In an embodiment, the one or more amino acids in the ligand-binding domain are deleted (i.e. removed entirely). In an embodiment, the one or more amino acids in the ligand-binding domain are changed to different amino acids (i.e. replaced). Such an amino change may be referred to as a substitution. Changing one or more amino acids in the ligand-binding domain of the EP402R protein may disrupt expression and/or activity of the EP402R protein. In an embodiment, changing one or more amino acids in the ligand-binding domain of the EP402R protein may disrupt haemadsorption mediated by the EP402R protein.

The amino acid changes in the EP402R protein are caused by one or more mutations in the sequence coding for the EP402R protein. In an embodiment, the one or more mutations may be a deletion or an interruption as described herein. For example, deletion of part of the coding sequence for the ligand-binding domain of the EP402R protein would result in the absence of the encoded amino acids (i.e. changing the amino acids) from the ligand-binding domain, which may disrupt ligand binding.

In an embodiment, one or more of the mutations may be a point mutation. Suitably one or more of the mutations may be point mutation that changes a single amino acid into a different amino acid. Changing even a single amino acid may disrupt expression and/or activity of the EP402R protein, such as ligand binding activity.

Changing one or more amino acids may disrupt folding of the EP402R protein. Disruption of folding may mean the EP402R protein cannot fold at all causing it to be degraded by the cellular protein degradation machinery. Alternatively, the disruption of folding may mean the EP402R protein is folded differently, impairing its function, or the EP402R protein may be folded more slowly and so is not correctly expressed (e.g. it is not expressed at the cell surface).

Changing a charged amino acid to an amino acid with the opposite charge may disrupt folding of the EP402R ligand-domain such that a binding pocket is deformed, which prevents ligand binding due to steric hindrance. Alternatively or additionally, substitution with an amino acid with the opposite charge may prevent electrostatic binding of a ligand. Another possibility is that changing an amino acid with a small side-chain to an amino acid with a bulky side-chain, or vice versa, may disrupt folding of the EP402R ligand-binding domain so that a binding pocket does not form which prevents ligand binding.

Thus in an embodiment the one or more changed amino acids in the ligand-binding domain may comprise a negatively charged amino acid that is changed to a positively charged amino acid. In an embodiment the one or more changed amino acids in the ligand-binding domain may comprise a positively charged amino acid that is changed to a negatively charged amino acid. Positively charged amino acids (i.e. amino acids that can have a positive charge) include histidine (H), lysine (K) and arginine (R). Negatively charged amino acids (i.e. amino acids that can have a negative charge) include aspartic acid (D) and glutamic acid (E).

In an embodiment the one or more changed amino acids in the ligand-binding domain may comprise an amino acid with a small side-chain that is changed to an amino acid with a bulky side-chain. In an embodiment the one or more changed amino acids in the ligand-binding domain may comprise an amino acid with a bulky side-chain that is changed to an amino acid with a small side-chain. Amino acids with a bulky side-chain include tryptophan (W). Amino acids with a small side-chain include glycine (G) and alanine (A).

In an embodiment the one or more changed amino acids in the ligand-binding domain may comprise an amino acid with a hydrophilic side-chain that is changed to an amino acid with a hydrophobic side-chain. In an embodiment the one or more changed amino acids in the ligand-binding domain may comprise an amino acid with a hydrophobic side-chain that is changed to an amino acid with a hydrophilic side-chain. Amino acids with a hydrophobic side-chain include alanine (A), valine (V), isoleucine (I), leucine (L), methionine (M), phenylalanine (F), tyrosine (Y) and tryptophan (W). Amino acids with a hydrophilic side-chain include arginine (R), histidine (H), lysine (K), aspartic acid (D), glutamic acid (E), serine (S), threonine (T), asparagine (N) and glutamine (Q).

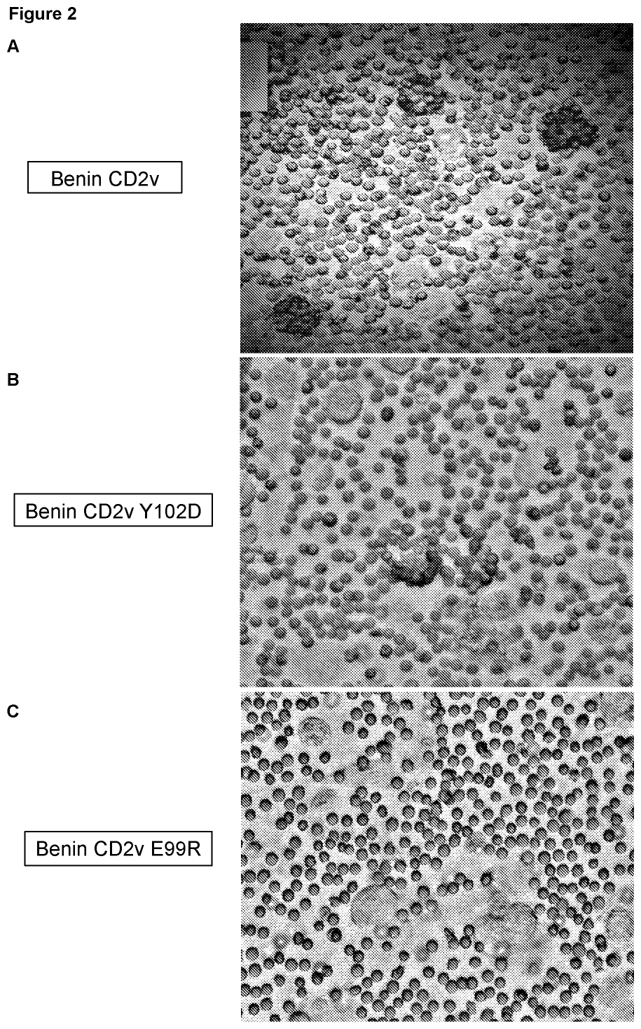

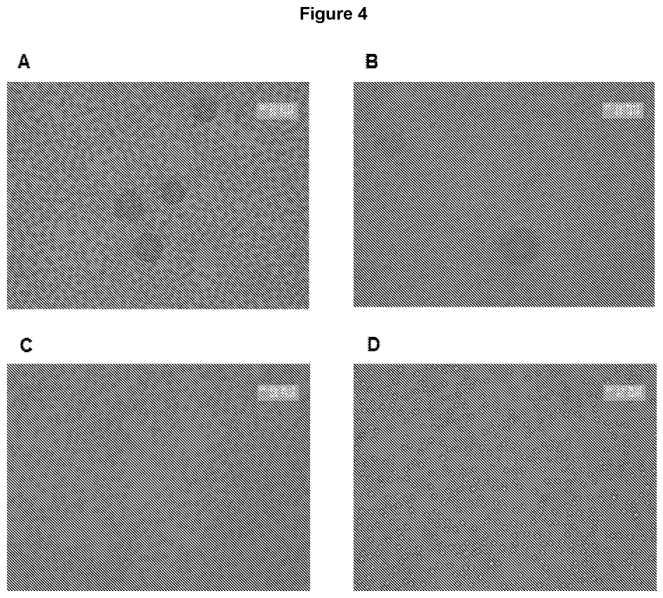

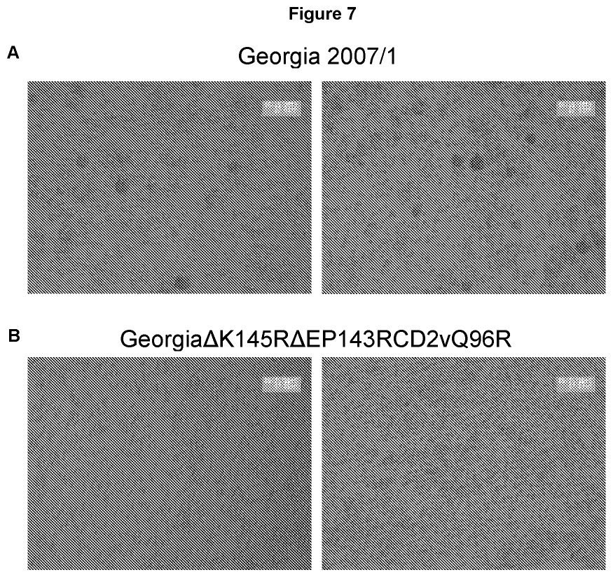

Changing amino acid E99 to R or amino acid Y102 to D disrupts the ability of EP402R protein from Benin 97/1 strain to mediate haemadsorption (see FIG. 2). Furthermore, changing the amino acid Q96 to R disrupts the ability of EP402R protein from Georgia 2007/1 strain to mediate haemadsorption (see FIGS. 4 and 7). Changing the amino acid W99 of Georgia 2007/1 EP402R to D may disrupt the folding of EP402R and thus lead to disrupted expression and/or activity. Accordingly, in an embodiment the EP402R protein of the invention comprises an amino acid change at a position which corresponds to Q96 and/or W99 of the Georgia 2007/1 EP402R protein (SEQ ID No. 24). Q96 of the Georgia 2007/1 EP402R protein corresponds to E99 of the Benin 97/1 EP402R protein, and W99 of the Georgia 2007/1 EP402R protein corresponds to Y102 of the Benin 97/1 EP402R protein. Thus, in an embodiment the EP402R protein of the invention comprises an amino acid change at a position which corresponds to E99 and/or Y102 of the Benin 97/1 EP402R protein (SEQ ID No. 21). In other words, the EP402R protein expressed by the attenuated ASF virus of the invention may be the EP402R protein from any ASF virus strain, and the amino acid that is changed corresponds to Q96 and/or W99 of the amino acid sequence of Georgia 2007/1 EP402R protein, which is presented herein as SEQ ID No. 24. Alternatively expressed, the EP402R protein expressed by the attenuated ASF virus of the invention may be the EP402R protein from any ASF virus strain, and the amino acid that is changed corresponds to E99 and/or Y102 of the amino acid sequence of the EP402R protein from the Benin 97/1 strain, which is presented herein as SEQ ID NO. 21. The positions Q96 and W99 refer to amino acids glutamine and tryptophan at positions 96 and 99 respectively of the Georgia 2007/1 EP402R protein. The positions E99 and Y102 refer to amino acids glutamic acid and tyrosine at positions 99 and 102 respectively of the Benin 97/1 EP402R protein.

Whether an amino acid corresponds to Q96 and/or W99 of the Georgia 2007/1 EP402R protein (or to E99 and/or Y102 of the Benin 97/1 EP402R protein) may be assessed by sequence alignment. For example, FIG. 3 shows an alignment of EP402R proteins from Benin, Mkuzi, Warmbaths, Tengnani, Warthog, Georgia and Malawi strains. The amino acid in the EP402R protein from each strain that corresponds to Q96 of Georgia 2007/1 EP402R protein and E99 of Benin 97/1 EP402R protein is the amino acid shown below the Benin E99 residue (highlighted in yellow). The amino acid in the EP402R protein from each strain that corresponds to W99 of Georgia 2007/1 EP402R protein and Y102 of Benin 97/1 EP402R protein is the amino acid shown below the Benin Y102 residue (three amino acids to the right of the amino acids highlighted in yellow). The amino acids in the EP402R protein from the strains in FIG. 3 that correspond to Q96 and/or W99 of the Georgia 2007/1 EP402R protein and E99 and Y102 in Benin 97/1 EP402R are given in Table 2 below.

TABLE 2

| | |
|---|---|
| Strain | Corresponding Amino acid |

| | | |
|---|---|---|
| Benin 97/1 | E99 | Y102 |
| Mkuzi | E101 | Y104 |
| Warmbaths | E98 | Y101 |
| Tengani | E100 | Y103 |
| Warthog | Q95 | Y98 |
| Georgia | Q96 | W99 |
| Malawi | Q97 | W100 |

Amino acids corresponding to Q96 and/or W99 of the Georgia 2007/1 EP402R protein and E99 and Y102 of Benin 97/1 EP402R exist in EP402R proteins from strains other than those shown in FIG. 3 and listed in Table 2 above (such as other strains listed in Table 1 herein). The skilled person can readily determine using sequence alignment which amino acid in an EP402R protein from a given strain, such as a strain from Table 1, corresponds to Q96 and/or W99 of the Georgia 2007/1 EP402R protein and E99 or Y102 of Benin 97/1 EP402R. The corresponding amino acid may be identical to Q96 or W99 of Georgia 2007/1 EP402R or to E99 or Y102 of Benin 97/1 EP402R. For example, an amino acid corresponding to Q96 of Georgia 2007/1 EP402R may also be a Q (glutamine). The corresponding amino acid may be similar to Q96 or W99. Suitably the corresponding amino acid has the same charge as Q96 or W99. Suitably the corresponding amino acid to Q96 is an E (glutamic acid). Suitably the corresponding amino acid to W99 is a Y (tyrosine). The expression “corresponding to Q96 and/or W99 of Georgia 2007/1 EP402R protein” encompasses Q96 and/or W99 of Georgia 2007/1 EP402R protein. The expression “corresponding to E99 and/or Y102 of Benin 97/1 EP402R protein” encompasses E99 and/or Y102 of Benin 97/1 EP402R protein.

Suitably the amino acid at the position which corresponds to Q96 is changed to R or to an amino acid that is a conservative replacement of R and/or the amino acid at the position which corresponds to W99 is changed to D or to an amino acid that is a conservative replacement of D. An amino acid that is “conservative replacement” has similar characteristics to the other amino acid. For example, the conservative replacement may have similar charge (positive or negative), similar hydrophobicity (hydrophilic or hydrophobic) or similar molecular size, be also aromatic, or have a combination of these characteristics. Suitably the amino acid at the position which corresponds to Q96 is changed to H, K or R and/or the amino acid at the position which corresponds to W99 is changed to D, E, N or Q. Suitably the amino acid at the position which corresponds to Q96 is changed to R and/or the amino acid at the position which corresponds to W99 is changed to D.

In an embodiment the EP402R protein of the invention comprises an amino acid sequence having at least 70% sequence identity, such as at least 75% identity, such as at least 80% identity, such as at least 85% identity, such as at least 90% identity, such as least 95% identity, such as least 96% identity, such as least 97% identity, such as least 98% identity, such as least 99% identity, with any of SEQ ID Nos 21 to 30 or SEQ ID Nos 242 to 246 (i.e. the sequences of EP402R protein from different ASFV strains as described herein). Suitably the EP402R protein of the invention comprises an amino acid sequence having at least 70%, 75%, 80%, 85%, 90%, 95%, 96%, 97%, 98% or 99% identity with SEQ ID No. 21. Suitably the EP402R protein of the invention comprises an amino acid sequence having at least 70%, 75%, 80%, 85%, 90%, 95%, 96%, 97%, 98% or 99% identity with SEQ ID No. 22. Suitably the EP402R protein of the invention comprises an amino acid sequence having at least 70%, 75%, 80%, 85%, 90%, 95%, 96%, 97%, 98% or 99% identity with SEQ ID No. 23. Suitably the EP402R protein of the invention comprises an amino acid sequence having at least 70%, 75%, 80%, 85%, 90%, 95%, 96%, 97%, 98% or 99% identity with SEQ ID No. 24. Suitably the EP402R protein of the invention comprises an amino acid sequence having at least 70%, 75%, 80%, 85%, 90%, 95%, 96%, 97%, 98% or 99% identity with SEQ ID No. 25. Suitably the EP402R protein of the invention comprises an amino acid sequence having at least 70%, 75%, 80%, 85%, 90%, 95%, 96%, 97%, 98% or 99% identity with SEQ ID No. 26. Suitably the EP402R protein of the invention comprises an amino acid sequence having at least 70%, 75%, 80%, 85%, 90%, 95%, 96%, 97%, 98% or 99% identity with SEQ ID No. 27. Suitably the EP402R protein of the invention comprises an amino acid sequence having at least 70%, 75%, 80%, 85%, 90%, 95%, 96%, 97%, 98% or 99% identity with SEQ ID No. 28. Suitably the EP402R protein of the invention comprises an amino acid sequence having at least 70%, 75%, 80%, 85%, 90%, 95%, 96%, 97%, 98% or 99% identity with SEQ ID No. 29. Suitably the EP402R protein of the invention comprises an amino acid sequence having at least 70%, 75%, 80%, 85%, 90%, 95%, 96%, 97%, 98% or 99% identity with SEQ ID No. 30. Suitably the EP402R protein of the invention comprises an amino acid sequence having at least 70%, 75%, 80%, 85%, 90%, 95%, 96%, 97%, 98% or 99% identity with SEQ ID No. 242. Suitably the EP402R protein of the invention comprises an amino acid sequence having at least 70%, 75%, 80%, 85%, 90%, 95%, 96%, 97%, 98% or 99% identity with SEQ ID No. 243. Suitably the EP402R protein of the invention comprises an amino acid sequence having at least 70%, 75%, 80%, 85%, 90%, 95%, 96%, 97%, 98% or 99% identity with SEQ ID No. 244. Suitably the EP402R protein of the invention comprises an amino acid sequence having at least 70%, 75%, 80%, 85%, 90%, 95%, 96%, 97%, 98% or 99% identity with SEQ ID No. 245. Suitably the EP402R protein of the invention comprises an amino acid sequence having at least 70%, 75%, 80%, 85%, 90%, 95%, 96%, 97%, 98% or 99% identity with SEQ ID No. 246.

In certain embodiments, the amino acids that differ between the reference sequence (i.e. SEQ ID Nos 21 to 30 or SEQ ID Nos 242 to 246) and the sequence that is less than 100% identical to the reference sequence are conservative replacements.

The amino acid sequence of the EP402R protein from Benin 97/1 strain comprising the E99R amino acid change is depicted below as SEQ ID No. 31.

Benin 97/1 EP402R E99R protein
SEQ ID No. 31
MIIIVIFLMCLKIVLNNIIIWSTLNQTVFLNNIFTINDTYGGLFW
NTYYDNNRSNFTYCGIAGNYCSCCGHNISLYNTTNNCSLIIFPNN
TEIFNRTYRLVYLDKKINYTVKLLKSVDSPTITYNCTNSLITCKN
NNGTNVNIYLIINNTIVNDINGDILNYYWNGNNNFTATCMINNTI
SSLNETENINCTNPILKYQNYLSTLFYIIIFIVSGLIIGIFISII
SVLSIRRKRKKHVEEIESPPPSESNEEDISHDDTTSIHEPSPREP
LLPKPYSRYQYNTPIYYMRPSTQPLNPFPLPKPCPPPKPCPPPKP
CPPPKPCPPPKPCSPPKPCRPPKPCPPPKPCPPPKPCPPPKPCPP
SKPCPSPESYSPPKPLPSIPLLPNIPPLSTQNISLIHVDRII

The amino acid sequence of the EP402R protein from Benin 97/1 strain comprising the Y102D substitution is depicted below as SEQ ID No. 32.

Benin 97/1 EP402R Y102D protein
SEQ ID No. 32
MIIIVIFLMCLKIVLNNIIIWSTLNQTVFLNNIFTINDTYGGLFW
NTYYDNNRSNFTYCGIAGNYCSCCGHNISLYNTTNNCSLIIFPNN
TEIFNRTYELVDLDKKINYTVKLLKSVDSPTITYNCTNSLITCKN
NNGTNVNIYLIINNTIVNDINGDILNYYWNGNNNFTATCMINNTI
SSLNETENINCTNPILKYQNYLSTLFYIIIFIVSGLIIGIFISII
SVLSIRRKRKKHVEEIESPPPSESNEEDISHDDTTSIHEPSPREP
LLPKPYSRYQYNTPIYYMRPSTQPLNPFPLPKPCPPPKPCPPPKP
CPPPKPCPPPKPCSPPKPCRPPKPCPPPKPCPPPKPCPPPKPCPP
SKPCPSPESYSPPKPLPSIPLLPNIPPLSTQNISLIHVDRII

The amino acid sequence of the EP402R protein from Georgia 2007/1 strain comprising the Q96R substitution is depicted below as SEQ ID No. 33.

Georgia 2007/1 EP402R Q96R protein
SEQ ID No. 33
MIILIFLIFSNIVLSIDYWVSENKTIILDSNITNDNNDINGVSWN
FENNSENTLATCGKAGNFCECSNYSTSIYNITNNCSLTIFPHNDV
FDTTYRVVWNQIINYTIKLLTPATPPNITYNCTNFLITCKKNNGT
NTNIYLNINDTFVKYTNESILEYNWNNSNINNFTATCIINNTIST
SNETTLINCTYLTLSSNYFYTFFKLYYIPLSIIIGITISILLISI
ITFLSLRKRKKHVEEIESPPPESNEEEQCQHDDTTSIHEPSPREP
LLPKPYSRYQYNTPIYYMRPSTQPLNPFPLPKPCPPPKPCPPPKP
CPPPKPCPSAESYSPPKPLPSIPLLPNIPPLSTQNISLIHVDRII

The amino acid sequence of the EP402R protein from Georgia 2007/1 strain comprising the W99D substitution is depicted below as SEQ ID No. 379.

Georgia 2007/1 EP402R W99D protein
SEQ ID No. 379
MIILIFLIFSNIVLSIDYWVSENKTIILDSNITNDNNDINGVSWN
FENNSENTLATCGKAGNFCECSNYSTSIYNITNNCSLTIFPHNDV
FDTTYQVVDNQIINYTIKLLTPATPPNITYNCTNFLITCKKNNGT
NTNIYLNINDTFVKYTNESILEYNWNNSNINNFTATCIINNTIST
SNETTLINCTYLTLSSNYFYTFFKLYYIPLSIIIGITISILLISI
ITFLSLRKRKKHVEEIESPPPESNEEEQCQHDDTTSIHEPSPREP
LLPKPYSRYQYNTPIYYMRPSTQPLNPFPLPKPCPPPKPCPPPKP
CPPPKPCPSAESYSPPKPLPSIPLLPNIPPLSTQNISLIHVDRII

In an embodiment the EP402R protein of the invention comprises the amino acid sequence of any of SEQ ID Nos 31, 32, 33 or 379. Suitably the EP402R protein of the invention comprises or consists of the amino acid sequence of SEQ ID No. 31 (Benin 97/1 EP402R protein with E99R amino acid change). Suitably the EP402R protein of the invention comprises or consists of the amino acid sequence of SEQ ID No. 32 (Benin 97/1 EP402R protein with Y102D amino acid change). Suitably the EP402R protein of the invention comprises or consists of the amino acid sequence of SEQ ID No. 33 (Georgia 2007/1 EP402R protein with Q96R amino acid change). Suitably the EP402R protein of the invention comprises or consists of the amino acid sequence of SEQ ID No. 379 (Georgia 2007/1 EP402R protein with W99D amino acid change).

In an embodiment the EP402R protein of the invention comprises one or more amino acid changes at positions in the EP402R protein corresponding to N16, 119, W21, Y76, E99, Y102 and/or N108 of Benin 97/1 EP402R protein (SEQ ID NO. 21). In an embodiment the one or more mutations change the amino acid at position N16, 119, W21, Y76, E99, Y102 and/or N108 of the EP402R protein of the Benin 97/1 strain, or the corresponding position in the EP402R protein of any other ASFV strain. In an embodiment the one or more mutations change an amino acid at a position in the EP402R protein corresponding to S15, W19, Q96, N104, and/or K108D of Georgia 2007/1 EP402R protein (SEQ ID NO. 24). In an embodiment the one or more mutations change the amino acid at position S15, W19, Q96, N104, and/or K108D of the EP402R protein of the Georgia 2007/1 EP402R protein (SEQ ID NO. 24), or the corresponding position in the EP402R protein of any other ASFV strain. In an embodiment the one or more mutations change an amino acid at a position in the EP402R protein corresponding to W20, Q112, N121 and/or R125 of N10 Genotype IX EP402R protein (SEQ ID NO. 27). In an embodiment the one or more mutations change the amino acid at position S15, W19, Q96, N104, and/or K108D of the EP402R protein of the N10 Genotype IX EP402R protein (SEQ ID NO. 27), or the corresponding position in the EP402R protein of any other ASFV strain. These amino acids are in the ligand-binding domain of the EP402R protein and are surface exposed.

Suitably, the mutation is selected from N16R, 119R, W21D, Y 76D, E99R, Y102D and/or N108R at a position corresponding to the position in the Benin 97/1 EP402R protein (SEQ ID NO. 11). Suitably, the mutation is a combination of E99R and N108R at the positions corresponding to the positions in the Georgia 2007/1 EP402R protein (SEQ ID NO. 11).

Suitably, the mutation is selected from S15R, W19D, Q96R, N104R and/or K108D at a position corresponding to the position in the Georgia 2007/1 EP402R protein (SEQ ID NO. 24). Suitably, the mutation is a combination of Q96R and N104R at the positions corresponding to the positions in the Georgia 2007/1 EP402R protein (SEQ ID NO. 24).

Suitably, the mutation is selected from W20D, Q112R, N121R and/or R125D at a position corresponding to the position in the N10 Genotype IX EP402R protein (SEQ ID NO. 27). Suitably, the mutation is a combination of Q112R and N121R at the positions corresponding to the positions in the N10 Genotype IX EP402R protein (SEQ ID NO. 27).

Suitably, where the attenuated ASFV is genotype I, the one or more mutations change an amino acid at a position in the EP402R protein corresponding to Y76, E99, Y102, and/or N108 of Benin 97/1 EP402R protein (SEQ ID NO. 11). Suitably, where the attenuated ASFV is genotype I, the mutation is selected from Y76D, E99R, Y102D, and/or N108R at a position corresponding to the position in the Benin 97/1 EP402R protein (SEQ ID NO. 11). Suitably, where the attenuated ASFV is genotype II, the one or more mutations change an amino acid at a position in the EP402R protein corresponding to S15, W19, Q96, N104, and/or K108D of Georgia 2007/1 EP402R protein (SEQ ID NO. 24). Suitably, where the attenuated ASFV is genotype II, the mutation is selected from S15R, W19D, Q96R, N104R and/or K108D at a position corresponding to the position in the Georgia 2007/1 EP402R protein (SEQ ID NO. 24). Suitably, where the attenuated ASFV is genotype II, the mutation may be a combination of Q96R and N104R at the positions corresponding to the positions in the Georgia 2007/1 EP402R protein (SEQ ID NO. 24).

The invention also provides a polynucleotide encoding the EP402R protein of the invention. Suitably the polynucleotide comprises a sequence having at least 70% identity, such as at least 75%, 80%, 85%, 90%, 95%, 96%, 97%, 98% or 99% identity, with any of SEQ ID Nos 229 to 241 (i.e. the coding sequences of the EP402R genes from different strains of ASFV). Suitably the polynucleotide comprises a sequence having at least 70% identity, such as at least 75%, 80%, 85%, 90%, 95%, 96%, 97%, 98% or 99% identity, with SEQ ID No. 229 (i.e. the coding sequences of the Georgia 2007/1 EP402R gene).

The invention also provides a vector comprising the polynucleotide of the invention. Suitable vectors are known in the art and include plasmids for protein expression in cells such as bacteria, yeast or vertebrate cells, among or other cell types known in the art.

### ASF Virus Comprising Mutated EP402R

In another aspect the invention provides an ASF virus comprising the EP402R protein of the invention, such as described above. In a similar aspect the invention provides an ASF virus comprising the polynucleotide of the invention, such as described above. In another aspect the invention provides an attenuated ASF virus as described herein, wherein the virus comprises an EP402R gene mutated to express an EP402R protein of the invention, such as the EP402R protein described above.

The skilled person will appreciate from the present disclosure that an ASF virus can be mutated to express an EP402R protein comprising any of the amino acid changes described herein by appropriately modifying the coding sequence of the EP402R gene using molecular biology techniques known in the art. Thus the invention encompasses ASF viruses comprising any EP402R protein of the invention disclosed herein. Additionally, the skilled person will appreciate from the present disclosure that any EP402R protein described herein may be combined in an ASF virus of the invention with the other modifications to the ASF virus described herein, in particular modification of the K145R and EP153R genes. Furthermore, the ASFV of the invention comprising the EP402R protein with amino acid changes of the invention may be attenuated.

In an embodiment of the ASF virus of the invention, the EP402R gene comprises one or more mutations that change one or more amino acids in the ligand-binding domain of the EP402R protein. Suitably the EP402R gene comprises one or more mutations (such as two or more, three or more, four or more or five or more mutations) that change one or more amino acids (such as two or more, three or more, four or more or five or more amino acids) in the ligand-binding domain of the EP402R protein.

In embodiments of the ASF virus of the invention, the ASF virus comprises one or more mutations in the EP402R gene that change one or more amino acids in the EP402R protein in any of the ways described herein. For example, in an embodiment the one or more mutations in the EP402R gene change the amino acid at position Q96 and/or W99 of the EP402R protein of the Georgia 2007/1 strain, or the corresponding position in the EP402R protein of any other ASFV strain. In an embodiment the one or more mutations in the EP402R gene change the amino acid at position Q96 of the EP402R protein of the Georgia 2007/1 strain (SEQ ID No. 24) to R.

### Activity of EP402R

In an embodiment of the ASFV of the invention, EP402R activity is disrupted.

In an embodiment the activity of EP402R that is disrupted is its ability to mediate haemadsorption. In other words, the ability of EP402R to mediate haemadsorption may be decreased. The ability of EP402R to mediate haemadsorption may be decreased by at least 50, 60, 70, 80 or 90%. The ability of EP402R to mediate haemadsorption may be completely abolished.

The activity of EP402R that is disrupted may be the ability of the EP402R protein to bind ligands via its extracellular N-terminal Ig-like domain (i.e. its ligand-binding domain). Thus in an embodiment the invention provides an ASF virus wherein the EP402R gene comprises one or more mutations that disrupt ligand binding by the EP402R protein. The ability of the EP402R protein to bind one or more ligands may be disrupted. The ability of EP402R to bind one or more ligands may be decreased by at least 50, 60, 70, 80 or 90%. The ability of the EP402R protein to bind one or more ligands may be completely abolished. The ability of the EP402R protein to bind one or more ligands may be completely abolished whilst retaining the ability to bind other ligands. In an embodiment, the invention provides an attenuated ASF virus wherein the EP402R gene comprises one or more mutations that disrupt ligand binding by the EP402R protein. Ligand binding may be measured using assays such as immunoprecipitation, surface plasmon resonance and/or isothermal calorimetry.

In an embodiment the invention provides an ASF virus wherein the changed amino acids in the EP402R protein directly inhibit the interaction between EP402R and its ligand by changing the binding surface on EP402R, as described herein.

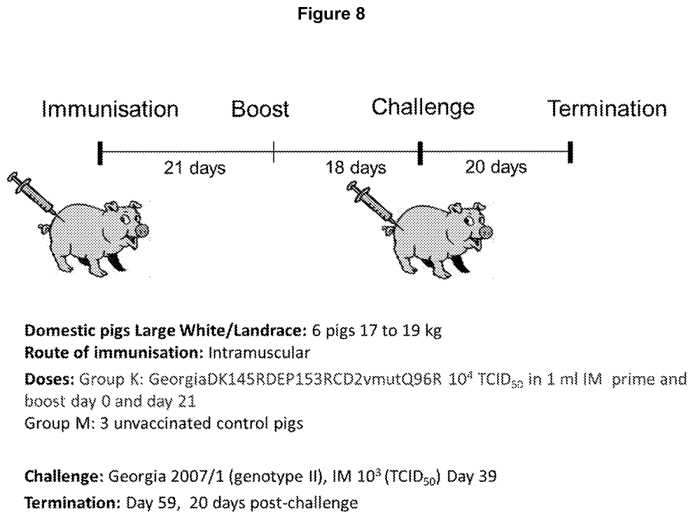

In an embodiment the invention provides an ASF virus wherein surface expression of the EP402R protein is reduced compared to a corresponding ASF virus in which the expression and/or activity of the EP402R gene is not disrupted. “Surface expression” of EP402R refers to expression of the EP402R protein on the surface of cells infected with ASF virus. Surface expression of EP402R may be measured by techniques known in the art, such as antibody staining of infected cells (see for example FIG. 8 and Example 3).

### EP153R

In certain aspects, the invention provides an ASF virus in which expression and/or activity of the EP153R gene has been disrupted.

The EP153R gene is expressed both early and late in infection. EP153R may also be referred to as 8CR. The EP153R protein is a C-type lectin. C-type animal lectins are found in serum, the extracellular matrix and cell membranes and are thought to act as receptors for carbohydrate ligands. The EP153R protein comprises a C-type lectin domain, a cell attachment sequence (RGD) and a transmembrane domain, and has similarity with CD44 molecules involved in T cell activation.

The gene (i.e. nucleotide) sequences and positions in the genome of EP153R genes from different ASFV strains are presented below.

Benin 97/1 EP153R (NC_044956.1: 67030-67491)
SEQ ID No. 204
ATGTATTTTAAGAAAAAATACATCGGTCTTATTGATAAGAACTGT
GAAAAAAAAATATTAGATGATTCTAGTACAATAAAAATTTGTTAC
ATATTAATTGGAATATTGATTGGAACTAATATGATAACTCTTATT
TATAATTTCATATTCTGGGATAATTATATAAAATGTTACCGAAAT
AATGATAAAATGTTTTACTGTCCTAATGATTGGGTTGGATATAAT
AATATTTGTTACTATTTTAGTAATGGTAGTTTTTCTAAAAATTAT
ACAGCTGCTAGTAATTTTTGTAGACAATTAAATGGTACACTTGCT
AATAATGATACTAATTTATTAAATCTAACTAAAATATATAATAAT
CAATCTATGTATTGGGTTAACAATACGGTAATATTACGTGGTGAT
AATAAATATAGTCAAAAAGTTAACTATACAGATTTATTATTTATT
TGTGGTAAATAA
Georgia 2007/1 EP153R (NC_044959.1: 73369-74451)
SEQ ID No. 205
ATGTTTTCTAACAAAAAGTACATCGGTCTTATCAATAAGAAGGAG
GGTTTGAAAAAAAAAATAGATGATTATAGTATATTAATAATTGGA
ATATTAATTGGAACTAACATCTTAAGCCTTATTATAAATATAATA
GGAGAGATTAATAAACCAATATGTTACCAAAATGATGATAAGATA
TTTTATTGCCCTAAAGATTGGGTTGGATATAATAATGTTTGTTAT
TATTTTGGCAATGAAGAAAAAAATTATAATAATGCAAGTAATTAT
TGTAAGCAATTAAATAGTACGCTTACTAATAATAATACTATTTTA
GTAAATCTTACTAAAACATTAAATCTTACTAAAACATATAATCAC
GAATCTAATTATTGGGTTAATTATTCTTTAATTAAAAATGAGTCA
GTACTATTACGTGATAGTGGATATTACAAAAAACAAAAACATGTA
AGTTTATTATATATTTGTAGTAAATAA
Ken05/Tk1 EP153R (NC_044945.1: 73544-74038)
SEQ ID No. 206
ATGTATTCTAAGAAAAAGTACATCGGTCTTATTGACAAGAACTGT
GAAAAAAAAATATTAGATGATGCTACTACAATAAAAATTTGTTAC
ATATTAATTGGAATATTGATTGGAACTAATATTATAACTCTTATC
TATAATTTCATATTTTGGGAGAATTATATAAAATGTATCCAAAAT
GATGATAAGATGTTTTACTGTCCTAAAGATTGGGTTGGGTATAAT
AATGCGTGTTATTATTTTAGTAATAACAATAAAAATTATACAGAT
GCAAATAATTATTGTAAAAATTCACATAATAGTACACTTGCTAAT
AATGATACTAAATTATTAAATCTTACTAAATTATTAAATCTTTCT
AAATTATATTACAATGATTCTACATATTGGGTTAAATATTCTTTA
CCTAAAAATAAAGCAGTAACATTACGTAACAGTACCTACAAATAT
GATAGAGTTAAATCTACAGAAACATTTTTTATTTGTAGTAACTAA
Ken06.Bus EP153R (NC_044946.1: 69244-69741)
SEQ ID No. 207
ATGTATTCTAAGAAAAAGTACATCGGTCTTATTGACAAAAGCTGT
GAAAAAAAAATATTAGATGATCCTACTACAATAAAAATTTGTTAC
ATATTAATTGGAATATTGATTGGAACTAATATTATAACTCTTATC
TATAATTTCATATTTTGGGAGAATTATATAAAATGTATCCGAAAT
GATGATAAGATGTTTTACTGCCCTAAAGATTGGGTTGGATATAAT
AATGTGTGTTATTATTTTAGTAACGATAATGGAAAAAATTATATG
AATGCAAGTAATTTTTGTAATAAAAAAAATAGCACGCTTGCTAAT
AATACGACTAATTTAGTAAATCTTACTAAAATATTAAATTTTACC
AAACAATATAATCATGAATCACTTTATTGGGTTAATTATTCTTTA
AGTAATAATATGTCATTAGCATTACAGAATAGTAATTTTCCTTCT
TCAGGAAGACGTAAATATATGGACTTATTATATATATGTAGTATA
TAA
L60 EP153R (NC_044941.1: 67379-67840)
SEQ ID No. 208
ATGTATTTTAAGAAAAAATACATCGGTCTTATTGATAAGAACTGT
GAAAAAAAAATATTAGATGATTCTAGTACAATAAAAATTTGTTAC
ATATTAATTGGAATATTGATTGGAACTAATATGATAACTCTTATT
TATAATTTCATATTCTGGGATAATTATATAAAATGTTACCGAAAT
AATGATAAAATGTTTTACTGTCCTAATGATTGGGTTGGATATAAT
AATATTTGTTACTATTTTAGTAATGGTAGTTTTTCTAAAAATTAT
ACAGCTGCTAGTAATTTTTGTAGACAATTAAATGGTACACTTGCT
AATAATGATACTAATTTATTAAATCTAACTAAAATATATAATAAT
CAATCTATGTATTGGGTTAACAATACGGTAATATTACGTGGTGAT
AATAAATATAGTCAAAAAGTTAACTATACAGATTTATTATTTATT
TGTGGTAAATAA
China/2018/AnhuiXCGQ EP153R
(MK128995.1: 72837-73313)
SEQ ID No. 209
ATGTTTTCTAACAAAAAGTACATCGGTCTTATCAATAAGAAGGAG
GGTTTGAAAAAAAAAATAGATGATTATAGTATATTAATAATTGGA
ATATTAATTGGAACTAACATCTTAAGCCTTATTATAAATATAATA
GGAGAGATTAATAAACCAATATGTTACCAAAATGATGATAAGATA
TTTTATTGCCCTAAAGATTGGGTTGGATATAATAATGTTTGTTAT
TATTTTGGCAATGAAGAAAAAAATTATAATAATGCAAGTAATTAT
TGTAAGCAATTAAATAGTACGCTTACTAATAATAATACTATTTTA
GTAAATCTTACTAAAACATTAAATCTTACTAAAACATATAATCAC
GAATCTAATTATTGGGTTAATTATTCTTTAATTAAAAATGAGTCA
GTACTATTACGTGATAGTGGATATTACAAAAAACAAAAACATGTA
AGTTTATTATATATTTGTAGTAAATAA
Malawi Lil-20/1 (1983) EP153R
(AY261361.1: 70776-71261)
SEQ ID No. 210
ATGTTTTCTAACAAAAAGTACATCGGTCTTATTGACAAGTACTGT
GAAAAAAAAATATTAGATGATTCTAGTACAATAAAAATTTGTTAC
ATATTAATTGGAATACTGATTGGAACTAATATGATAACTCTTATT
TATAATTTCATATTTTGGGAGAATTATATAACATGTAACCAAAAA
GATAAGACGTTTTACTGTCCTAAAGATTGGGTTGGATATAATAAT
GTTTGTTATTATTTTGGCAATGATGAAAAAAATTATAATAATGCA
AGTAATTATTGTAAGCAATTAAATAGTACGCTTACTAATAATAAT
ACTAATTTAGTAAATCTTACTAAAACATTAAATCTTACTAAAACA
TATAATCACGAATCTAATTATTGGGTTAATTATTCTTTAATTAAA
AATGAGTCAGTACTATTACGCAATAGTGGATATTATAAAAAACAA
AAACATGTAAGTTTATTATATATTTGTAGTAAATAA
Warthog EP153R (AY261366. 1: 69453-69944)
SEQ ID No. 211
ATGTTTTCTAATAAAAAGTACATTGGTCTTATCAATAAGAAGGAG
GGATTGAAAAAAAAAATAGATGATTATAGTATATTAATAATTGGA
ATATTAATTGGAACTAACATCTTAAGCCTTATTATAAATATAATA
GGAGAGATTAATAAACCAATATGTTACCAAAATAATGATAAGATA
TTTTATTGCCCTAAAGATTGGGTTGGATATAATAATGTTTGTTAT
TATTTTAGTAATGATAATGGCAATAATTATACAACTGCGGATAAT
AAATGTAAACAATTAAATAATAGTACACTGGCTAATAATCTTACT
GATTTATTAAATCTTACGAGTTTCTTAAATCTTACTAAATTATAT
CATCATCATTCTCATTATTGGGTTAATTATTCTTTAAATAATAAT
TATTCAGTACCATTAATTGATAGTAAATATAATTTAAATAGGAAA
AAAAGTCACTATACAGATTTACTATTTATTTGCAGCAAATAA
Mkuzi 1979 EP153R (AY261362.1: 74549-75013)
SEQ ID No. 212
ATGTATTTTAAGAAAAAATACATCGGTCTTATTGATAAGAACTGT
GAAAAAAAAATATTAGATGATTCTAGTACAATAAAAATTTGTTAC
ATATTAATTGGAATATTGATTGGAACTAATATGATAACTCTTATT
TATAATTTCATATTCTGGGATAATTATATAAAATGTTACCGAAAT
AATGATAAAATGTTTTACTGTCCTAATGATTGGGTTGGATATAAT
AATGTTTGTTACTATTTTAGTAATGGTAGTTTTTTTAAAAATTAT
ACAGATGCTAATAATTATTGTAAAAAAATAAATAGTACTCTTGCT
AATAATGATACTAATTTATTAAGTTTAACTAAAATATACAATAAT
CAATCTATGTATTGGGTTAACAATACAGTAGCATTACGTGGTGAT
AGTGTATATGCACAAAGAAAAGTTAATAATTCAGATCTATTATTT
ATTTGTAGTAAATAA
Warmbaths EP153R (AY261365.1: 72727-73191)
SEQ ID No. 213
ATGTATTTTAAGAAAAAGTACATCGGTCTTATTGATAAGAACTGT
GAAAAAAAAATATTAGATGATTGTACTACAATAAAAATTTGTTAC
ATATTAATTGGAATATTGATTGGAACTAATATGATAACTCTTATT
TATAATTTCATATTTTGGGATCATTATATGACATGTAACAAAAAA
GATAAGATGTTTTACTGTCCTAAAGATTGGGTTGGATATAATAAT
GTTTGTTATTATTTTAGTAATGATAGTTTTACTAAGAATTATACA
TATGCAAGTAATTCTTGTAAAAAAATAAATAGTACACTTGCTAAT
AATGATACTAATTTACTAAATCTTATTAAAGTATATAATAATGTA
TCTGAGTATTGGGTTAATAATACAGTAACATTACGTGGTGATCAT
AAATACATAAATAAGCAAAATAATTGTAATACAGAATTTTTATTT
ATTTGTAGTAGATAG
Tengani 62 EP153R (AY261364.1: 68134-68604)
SEQ ID No. 214
ATGTTTTCTAACAAAAAGTATCCGAGTCTTATCGAAAAAAAAATG
GATGACTTGATGACTTTAAAGTTTTGTTACATCATTATTGCAATC
TTGATCATGACTAATATTTTTAGCCTTGTTGTGAATATATGGGGG
GGGGGTGATCGACAATCTTATGAGAATGTATTTTATTGTCCTAAA
GATTGGGTTGGATATAATAATGCATGTTATTATTTTAGTAATGAT
ATGAAAAATTATACAGAAGCAAGTAATTATTGTAAAAATTTATAT
AATAGTACAATTGTTAATAATAATACTAATATAGTAAATCTTACT
AAAACATTAAATCTTACTAAAATATATAATCATGAAAGTAATTAT
TGGGTTAATTATTCTTTAATTAATAATGAGTCATTAACATTACGG
GACAGTAATTTTCCTTCTTCAGGAAAACGTACATATATAGATTTA
TTATATATATGTAGTATATAA
Pretorisuskop/96/4 EP153R
(AY261363.1: 73562-74029)
SEQ ID No. 215
ATGTATTTTAAGAAAAAGTACATCGGTCTTATTGATAAGAACTGT
GAAAAAAAAATATTAGATGATTGTACTACAATAAAAATTTGTTAC
ATATTAATTGGAATATTGATTGGAACTAATATGATAACTCTTATT
TATAATTTCATATTTTGGGATCATTATATGACATGTAACAAAAAA
GATAAGATGTTTTACTGTCCTAAAGATTGGGTTGGATATAATAAT
GTTTGTTATTATTTTAATAATGATAGTAAGAATTATACAACTGCT
ACTAATTCTTGTAAACAATTAAATAGTACACTTGCTAATAATGAT
ACTAATTTATTAAATCTTACTAAAGTATATCACCATGATAAATTA
TATTGGGTTAACTATTCTTTAAATGACAATTTTTCATTATCATTA
CGTAATAGTACATATGAAAAAAGATCTAAATATTTGCCATTATTA
TTTATTTGCAGTAAATAA
Kenya 1950 EP153R (AY261360.1: 76255-76731)
SEQ ID No. 216
ATGTTTTTGAACAAAAAGTATCCGAGTCTTATCGAAAAAAAAATG
GATGATTTGATGACTTTAAAGTTTTGTTACTTAATAATCACATTT
TTAATCATAACCAATATTTTTAGCCTTGCTATAAATATATGGGGG
GGGGGTGATATGATAGATCGACAAAGTTGTGAAAATATATTTTAC
TGTCCTAAAGATTGGGTTGGCTATAATAATGCGTGTTATTATTTT
AGTAATAACAATAAAAATTATACAGATGCAAATAATTATTGTAAA
AATTCACATAATAGTACACTTGCTAATAATGATACTAAATTATTA
AATCTTACTAAATTATTAAATCTTTCTAAATTATATTACAATGAT
TCTACATATTGGGTTAAATATTCTTTACCTAAAAATAAAGCAGTA
ACATTACGTAACAGTACCTACAAATATGATAGAGTTAAATATACA
GAAACATTTTTTATTTGTAGTAACTAA

In an embodiment the invention provides an ASF virus in which the expression and/or activity of the EP153R gene is disrupted. Suitably, the EP153R gene comprises the sequence of SEQ ID No. 204, 205, 206, 207, 208, 209, 210, 211, 212, 213, 214, 215 or 216. Suitably, the EP153R gene comprises a sequence having at least 70%, at least 80%, at least 90% or at least 95% identity with SEQ ID No. 204, 205, 206, 207, 208, 209, 210, 211, 212, 213, 214, 215 or 216. Suitably, the EP153R gene consists of the sequence of SEQ ID No. 204, 205, 206, 207, 208, 209, 210, 211, 212, 213, 214, 215 or 216.

In an embodiment of the ASFV of the invention the EP153R gene may be partially or completely deleted. Suitably part or all of the sequence of SEQ ID No. 204, 205, 206, 207, 208, 209, 210, 211, 212, 213, 214, 215 or 216 is removed from the ASFV genome. Suitably the ASFV genome lacks any of these sequences.

The accession numbers of EP153R proteins from different strains of ASFV are listed below in Table 3.

TABLE 3

| Strain | EP153R protein accession number |
|---|---|
| BA71V | Q65150 |
| Benin 97/1 | A9L06 |
| E75 | D415P5 |
| Georgia 2007/1 | E0WMJ5 |
| Kenya | P0CA63 |
| Malawi | P0CA64 |
| Portugal/OURT88/1988 | A9JLS3 |
| PretorisuskopPr4/1996 | O89335 |
| Warthog | P0CA65 |

The amino acid sequences of the EP153R proteins from different ASFV strains are depicted below.

Benin 97/1 EP153R protein
SEQ ID No. 20
MYFKKKYIGLIDKNCEKKILDDSSTIKICYILIGILIGTNMITLI
YNFIFWDNYIKCYRNNDKMFYCPNDWVGYNNICYYFSNGSFSKNY
TAASNFCRQLNGTLANNDTNLLNLTKIYNNQSMYWVNNTVILRGD
NKYSQKVNYTDLLFICGK
Georgia 2007/1 EP153R protein
SEQ ID No. 217
MFSNKKYIGLINKKEGLKKKIDDYSILIIGILIGTNILSLIINII
GEINKPICYQNDDKIFYCPKDWVGYNNVCYYFGNEEKNYNNASNY
CKQLNSTLTNNNTILVNLTKTLNLTKTYNHESNYWVNYSLIKNES
VLLRDSGYYKKQKHVSLLYICSK*
Ken05/Tk1 EP153R protein
SEQ ID No. 218
MYSKKKYIGLIDKNCEKKILDDATTIKICYILIGILIGTNIITLI
YNFIFWENYIKCIQNDDKMFYCPKDWVGYNNACYYFSNNNKNYTD
ANNYCKNSHNSTLANNDTKLLNLTKLLNLSKLYYNDSTYWVKYSL
PKNKAVTLRNSTYKYDRVKSTETFFICSN*
Ken06.Bus EP153R protein
SEQ ID No. 219
MYSKKKYIGLIDKSCEKKILDDPTTIKICYILIGILIGTNIITLI
YNFIFWENYIKCIRNDDKMFYCPKDWVGYNNVCYYFSNDNGKNYM
NASNFCNKKNSTLANNTTNLVNLTKILNFTKQYNHESLYWVNYSL
SNNMSLALQNSNFPSSGRRKYMDLLYICSI*
L60 EP153R protein
SEQ ID No. 220
MYFKKKYIGLIDKNCEKKILDDSSTIKICYILIGILIGTNMITLI
YNFIFWDNYIKCYRNNDKMFYCPNDWVGYNNICYYFSNGSFSKNY
TAASNFCRQLNGTLANNDTNLLNLTKIYNNQSMYWVNNTVILRGD
NKYSQKVNYTDLLFICGK*
China/2018/AnhuiXCGQ EP153R protein
SEQ ID No. 221
MFSNKKYIGLINKKEGLKKKIDDYSILIIGILIGTNILSLIINII
GEINKPICYQNDDKIFYCPKDWVGYNNVCYYFGNEEKNYNNASNY
CKQLNSTLTNNNTILVNLTKTLNLTKTYNHESNYWVNYSLIKNES
VLLRDSGYYKKQKHVSLLYICSK*
Malawi Lil-20/1 (1983) EP153R protein
SEQ ID No. 222
MFSNKKYIGLIDKYCEKKILDDSSTIKICYILIGILIGTNMITLI
YNFIFWENYITCNQKDKTFYCPKDWVGYNNVCYYFGNDEKNYNNA
SNYCKQLNSTLTNNNTNLVNLTKTLNLTKTYNHESNYWVNYSLIK
NESVLLRNSGYYKKQKHVSLLYICSK*
Warthog EP153R protein
SEQ ID No. 223
MFSNKKYIGLINKKEGLKKKIDDYSILIIGILIGTNILSLIINII
GEINKPICYQNNDKIFYCPKDWVGYNNVCYYFSNDNGNNYTTADN
KCKQLNNSTLANNLTDLLNLTSFLNLTKLYHHHSHYWVNYSLNNN
YSVPLIDSKYNLNRKKSHYTDLLFICSK*
Mkuzi 1979 EP153R protein
SEQ ID No. 224
MYFKKKYIGLIDKNCEKKILDDSSTIKICYILIGILIGTNMITLI
YNFIFWDNYIKCYRNNDKMFYCPNDWVGYNNVCYYFSNGSFFKNY
TDANNYCKKINSTLANNDTNLLSLTKIYNNQSMYWVNNTVALRGD
SVYAQRKVNNSDLLFICSK*
Warmbaths EP153R protein
SEQ ID No. 225
MYFKKKYIGLIDKNCEKKILDDCTTIKICYILIGILIGTNMITLI
YNFIFWDHYMTCNKKDKMFYCPKDWVGYNNVCYYFSNDSFTKNYT
YASNSCKKINSTLANNDTNLLNLIKVYNNVSEYWVNNTVTLRGDH
KYINKQNNCNTEFLFICSR*
Tengani 62 EP153R protein
SEQ ID No. 226
MFSNKKYPSLIEKKMDDLMTLKFCYIIIAILIMTNIFSLVVNIWG
GGDRQSYENVFYCPKDWVGYNNACYYFSNDMKNYTEASNYCKNLY
NSTIVNNNTNIVNLTKTLNLTKIYNHESNYWVNYSLINNESLTLR
DSNFPSSGKRTYIDLLYICSI*
Pretorisuskop/96/4 EP153R protein
SEQ ID No. 227
MYFKKKYIGLIDKNCEKKILDDCTTIKICYILIGILIGTNMITLI
YNFIFWDHYMTCNKKDKMFYCPKDWVGYNNVCYYENNDSKNYTTA
TNSCKQLNSTLANNDTNLLNLTKVYHHDKLYWVNYSLNDNFSLSL
RNSTYEKRSKYLPLLFICSK*
Kenya 1950 EP153R protein
SEQ ID No. 228
MFLNKKYPSLIEKKMDDLMTLKFCYLIITFLIITNIFSLAINIWG
GGDMIDRQSCENIFYCPKDWVGYNNACYYFSNNNKNYTDANNYCK
NSHNSTLANNDTKLLNLTKLLNLSKLYYNDSTYWVKYSLPKNKAV
TLRNSTYKYDRVKYTETFFICSN*

Suitably, the EP153R gene encodes a protein comprising the sequence of SEQ ID No. 20, 217, 218, 219, 220, 221, 222, 223, 224, 225 226, 227 or 228. Suitably, the EP153R gene encodes a protein comprising a sequence having at least 70%, at least 80%, at least 90% or at least 95% identity with SEQ ID No. 20, 217, 218, 219, 220, 221, 222, 223, 224, 225 226, 227 or 228. Suitably, the EP153R gene encodes a protein consisting of the sequence of SEQ ID No. 20, 217, 218, 219, 220, 221, 222, 223, 224, 225 226, 227 or 228.

In an embodiment the ASFV of the invention does not express EP153R protein. In other words, the ASFV of the invention does not express any proteins with sequences of SEQ ID Nos 20, 217, 218, 219, 220, 221, 222, 223, 224, 225 226, 227 or 228.

EP153R is immunogenic (i.e. evokes an immune response) (Burmakina et al. 2019 J. Gen. Virol. 100: 259-265). EP153R inhibits capsase-3 activation during ASFV infection and thereby has an inhibitory effect on apoptosis. EP153R is required for haemadsorption. EP153R also inhibits MHC-I expression in infected cells.

### Activity of EP153R

In an embodiment of the attenuated ASFV of the invention, EP153R activity may be disrupted.

In an embodiment the activity of EP153R that may be disrupted is its ability to mediate haemadsorption. In other words, the ability of EP153R to mediate haemadsorption may be decreased. The ability of EP153R to mediate haemadsorption may be decreased by at least 50, 60, 70, 80 or 90%. The ability of EP153R to mediate haemadsorption may be completely abolished.

In an embodiment the activity of EP153R that may be disrupted is its ability to inhibit caspase-3. In other words, the ability of EP153R to inhibit caspase-3 may be decreased. The ability of EP153R to inhibit caspase-3 may be decreased by at least 50, 60, 70, 80 or 90%. The ability of EP153R to inhibit caspase-3 may be completely abolished. Caspase-3 activity may be measured by assays known in the art, such as described by Hurtado et al. (Hurtado et al. 2004 Virology 326: 160-170). For example, the cleaved active caspase-3 fragment of 17 kDa and the inactive procaspase-3 protein of 34 kDa may be quantified using Western blot or mass spectrometry and compared to ascertain the degree of activation of caspase-3. Alternatively or additionally, the ability of caspase-3 in cell extract to cleave a fluorescent substrate may be measured using high performance liquid chromatography.

In an embodiment the activity of EP153R that may be disrupted is its ability to inhibit MHC-I expression. In other words, the ability of EP153R to inhibit MHC-I expression may be decreased. The ability of EP153R to inhibit MHC-I expression may be decreased by at least 50, 60, 70, 80 or 90%. The ability of EP153R to inhibit MHC-I expression may be completely abolished. MHC-I expression may be measured by assays known in the art, such as described by Hurtado et al. (Hurtado et al. 2011 Arch. Virol. 156(2): 219-234). For example, cell surface expression of MHC-I may be measured using antibody staining of non-permeabilised cells followed by flow cytometry.

### DIVA Mutations

In an embodiment the ASFV of the invention comprises a Differentiation of Infected from Vaccinated Animals (DIVA) mutation. A DIVA mutation is a mutation in the ASF virus that enables a vaccine comprising the ASF virus to function as a DIVA vaccine (i.e. subjects vaccinated with the DIVA vaccine can be differentiated from subjects infected with a wild-type ASF virus).

In an embodiment the invention provides an attenuated ASF virus in which the expression and/or activity of the genes EP153R and EP402R is disrupted;

which comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and MGF 505 1R, 2R and 6R;
and which comprises a DIVA mutation.

In an embodiment the invention provides an attenuated ASF virus in which the expression and/or activity of the genes EP153R and EP402R is disrupted;

and which comprises a functional version of one or more of the following genes:
multigene family (MGF) 110 11L and 12L,
MGF 360 6L, 10L, 11L, 12L, 13L, and 14L, and
MGF 505 1R and 2R;
and which comprises a DIVA mutation.

In an embodiment the invention provides an attenuated ASF virus in which the expression and/or activity of the genes EP153R and EP402R is disrupted;

and which comprises a functional version of one or more of the following genes:
multigene family (MGF) 110 5L, 6L, 8L and 12L,
MGF 360 6L, 10L, 11L, 12L, 13L, 14L and 21R, and
MGF 505 1R and 2R;
and which comprises a DIVA mutation.

In an embodiment the invention provides an ASF virus comprising an EP402R protein comprising one or more amino acid change in the ligand-binding domain wherein the amino acid change disrupts ligand-binding of the EP402R protein (i.e. an EP402 protein of the invention) and/or a polynucleotide encoding said EP402R protein, and further comprising a DIVA mutation.

In an embodiment the DIVA mutation disrupts expression of the K145R gene and/or the B125R gene. Suitably the DIVA mutation disrupts expression of the K145R gene. Suitably the K145R gene is partially deleted. Suitably the K145R gene is completely deleted. Suitably the B125R gene is partially deleted. Suitably the B125R gene is completely deleted.

### K145R

The K145R gene is a late gene.

The gene (i.e. nucleotide) sequences of K145R genes from different ASFV strains are given below.

Benin 97/1 K145R (NC_044956.1: 58961-59398)
SEQ ID No. 327
ATGGATCATTATCTTAAAAAATTACAAGATATTTATACGAAGCTC
GAGGGCCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCCATTGGGTTTATTAACTATATTAAAACGAGTAAACAAGAGTAT
TTATGCCTGTTGATTAATCCTAAACTCGTTACTAAGTTTTTAAAA
ATAACGAGCTTTAAAATTTACATTAATTTCAGACTGAAAACTTTT
TATATAAGTCCTAATAAGTATAATAATTTTTACACTGCTCCCTCT
GAAGAAAAGACTAACCATCTCCTAAAAGAAGAAAAAACTTGGGCA
AAGATTGTTGAAGAAGGAGGAGAAGAATCCTAA
China/2018/AnhuiXCGQ K145R
(MK128995.1: 64748-65185)
SEQ ID No. 328
ATGGATCATTATCTTAAAAAATTACAAGATATTTATACGAAGCTC
GAGGGTCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCCATTGGGTTTATTAACTATATTAAAACGAGTAAACAAGAGTAT
TTATGCCTGTTAATTAATCCTAAACTCGTTACTAAGTTTTTAAAA
ATAACGAGCTTTAAAATTTACATTAATTTCAGGCTGAAAACTTTT
TATATAAGTCCTAATAAGTATAATAATTTTTACACCGCTCCCTCT
GAAGAAAAGACTAACCATCTTCTAAAAGAAGAAAAAACTTGGGCA
AAGATTGTTGAAGAAGGAGGAGAAGAATCCTAA
Georgia 2007/1 K145R
(NC_044959.1: 64734-65171)
SEQ ID No. 329
ATGGATCATTATCTTAAAAAATTACAAGATATTTATACGAAGCTC
GAGGGTCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCCATTGGGTTTATTAACTATATTAAAACGAGTAAACAAGAGTAT
TTATGCCTGTTAATTAATCCTAAACTCGTTACTAAGTTTTTAAAA
ATAACGAGCTTTAAAATTTACATTAATTTCAGGCTGAAAACTTTT
TATATAAGTCCTAATAAGTATAATAATTTTTACACCGCTCCCTCT
GAAGAAAAGACTAACCATCTTCTAAAAGAAGAAAAAACTTGGGCA
AAGATTGTTGAAGAAGGAGGAGAAGAATCCTAA
Ken05/Tk1 K145R (NC_044945.1: 65442-65879)
SEQ ID No. 330
ATGGATCATTATCTTAAAAAATTACAGGATATTTATAAGAAGCTT
GAGGGTCACCCCTTTCTTTTTAGTCCGTCGAAAACCAATGAAAAA
GAGTTTATTACTCTGCTAAACCAAGCCTTGGCCTCGACGCAGCTT
TACCGCAGCATACAACAGCTGTTTTTAACGATGTATAAGCTAGAT
CCCATTGGGTTTGTTAACTATATTAAAGCGAGTAAACAAGAGTAT
TTATGTCTGTTGATTAATCCTAAACTAGTCACTAAGTTTTTAAAA
ATAACGAGCTTTAAAATTTACATTAATTTTAGGCTGAAAACTTTT
TATATAAGTCCTAATAAGTATAATAATTTTTACATCGCTCCCTCT
GAAGAAAAGGCCAATCATCTCCTAAAAGAAGAAAAAACCTGGGCA
AAGATTGTTGAAGAAGGAGGAGAAGAATCCTAA
Ken06. Bus K145R (NC_044946.1: 61226-61663)
SEQ ID No. 331
ATGGATCATTATCTTAAAAAATTACAGGATATTTATAAGAAGCTT
GAGGGTCACCCCTTTCTTTTTAGTCCGTCGAAAACCAATGAAAAA
GAGTTTATTACTCTGCTAAACCAAGCCTTGGCCTCGACGCAGCTT
TACCGCAGCATACAACAGCTGTTTTTAACGATGTATAAGCTAGAT
CCCATTGGGTTTGTTAACTATATTAAAGCGAGTAAACAAGAGTAT
TTATGTCTGTTGATTAATCCTAAACTAGTCACTAAGTTTTTAAAA
ATAACGAGCTTTAAAATTTACATTAATTTCAGGCTAAAAACTTTC
TATATAAGTCCTAATAAGTATAATAATTTTTACATCGCTCCCTCT
GAAGAAAAGGCCAATCATCTCCTAAAAGAAGAAAAAACCTGGGCA
AAGATTGTTGAAGAAGGAGGAGAAGAATCCTAA
Kenya 1950 K145R (AY261360.1: 68066-68503)
SEQ ID No. 332
ATGGATCATTATCTTAAAAAATTACAGGATATTTATAAGAAGCTT
GAGGGTCACCCCTTTCTTTTTAGTCCGTCGAAAACCAATGAAAAA
GAGTTTATTACTCTGCTAAACCAAGCCTTGGCCTCGACGCAGCTT
TACCGCAGCATACAACAGCTGTTTTTAACGATGTATAAGCTAGAT
CCCATTGGGTTTGTTAACTATATTAAAGCGAGTAAACAAGAGTAT
TTATGTCTGTTGATTAATCCTAAACTAGTCACTAAGTTTTTAAAA
ATAACGAGCTTTAAAATTTACATTAATTTTAGGCTGAAAACTTTT
TATATAAGTCCTAATAAGTATAATAATTTTTACATCGCTCCCTCT
GAAGAAAAGGCCAATCATCTCCTAAAAGAAGAAAAAACCTGGGCA
AAGATTGTTGAAGAAGGAGGAGAAGAATCCTAA
L60 K145R (NC_044941.1: 59310-59747)
SEQ ID No. 333
ATGGATCATTATCTTAAAAAATTACAAGATATTTATACGAAGCTC
GAGGGCCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCCATTGGGTTTATTAACTATATTAAAACGAGTAAACAAGAGTAT
TTATGCCTGTTGATTAATCCTAAACTCGTTACTAAGTTTTTAAAA
ATAACGAGCTTTAAAATTTACATTAATTTCAGACTGAAAACTTTT
TATATAAGTCCTAATAAGTATAATAATTTTTACACTGCTCCCTCT
GAAGAAAAGACTAACCATCTCCTAAAAGAAGAAAAAACTTGGGCA
AAGATTGTTGAAGAAGGAGGAGAAGAATCCTAA
Malawi Lil-20/1 (1983) K145R
(AY261361.1: 62661-63098)
SEQ ID No. 334
ATGGATCATTATCTTAAAAAATTAGAGGATATTTATAAAAAGCTT
GAGGGTCATCCCTTTCTTTTTAGTCCGTCGAAAACCAATGAAAAA
GAGTTTATTACTCTGTTAAACCAAGCCTTGGCCTCAACGCAGCTT
TATCGCAGCATACAACAGCTGTTTTTAACGATGTATAAGCTAGAT
CCCATTGGGTTTATTAACTATATTAAAACGAGTAAACAAGAGTAT
TTATGCCTGTTGATCAATCCTAAACTAGTTACGAAGTTTTTAAAA
ATAACGAGTTTTAAAATTTACATTAATTTCAGGCTGAAAACTTTT
TATATAAGTCCTAATAAATATAATAATTTTTACACCGCTCCCTCT
GAAGAAAAGGCCAATCATCTCCTAAAAGAAGAAAAAACCTGGGCA
AAGATTGTTGAAGAAGGAGGAGAAGAATCCTAA
Mkuzi 1979 K145R (AY261362.1: 66481-66921)
SEQ ID No. 335
ATGGATCATTATCTTAAAAAATTACAAGATATTTATACGAAGCTC
GAGGGCCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCTATAGGGTTTATTAACTATATTAAAACGAGTAAACAAGAGTAT
TTATGCCTGTTGATTAATCCTAAACTCGTTACTAAGTTTTTAAAA
ATAACGAGCTTTAAAATTTACATTAATTTCAGACTGAAAACTTTT
TATATAAGTCCTAATAAGTATAATAATTTTTACACCGCTCCCTCT
GAAGAAAAGACCAACCATCTCCTAAAAGAAGAAAAAACTTGGGCA
AAGATTGTTGAAGAAGGAGGAGGAGAAGAATCCTAA
Pretorisuskop/96/4 K145R
(AY261363.1: 65440-65877)
SEQ ID No. 336
ATGGATCATTATCTTAAAAAATTACAAGAAATTTATACGAAGCTC
GAGGGCCATCCCTTTCTTTTTAGCCCGTCGAAAACCAATGAAAAA
GAGTTTATTACTCTGCTAAACCAGGCCTTGGCCTCAACGCAGCTT
TATCGCAGCATACAACAGCTGTTTTTAACGATGTATAAGCTAGAT
CCCATTGGGTTTATTAACTATATTAAAACGAGTAAACAAGAGTAT
TTATGCCTGTTGATTAATCCTAAACTCGTCACTAAGTTTTTAAAA
ATAACGAGCTTTAAAATTTACATTAATTTCAGACTGAAAACTTTT
TATATAAGTCCTAATAAGTATAATAATTTTTACACCGCTCCCTCT
GAAGAAAAGACCAACCATCTCCTAAAAGAAGAAAAAACTTGGGCA
AAGATTGTTGAAGAAGGAGGAGAAGAATCCTAA
Tengani 62 K145R (AY261364.1: 60111-60548)
SEQ ID No. 337
ATGGATCATTATCTTAAAAAATTACAAGATATTTATACGAAGCTC
GAGGGTCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCCATTGGGTTTATTAACTATATTAAAACGAGTAAACAAGAGTAT
TTATGCCTGTTGATTAATCCTAAACTCGTTACTAAGTTTTTAAAA
ATAACGAGCTTTAAAATTTACATTAATTTCAGACTGAAAACTTTT
TATATAAGTCCTAATAAGTATAATAATTTTTACACCGCTCCCTCT
GAAGAAAAGACTAACCATCTCCTAAAAGAAGAAAAAACTTGGGCA
AAGATTGTTGAAGAAGGAGGAGAAGAATCCTAA
Warmbaths K145R (AY261365.1: 64642-65082)
SEQ ID No. 338
ATGGATCATTATCTTAAAAAATTACAAGATATTTATACGAAGCTC
GAGGGCCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCTATAGGGTTTATTAACTATATTAAAACGAGTAAACAAGAGTAT
TTATGCCTGTTGATTAATCCTAAACTCGTTACTAAGTTTTTAAAA
ATAACGAGCTTTAAAATTTACATTAATTTCAGACTGAAAACTTTT
TATATAAGTCCTAATAAGTATAATAATTTTTACACCGCTCCCTCT
GAAGAAAAGACCAACCATCTCCTAAAAGAAGAAAAAACTTGGGCA
AAGATTGTTGAAGAAGGAGGAGGAGAAGAATCCTAA
Warthog K145R (AY261366.1: 61399-61836)
SEQ ID No. 339
ATGGATCATTATCTTAAAAAATTACAAGATATTTATACGAAGCTC
GAGGGCCATCCCTTTCTTTTTAGCCCGTCGAAAACCAATGAAAAA
GAGTTTATTACTCTGCTAAACCAGGCCTTGGCCTCAACGCAGCTT
TACCGCAGCATACAACAGCTGTTTTTAACGATGTATAAGCTAGAT
CCCATTGGGTTTATTAACTATATTAAAACGAGTAAACAAGAGTAT
TTATGCCTGTTGATTAATCCTAAACTCGTCACTAAGTTTTTAAAA
ATAACGAGCTTTAAAATTTACATTAATTTCAGACTGAAAACTTTT
TATATAAGTCCTAATAAGTATAATAATTTTTACACCGCTCCCTCT
GAAGAAAAGACCAACCATCTCCTAAAAGAAGAAAAAACTTGGGCA
AAGATTGTTGAAGAAGGAGGAGAAGAATCCTAA

In an embodiment an ASFV of the invention comprises a DIVA mutation that disrupts expression of the K145R gene. In other words, the ASFV lacks a functional version of the K145R gene. Suitably the K145R gene comprises the sequence of SEQ ID No. 327, 328, 329, 330, 331, 332, 333, 334, 335, 336, 337, 338 or 339. Suitably the K145R gene comprises a sequence having at least 70%, at least 80%, at least 90% or at least 95% identity with SEQ ID No. 327, 328, 329, 330, 331, 332, 333, 334, 335, 336, 337, 338 or 339. Suitably the K145R gene consists of the sequence of SEQ ID No. 327, 328, 329, 330, 331, 332, 333, 334, 335, 336, 337, 338 or 339.

In an embodiment of the ASFV of the invention the K145R gene may be partially or completely deleted. Suitably part or all of the sequence of SEQ ID No. 327, 328, 329, 330, 331, 332, 333, 334, 335, 336, 337, 338 or 339 is removed from the ASFV genome. Suitably the ASFV genome lacks any of these sequences.

The accessions numbers of K145R proteins from different strains of ASFV are listed below in Table 4.

TABLE 4

| Strain | K145R protein accession number |
|---|---|
| BA71V | Q07385 |
| PretoriuskopPr4/1996 | P0CA50 |
| Warthog | P0CA51 |
| Malawi-Lil/20 | P0CA49 |
| Kenya 1950 | P0CA48 |
| Georgia 2007/1 | E0WM19 |
| Portugal/OURT88/1988 | A9JLR1 |
| E75 | D4I5N9 |
| Benin 97/1 | A9JKZ8 |

The amino acid sequence of K145R proteins from different ASFV strain are depicted below.

Benin 97/1 K145R protein
SEQ ID No. 340
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGEES*
China/2018/AnhuiXCGQ protein
SEQ ID No. 341
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGEES*
Georgia 2007/1 K145R protein
SEQ ID No. 342
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGEES
Ken05/Tk1 K145R protein
SEQ ID No. 343
MDHYLKKLQDIYKKLEGHPFLFSPSKTNEKEFITLLNQALASTQL
YRSIQQLFLTMYKLDPIGFVNYIKASKQEYLCLLINPKLVTKFLK
ITSFKIYINFRLKTFYISPNKYNNFYIAPSEEKANHLLKEEKTWA
KIVEEGGEES*
Ken06. Bus K145R protein
SEQ ID No. 344
MDHYLKKLQDIYKKLEGHPFLFSPSKTNEKEFITLLNQALASTQL
YRSIQQLFLTMYKLDPIGFVNYIKASKQEYLCLLINPKLVTKFLK
ITSFKIYINFRLKTFYISPNKYNNFYIAPSEEKANHLLKEEKTWA
KIVEEGGEES*
Kenya 1950 K145R protein
SEQ ID No. 345
MDHYLKKLQDIYKKLEGHPFLFSPSKTNEKEFITLLNQALASTQL
YRSIQQLFLTMYKLDPIGFVNYIKASKQEYLCLLINPKLVTKFLK
ITSFKIYINFRLKTFYISPNKYNNFYIAPSEEKANHLLKEEKTWA
KIVEEGGEES*
L60 K145R protein
SEQ ID No. 346
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGEES*
Malawi Lil-20/1 (1983) K145R protein
SEQ ID No. 347
MDHYLKKLEDIYKKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKANHLLKEEKTWA
KIVEEGGEES*
Mkuzi 1979 K145R protein
SEQ ID No. 348
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGGEES*
Pretorisuskop/96/4 K145R protein
SEQ ID No. 349
MDHYLKKLQEIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGEES*
Tengani 62 K145R protein
SEQ ID No. 350
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGEES*
Warmbaths K145R protein
SEQ ID No. 351
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGGEES*
Warthog K145R protein
SEQ ID No. 352
MDHYLKKLQDIYTKLEGHPFLFSPSKTNEKEFITLLNQALASTQL
YRSIQQLFLTMYKLDPIGFINYIKTSKQEYLCLLINPKLVTKFLK
ITSFKIYINFRLKTFYISPNKYNNFYTAPSEEKTNHLLKEEKTWA
KIVEEGGEES*

In an embodiment the ASFV of the invention does not express K145R protein. In other words, the ASFV of the invention does not express any proteins with sequences of SEQ ID Nos 340, 341, 342, 343, 344, 345, 346, 347, 348, 349, 350, 351 or 352.

K145R inhibits the host endoplasmic reticulum (ER) stress response (Barber 2015 Stress modulators encoded by African swine fever virus; PHD thesis, St Georges, University of London, 2016). This response is caused by the accumulation of unfolded proteins and may be activated during viral infections due to the substantial amounts of viral proteins being produced. ER stress leads to the increase in expression of the transcription factor CCAAT-enhancer-binding protein homologous protein (CHOP) and its accumulation in the nucleus of the cells. CHOP activity ultimately results in cell apoptosis, thus limiting viral replication.

K145R function may be tested by methods including immunofluorescence using an antibody against CHOP and assessment of its presence in the nucleus of cells following induction of ER stress, and luciferase reporter assay, where the luciferase gene is under control of the CHOP promoter.

### B125R

The gene (i.e. nucleotide) sequences of B125R genes from different ASFV strains are given below.

Benin 97/1 B125R (NC_044956.1:100206-100583)
SEQ ID No. 353
ATGGCGGTTTATGCGAAGGATCTTGATAATAACAAAGAGTTAAACCAAAAATTAATTAACGATCAGCTTAAAATTATTGACAC
GCTCTTGCTAGCAGAAAAAAAAAACTTTTTGGTGTATGAATTGCCTGCCCCTTTTGACTTTTCCTCCGGCGACCCTTTGGCCA
GTCAGCGCGACATATACTATGCCATCATAAAAAGCCTCGAGGAGCGCGGGTTTACTGTCAAAATATGTATGAAAGGGGATCGT
GCCCTCCTTTTCATCACTTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA
China/2018/AnhuiXCGQ B125R (MK128995.1:105587-105964)
SEQ ID No. 354
ATGGCGGTTTATGCGAAGGATCTTGATAATAACAAAGAGTTAAACCAAAAATTAATTAACGATCAGCTTAAAATTATTGACAC
GCTCTTGCTGGCAGAAAAAAAAAACTTTTTGGTGTATGAACTACCTGCCCCTTTTGACTTTTCCTCCGGCGACCCTTTGGCCA
GTCAGCGCGACATATACTATGCCATCATAAAAAGCCTCGAGGAGCGCGGGTTTACTGTCAAAATATGTATGAAAGGGGATCGT
GCCCTCCTTTTCATCACCTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA
Georgia 2007/1 B125R (NC_044959.1:105570-105947)
SEQ ID No. 355
ATGGCGGTTTATGCGAAGGATCTTGATAATAACAAAGAGTTAAACCAAAAATTAATTAACGATCAGCTTAAAATTATTGACAC
GCTCTTGCTGGCAGAAAAAAAAAACTTTTTGGTGTATGAACTACCTGCCCCTTTTGACTTTTCCTCCGGCGACCCTTTGGCCA
GTCAGCGCGACATATACTATGCCATCATAAAAAGCCTCGAGGAGCGCGGGTTTACTGTCAAAATATGTATGAAAGGGGATCGT
GCCCTCCTTTTCATCACCTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA
Ken05/Tk1 B125R (NC_044945.1:106516-106893)
SEQ ID No. 356
ATGGCGGTTTATGCGAAGGACCTTGATAATAACAAAGAGTTAAACCAAAAATTGATCAACGATCAGCTTAAAATCATTGACAC
GCTTTTGCTAGCAGAAAAAAAAAACTTTTTGGTGTACGAACTACCTGCCCATTTTGACTTTTCCTCCGGCGACCCTTTGGGCA
GTCAGCGCGACATTTACTATGCCATCATAAAAAGTCTCGAGGAGCGCGGATTTACTGTCAAAATATGTATGAAGGGAGACCGC
GCCCTTCTTTTCATCACCTGGAAAAAAATACAATCCATTGAGATCAACAAAAAAGAAGAATATCTGCGCATGCACTTCATACA
AGACGAAGAGAAAGCGTTTTATTGTAAATTTTTAGAGTCTAGATGA
Ken06.Bus B125R (NC_044946.1:102432-102809)
SEQ ID No. 357
ATGGCGGTTTATGCGAAGGACCTTGATAATAACAAAGAGTTAAACCAAAAATTGATCAACGATCAGCTTAAAATTATTGACAC
GCTTTTGCTGGCAGAAAAAAAAAACTTTTTGGTGTACGAACTACCTGCCCATTTTGACTTTTCCTCCGGCGACCCTTTGGGCA
GTCAGCGCGACATTTACTATGCCATCATAAAAAGTCTCGAGGAGCGCGGATTTACTGTCAAAATATGTATGAAGGGAGACCGC
GCCCTTCTTTTCATCACCTGGAAAAAAATACAATCCATTGAGATCAACAAAAAAGAAGAATATCTGCGCATGCACTTCATACA
AGACGAAGAGAAAGCGTTTTATTGTAAATTTTTAGAGTCTAGATGA
Kenya 1950 B125R (AY261360.1:109401-109778)
SEQ ID No. 358
ATGGCGGTTTATGCGAAGGACCTTGATAATAACAAAGAGTTAAACCAAAAATTGATCAACGATCAGCTTAAAATCATTGACAC
GCTTTTGCTAGCAGAAAAAAAAAACTTTTTGGTGTACGAACTACCTGCCCATTTTGACTTTTCCTCCGGCGACCCTTTGGGCA
GTCAGCGCGACATTTACTATGCCATCATAAAAAGTCTCGAGGAGCGCGGATTTACTGTCAAAATATGTATGAAGGGAGACCGC
GCCCTTCTTTTCATCACCTGGAAAAAAATACAATCCATTGAGATCAACAAAAAAGAAGAATATCTGCGCATGCACTTCATACA
AGACGAAGAGAAAGCGTTTTATTGTAAATTTTTAGAGTCTAGATGA
L60 B125R (NC_044941.1:100308-100685)
SEQ ID No. 359
ATGGCGGTTTATGCGAAGGATCTTGATAATAACAAAGAGTTAAACCAAAAATTAATTAACGATCAGCTTAAAATTATTGACAC
GCTCTTGCTAGCAGAAAAAAAAAACTTTTTGGTGTATGAATTGCCTGCCCCTTTTGACTTTTCCTCCGGCGACCCTTTGGCCA
GTCAGCGCGACATATACTATGCCATCATAAAAAGCCTCGAGGAGCGCGGGTTTACTGTCAAAATATGTATGAAAGGGGATCGT
GCCCTCCTTTTCATCACTTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA
Malawi Lil-20/1 (1983) B125R (AY261361.1:103937-104314)
SEQ ID No. 360
ATGGCAGTTTATGCGAAAGATCTTGATAATAACAAAGAGTTAAACCAAAAATTAATTAACGATCAACTTAAAATTATTGACAC
GCTGTTGCTGGCAGAAAAAAAAAACTTTTTGGTGCATGAACTACCTGCCCACTATGACTTTTCCTCCGGCGACCCTTTGGCCA
GTCAGCGCGACATATACTATGCCATCATAAAAAGTCTCGAGGAGCGCGGATTTACTGTCAAAATTTGTATGAAAGGAGATCGC
GCCCTTCTTTTCATCACCTGGAAAAAAATACAATCCATTGAGATAAACAAAAAAGAAGAATATCTGCGCATGCACTTCATACA
AGATGAAGAGAAAGCATTTTATTGTAAATTTTTAGAGTCTAGATGA
Mkuzi 1979 B125R (AY261362.1:107379-107756)
SEQ ID No. 361
ATGGCGGTTTATGCGAAGGATCTTGATAATAACAAAGAGTTAAACCAAAAATTAATTAACGATCAGCTTAAAATTATTGACAC
GCTCTTGCTAGCAGAAAAAAAAAACTTTTTGGTGTATGAATTGCCTGCCCCTTTTGACTTTTCCTCCGGCGACCCTTTGGCCA
GTCAGCGCGACATATACTATGCCATCATAAAAAGCCTCGAGGAGCGCGGGTTTACTGTCAAAATATGTATGAAAGGGGATCGT
GCCCTCCTTTTCATCACTTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA
Pretorisuskop/96/4 B125R (AY261363.1:106504-106881)
SEQ ID No. 362
ATGGCGGTTTATGCGAAGGATCTTGATAATAACAAAGAGTTAAACCAAAAATTAATTAACGATCAGCTTAAAATTATTGACAC
GCTCTTGCTGGCAGAAAAAAAAAACTTTTTGGTGTATGAACTACCTGCCCCTTTTGACTTTTCCTCCGGCGACCCTTTGGCCA
GTCAGCGCGACATATACTATGCCATCATAAAAAGTCTCGAGGAGCGCGGGTTTACTGTCAAAATATGTATGAAAGGGGATCGC
GCCCTCCTTTTCATCACCTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA
Tengani 62 B125R (AY261364.1:100837-101214)
SEQ ID No. 363
ATGGCGGTGTATGCGAAGGACCTTGATAATAACAAAGAGTTAAACCAAAAATTAATTAACGATCAGCTTAAAATTATTGACAC
GCTCTTGCTGGCAGAAAAAAAAAACTTTTTGGTGTATGAACTACCTGCCCCTTTTGACTTTTCCTCCGGCGACCCTTTGGCCA
GTCAGCGTGACATATACTATGCCATCATAAAAAGTCTTGAGGAGCGCGGGTTTACTGTCAAAATATGTATGAAAGGGGATCGT
GCCCTCCTTTTCATCACCTGGAAAAAAATACAATCCATTGAAATAAACAAAAAAGAAGAATATCTGCGCATGCACTTCATACA
AGACGAAGAGAAATCATTTTATTGTAAATTTTTAGAGTCTAGATGA
Warmbaths B125R (AY261365.1:105532-105909)
SEQ ID No. 364
ATGGCGGTTTATGCGAAGGATCTTGATAATAACAAAGAGTTAAACCAAAAATTAATTAACGATCAGCTTAAAATTATTGACAC
GCTCTTGCTGGCAGAAAAAAAAAACTTTTTGGTGTATGAACTACCTGCCCCTTTTGACTTTTCCTCCGGCGACCCTTTGGCCA
GTCAGCGCGACATATACTATGCCATCATAAAAAGTCTCGAGGAGCGCGGGTTTACTGTCAAAATATGTATGAAAGGGGATCGC
GCCCTCCTTTTCATCACCTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA
Warthog B125R (AY261366.1:102258-102635)
SEQ ID No. 365
ATGGCGGTTTATGCGAAGGACCTTGATAATAACAAAGAGTTAAACCAAAAATTAATTAACGATCAGCTTAAAATTATTGACAC
GCTCTTGCTGGCAGAAAAAAAAAACTTTTTGGTGTATGAACTACCTGCCCCTTTTGACTTTTCCTCCGGCGACCCTTTGGCCA
GTCAGCGCGACATATACTATGCCATCATAAAAAGTCTCGAGGAGCGCGGGTTTACTGTCAAAATATGTATGAAAGGGGATCGC
GCCCTCCTTTTCATCACCTGGAAAAAAATACAATCCATTGAGATAAACAAAAAAGAAGAATATCTGCGCATGCACTTCATACA
AGACGAAGAGAAAGCATTTTATTGTAAATTTTTAGAGTCTAGATGA

In an embodiment an ASFV of the invention comprises a DIVA mutation that disrupts expression of the B125R gene. In other words, the ASFV lacks a functional version of the B125R gene. Suitably the B125R gene comprises the sequence of SEQ ID No. 353, 354, 355, 356, 357, 358, 359, 360, 361, 362, 363, 364 or 365. Suitably the B125R gene comprises a sequence having at least 70%, at least 80%, at least 90% or at least 95% identity with SEQ ID No. 353, 354, 355, 356, 357, 358, 359, 360, 361, 362, 363, 364 or 365. Suitably the B125R gene consists of the sequence of SEQ ID No. 353, 354, 355, 356, 357, 358, 359, 360, 361, 362, 363, 364 or 365.

In an embodiment of the ASFV of the invention the B125R gene may be partially or completely deleted. Suitably part or all of the sequence of SEQ ID No. 353, 354, 355, 356, 357, 358, 359, 360, 361, 362, 363, 364 or 365 is removed from the ASFV genome. Suitably the ASFV genome lacks any of these sequences.

The accessions numbers of B125R proteins from different strains of ASFV are listed below in Table 5.

TABLE 5

| Strain | B125R protein accession number |
|---|---|
| BA71V | Q65171 |
| PretoriuskopPr4/1996 | P0CA34 |
| Warthog | P0CA35 |
| Georgia 2007/1 | E0WMM1 |
| Portugal/OURT88/1988 | A9JLX0 |
| E75 | D4I5S1 |
| Benin 97/1 | A9JL41 |

The amino acid sequences of B125R proteins from different ASFV strains are depicted below.

Benin 97/1 B125R protein
SEQ ID No. 366
MAVYAKDLDNNKELNQKLINDQLKIIDTLLLAEKKNFLVYELPAPFDESSGDPLASQRDIYYAIIKSLEERGFTVKICMKGDR
ALLFITWKKIQSIEINKKEEYLRMHFIQDEEKAFYCKFLESR*
China/2018/AnhuiXCGQ B125R protein
SEQ ID No. 367
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKAFYCKFLESR*
Georgia 2007/1 B125R protein
SEQ ID No. 368
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKAFYCKFLESR
Ken05/Tk1 B125R protein
SEQ ID No. 369
MAVYAKDLDNNKELNQKLINDQLKIIDTLLLAEKKNFLVYELPAHFDFSSGDPLGSQRDIYYAIIKSLEERGFTVKICMKGDR
ALLFITWKKIQSIEINKKEEYLRMHFIQDEEKAFYCKFLESR*
Ken06.Bus B125R protein
SEQ ID No. 370
MAVYAKDLDNNKELNQKLINDQLKIIDTLLLAEKKNFLVYELPAHFDFSSGDPLGSQRDIYYAIIKSLEERGFTVKICMKGDR
ALLFITWKKIQSIEINKKEEYLRMHFIQDEEKAFYCKFLESR*
Kenya 1950 B125R protein
SEQ ID No. 371
MAVYAKDLDNNKELNQKLINDQLKIIDTLLLAEKKNFLVYELPAHFDFSSGDPLGSQRDIYYAIIKSLEERGFTVKICMKGDR
ALLFITWKKIQSIEINKKEEYLRMHFIQDEEKAFYCKFLESR*
L60 B125R protein
SEQ ID No. 372
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKAFYCKFLESR*
Malawi Lil-20/1 B125R protein
SEQ ID No. 373
MAVYAKDLDNNKELNQKLINDQLKIIDTLLLAEKKNFLVHELPAHYDFSSGDPLASQRDIYYAIIKSLEERGFTVKICMKGDR
ALLFITWKKIQSIEINKKEEYLRMHFIQDEEKAFYCKFLESR*
Mkuzi 1979 B125R protein
SEQ ID No. 374
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKAFYCKFLESR*
Pretorisuskop/96/4 B125R protein
SEQ ID No. 375
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKAFYCKFLESR*
Tengani 62 B125R protein
SEQ ID No. 376
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKSFYCKFLESR*
Warmbaths B125R protein
SEQ ID No. 377
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKAFYCKFLESR*
Warthog B125R protein
SEQ ID No. 378
MAVYAKDLDNNKELNQKLINDQLKIIDTLLLAEKKNFLVYELPAPFDFSSGDPLASQRDIYYAIIKSLEERGFTVKICMKGDR
ALLFITWKKIQSIEINKKEEYLRMHFIQDEEKAFYCKFLESR*

In an embodiment the ASFV of the invention does not express B125R protein. In other words, the ASFV of the invention does not express any proteins with sequences of SEQ ID Nos 366, 367, 368, 369, 370, 371, 372, 373, 374, 375, 376, 377 or 378.

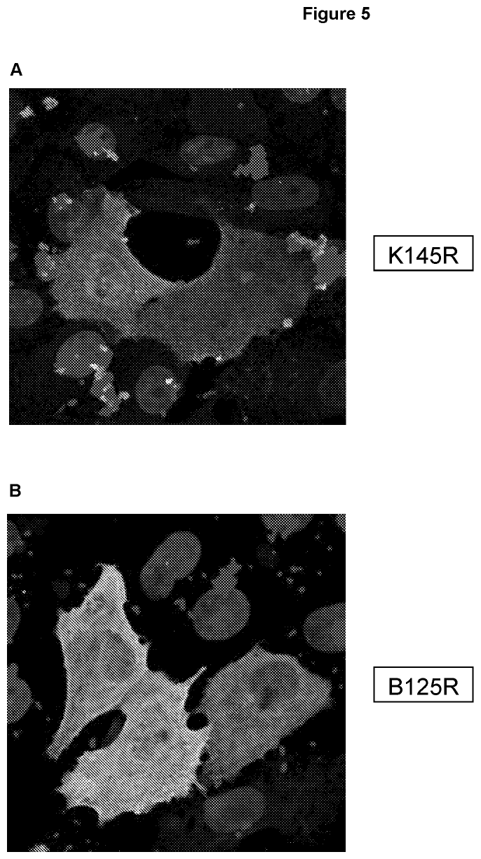

B125R was identified as one of the most abundant viral proteins expressed in infected wild boar cells (WSL-R) (Kaβler et al. 2018 Sci. Rep. 8: 1471). As shown in FIG. 5, B125R expression can be detected at the cell surface indicating that B125R is likely to be exposed to antibodies and likely to induce a strong antibody response.

### Multigene Families (MGFs)

ASFV contains five multi-gene families which are present in the left and right variable regions of the genome. The MGFs are named after the average number of codons present in each gene: MGF100, 110, 300, 360 and 505/530. The N-terminal regions of members of MGFs 300, 360 and 505/530 share significant similarity with each other. It has been shown the MGF 360 and 505 families encode genes essential for host range function that involves promotion of infected-cell survival and suppression of type I interferon response.

An attenuated ASFV according to the present invention comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

In a further aspect the invention provides an attenuated African Swine Fever (ASF) virus in which the expression and/or activity of the genes EP153R and EP402R is disrupted;

and which comprises a functional version of one or more of the following genes:
multigene family (MGF) 110 11L and 12L,
MGF 360 6L, 10L, 11L, 12L, 13L, and 14L, and
MGF 505 1R and 2R.

Suitably, in one embodiment the invention provides an ASFV in which the expression and/or activity of the genes EP153R and EP402R is disrupted;

and which comprises a functional version of one or more of the following genes:
multigene family (MGF) 110 5L, 6L, 8L, and 12L, and
MGF 360 6L, 10L, 11L, 12L, 13L, 14L and 21R, and
MGF 505 1R and 2R.

Suitably, in one embodiment the invention provides an ASFV in which the expression and/or activity of the genes EP153R and EP402R is disrupted;

and which comprises a functional version of one or more of the following genes:
multigene family (MGF) 110 6L, 8L, and 12L, and
MGF 360 6L, 10L, 11L, 12L, 13L, 14L and 21R, and
MGF 505 1R and 2R.

Suitably, in one embodiment the invention provides an ASFV in which the expression and/or activity of the genes EP153R and EP402R is disrupted;

and which comprises a functional version of one or more of the following genes:
multigene family (MGF) 110 12L, and
MGF 360 6L, 10L, 11L, 12L, 13L, 14L, and
MGF 505 1R and 2R.

Suitably, in one embodiment the invention provides an ASFV in which the expression and/or activity of the genes EP153R and EP402R is disrupted;

and which comprises a functional version of one or more of the following genes:
multigene family (MGF) 360 6L, 10L, 11L, 12L, 13L, and 14L, and
MGF 505 1R and 2R.

The location of some of these genes in the genomes of a variety of ASFV strains is provided below in Table 6. The sequence identity of each gene to the corresponding Benin 97/1 gene is also provided.

TABLE 6a
MGF 360-10L

| | Start | Stop | % |
|---|---|---|---|
| | Nucleotide | Nucleotide | Nucleotide |
| Strain | Number | number | Identity |
| Benin 97/1 | 20677 | 19607 | 100 |
| Georgia 2007/1 | 26438 | 25368 | 93 |
| BA71V | Deleted | | |
| OURT88/3 | Deleted | | |
| Kenya 1950 | 30628 | 29563 | 82 |
| Malawi-Lil/20 | 24802 | 23763 | 91 |
| Mkuzi 1979 | 28139 | 27069 | 99 |
| Pretorisuskop/96/4 | 27186 | 26116 | 96 |
| Tengani 62 | 21600 | 20530 | 92 |
| Warmbaths | 26361 | 25322 | 94 |
| Warthog | 23130 | 22091 | 95 |
| E75 | 19981 | 18911 | 100 |

TABLE 6b
MGF 360-11L

| | Start | Stop | % |
|---|---|---|---|
| | Nucleotide | Nucleotide | Nucleotide |
| Strain | Number | number | Identity |
| Benin 97/1 | 21764 | 20703 | 100 |
| Georgia 2007/1 | 27526 | 26465 | 97 |
| BA71V | Deleted | | |
| OURT88/3 | Deleted | | |
| Kenya 1950 | 31716 | 30655 | 84 |
| Malawi-Lil/20 | 25892 | 24831 | 86 |
| Mkuzi 1979 | 29225 | 28164 | 99 |
| Pretorisuskop/96/4 | 28275 | 27214 | 97 |
| Tengani 62 | 22688 | 21627 | 97 |
| Warmbaths | 27449 | 26388 | 97 |
| Warthog | 24218 | 23157 | 97 |
| E75 | 21068 | 20007 | 100 |

TABLE 6c
MGF 360-12L

| | Start | Stop | % |
|---|---|---|---|
| | Nucleotide | Nucleotide | Nucleotide |
| Strain | Number | number | Identity |
| Benin 97/1 | 24668 | 23616 | 100 |
| Georgia 2007/1 | 30434 | 29382 | 96 |
| BA71V | Deleted | | |
| OURT88/3 | Deleted | | |
| Kenya 1950 | 34566 | 33549 | 90 |
| Malawi-Lil/20 | 28731 | 27682 | 92 |
| Mkuzi 1979 | 32125 | 31073 | 97 |
| Pretorisuskop/96/4 | 31150 | 30098 | 95 |
| Tengani 62 | 25592 | 24540 | 96 |
| Warmbaths | 30346 | 29294 | 96 |
| Warthog | 27088 | 26036 | 96 |
| E75 | 23971 | 22920 | 99 |

TABLE 6d
MGF 360-13L

| | Start | Stop | % |
|---|---|---|---|
| | Nucleotide | Nucleotide | Nucleotide |
| Strain | Number | number | Identity |
| Benin 97/1 | 25901 | 24840 | 100 |
| Georgia 2007/1 | 31656 | 30595 | 97 |
| BA71V | Deleted | | |
| OURT88/3 | Deleted | | |
| Kenya 1950 | 35812 | 34757 | 90 |
| Malawi-Lil/20 | 29980 | 28925 | 91 |
| Mkuzi 1979 | 33347 | 32286 | 99 |
| Pretorisuskop/96/4 | 32368 | 31307 | 95 |
| Tengani 62 | 26814 | 25753 | 95 |
| Warmbaths | 31559 | 30498 | 95 |
| Warthog | 28338 | 27277 | 95 |
| E75 | 25204 | 24143 | 99 |

TABLE 6e
MGF 360-14L

| | Start | Stop | % |
|---|---|---|---|
| | Nucleotide | Nucleotide | Nucleotide |
| Strain | Number | number | Identity |
| Benin 97/1 | 27146 | 26073 | 100 |
| Georgia 2007/1 | 32913 | 31840 | 99 |
| BA71V | Deleted | | |
| OURT88/3 | Deleted | | |
| Kenya 1950 | 37194 | 36121 | 92 |
| Malawi-Lil/20 | 31266 | 30193 | 92 |
| Mkuzi 1979 | 34620 | 33547 | 99 |
| Pretorisuskop/96/4 | 33598 | 32525 | 97 |
| Tengani 62 | 28056 | 26983 | 97 |
| Warmbaths | 32820 | 31747 | 96 |
| Warthog | 29568 | 28495 | 97 |
| E75 | 26449 | 25376 | 100 |

TABLE 6f
MGF 505-1R

| | Start | Stop | % |
|---|---|---|---|
| | Nucleotide | Nucleotide | Nucleotide |
| Strain | Number | number | Identity |
| Benin 97/1 | 21971 | 23566 | 100 |
| Georgia 2007/1 | 27734 | 29329 | 95 |
| BA71V | Deleted | | |
| OURT88/3 | Deleted | | |
| Kenya 1950 | 31904 | 33496 | 91 |
| Malawi-Lil/20 | 26041 | 27627 | 93 |
| Mkuzi 1979 | 29425 | 31020 | 96 |
| Pretorisuskop/96/4 | 28449 | 30044 | 94 |
| Tengani 62 | 22891 | 24486 | 95 |
| Warmbaths | 27651 | 29246 | 95 |
| Warthog | 24387 | 25982 | 95 |
| E75 | 21275 | 22870 | 100 |

TABLE 6g
MGF 505-2R

| | Start | Stop | % |
|---|---|---|---|
| | Nucleotide | Nucleotide | Nucleotide |
| Strain | Number | number | Identity |
| Benin 97/1 | 27352 | 28932 | 100 |
| Georgia 2007/1 | 33119 | 34699 | 99 |
| BA71V | 17725 | 19304 | 99 |
| OURT88/3 | 29532 | 29981 | 76 |
| Kenya 1950 | 37419 | 38985 | 90 |
| Malawi-Lil/20 | 31541 | 33121 | 93 |
| Mkuzi 1979 | 34826 | 36406 | 99 |
| Pretorisuskop/96/4 | 33795 | 35374 | 97 |
| Tengani 62 | 28261 | 29830 | 98 |
| Warmbaths | 33029 | 34597 | 96 |
| Warthog | 29773 | 31352 | 97 |
| E75 | 26655 | 28236 | 99 |

TABLE 6h
MGF 505-3R

| | Start | Stop | % |
|---|---|---|---|
| | Nucleotide | Nucleotide | Nucleotide |
| Strain | Number | number | Identity |
| Benin 97/1 | 29019 | 29861 | 100 |
| Georgia 2007/1 | 34786 | 35625 | 96 |
| BA71V | 20398 | 21915 | 100 |
| OURT88/3 | 20850 | 22367 | 100 |
| Kenya 1950 | 40295 | 41815 | 89 |
| Malawi-Lil/20 | 34322 | 35842 | 89 |
| Mkuzi 1979 | 37500 | 39017 | 99 |
| Pretorisuskop/96/4 | 36480 | 37985 | 89 |
| Tengani 62 | 30926 | 32443 | 90 |
| Warmbaths | 35712 | 37232 | 93 |
| Warthog | 32449 | 33954 | 90 |
| E75 | 29330 | 30847 | 100 |

The gene (i.e. nucleotide) sequences of these genes from different strains are given below.

### MGF 110 5L Gene Sequences

China/2018/AnhuiXCGQ (genotype II) 9145-8528 MGF 110 5L/6L fusion
SEQ ID No. 266
ATGTTGGTGATCTTCTTGGGAATTCTTGGCCTTCTGGCCAGCCAGGTTTCAAGTCAGCTCGTTGGACAACTTCGTCCAACAGA
GGAGCCTCCAGAGGAAGAACTCGAATACTGGTGTGCCTACATGGAAAGTTGCCAATTTTGCTGGGACTGCCAAGACGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAGGGTATCTACCATACCATGAATTGCAACCAGGTCTTAGGGCTACCTAATCAACCAGCTGGACAACT
TCATCCAACGGATAATCCTCCACAAGAAGAACTTGAATACTGGTGCACTTATACAGAAAACTGCAAGTTTTGCTGGAATTGTC
AAAATGGCCTTTGTGAGGGCAAATTGGAGAATACAACAATTCTTGAAAATGAGTATGTGCAATCATGTATAGTTTCCCGCTGG
CTAAATAAATGTATGTATGATCTAGGACAGGGGATTCACCATGTAATGGCCTGTTCTGAACCAAAGCCCTGGAATCCTTACAA
AATCTTAAAGAGAGAGTGGAAAGAAAATAATAGTTAA
Georgia 2007/1 (genotype II) 9140-8523 MGF 110 5L/6L fusion
SEQ ID No. 267
ATGTTGGTGATCTTCTTGGGAATTCTTGGCCTTCTGGCCAGCCAGGTTTCAAGTCAGCTCGTTGGACAACTTCGTCCAACAGA
GGAGCCTCCAGAGGAAGAACTCGAATACTGGTGTGCCTACATGGAAAGTTGCCAATTTTGCTGGGACTGCCAAGACGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAGGGTATCTACCATACCATGAATTGCAACCAGGTCTTAGGGCTACCTAATCAACCAGCTGGACAACT
TCATCCAACGGATAATCCTCCACAAGAAGAACTTGAATACTGGTGCACTTATACAGAAAACTGCAAGTTTTGCTGGAATTGTC
AAAATGGCCTTTGTGAGGGCAAATTGGAGAATACAACAATTCTTGAAAATGAGTATGTGCAATCATGTATAGTTTCCCGCTGG
CTAAATAAATGTATGTATGATCTAGGACAGGGGATTCACCATGTAATGGCCTGTTCTGAACCAAAGCCCTGGAATCCTTACAA
AATCTTAAAGAGAGAGTGGAAAGAAAATAATAGTTAA
Ken05/Tk1 9698-9327 MGF 110 5L
SEQ ID No. 268
ATGTTGGTGATCTTCTTGGGACTCCTTGGCCTGCTGGCCAACCAGGTCTTAGGGCTACCTAATCAACCAGCTGGACAACTTCA
TCCAACGGATAATCCTCCAAAAGAAGAACTTGGATACTGGTGCACTTACACGGAAAGCTGTAAATTTTGCTGGAACTGTCAAA
ATGGCCTTTGTGAGGGCAAATTGGAGAATACAACAATTCTTGAAAATGAGTATGTGCAATCATGTATAGTTTCCCGCTGGCTA
AATAAATGTATGTATGATCTAGGACAGGGGATTGACCATGTAATGGCCTGTTCTGAACCAAAGCCCTGGAATCCTTACAAAAT
CTTAAAGAGAGAGTGGAAAAAAAATGTCAGCCAAAATTAA
Ken06.Bus 6224-5853 MGF 110 5L
SEQ ID No. 269
ATGTTGGTGATCTTCTTGGGACTCCTTGGCCTGCTGGCCAACCAGGTCTTAGGGCTACCTAATCAACCAGCTGGACAACTTCA
TCCAACGGATAATCCTCCAAAAGAAGAACTTGGATACTGGTGCACTTACACGGAAAGCTGTAAATTTTGCTGGAACTGTCAAA
ATGGCCTTTGTGAGGGCAAATTGGAGAATACAACAATTCTTGAAAATGAGTATGTGCAATCATGTATAGTTTCCCGCTGGCTA
AATAAATGTATGTATGATCTAGGACAGGGGATTCACCATGTAATGCCCTGTTCTGAACCAAAGCCCTGGAATCCTTACAAAAT
CTTAAAGAAAGAGTGGAAAGAAAATGTCAGCCAAAATTAA
Malawi Lil-20/1/1983 7878-7516 MGF 110 5L
SEQ ID No. 270
ATGTTGGTTATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAGATGGCCTTT
GTGTGAATAAGTTGGGAAATACAACAATTCTTGAAAATGAGTATGTGCATCCATGTATAGTTTCCCGCTGGCTAAATAAATGT
ATGTATGATCTGGGTCAAGGCATTGATCATGTAATGGTCTGTTCTCAACCAAAGTACTGGAATCCTTATAAAATCTTAAAGAA
AGAGTGGAAAGAAAATAACAGCCAAAATTAA
Mkuzi 1979 (genotype XII) 10334-9972 MGF 110 5L
SEQ ID No. 271
ATGTTAGTAATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAGATGGCCTTT
GTGTGAATAAGTTGGGAAATACAACAATTCTTGAAAATGAGTATGTGCATCCATGTATAGTTTCCCGCTGGCTAAATAAATGC
ATGTATGATCTGGGTCAAGGCATTGATCATGTAATGGTCTGTTCTCAACCAAAGTACTGGAATCCTTATAAAATCTTAAAGAA
AGAGTGGAAAGAAAATAACAGTCAAAATTAA
Pretorisuskop/96/4 (genotype XX/I) 9424-9059 MGF 110 5L
SEQ ID No. 272
ATGTTAGTAATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTATGATCTGGGTCAAGGCATTGATCATGTAATGGTCTGTTCTCAGCCAAAATACTGGAATCCTTATAAAATCTTAAAGAA
AGAGTGGAAAGAAAATAACAGCCAAAATAAATAA
Tengani62 (genotype V/I) 8803-8447 MGF 110 5L
SEQ ID No. 273
ATGTTAGTCATCTTCTTGGGAATTCTTGGCCTTCTGGTCAACCAGGTCTCAAGCCAGCCAGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTATGATCTGGGTCAAGGCATTGATCATGTAATGGTCTGTTCTCAACCAAAATACTGGAATCCTTATAAAATCTTAAAGAA
AGAGTGGAAAGAAAATAATAGTTAA
Warmbaths (genotype III/I) 8649-8284 MGF 110 5L
SEQ ID No. 274
ATGTTAGTAATCTTTTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTACGATCTGGGTCAAGGCATTGATCATGTAATGGTCTGTTCTCAACCAAAATACTGGAATCCTTATAAAATCTTAAAGAA
AGAGTGGAAAGAAAATAACAGCCAAAATAAATAA
Warthog (genotype IV) 7349-6984 MGF 110 5L
SEQ ID No. 275
ATGTTAGTAATCTTCTTGGGAATTCTTGGCCTTCTGGCCAACCAGGTCTCAAGCCAGCTCGTTGGACAACTTCATCCAACGGA
AAATCCTTCAGAGAATGAACTTGAATACTGGTGCACTTACATGGAATGTTGCCAGTTTTGCTGGGACTGTCAAAATGGCCTTT
GTGTGAATAAGTTGGGAAATACAACAATTCTTGAAAATGAGTATGTGCATCCATGTATAGTTTCCCGCTGGCTAAATAAATGT
ATGTACGATCTGGGTCAAGGCATTGATCATGTAATGGTCTGTTCTCAACCAAAATACTGGAATCCTTATAAAATCTTAAAGAA
AGAGTGGAAAGAAAATAACAGCCAAAATAAATAA

In an embodiment the attenuated ASFV of the invention comprises a functional version of MGF 110 5L. Suitably the functional version of MGF 110 5L comprises the sequence of SEQ ID No. 266, 267, 268, 269, 270, 271, 272, 273, 274 or 275. Suitably the functional version of MGF 110 5L comprises a sequence having at least 70%, at least 80%, at least 90% or at least 95% identity with SEQ ID No. 266, 267, 268, 269, 270, 271, 272, 273, 274 or 275. Suitably the functional version of MGF 110 5L consists of the sequence of SEQ ID No. 266, 267, 268, 269, 270, 271, 272, 273, 274 or 275.

### MGF 110 6L Gene Sequences

Ken05/Tk1 MGF 110 6L
SEQ ID No. 35
ATGTTGGTAATCTTTTTGGGAATTCTTGGCCTTCTGGCCAGCCAGGTCTCAAGTCAACCAGATGGACAACTTCGTCCAACAGA
GGATCCTCCAGAAGAAGAACTTAAATATTGGTGCACCTACATGGAAAGTTGCCAGTTTTGTTGGGACTGCCAAGATGGCAATT
GTATAAACAAAGTAGATGGGTCAGTCATTTATAAAAATGAGTTTGTGCGACCATGTTCAGTTTCCCGCTGGATGAATAAATGT
ATGTATGATTTAAATAAGGGTATCTATCATACAATGAATTGTTCTCAGCCACAGTCTTGGAATCCCTACAAATACTTCAGGAA
GGAGTGGAAAAAAGATGAACTCTAG
Ken06.Bus MGF 110 6L
SEQ ID No. 36
ATGTTGGTGATCTTTTTGGGAATTCTTGGCCTTCTGGCCAGCCAGGTCTCAAGTCAGCCAGTTGGACAACTTCGTCCAACAGA
GGACCCTCCAGAGAATGAACTCGAATATTGGTGCACCTACATGGAAAGTTGCCAGTTTTGCTGGGACTGTCAAGATGGCAATT
GTATAAACAAAGTAGATGGGTCAGTCATTTATAAAAATGAGTATGTGCAACCATGTTCAGTTTCCCGCTGGCTAAATAAATGT
ATGTATGATTTAGATAAGGGTATCTATCATACAATGAACTGTTCTCAGCCACGGTCTTGGAATCCCTACAAATGTTTCAGGAA
GGAGTGGAAAAAAGATGAACTCTAG
Kenya 1950 MGF 110 6L
SEQ ID No. 37
ATGTTGGTGATCTTTTTGGGAATTCTTGGCCTTATGGCCAGCCAGGTCTTAGGGCTACCATCTAATCAACCAACTGGACAACT
TCGTCCAACAGAGGATCCTCCAGAGGAAGAACTCGAATACTGGTGTGCCTACATGGAAAGTTGCCAGTTTTGCTGGGACTGCC
AAGATGGCAATTGCATAAACAAAATAGATGGGTCAGTCATTTATAAAAATGAGTTTGTGCGACCATGTTCAGTTTCCCGCTGG
ATGGATAAATGTATGTATGATTTAAATAAGGGTATCTATCATACAATGAATTGTTCTCAGCCACAGTCTTGGAATCCCTACAA
ATACTTCAGGAAGGAGTGGAAAAAAGATGAACTCTAG
Malawi Lil-20/1 (1983) MGF 110 6L
SEQ ID No. 38
ATGTTGGTGACCTTTTTGGGAATTCTTGGCCTTCTGGCCAGCCAGGTCTCAAGTCAGCTCGTTGGACAACTTCGTCCAACAGA
GGATCCTCCAGAGGAAGAACTCGAATACTGGTGCGCCTACATGGAAAGTTGCCAGTTTTGCTGGGACTGCCAAGATGGCAATT
GTATAAACAAAATAGATGGGTCAGTCATTTATAAAAATGAGTATGTGCGACCATGTTCAGTTTCCCGTTCGATGGATAAATGT
ATGTATGATTTAAATAAGGGTATCTACCATAGCATGAGCTGTTCTGACCCAAAGGCCTGGAATCCCTACAAATACTTCAGGAA
GGAATGGAAAAAAGATGAACTCTAG
Mkuzi 1979 MGF 110 6L
SEQ ID No. 39
ATGTTGGTGATCTTTTTGGGAATTCTTGGCCTTCTGGCCAGCCAGGTTTCAAGTCAACTCGTTGGACAACTTCGACCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAAGCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG
Pretorisuskop/96/4 MGF 110 6L
SEQ ID No. 40
ATGTTGGTGATCTTCTTGGGAATTCTTGGCCTTCTGGCCAGCCAGGTTTCAAGTCAGCTCGTTGGACAACTTCGTCCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG
Tengani62 MGF 110 6L
SEQ ID No. 41
ATGTTGGTGATCTTCTTGGGAATTTTTGGCCTTCTGGCCAGCCAGGTTTCAAGTCAGCTCGTTGGACAACTTCGTCCAACAGA
GGATCCTCCAGAGGAAGAACTCGAATACTGGTGTGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAGGGTATCTACCATACCATGAATTGTTCTCAGCCATGGTCTTGGAATCCTTACAAATACTTCAGGAA
GGAATGGAAAAAAGATGAACTCTAG
Warmbaths MGF 110 6L
SEQ ID No. 42
ATGTTGGTGATCTTCTTGGGAATTCTTGGCCTTCTGGCCAGCCAGGTTTCAAGTCAGCTCGTTGGACAACTTCGTCCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG
Warthog MGF 110 6L
SEQ ID No. 43
ATGTTGGTGATCTTCTTGGGAATTCTTGGCCTTCTGGCCAGCCAGGTTTCAAGTCAGCTCGTTGGACAACTTCGACCAACAGA
GGATCCTCCAGAGGAAGAACTCGAATACTGGTGCGCCTACATGGAAAGTTGCCAATTTTGCTGGGACTGCCAAGATGGCACTT
GTATAAACAAAATAGATGGGTCAGTAATTTACAAGAATGAGTATGTGAAATCATGTCTGGTTTCCCGTTGGCTGGATAAATGT
ATGTATGATTTAGATAAAGGTATCTACCATACCATGAATTGTTCTCAGCCATGGTCTTGGAATCCTTACAAATACTTCAGGAA
GGAATGGAAAAAAGATGAACTCTAG

In an embodiment the attenuated ASFV of the invention comprises a functional version of MGF 110 6L. Suitably the functional version of MGF 110 6L comprises the sequence of SEQ ID No. 35, 36, 37, 38, 39, 40, 41, 42 or 43. Suitably the functional version of MGF 110 6L comprises a sequence having at least 70%, at least 80%, at least 90% or at least 95% identity with SEQ ID No. 35, 36, 37, 38, 39, 40, 41, 42 or 43. Suitably the functional version of MGF 110 6L consists of the sequence of SEQ ID No. 35, 36, 37, 38, 39, 40, 41, 42 or 43.

### MGF 110 7L Gene Sequences

China/2018/AnhuiXCGQ MGF 110 7L
SEQ ID No. 247
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGAACCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTATTGAGAATGACTATGTAAAATATTGT
GAGGTTTCCCGCTGGGGTGACCTATGTAGATATGATGTGGAGGAGCACATTTACCATAGCATGAACTGTTCTGACCCAAAGCC
CTGGAATCCTTATAAAATTGCAAGGAAGGAGTGGAAAAAGAATGAACATCCCAGAAAGGATTTGAAAAAAGATGAATTCTAG
Georgia 2007/1 MGF 110 7L
SEQ ID No. 248
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGAACCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTATTGAGAATGACTATGTAAAATATTGT
GAGGTTTCCCGCTGGGGTGACCTATGTAGATATGATGTGGAGGAGCACATTTACCATAGCATGAACTGTTCTGACCCAAAGCC
CTGGAATCCTTATAAAATTGCAAGGAAGGAGTGGAAAAAGAATGAACATCCCAGAAAGGATTTGAAAAAAGATGAATTCTAG
Ken05/Tk1 MGF 110 7L
SEQ ID No. 249
ATGCTGGTGATTATCCTGGGAGTTATTGGCCTGCTGGCCAGTAGCAACTTGGTTTCATCCTCCACTTCTACTCGGATAGGTGG
ACATCTTCCTCTAACATTTGATCCTCCAGAGAATGAACTTGGATACTGGTGCACTTATGTGGAAAGCTGTCGGTTCTGCTGGG
ATTGTGAAGATGGGGTCTGTACTAGCAGGATCTGGGGGAACAACTCCACAAGTATTGTTGAGAACAGCTATGTAAAATATTGT
GAAGTTTCCCGCTGGGGTGACCAGTGTAGATATGATGTGGAGGAGCGCATTTACCATACCATGAACTGTTCTGACCCAAAACC
CTGGAATCCTTATAAAATTACAAGGGTGGAGTGGAAAAAGAATGAACATTTCAGAAAGGATTTGAAAAAAGATGAATTCTAG
Kenya 1950 MGF 110 7L
SEQ ID No. 250
ATGCTGGTGATTATCCTGGGAGTTATTGGCCTGCTGGCCAGTAGCAACTTGGTTTCATCCTCCACTTCTACTCGGGTAGGTGG
ACATCTTCCTCTAACATTTGATCCTCCAGAGAATGAACTTGGATACTGGTGCACTTATGTGGAAAGCTGTCGGTTCTGCTGGG
ATTGTGAAGATGGGGTCTGTACTAGCAGGATCTGGGGGAACAACTCCACAAGTATTGTTGAGAACAGCTATATAAAATATTGT
GAAGTTTCCCGCTGGGGTGACCAGTGTAGATATGATGTGGAGGAGCACATTTACTATACCATGAACTGTTCTGACCCAAAGCC
CTGGAATCCTTATAAAATTGCAAGGAAGGAGTGGAAAAAGAATGAACATTTCAGAAAGGATTTGAAAAAAGATGAATTCTAG
Malawi Lil-20/1 (1983) MGF 110 7L
SEQ ID No. 251
ATGCTGGTGATTATCCTGGGAGTTATTGGCCTGCTGGCCAGTAGCAACTTGGTTTCATCCTCCACTTCTACTCGGGTAGGTGG
ACATCTTCCTCTAACATTTGATCCTCCAGAGAATGAACTTGGATACTGGTACACTTATGTGGAAAGTTGTCGGTTCTGCTGGG
ATTGTGAAGATGGGGTCTGTACTAGCAGGGTTTGGGGAAACAACTCCACAAGTATTGTTGAGAATGACTATGTAAAATATTGT
GAGGTTTCTCGCTGGGGTGACCAATGTAGATATGATGTGGAGGAGCACATTTACTATACCATGAACTGTTCTGACCCAAAGCC
CTGGAATCCTTATAAAATTGCAAAGGAAGGAGTGGAAAAAGGATGA
Mkuzi 1979 MGF 110 7L
SEQ ID No. 252
ATGCTGGTGATTATCCTGGGAGTTATTGGCCTGCTGGCCAGTAGCAACTTGGTTTCATCCTCCACTTCTACTCGGGTAGGTGG
ACATCTTCCTCTAACAGAGGATCCTCCAGAGAATGAACTTGGATACTGGTGCACTTATGTGGAAAGCTGTCGGTTCTGCTGGG
ATTGTGAAGATGGGATTTGTACTAGCAGGGTTTGGGGAAACAACTCCACAAGTATTGTTGAGAACGACTATGTAAAATATTGT
GAGGTTTCTCGCTGGGGTGACCAATGTAGATATGATGTAGAGGAGCACATTTACTATACCATGAACTGTTCTGACCCAAAGCC
CTGGAATCCTTATAAAATTGCAAGGAAGGAGTGGAAAAAGGATGAACATTCCAGAAAGAATTTGAAAAAAGATGAATTCTAG
Pretorisuskop/96/4 MGF 110 7L
SEQ ID No. 253
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGATCCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTGTTGAGAATGACTATGTAAAATATTGT
GAGGTTTCCCGCTGGGGAAACCTATGTAGATATGATGTGGAGGAGCACATTTACTATAGCATGAACTGTTCTGACCCAAAGCC
CTGGAATCCTTATAAAATTGCAAGGAAGGAGTGGAAAAAGAATGAATATCTCAGAAAGGATTTGAAAAAAGATGAATTCTAG
Tengani 62 MGF 110 7L
SEQ ID No. 254
ATGAACTGTTCTGACCCAAAGCCCTGGAATCCTTATAAAATTGCAAGGAAGGAGTGGAAAAAGAATGAACATCCCAGAAAGGA
TTTGAAAAAAGATGAATTCTAG
Warmbaths MGF 110 7L
SEQ ID No. 255
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGATCCCCCAGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTGTTGAGAATGACTATGTAAAATATTGT
GAGGTTTCCCGCTGGGGTAACCTATGTAGATATGATGTGGAGGAGCACATTTACCATAGCATGAACTGCTCTGACCCAAAGCC
CTGGAATCCTTATAAAATTGCAAGGAAGGAGTGGAAAAAGAATGAACATCCCAGAAAGGATTTGAAAAAAGATGAATTCTAG
Warthog MGF 110 7L
SEQ ID No. 256
ATGCTGGTGATTATCCTGGGAATTATTGGCCTGCTGGCCAGTAGCAACCTGGTTTCATCCTCCACTTCTACTCGGGTAGGTGG
ACATCTTCCTCTAACATTTGATCCCCCCGAGAATGAACTTGGATACTGGTGCACTTATGTGGAAAGCTGTCGGTTCTGCTGGG
ATTGTGAAGATGGGATTTGTACCAGCAGGGTTTGGGGAAACAACTCCACAAGTATTGTTGAGAATGACTATGTAAAATATTGT
GAGGTTTCCCGCTGGGGTAACCTATGTAGATATGATGTGGAGGAGCACATTTACCATAGCATGAACTGCTCTGACCCAAAGCC
CTGGAATCCTTATAAAATTGCAAGGAAGGAGTGGAAAAAGAATGAACATCCCAGAAAGGATTTGAAAAAAGATGAATTCTAG

In an embodiment the attenuated ASFV of the invention comprises a functional version of MGF 110 7L. Suitably the functional version of MGF 110 7L comprises the sequence of SEQ ID No. 247, 248, 249, 250, 251, 252, 253, 254, 255 or 256. Suitably the functional version of MGF 110 7L comprises a sequence having at least 70%, at least 80%, at least 90% or at least 95% identity with SEQ ID No. 247, 248, 249, 250, 251, 252, 253, 254, 255 or 256. Suitably the functional version of MGF 110 7L consists of the sequence of SEQ ID No. 247, 248, 249, 250, 251, 252, 253, 254, 255 or 256.

### MGF 110 8L Gene Sequences

Ken05/Tk1 MGF 110 8L
SEQ ID No. 44
ATGAAGGTGCTGATTCTAGTACTACTGGGGGTCGTTATCCTTCAGGCTGCTCCTATACGTAAATTAGAAGACCTACTGCCAAC
CCGTAATCCTCCCCAAAATGAGCTAGTTTACTGGTGCACCTACGCAAACCAATGTGACTTTTGCTGGGAATGTGTACACGGAA
TCTGCCGAAACAGAATTCAAGCAGATTGGCCTGTGATTCACCAAAATGACTGGATTATAAATTGCACCGTTTCCCGCTGGAAT
GGTATATGTAGGTATTATGAAGGACCCAGAGTCCATATAGATCATGAAATGGACTGTGCAAATCCAACATCCCATACTTATCC
ACACATTGAATACATGAAGATCTATGAAAGAGATGACCTATGA
Kenya 1950 MGF 110 8L
SEQ ID No. 45
ATGAAGGTGCTGATTCTAGTACTACTGGGGGTCGTTATCCTTCAGGCTGCTCCTATACGTAAATTAGAAGACCTACTGCCAAC
CCGTAATCCTCCCCAAAATGAGTTAGTTTATTGGTGCACCTACGCAAACCAATGTGATTTTTGCTGGGAATGTATACACGGAA
TCTGCCGAAACAGAATTCAAGCAGATTGGCCTGTGATTCACCAAAATGACTGGATTATAAATTGCACCGTTTCCCGCTGGAAT
GGTATATGTAGGTATTATGAAGGACCCAGAGGCCATATAGATCATGAAATGGACTGTGCAAATCCAACATACCATACTTATCC
ACACATTGAATACATGAAGATTTATGTAAGAGATGACCTATGA
Malawi Lil-20/1 (1983) MGF 110 8L
SEQ ID No. 46
ATGAAGGTGCTGATTCTAGTACTGCTGGCGGTGGTTATCCTTCAAGCCGCTCCTATACGTAAATTAGAAGATCTACTACCAAC
CCGTTATCCTCCTGACCATGAGCTAGTTTATTGGTGCACCTACGCAAACCAATGTGACTTTTGCTGGGAATGCGTACACGGTA
TCTGCCGAAATAGGATTCAAGCAGATTGGCCGGTTATTCACCAAAATGACTGGATTATAAATTGCACGGTTTCCCGCTGGAAT
GGTATATGTAGTTATTATGAAGGACCCAGAAACCATACAGATCATCAAATGGACTGTGCAAATCCAACATCCCATACCTATCC
ACATAGAGAATACATGAAAATTTATGAAAGAGATGACTTATAA
Mkuzi 1979 MGF 110 8L
SEQ ID No. 47
ATGAAGGTGCTGATTCTAGTACTACTGGGGGGGGGGGGTATTATTCTTCAGGCCGCTCCTATACGTAAATTAGAAGATCTACT
ACCAACCCGTTATCCTCCTGAACATGAGCTGGTTTATTGGTGCACCTACGCAAACCAATGTGACTTTTGCTGGGAATGCGTCC
ACGGTATTTGCCGAAACAGGATTCAAGCAGATTGGCCGGTTATTCATCAAAATGACTGGATTATAAATTGCACGGTTTCCCGC
TGGAATGGCCAATGTCATTATTATGAAGGTTCTCAACAATATCTACATCATGAAATGGACTGTACAAATCCAACATCCCATAC
CTATCCACATACAGAATACATGAAGATCTATGAAAGAGATGACTTATGA
Pretorisuskop/96/4 MGF 110 8L
SEQ ID No. 48
ATGAAGGTGCTAATTCTAGTACTACTGGGGGTGGTTATCCTTCAGGCCGCTCCTATACGTAAATTAGAAGATCTACTACCAAC
CCGTTATCCTCCTGAACATGAGCTAGTTTATTGGTGCACTTACGCAAACCAATGTGACTTTTGCTGGGAATGCGTCCACGGTA
TCTGCCGAAACAGGATTCAAGCAGATTGGCCAGTTATTCATCAAAATGACTGGATTATAAATTGCACGGTTTCCCGCTGGAAT
GGTATATGTAGTTATTATGAAGGACCCAGAAACCATACAGATCATCAAATGGACTGTGCAAATCCAACATCCCATACTTATCC
ACATAGAGAATACATGAAGATCTATGAAAGAGATGACTTATGA
Warmbaths MGF 110 8L
SEQ ID No. 49
ATGAAGGTGCTAATTCTAGTACTACTGGGGGTGGTTATTCTTCAGGCCGCTCCTATACGTAAATTAGAAGATCTACTACCAAC
CCGTTATCCTCCTGAACATGAGCTAGTTTATTGGTGCACTTACGCAAACCAATGTGACTTTTGCTGGGAATGCGTCCACGGTA
TCTGCCGAAACAGGATTCAAGCAGATTGGCCAGTTATTCACCAAAATGACTGGATTATAAATTGCACAGTTTCTCGCTGGAAT
GGTATATGTAGTTATTATGAAGGACCCAGAAACCATACAGATCATCAAATGGACTGTGCAAATCCAACATCCCATACTTATCC
ACATAGAGAATACATGAAGATCTATGAAAGAGATGACTTATGA
Warthog MGF 110 8L
SEQ ID No. 50
ATGAAGGTGCTAATTCTAGTACTACTGGGGGTGGTTATTCTTCAGGCCGCTCCTATACGTAAAGTAGAAAATCTACTGCCAAC
CCGTAATCCTCCCCAAAATGAATTAGTTTATTGGTGCACCTATGCAAACCAATGTGACTTTTGCTGGGAATGCATTCACGGCA
TCTGCCGAAACAGGATTCAAGCAGATTGGCCAGTTATTCATCAAAATGACTGGATTATAAATTGCACGGTTTCCCGCTGGAAT
GGCCAATGTCATTATTATGAAGGTTCTCAACAATATCTGCATCATGAAATGGACTGTATAAATCCAACATCCCATACCTATCC
ACATACAGAATACATGAAGATCTATGAAAGAGATGACTTATGA

In an embodiment the attenuated ASFV of the invention comprises a functional version of MGF 110 8L. Suitably the functional version of MGF 110 8L comprises the sequence of SEQ ID No. 44, 45, 46, 47, 48, 49 or 50. Suitably the functional version of MGF 110 8L comprises a sequence having at least 70%, at least 80%, at least 90% or at least 95% identity with SEQ ID No. 44, 45, 46, 47, 48, 49 or 50. Suitably the functional version of MGF 110 8L consists of the sequence of SEQ ID No. 44, 45, 46, 47, 48, 49 or 50.

### MGF 110 12L Gene Sequences

Benin 97/1 (genotype I) 7643-7332 MGF 110 12L
SEQ ID No. 276
ATGAAGGTTTTTCTAGGACTTTTACTAGGTTATTCAACCATCCTCATTCTTACATATCAATCACCAACAACCCAGTGGTGTTT
TTATGAAATATCACTTAAAATACTTAATCATCATAGCATGGAAAAATGGAGGGATAAGAATTGGTCAATCATTATAAGGTATT
ATTGTTTTTACCTTGTATTTAGCTTTGCATTTGCTGGTTGCGTTGCATTTGCGATCTGCAAAAATCTACGACTGTGTACAACC
ATGAAATTACTTATGCTTTTGAATATTTTGGTTTTGTTATCTCAGCCAATTTTGAATAATTGA
China/2018/AnhuiXCGQ (genotype II) 14155-13796 MGF 110 12L
SEQ ID No. 277
ATGAAGGTTTTTCTAGGACTTTTACTAGGTTATTCAACCATCCTTATTCTTACCTATCAATCACCAACAACCCCGTGGTGTTT
TTATGAAATATCACTTAAAATACCTAATCATCATAGCATGAAATGTTGTTCGTATCCTAGACTTTATGAACATGAAATGTTCA
TGGAAAAATGGAGGGATAAGAATTGGCCAATCATTATAAGGTATTATTGTTTTTACCTTGTATTTAGCTTTGTATTTGCTGGT
TGCGTTGCATTTGCGATCTGCAAAAATCTACGACTGAGTACAACCATGAAATTACTTATGCTTTTGAGTATTTTGGTTTTGTT
GTTATCTCAGCCAATCTTGAATAATTGA
Georgia 2007/1 (genotype II) 14151-13792 MGF 110 12L
SEQ ID No. 278
ATGAAGGTTTTTCTAGGACTTTTACTAGGTTATTCAACCATCCTTATTCTTACCTATCAATCACCAACAACCCCGTGGTGTTT
TTATGAAATATCACTTAAAATACCTAATCATCATAGCATGAAATGTTGTTCGTATCCTAGACTTTATGAACATGAAATGTTCA
TGGAAAAATGGAGGGATAAGAATTGGCCAATCATTATAAGGTATTATTGTTTTTACCTTGTATTTAGCTTTGTATTTGCTGGT
TGCGTTGCATTTGCGATCTGCAAAAATCTACGACTGAGTACAACCATGAAATTACTTATGCTTTTGAGTATTTTGGTTTTGTT
GTTATCTCAGCCAATCTTGAATAATTGA
Ken05/Tk1 (genotype IX) 15456-14950 MGF 110 12L
SEQ ID No. 279
ATGAAGGTTTTTCTAGGACTTTTACTAGGTTTTTCAATCATCCTCATTCTTACATATCAATCACCGACAACTCAGCATCCTCC
TAAGGAAGAGCTTGCGTACTGGTGCACTTATGCAAAATCTTGTGACTTCTGCTGGGATTGCCAAAATGACACTTGTATAAATA
AGGTAATAAATGAGTCTATTTCGATAACTTCGATTGTAAACTGTAGAGTTACTCGCGATTCCCAATCCTGTTTTTATGAAATA
TCAGTTAAAATACCTAATCATCATAGCATGGAATGTTCATATCCTAGACTTTATGAACATGAAATGTTTATGGAAAAATGGAG
GGATGAGTATTGGCCAATCATTATAAAACAGTGTTGTTTTTACCTTGTATTTAGCATTGCATTTGCTGGATGCGTTGCATTTG
CGATCTGCAAAAATCTACGACTGCGTACAACTATAAAACTACTTATTCTTTTGAGTATTTTGGTTTGGTTGTCTCAACCAGTC
TTGAATTGA
Ken06.Bus (genotype X) 11961-11446 MGF 110 12L
SEQ ID No. 280
ATGAAGGTTTTTCTGGCACTTTTACTAGGTTATTTAACTATCCTCATTCTTACATATCAAACACCAACAACCCAGCATCCTCC
TAAGGAGGAGCTTCCATATTGGTGTACGTATGTAAAGAATTGCGACCTCTGTTGGGATTGTCAAGATAGCATCTATTGGAACA
AGGTAATAAGTGAGTCTATTTCGATAAATTCGATTATAAACTGTAGAGTTACTTGCGATTCCCAATCTCAGTCCTGCTTTTAT
GAAATATTACTTAAAATACCTAATCATCATAGCATGGAATGTTCTTATCCTGGATCGTATGAAAATGAAATGTTCATGGAAAA
ATGGAGGGATGAGAATTGGTCAATCATTATAAAACATTATTGTTTTTACCTTGTATTTAGCTTTGCATTTGCTGGTTGCGTTG
CATTTGCGATCTGCAAAAATCTACGACTGAGTACAACTATGAAATTACTTATGCTTTTGAGTATTTTGGTTTGCTTATCTCAG
CCAATCTTGAATAATTGA
Kenya 1950 (genotype X) 16240-15731 MGF 110 12L
SEQ ID No. 281
ATGAAGGTTTTTCTAGGACTTTTACTAGGTTTTTCAATCATCCTCATTCTTACATATCAATCACCGACAACCCAGCATCCTCC
TAAGGAAGAGCTTGCGTACTGGTGCACTTATGCAAAATCTTGTGACTTCTGCTGGGATTGCCAAAATGACACTTGTATAAATA
AGGTAATAAATGAGTCTATTTCGATAACTTCGATTGTAAACTGTAGAGTTACTCGCGATTCCCAGTCCTGTTTTTATGATATA
TCAGTTAAAATACCTAATCATCATAGCATGGAATGTTCATATCCTAGACTTTATGAACATGAAATGTTTATGGAAAAATGGAG
GGATGAGTATTGGCCAATCATTATAAAACAGTGTTGTTTTTACCTTGTATTTAGCTTTGCATTTGCTGGTTGCGTTGCATTTG
CGATCTGCAAAAACCTACGACTGCGTACAACTATAAAACTACTTATTCTTTTGAGTATTTTGGTTTGGTTATCTCAGCCAATC
TTGAATAATTGA
L60 (genotype I) 7993-7682 MGF 110 12L
SEQ ID No. 282
ATGAAGGTTTTTCTAGGACTTTTACTAGGTTATTCAACCATCCTCATTCTTACATATCAATCACCAACAACCCAGTGGTGTTT
TTATGAAATATCACTTAAAATACTTAATCATCATAGCATGGAAAAATGGAGGGATAAGAATTGGTCAATCATTATAAGGTATT
ATTGTTTTTACCTTGTATTTAGCTTTGCATTTGCTGGTTGCGTTGCATTTGCGATCTGCAAAAATCTACGACTGTGTACAACC
ATGAAATTACTTATGCTTTTGAATATTTTGGTTTTGTTATCTCAGCCAATTTTGAATAATTGA
Malawi Lil-20/1/1983 (genotype VIII) 12584-12069 MGF 110 12L
SEQ ID No. 283
ATGAAGGTTTTTCTAGGACTTTTACTAGGTTATTCAACCATCCTCATTCTTACATATCAATCACCAACAACCCAGCATCCTCC
TAAGGAAGAGCTTGAGTATTGGTGCACTTATGCAAAAACTTGTGACTTCTGCTGGGATTGCCAAAATGACACTTGTATAAATA
AGGTAATAAATGAATCTATTTCGATGAATTCGATTGTAAACTGTAGAGTTACTCGCGATTCCCAATCCCAGTCCTGTTTTTAT
GAAATATCACTTAAAATACCTAATTATCATAGCATGGAATGTTCATATCCTAGACTTTATAAACATTTCATGTCCATGGAAAA
ATGGAGGGATGAGAATTGGCCAATCCTTATAAGACATTATTGTTTTTACCTTGTATTTAGCTTTGCATTTGCTGGTTGCGTTG
CATTTGCGATCTGCAAAAATCTACGACTGCGTACAACCATGAAATTACTTATGCTTTTGAGTATTTTGGTTTTGTTATCTCAG
CCAATCTTGAATAATTGA
Mkuzi 1979 (genotype XII) 15894-15571 MGF 110 12L
SEQ ID No. 284
ATGAACGCAACCATGAAGGTTTTTCTAGGACTTTTACTAGGTTATTCAACCATCCTCATTCTTACATATCAATCACCGACAAC
CCAGTGGTGTTTTTATGAAATATCACTTAAAATACTTAATCATCATAGCATGGAAAAATGGAGGGATAAGAATTGGTCAATCA
TTATAAGGTATTATTGTTTTTACCTTGTGTTTAGCTTTGCATTTGCCGGTTGCGTTGCATTTGCGATCTGCAAAAATCTACGA
CTGTGTACAACCATGAAATTACTTATGCTTTTGGGTATTTTGGTTTTGTTATCTCAGCCAATCTTGAATAATTGA
Warmbaths (genotype III/l) 14212-13859 MGF 110 12L
SEQ ID No. 285
ATGAAGGTTTTTCTAGGACTTTTACTAGGTTATTCAACCATCCTCATTCTTACATATCAATCACCAGCAACCCAGTGGTGTTT
TTATGAAATATCACTTAAAATACCTAATCATCATAGCATGGAATGTTCATATCCTAGACTTTATAAACATTTCATATTCATGG
AAAAATGGAGGGATAAGAATTGGTCAATCATTATAAGGTATTATTGTTTTTACCTTGTATTTAGTTTTGCATTTGCTGGTTGC
ATTGCATTTGCGATCTGCAAAAATCTACGACTGTGTACAACTATGAAATTACTTATGCTTTTGAGTATTTTGGTTTTGTTATC
TCAGCCAATCTTGAATAATTGA
Warthog (genotype IV) 12897-12544 MGF 110 12L
SEQ ID No. 286
ATGAAGGTTTTTCTAGGACTTTTACTAGGTTATTCAACCATCCTCATTCTTACATATCAATCACCAGCAACCCAGTGGTGTTT
TTATGAAATATCACTTAAAATACCTAATCATCATAGCATGGAATGCTCATATCCTAGACTTTATAAACATTTCATGTTCATGA
AAAAATGGAGGGATAAGAATTGGTCAATCATTATAAGGTATTATTGTTTTTACCTTGTATTTAGTTTTGCATTTGCTGGTTGC
ATTGCATTTGCGATCTGCAAAAATCTACGACTGTGTACAACTATGAAATTACTTATGCTTTTGAGTATTTTGGTTTTGTTATC
TCAGCCAATCTTGAATAATTGA

In an embodiment the attenuated ASFV of the invention comprises a functional version of MGF 110 12L. Suitably the functional version of MGF 110 8L comprises the sequence of SEQ ID No. 276, 277, 278, 279, 280, 281, 282, 283, 284, 285 or 286. Suitably the functional version of MGF 110 12L comprises a sequence having at least 70%, at least 80%, at least 90% or at least 95% identity with SEQ ID No. 276, 277, 278, 279, 280, 281, 282, 283, 284, 285 or 286. Suitably the functional version of MGF 110 12L consists of the sequence of SEQ ID No. 276, 277, 278, 279, 280, 281, 282, 283, 284, 285 or 286.

### MGF 360 6L Gene Sequences

Georgia 2007/1 MGF 360 6L (NC_044959.1:17222-18349)
SEQ ID No. 51
TTACCGCAAATTGTCTCTCAGCGAAGAAAATGAATGAAACGTTTCTGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATCATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCCGTATTGATAT
TTGCGCCTAAGGCAAAACAATAGTATATACCCAATTTATTTTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGGTGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACGATTGTAAATAAAGTCTATCAACACGTAAAAA
TCATGGCTATCAAAACTGTCGAGAATCGAAATATTGTCATAATAAATATCTATAGCTAATAAGACCTTTTGTTGTTTAATTAG
ATCAACAAACATATTATACAACCCTACATCTAAAAATTTTGGATCAGCTCCTAGTTGAATACACAGAACTTTCGTCCTTTCCG
TCTTGGCACATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACATCAGAACATGACATAGAGCCACTGGTTACCATTTTACATTGTTT
ACAAAAACCTATGGGTCCGTTTTCCCACCATAATCCAAGCTGCTGTAAAATAAAAATATCATCCTCATGATAATTTGAAAAAG
CCTTGTTTTCTATCAAGACTTTTTTTGTAAGAACCTGTAAAGAATTCAT
China/2018/AnhuiXCGQ MGF 360 6L (MK128995.1:17221-18348)
SEQ ID No. 52
TTACCGCAAATTGTCTCTCAGCGAAGAAAATGAATGAAACGTTTCTGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATCATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCCGTATTGATAT
TTGCGCCTAAGGCAAAACAATAGTATATACCCAATTTATTTTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGGTGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACGATTGTAAATAAAGTCTATCAACACGTAAAAA
TCATGGCTATCAAAACTGTCGAGAATCGAAATATTGTCATAATAAATATCTATAGCTAATAAGACCTTTTGTTGTTTAATTAG
ATCAACAAACATATTATACAACCCTACATCTAAAAATTTTGGATCAGCTCCTAGTTGAATACACAGAACTTTCGTCCTTTCCG
TCTTGGCACATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACATCAGAACATGACATAGAGCCACTGGTTACCATTTTACATTGTTT
ACAAAAACCTATGGGTCCGTTTTCCCACCATAATCCAAGCTGCTGTAAAATAAAAATATCATCCTCATGATAATTTGAAAAAG
CCTTGTTTTCTATCAAGACTTTTTTTGTAAGAACCTGTAAAGAATTCAT
Warmbaths MGF 360 6L (AY261365.1:17236-18363
SEQ ID No. 53
TTACCGCAAATTGTCTCTCAGTAAAGAAAATGAATGAAACGTTTCTGTATATTCATAGGTTGAAATTATTTTACGCACTTCAC
CAGGTTCTAATATTTTTTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCAGTATTGATAT
TTGCACCTAAGGCAAAACAATAGTATATACCTAATTTATTCTGAGGGTACATACAAGCAAGCGGCATCATGTCATTTGGATCT
AAACGTATATTTTCCTGAAAATATGCATGATGGATTTCATCAACATTACCTAAGTATACAGCCGTTTTTAAACGCCAATAATC
TAGGTAAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAACACGTAAAAA
TCATGGCTATCAAACCTGTCGAGAATCGAAATATTATCATAATAAATATCTATAGCTAATAAGACCTTTTGTTGTTTAATTAG
ATCAACAAACATATTATACAACCCTACATCTAAAAATTTTGGATCAGCTCCTAGTTGAATACACAGAGCTTTCATCCTTTCTG
TCTTGGCGCATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACGTCAGAGCATGACATAGCGCCACTAGTTACCATTTTACATTGTTT
ACAAAAACCTATGGGACCGTTTTCCCACCATAGTCCAAGCTGCTGTAAAATAAAAATATCATCTTCATGATATTTTGAAAAAG
CCTTGTTTTCTATCAAGACTTTTTTTGTAAGAACCTGTAAAGAATTCAT
L60 MGF 360 6L (NC_044941.1:11041-12168)
SEQ ID No. 54
TTACCGCAAATTGTCTCTCAGCAAAGAAAATGAATGAAACGTTTCCGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCAGTATTGATAT
TTGCGCCTAAGGCAAAACAATAGTATATACCTAATTTATTCTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGATGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAATACGTAAAAA
TCATGGCTATCAAAACTGTCGAGAATCGAAATATTATCATAATAAATATCTATAGCTAATAAGACCTTTTGTTGTTTAATTAG
ATCAACAAACATATTATACAATCCTACATCTAAAAATTCTGGATTGGCTCCTAGTTCAATACACAGGTCTTTCGTCCTTTCCG
TCTTGGCACATATGATGCCATAATTAATGTTGGCACCCCATAAAACAAACAACTTGATTAGATCAGTCTGGTTTTTCTTCACA
GCCCTTACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCACAATCCAAGCTGCTGTAAAATAAAAATATCATCCTCATGATATTCTGAAAAAG
CCTTGTTTTCTATCAAGACTTTTTTTGTAAGAACCTGTAAAGAATTCAT
Benin 97/1 MGF 360 6L (NC_044956.1:10692-11819)
SEQ ID No. 55
TTACCGCAAATTGTCTCTCAGCAAAGAAAATGAATGAAACGTTTCCGTATATTCATAGGTTGAAATTATTTTACGCACTTCAC
TAGGTTCTAATATTTTCTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACCTATCAACGTCTCTAAGGCAGTATTGATAT
TTGCGCCTAAGGCAAAACAATAGTATATACCTAATTTATTCTGAGGGTACATACAAGCAAGCGACATCATGTCATTTGGATCT
AAACGTATATTTTCCTGAAAATATGCATGATGGATTTCATCAACATTACCTAAGTATACAGCCGTTTTTAAACGCCAATAATC
TAGATGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAATACGTAAAAA
TCATGGCTATCAAAACTGTCGAGAATCGAAATATTATCATAATAAATATCTATAGCTAATAAGACCTTTTGTTGTTTAATTAG
ATCAACAAACATATTATACAATCCTACATCTAAAAATTCTGGATTGGCTCCTAGTTCAATACACAGGTCTTTCGTCCTTTCCG
TCTTGGCACATATGATGCCATAATTAATGTTGGCACCCCATAAAACAAACAACTTGATTAGATCAGTCTGGTTTTTCTTCACA
GCCCTTACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCACAATCCAAGCTGCTGTAAAATAAAAATATCATCCTCATGATATTCTGAAAAAG
CCTTGTTTTCTATCAAGACTTTTTTTGTAAGAACCTGTAAAGAATTCAT
Pretorisuskop/96/4 MGF 360 6L (AY261363.1:17123-18250)
SEQ ID No. 56
TTACCGCAAATTGTCTCTCAGCAAAGAAAATGAATGAAACGTTTCCGTATATTCGTAGGTTGAAATTATTTTACGCACTTCAC
TAGGTTCTAATATTTTCTTATGAAGTATTGAATGGGGGCTTAAAAGTCCTTTCCTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCCATTTACTTCATGATTAATGTAACGTATCAATGTCTCTAAGGCAGTATTGATAT
TTGCGCCTAAGGCAAAACAATAGTATATACCTAATTTATTCTGAGGGTACATACAAGCAAGCGGCATCATGTCATTTGGATCT
AAACGTATATTTTCCTGAAAATATGCATGATGGATTTCATCAACATTACCTAAGTATACAGCCGTTTTTAAACGCCAATCATC
TAGGTGAGGAAATTTCTTACTAAGAAAACGAATAGGTTTTATAAGATTAAACTCTATGGCGATCTTAAACCAAAATTTTAATA
CATATGTATTTTTTATCATTTTTTCTTTTTCATCTAAATTTAAGATAAAACAATTGTAAATAAAGTCTATCAACACGTAAAAA
TCATGACTATCAAACCTGTCGAGAATCGAAATATTATCATAATAAATATCTATAGCTAATAAGACCTTTTGTTGTTTAATTAG
ATCAACAAACAAATTATACAACCCTACATCTAAAAATTTTGGATCAGCTCCTAGTTGGATACATAGAACTTTCGTCCTTTCCG
TCTTGGCACATATGATGCCATAATTAATGTTGGCACCCCATAAAACAAATAACTTGATTAGATCAGTCTGGTTTTTCTTCACA
GCCCTCACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCATAGTCCAAGCTGCTGTAAAATAAAAATATCATCTTCATGATAATTTGAAAAAG
CCTTGTTTTCTATCAAGACTTTTTTTGTAAGAACCTGTAAAGAATTCAT
Mkuzi 1979 MGF 360 6L (AY261362.1:18925-20052)
SEQ ID No. 57
TTACTGCAAATTGTCTCTCAGCAAAGAAAATGAATGAAACGTTTCTGTATATTCATAGGTTGAAATTATTTTACGCACTTCAT
TAGGTTCTATTATTTTCTTATGAAGTATTGAATGGGGGCTTAAAAGTCCTTTCTTAAAAAGAAGTTTCATTATAACATTCTTT
TCTTGTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTCCTTCATGATTAATGAAACCTATCAACGTCTCTAAGGCAGTATTGATAT
TTGCGCCTAAGGCAAAACAATAGTATATACCTAATTTATTCTGAGGGTACATACAAGCAAGCGACATCATGTCATTTGGATCT
AAACGTATATTTTCTTGAAAATATGCATGATGGATTTCATCAACATTACCTAAGTATACAGCCGTTTTTAAACGCCAATCATC
TAGGTGAGGAAATTTCTTACTAAGAAAACGAATAGGTTTTATAAGATTAAACTGTATGGCGAACTTAAACCAAAATTTTAATA
CAAGTGTATTTTTTGTCATTTCTTCTTTTTCATCTAAGTATAGGATAAAACGATTGTACATAAAGTCTATCAACACGTAAAAA
TCATGGCTATCAAAACTGTCGAGAATCGAAACATTATCATAATAAATATCTATAGCTAATAAGACCTTTTGTTGTTTAAGTAG
ATCAACAAACATATTATACAACCCTACATCTAAAAATTTTGGATCAGCTCCTAGTTGAATACACAGAACTTTCGTCCTTTCCG
TCTTGGCACATATGATGCCATAATTAATGTTGGCACCCCATAAAATAAATAACTTGATTAGATCAGTCTGGTTTTTCTTCACA
GCCCTCACCAAGGCTCTGTCAAGCTCATAGCTGTCAACGTCAGAGCATGACATAGAGCCACTGGTTACCATTTTACATTGTTT
ACAAAAACCTATGGGGCCATTATGCCACCATAGTCCAAGCTGCTGTAAAATAAAAATATCATCTTCATGATATTCTGAAAAAG
CCTTGTTTTCTATCAAGACTTTTTTTGTAAGAACCTGTAAAGAATTCAT
Ken05/Tk1 MGF 360 6L (NC_044945.1:19166-20293)
SEQ ID No. 58
TTATCGTAAATTGTCTCTTAGCGAAGAAAATGAATGAACCGTTTCCGTATATTGATAGGTTGAAATTATTTTACGCACTTCAC
TAGGTTCTAATATTTTCTTATGAAGTATTGAATGGGGGCTTAAAAGTCCTTTCTTAAAAAGTAGTTTCATTATAACATTCTTT
TCTTCTCTAAGAAGAGTTTCTTGTATTTTTTTTGTATAAGGATTGGCACCCAAACTTATACAAAAATGTACATTACTCCAAAT
ACCATAATTTGAAAAGAAAGTTATTTCCCTATTTACTTCATGATTAATGAAACGTATCAACGTCTCTAAGGCAGTATTGATAT
TTGCCCCTAAGGCAAAACAATAGTATATACCTAACTTATTTTGAGGGTACATACAAGCAAGCGACATCATGTTATTTGGATCT
AAACGTATATTTTCCTGAAAATATGCATGATGGATTTCATCAACATTACCTAAGTATACAGCCGTTTTTAAACGCCAATGATC
TAGGTGAGGAAATTTCTTACTAAGAAAACGAATAGGTTTTATAAGATTAAACTCTATCGCGAACTTATACCAAAATTTTAATA
CAAGTGTATTTCTTGTCATTTCTTCTTTTTCATCTAAATTTAAGATAAAACGATTGTAAATAAAGTCTATCAACGTGTAAAAA
TCATGGTCATCAAAACTATCGAGAATCGAAATATTATCATAATAAATATCTATAGCTAATAAGACTTTTTGTTGTTTAAGTAG
ATCAACAAACATATTATACAACCCTACATCTAAAAATTTTGGATCAGCTCCTAGTTGAAGACACAGAACTTTTGTCCTTTCCG
TCTTGGCACACATAATGCCATAATTAACGTTGGCACCCCATAAAACAAATAACTTGATTAGATCAGTCTGGTTTTTCTTCACA
GCCGTCACCAAGGCTCTGTCAAGCTCATAGCTGTCAACATCAGAGCATGACATAGAGCCACTGGTTACCATTTTACATTGTTT
ACAAAAACCTATGGGGCCATCATGCCACCACAACCCTAGCTGCTGTAAAATAAAAATATCATCCTCATGATAATTTGAAAAGG
CCTTGGTTTCTATCAAGACTTTTTTTGTAAGAACCTGTAAAGAATTCAT
Malawi Lil-20/1 (1983) MGF 360 6L (AY261361.1:15478-16605)
SEQ ID No. 59
TTACCGCAAATTGTCTCTCAGCGAAGAAAATGAATGAAACGTTTCCGTATATTCATAGGTTGAAATTATTTTACGCACTTCAC
CAGGTTCTAATATTTTTTTATGAAGTATTGAATGGGAGGTTAAAAGTCCTTTCTTAAAAAAGAGTTTCATTATAACATTCTTT
TCTTCTCTAAGAAGAGTTTCTTGTATTTTTTTTATATAAGGATTGGCACCCAAACTTATACAAAAATGTACATTACTCCAAAT
ACCATAATTTGAAAAGAAAGTTATTTCCCTATCTACTTCATGATTAATGAAACGTATCAACGTCTCTAAGGCAGTATTGATAT
TTGCACCTAAGGCAAAACAATAGTATATACCTAATTTATTTTGAGGGTACATACAAGCAAGCGACATCATGTGATTTGGATCT
AAACGTATATTTTCCTGAAAATATGCATGATGGATTTCATCAACATTACCTAAGTATACAGCCATTTTTAAACGCCAATCATC
TAGGTGAGGAAATTTCTTACTAAGAAAACGAATAGGTTTTATAAGATTAAACTCTATGGCGATCTTAAACCAAAATTTTAATA
CAAGTGTATTTCTTGTCATTTCTTCTTTTTCATCTAAGTATAGGATATAACGATTGTAAATAAAGTTTATCAATGCGTAAAAA
TCATGGCTATCAAAACTGTCGAGAATGGGAATGTTTTCATAATAAATATCTATAGCTAATAAGACCTTTTGTTGTTTAAATAG
ATCAACAAACATAGTATACAACCCTACCTCTAAAAATGTTGGATCAGCTCCTAGTTGAATACACAGAACTTTCGTCCTTTCCA
TCTTGGCACATATGATGCCATAATTGATGTTGGCACCCCATAAAACAAATAACTTGATTAGATCAGTCTGATTTTTCTTTACA
GCCTTCACCAAGGCTCTGTCAAGCTCATAGCTGTCAACGTCAGAGCATGACATAGAGCCACTGGTTACCATTTTACATTGTTT
ACAAAAACCTATAGGTCCGTTTTCCCACCATAATTCTAGCTGCTGTAAAATAAAAACATCATCTTCATGATAATTTGAAAAAG
CCTTGTTTTCTATCAAGACTTTTTTTGTAAGAACCTGTAAAGAATTCAT
Kenya 1950 MGF 360 6L (AY261360.1:19341-20462)
SEQ ID No. 60
TTACCGCACATTGTCTCTCAGCGAAGAAAATGAATGAAACGTTTCCGTATATTGATAGGTTGAAATTATTTTACGCACTTCAC
TAGGTTCTAATATTTTCTTATGAAGTATTGAATGGGGGCTTAAAAGTCCTTTCTTAAAAAGTAGTTTCATTATATCATACTTT
TCTTCTCTAAGAAGAATTTCTTGTATTTTTTTTGTATAAGGATTGGCACCCAAACTTATACAAAAATGTACATTACTCCAAAT
ACCATAATTTGAAAAGAAAGGTCTATTTAGTTCATGATTAATGAAACGTATCAACGCCTCTAAGGCAGTATTGATATTTGCCC
CTAAGGCAAAACAATAGTATATACCTAATTTATTCTGAGGATACATACAAGCAAGCGACATCATGTGATTTGGATCTAAACGT
ATATTTTCCTGAAAATATGCATGATGGATTTCATCAACATTCCCTAAGTATACAGCCGTTTTTAAACGCCAATCATCTAGGTG
AGGAAATTTCTTACCAAGAAAACGAATAGGTTTTACAAGATTAAACTCTATGGCAAACTTATACCAAAATTTTAATACAAGTG
TATTTTTTGTTATTTTTTCTTTTTCATCTAAATTTAAGATAAAACCATTGTAAATAAAGTCTATCAACACGTAAAAATCATGG
CTATCAAAACTGTCGAGAATCGAAATATTATCATAATAAATATCTATAGCTAATAAGACCTTTTGTTGTTTAATTAGATCAAC
AAACATATTATACAACCCTACATCTAAAAATTTTGGATCAGCTCCTAGTTGAATACATAGAACTTTCGTCCTTTCCGTCTTGG
CACACACGATGCCAAAATTAATTTCGGCGCCCCATAAAACAAATAACTTGATTAGATCAGTTTGGTTTTCCTTCACAGCTTTT
ACTAAGGCTCTGTCAAGCTCATAGCTGTCGACATCAGAACATGACATAGAGCCACCGGTTACCATTTTACATTGTTTACAAAA
ACCTATGGGTCCGTTTTCCCACCACAGTCCAAGCTGCTGTAAAATGAAACTATCATCTTCATGATAATTTGAAAAAGCCTTGT
TTTCTATCAAGACTTTTTTTGTAAGAACCTGTAAAGAGTTCAT
Ken06.Bus MGF 360 6L (NC_044946.1:15081-16208)
SEQ ID No. 61
TTACCGCAAATTATCTCTCAGTGAAGAAAATGAATGAAACGTTTCCGTATGTTCATAGGTTGAAATTATTTTACGCACCTCAC
TAGGTTCTAATATTTTTTTATGAAGTATTGAATGGGGGCTTAAAAGTCCTTTTTTAAAAAGTAGTTTCATTATAACATTCTTT
TCTTCTCTAAGAAGAGTTTCTTGTATTTTTTTTGTATCAGGGTTGGCACCCAAACTTATACAAAAATGTATATTACTCCAAAT
ACCATAATTTGAAAAGAAAGTTATTTCCCTATTTACTTCATGATTAATGAAACGTATCAACGTCTCTAAGGCAGTATTGATAT
TTGCGCCTAAGGCAAAACAATAGTATATACCTAATTTATTCTGAGGGTACATACAAGCAAACGACATCATGTGATTTGGATCT
AAACGTATATTTTCTTGAAAATATGCATGATGGATTTCATCAACATTCCCTAAGTATACAGCCGTTTTTAAACGCCAATCATC
TAGGTGAGGAAATTTCTTACTAAGATAACGGATAGGTTTTACAAGATTAAACTCTATGGCGAACTTAAACCAAAATTTTAATA
CAAATGTGTTTCTTGTCATTTTTTCTTTTTCATCTAAATTTAGGATAAAACGATTGTAAATAAAGTTTATCAACGCGTGAAAA
TCATGGCTATTAAAGCTGTCGAGAATCAAAATATTATCATAATAAATATCTATGGCTAATAAGACCTTTTGTTGTTTAATTAG
ATTAACAAACATATTATACAGCTTTTTATCTAAAAATTCTGGATCGGCTCCTAGTTGAATACACAGGTCTTTCGTCCTTTCCA
TCTTAGCACACATAATGCCATAATTAATGTTGGCACCCCATAAAACAAATAATATGATTAGATCAGTTTGGTTTTCCTTCACA
GCTTTTACTAAGGCCCTGTCAAGCTCATAGCTGTCAACATCAGAGCATGACATTGAGCCAATGGTTACTATTTTACATTGTTT
ACAAAAACCTATGGGGCCGTTTTCCCACCACAATCCTAGCTGCTGTAAAATAAAACTATCATCTTCATGATAATTTGAAAAGG
CCTTGTTTTCTATTAAGACTTTTTTTGTAAGAACCTGTAAAGAATTCAT

In an embodiment the attenuated ASFV of the invention comprises a functional version of MGF 360 6L. Suitably the functional version of MGF 360 6L comprises the sequence of SEQ ID No. 51, 52, 53, 54, 55, 56, 57, 58, 59, 60 or 61. Suitably the functional version of MGF 360 6L comprises a sequence having at least 70%, at least 80%, at least 90% or at least 95% identity with SEQ ID No. 51, 52, 53, 54, 55, 56, 57, 58, 59, 60 or 61. Suitably the functional version of MGF 360 6L consists of the sequence of SEQ ID No. 51, 52, 53, 54, 55, 56, 57, 58, 59, 60 or 61.

### MGF 360 10L Gene Sequences

Georgia 2007/1 MGF 360 10L (LR743116.1:26373-27410)
SEQ ID No. 62
CTAGGCCAACATACTTTTTAATTTATAGTTTTTTAATAGATGATATATTTTGTTAGGATCTGCTTCTTTTAACGTTAATAGCG
AGGAGTCTGCACTATAAATGTCTAATGATAAATGATGAGATATCAAATAGTAATTCCGTTGCTCTGCTAGGGCCTTTGCCTCT
TCAAAGGCGTCGGCTCCCAGATCTATACAAAAGAACAAGTTATCCATATTATAAAATCGTACGCAGGCAAGCATAGCTGAATT
AATATTAGCTCCTAAGAGAAAACAATAATATATGGTTAAAAAATTGTTATCTTTTGTGCAGGCCATCCGCATCATTTCATCCA
CGTCCATGCGGATCTTTTCCTTTTCATACAAATTATGTAGGTCAAACAGCTTATTAAAACAAAGAGCACAGATTAACCACCAC
GTATTTAGATACTTAAAATGTTGGTAAACATAAGAAATGGCCTCCCTAAGATTATCCTGCAATGCCACTATAAAACAGTATAT
CGTTAACATATCACCATCCGACATATTACTTAATATGTCGGTGTCTTCTACTAACCTTTTCAACTTCCAATATATGGATGACC
TTATTTCCCTTATAATGACATAGGCTGGAAAGGGATTATCATTAAAAAGTTTAAGACATAAGATAATATTACTGCTAGTAGTG
CCAGGGTGTATTAATTTAAAGAACATGTGCATAATCTTCTTTTTATCCACGCGGTACTTGGCTCCTAATTCCCAGCAAAATTC
TCGAACAGGCGGCGTATTGGCGCAAATTAACCCATAGTTGATGTCTGCGCCCCATTCTGTAAACAGTTTTATTAACTGATAGT
TGTTTTCCTTTGTAGCCAACATTAGTGCCGTATTAAGGTCCAAGCCGTCTGCAAAGCTTGGCAGCTTTATCAGCATATGTTTG
CAATCAAGGGAAATTGGGGCCTTATACCACCATAGTCCGCAGCGTTCTAAGATAACATGGTACTCAATAGATACTTGCTGTCT
GGCTAGTACCTTTTTGGCGAAGGATTGTAAGGAAGGAAACAT
China/2018/AnhuiXCGQ MGF 360 10L (MK128995.1:25402-26439)
SEQ ID No. 63
CTAGGCCAACATACTTTTTAATTTATAGTTTTTTAATAGATGATATATTTTGCTAGGATCTGCTTCTTTTAACGTTAATAGCG
AGGAGTCTGCACTATAAATGTCTAATGATAAATGATGAGATATCAAATAGTAATTCCGTTGCTCTGCTAGGGCCTTTGCCTCT
TCAAAGGCGTCGGCTCCCAGATCTATACAAAAGAACAAGTTATCCATATTATAAAATCGTACGCAGGCAAGCATAGCTGAATT
AATATTAGCTCCTAAGAGAAAACAATAATATATGGTTAAAAAATTGTTATCTTTTGTGCAGGCCATCCGCATCATTTCATCCA
CGTCCATGCGGATCTTTTCCTTTTCATACAAATTATGTAGGTCAAACAGCTTATTAAAACAAAGAGCACAGATTAACCACCAC
GTATTTAGATACTTAAAATGTTGGTAAACATAAGAAATGGCCTCCCTAAGATTATCCTGCAATGCCACTATAAAACAGTATAT
CGTTAACATATCACCATCCGACATATTACTTAATATGTCGGTGTCTTCTACTAACCTTTTCAACTTCCAATATATGGATGACC
TTATTTCCCTTATAATGACATAGGCTGGAAAGGGATTATCATTAAAAAGTTTAAGACATAAGATAATATTACTGCTAGTAGTG
CCAGGGTGTATTAATTTAAAGAACATGTGCATAATCTTCTTTTTATCCACGCGGTACTTGGCTCCTAATTCCCAGCAAAATTC
TCGAACAGGCGGCGTATTGGCGCAAATTAACCCATAGTTGATGTCTGCGCCCCATTCTGTAAACAGTTTTATTAACTGATAGT
TGTTTTCCTTTGTAGCCAACATTAGTGCCGTATTAAGGTCCAAGCCGTCTGCAAAGCTTGGCAGCTTTATCAGCATATGTTTG
CAATCAAGGGAAATTGGGGCCTTATACCACCATAGTCCGCAGCGTTCTAAGATAACATGGTACTCAATAGATACTTGCTGTCT
GGCTAGTACCTTTTTGGCGAAGGATTGTAAGGAAGGAAACAT
Ken06.Bus MGF 360 10L (NC_044946.1:23437-24507)
SEQ ID No. 64
TTACGACGTTGTATCATTGATGTGATAATCCAAATAGGCCAACATACTTTTTGATTTATAGTTGTTTAATAGATGGTATATTT
TTTTAGGGTTCGCTTCTTTTAGCGTGAATAGAGAGGGATCTGGATGATAGATATCTAATGACAAACGTTGTGATATTAAATAA
TAGTCCCGCTGCTCTGCTAGGGCTTTTGCCTCTTCGAAGGCATTGGCTCCCAAGTCTATACAAAAGAACAGATTATCTATATT
AAAGAACTGTATGGCAGCAAGCATGGCCATATTGATATCGGCCCCTAAAATAAAACAATAATACATCGTTAAAAAGTTATTAT
CTTTCGTGCAAGCTATGCGCATCATTTCATTAATGTCTATGCGAATCTTTTCCTGCTCATAGAGATAATGGAGGTCAGACAGT
TTATTAAAATACAGAGCACAGATTAACCACCACGTATTCATGTGCTTAAAATGTTCGTAAAAATACGAGACGGCCTCCCTAAG
ATCATTCTGCAAGGCCACCATAAAACAATATATAGTTAGCATATCGCCGTCCGAAACATCACGTAGTAGGTCTTCATCTTCTA
CTAACCTCCTCAGCCTCCAATAAAGAGACGACTTTATTTCCCTTATAATCACATAGGTAGGAAAAGGATTCTCATTAAAAAGC
TTATGACATAAGATAATATATTGACTTGTTTTGTTCTCTATAAATTTAAAGAACATGTGTATCACCTTTTTTTTATCCATGCG
ATAGTTCGCTCCTAATTCCCAACAAAACTCTCGGGTAGGCTGTGTATTGGCACAAATGTATCCATAGTTGATGTTTGCACCCC
ACTCCGTGAATAGTTTTATTAATTGATAGTTGTTTTCCTTTGCAGCTATCATCAATGCCGTATTGAGATCCAGGCCATCCGCA
AAGTATGGCAACCTTATTAACATGTGTTTGCAATCAAGTGAAATTGGAGCCGTATACCACCATAACCCGCAGTATTTCAAAAT
ATGATAGTAGTCTTCAGGCAGCACATGCTGACTGGCTAATATCTTTTTGGCGAAAGACTGTAAAGAAGGAAACAT
Ken05/Tk1 MGF 360 10L (NC_044945.1:27310-28380)
SEQ ID No. 65
TTACGACGTTGTATCATTATTGTGATAATCCAAATAGGCCGACATACTTTTTGATTTATAATTGTTTAATAGATGGTATATTT
TTTTAGGGTTCGCTTCTTTTAGCGTGAATAGAGAGGGATCTGGATGATAGATATCTAATGACAAACGATGTGATATTAAATAA
TAGTCCCGCTGCTCTGCTAGGGCTTTTGCCTCTTCGAAGGCATTGGCCCCCAAGTCTATACAAAAGAACAGATTATCTATATT
AAAAAACTGTATGGCAGCAAGCATCGCCATATTGATATCGGCCCCTAAAATAAAACAATAATACATCGTTAAAAAGTTGTTAT
CTTTCGTGCAAGCTATGCGCATCATTTCATTAATGTCTATGCGAATTTTTTCCTGCTCATAGAGATAATGGAGGTCAAACAGT
TTATTAAAACACAGAGCACAGATTAACCACCACGTATTCAGGTGCTTAAAATGTTGGTAAAAATACGAGACGGCCTCCCTAAG
ACCATTCTGCAAGGCCACCATAAAACAATATAAAGTTAGCATATCGCCGTCCGAAACATCGCGTAGTAGGTCTTCGTCTTCTA
CTAACCTCCTCAGCCTCCAATAAAGAGACGACTTTATTTCCCTTATAATCACATAGGTAGGAAAAGGATTCTCATTAAAAAGC
TTATGACATAAGATAATATATTGACTCGTTTTGTTCTCTATAAATTTAAAAAACATATGCATCACCTTTTTTTTATCTAAGCG
ATAGTTCGCTCCTAATTCCCAACAAAACTCTCGAGTAGGCTGTGTATTGGCACAAATGTATCCATAGTTGATGTTTGCACCCC
ACTCCGTGAACAGTTTTATTAATTGATAGTTGTTTTCCTTTGCAGCTATCATCAATGCCGTATTGAGGTCCAGGCCATCCGCA
AAGTATGGCAACCTTATTAACATGTGTTTACAATCAAGTGAAATTGGAGCCTTATACCACCATAACCCGCAGCGCTTCAAAAT
ATGATAGTAGTCTTCAGGCAGCACATGCTGGCTGGCTAATATCTTTTTGGCGAAAGATTGCAGGGAAGGAAACAT
Kenya 1950 MGF 360 10L (AY261360.1:29558-30628)
SEQ ID No. 66
TTACGACGTTGTATCATTAATGTGATAATCCAAATAGGCCGACATACTTTTTGATTTATAGTTGTTTAATAGATGGTATATTT
TTTTAGGGTTCGCTTCTTTTAGCGTGAATAGAGAGGGATCTGGATGATAGATATCTAATGACAAACGATGTGATATTAAATAA
TAGTCCCGCTGCTCTGCTAGGGCTTTTGCCTCTTCGAAGGCATTGGCCCCCAAGTCTATACAAAAGAACAGATTATCCATATT
AAAGAACTGTATGGCAGCAAGCATGGCCATATTGATATCGGCCCCTAAAATAAAACAATAATACATCGTTAAAAAGTTGTTAT
CTTTCGTGCAAGCTATGCGCATCATTTCATTAATGTCTATGCGAATTTTTTCCTGCTCATAGAGATAATGGAGGTCAAACAGT
TTATTAAAACACAGAGCACAGATTAACCACCACGTATTCAGGTGCTTAAAATGTTGGTAAAAATACGAGACGGCCTCCCTAAG
ATCATTCTGCAAGGCCACCATAAAACAATATAAAGTTAGCATATCGTCGTCCGAAACATCGCGTAGTAGGTCTTCGTCTTCTA
CTAATCTCCTCAGCCTCCAATAAAGAGACGACTTTATTTCCCTTATAATCACATAGGTAGGAAAAGGATTCTCATTAAAAAGC
TTATGACATAAGATAATATACTGACTCGTTTTGTTCTCTATAAATTTAAAAAACATATGCATCACCTTTTTTTTATCTAAGCG
ATAGTTTGCTCCTAATTCCCAACAAAACTCTCGAGTAGGCTGTGTATTGGCACAAATGTATCCATAGTTGATGTTTGCACCCC
ACTCCGTGAACAGTTTTATTAATTGATAGTTGTTTTCCTTTGCAGCTATCATCAATGCCGTATTGAGGTCCAGGCCATCCGCA
AAGTATGGCAACCTTATTAACATGTGTTTACAATCAAGTGAAATTGGAGCCTTATACCACCATAACCCGCAGCGCTTCAAAAT
ATGATAGTAGTCTTCAGGCAGCACATGCTGGCTGGCTAATATCTTTTTGGCGAAAGATTGCAGGGAAGGAAACAT
L60 MGF 360 10L (NC_044941.1:19956-21026)
SEQ ID No. 67
TTATAACGATGTATCATTGATGTCATAATTCAAATAGGCCAACATACTTTTTGATTTATAGTTTTTTAATAGACGATATATTT
TGTTAGGATCTGCTTCTTTTAACGTTAATAGCGAGGAGTCTGGACTATAAATGTCTAATGATAAACGATGAGATATCAAATAG
TAATTCTGTTGCTCTGCCAAGGCTTTTGCCTCTTCAAAGGCATCGGCCCCCAGATCTATACAAAAGAACAGGTTATCCATATT
ATAGAATCGTATGGAGGCAATCATGGCCAAATTAATATTAGCTCCTAAGATAAAACAATAATATATAGTTAAAAAATTGTTAT
CTTTTGTGCAGGCTATCCGCATCATTTCATCCATGTCCATACGGATTTTTTCTTTTTCGTACAAATTATGTAGGTCAAACAGC
TTATTATAGCAAAGAGCACATGTTAACCACCACGTATTCAGATACTTAAAATGTTGGTAAACATAAGAAATGGCCTCCCTAAG
ATTATCCTGCAATGCCACGATAAAACAGTATATAGTTAACATATCACCATCCGAAATATTACTTAATACGTTGGTATCTTCTG
CTAAATTTTTTAGCTTCCAATGTATACACGACTTTATTTCCCTTATAATGACATAGGCTGAAAAGGGATTGTCATTAAAAAAT
TTAAGACATAAGATAATATTATTACTAGTAGTGTTAGGATGTATTAATTTAAAGAATATGTGCATAATCTTCTTTTTATCCAC
TTGGTACTTGGCTCCTAATTCCCAGCAAAATTCTCGAATAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAACATGGTAATCAATAGATACATGCTGACTGGCTAATACCTTTTTGGCGAAGGATTGCAAGGAAGGAACCAT
Benin 97/1 MGF 360 10L (NC_044956.1:19607-20677)
SEQ ID No. 68
TTATAACGATGTATCATTGATGTCATAATTCAAATAGGCCAACATACTTTTTGATTTATAGTTTTTTAATAGACGATATATTT
TGTTAGGATCTGCTTCTTTTAACGTTAATAGCGAGGAGTCTGGACTATAAATGTCTAATGATAAACGATGAGATATCAAATAG
TAATTCTGTTGCTCTGCCAAGGCTTTTGCCTCTTCAAAGGCATCGGCCCCCAGATCTATACAAAAGAACAGGTTATCCATATT
ATAGAATCGTATGGAGGCAATCATGGCCAAATTAATATTAGCTCCTAAGATAAAACAATAATATATAGTTAAAAAATTGTTAT
CTTTTGTGCAGGCTATCCGCATCATTTCATCCATGTCCATACGGATTTTTTCTTTTTCGTACAAATTATGTAGGTCAAACAGC
TTATTATAGCAAAGAGCACATGTTAACCACCACGTATTCAGATACTTAAAATGTTGGTAAACATAAGAAATGGCCTCCCTAAG
ATTATCCTGCAATGCCACGATAAAACAGTATATAGTTAACATATCACCATCCGAAATATTACTTAATACGTTGGTATCTTCTG
CTAAATTTTTTAGCTTCCAATGTATACACGACTTTATTTCCCTTATAATGACATAGGCTGAAAAGGGATTGTCATTAAAAAAT
TTAAGACATAAGATAATATTATTACTAGTAGTGTTAGGATGTATTAATTTAAAGAATATGTGCATAATCTTCTTTTTATCCAC
TTGGTACTTGGCTCCTAATTCCCAGCAAAATTCTCGAATAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAACATGGTAATCAATAGATACATGCTGACTGGCTAATACCTTTTTGGCGAAGGATTGCAAGGAAGGAACCAT
Mkuzi 1979 MGF 360 10L (AY261362.1:27069-28139)
SEQ ID No. 69
TTATAACGATGTATCATTGATGTCATAATTCAAATAGGCCAACATACTTTTTGATTTATAGTTTTTTAATAGACGATATATTT
TGTTAGGATCTGCTTCTTTTAACGTTAATAGCGAGGAGTCTGGACTATAAATGTCTAATGATAAACGATGAGATATCAAATAG
TAATTCTGTTGCTCTGCCAAGGCCTTTGCCTCTTCAAAGGCATCGGCCCCCAGATCTATACAAAAGAACAGGTTATCCATATT
ATAGAATCGTATGGAGGCAATCATGGCCAAATTAATATTAGCTCCTAAGATAAAACAATAATATATAGTTAAAAAATTGTTAT
CTTTTGTGCAGGCTATCCGCATCATTTCATCCATGTCCATACGGATTTTTTCTTTTTCGTACAAATTATGTAGGTCAAACAGC
TTATTATAGCAAAGAGCACATGTTAACCACCACGTATTCAAATACTTAAAATGTTGGTAAACATAAGAAATGGCCTCCCTAAG
ATTATCCTGCAATGCCACGATAAAACAGTATATAGTTAACATATCACCATCCGAAATATTACTTAATACGTTGGTATCTTCTG
CTAAATTTTTTAGCTTCCAATGTATACACGACTTTATTTCCCTTATAATGACATAGGCTGAAAAGGGATTGTCATTAAAAAAT
TTAAGACATAAGATAATATTACTACTAGTAGTGCTAGGATGTATTAATTTAAAGAATATGTGCATAATCTTCTTTTTATCCAC
TTGGTGCTTGGCTCCTAATTCCCAGCAAAATTCTCGAATAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAACATGGTGATCAATAGATACATGCTGACTGGCTAATACCTTTTTGGCGAAGGATTGCAAGGAAGGAAACAT
Pretorisuskop/96/4 MGF 360 10L (AY261363. 1:26116-27186)
SEQ ID No. 70
TTATAACGTTGTATTATTGACGTCATAATTCAAATAGGCCAATATACTTTTTGATTTATAGTTTTTTAATAGATGATATATTT
TGTTAGGATCCGCTTCTTTTAACGTTAATAACGAGGAATCTGGACTATAAATGTCTAATGATAAACAATGAGATATCAAAAAG
TAATTCCGTTGCTCTGCCAGGGCTTTTGCCTCCTCAAAGGCATCGGCCCCCAGGTCTATACAAAAGAATAAGTTATCTATGTT
ATAGAATTGTATGGAGGCAATCATAGCTAAATTAATATTAGCTCCTAAGATAAAACAATAATATATGGTTAAAAAATTGTTAT
CTTTTGTGCAGGCTATCCGCATCATTTCATCCATGTCCATGCGGATCTTTTCCTTTTCATACAAATGATGTAGGTCAAACACC
TTATTATAGCAAAGAACACATGTTAACCACCACGTATTCAGATACTTAAAATGTTGGTAAACATAAGAAATGGCCTCCCTAAG
ATTATCCTGCAATGCCACGATAAAACAGTATATAGTTAACATATCTCCATCCGAAATATTACTTAATACGTTGGTATCTTCTG
CTAACTTTTTTAGCTTCCAATGTATACACGACTTTATTTCCCTTATAATGACATAGGCTGAAAAGGGATTGTCATTAAAAAGT
TTAAGACATAAGATAATATTACTACTAGTAGTGCTAGGATGTATTAATTTAAAGAATATGTGCATAATCTTCTTTTTATCCAC
TTGGTACTTGGCTCCTAATTCCCAGCAAAATTCTCGAACAGGTGGCGTATTCGCGCAAATTAACCCATAGTTGATGTCTGCGC
CCCAATCCGTAAACATTTTTATTAATTGATAGTTGTTTTCCTTTGTAGCCAACATTAGTGCCGTATTAAGGTCCAAGCCATCT
GCAAAGTTTGGCAGCTTTATCAGCATATGTTTGCAATCAAGTGAAATTGGAGCCTTATACCACCATAGCCCGCAGCGTTCTAA
GATAATATGGTGATCAATGGATACATGCTGTCTGGCTAGTACCTTTTTGGCGAAGGATTGTAAGGAAGGAAACAT
Tengani 62 MGF 360 10L (AY261364.1:20530-21600)
SEQ ID No. 71
TTATAACAATGTATCATTGATATCATCATTCAAATAGGCCAACATATTTTTTGATTTATAGTTTTTTAATAGATGATTTATTT
TGTTAGGATCTGTTTCTTTTAACGTTAATAGCAAGGAGTCTGGCTTATAAATGTCTAATGATAAACGATGAGATATTAAATAG
TAATTCCGTTGCTCTGCCAGGGCTTTTGCCTCTTCAAAGGCGTCGGCTCCCAGATCTATACAAAAGAACAAGTTATCCATATT
ATAAAATCGTACGCAGGCAAGCATAGCTGAATTAATATTAGCTCCTAAGAGAAAACAATAATATATAGTTAAAAAATTGTTAT
CTTTTGTGCAGGCCATCCGCATCATTTCATCCACGTCCATGCGGATCTTTTCCTTTTCATACAAATTATGTAGGTCAAACAGC
TTATTAAAACAAAGAGCACAGATTAACCACCACGTATTTAGATACTTAAAATGTTGGTAAACATAAGAAATGGCCTCCCTAAG
ATTATCCTGCAATGCCACTATAAAACAGTATATCATTAACATATCACCATCCGACATATTACTTAATATGTCGGTGTCTTCTA
CTAACCTTTTCAGCTTCCAATATATGGATATCCTTATTTCCCTTATAATGACATAGGCTGGAAAGGGATTATCATTAAAAAGT
TTAAGACATAAGATAATATTACTGCTAGTAGTGCCAGGATGTATTAATTTAAAGAACATGTGTATAATCTTCTTTTTATCCAC
GCGGTGCTTGGCTCCTAATTCCCAGCAAAATTCTCGAACAGACGGCGTATTGGCGCAAATTAACCCATAGTTGATGTCTGCGC
CCCATTCCGTAAACAGTTTTATTAACTGATAGTTATTTTCCTTTGTAGCCAACATTAGTGCCGTATTAAGGTCCAAGCCGTCT
GCAAAGCTTGGCAGCTTTATCAGCATATGTTTGCAGTCAAGTGAAATTGGAGCCTTATACCACCATAGTCCGCAACGTTCTAA
GATAACATGGTACTCAATAGATACTTGCTGTCTGGCTAGTACCTTTTTGGCGAAGGATTGTAAGGAAGGAAACAT
Warthog MGF 360 10L (AY261366.1:22069-23130)
SEQ ID No. 72
TCATATTGTATCATCATGGTTAAAATATGCCGTCATATTTTTTGATTTATAGTTTTTTAATAGATGATATATTTTGTTAGGGT
CCGCTTCTTTTAACGTTAATAGCGAGGAGTCTGGACTATAAATGTCTAATGATAAACGATGAGATATCAAAAAGTAATTCCGT
TGCTCTGCCAGGGCTTTTGCCTCTTCAAAGGCATCGGCCCCCAAGTCTATACAAAAGAACAGGTTATCCATATTATAAAATCG
TATGGAGGCAATCATGGCCAAATTAATATTAGCTCCTAAGATAAAACAATAATATATAGTTAAAAAATTGTTATCTTTTGTGC
AGGCTATCCGCATCATTTCATCCATGTCCATGCGGATTTTTTCCTTTTCGTACAAATTATGTAGGTCAAACAGCTTATTAAAA
CAAAGAGCACAGATTAGCCACCACGTATTCAGATATTTAAAATGTTGGTAAACATAAGAAATGGCCTCCCTAAGATTATCCTG
CAATGCCACCATAAAACAGTATATCGTTAACATATCACCATCCGAAATATTACTTAATACGTCGGTGTCTTCTACTAACTTTT
TCAGCTTCCAATATATGGATGACTTTATTTCCCTTATAATGACATAGGCTGAAAAGGGGTTATCATTAAAAAGTTTAAGACAT
AAGATAATATTACTGCTAGTAGTGCCAGGATGTATTAATTTAAAGAACATGTGCATAATCTTCTTTTTATCCACGCGGTACTT
GGCTCCTAGTTCCCAGCACAATTCTCGAACAGGCGGCGTATTGGCGCAAATTAACCCATAGTTGATGTCTGCGCCCCATTCCG
TAAACAGTTTTATTAACTGATAGTTGTTTTCCTTTGTAGCCAACATTAGTGCCGTATTAAGGTCCAAGCCATCTGCAAAGCTT
GGCAGCTTTATCAGCATATGTTTGCAATCAAGGGAAATTGGAGCCTTATACCACCATAGTCCGCAGCGTTCTAAGATAACATG
GTAATCAATAGATACATGCTGTCTGGCTAGTACCTTTTTGGCGAAGGATTGTAA
GGAAGGAAACAT
Warmbaths MGF 360 10L (AY261365.1:25300-26361)
SEQ ID No. 73
TCATATTGTATCATCATGGTTAAAATATGCCGTCATATTTTTTGATTTATAGTTTTTTAATAGATGATATATTTTGTTAGGGT
CCGCTTCTTTTAACGTTAATAGCGAGGAGTCTGGACTATAAATGTCTAATGATAAACGATGAGATATCAAAAAGTAATTCCGT
TGCTCTGCCAGAGCCTTTGCCTCTTCAAAGGCGTCGGCCCCCAGGTCTATACAAAAGAACAGGTTATCCATATTATAGAATCG
TATGGAGGCAATCATGGCCAAATTAATATTAGCTCCTAAGATAAAACAATAATATATAGTTAAAAAATTGTTATCTTTTGTGC
AGGCTATCCGCATCATTTCATCCATGTCCATGCGGATTTTTTCCTTTTCGTACAAATTATGTAGGTCAAACAGCTTATTAAAA
CAAAGAGCACAGATTAGCCACCACATATTCAGATACTTAAAATGTTGGTAAACATAAGAAATGGCCTCCCTAAGATTATCCTG
CAATGCCACCATAAAACAGTATATCGTTAACATATCACCATCTGAAATATCACTTAATACGTCGGTGTCTTCTACTAACTTTC
TCAGCTTCCAATATATGGATGACTTTATTTCCCTTATAATGACATAGGCTGAAAAGGGGTTATCATTAAAAAGTTTAAGACAT
AAGATAATATTACTGCTAGTAGTGCCAGGATGTATTAATTTAAAGAACATGTGCATAATCTTCTTTTTATCCACGCGGTACTT
GGCTCCTAGTTCCCAGCACAATTCTCGAACAGGCGGTGTATTGGCGCAAATTAACCCATAGTTGATGTCTGCGCCCCATTCCG
TAAACAGTTTTATTAACTGATAGTTGTTTTCCTTTGTAGCCAACATTAGTGCCGTATTAAGGTCCAAGCCATCTGCAAAGCTT
GGCAGCTTTATCAGCATATGTTTGCAATCAAGTGAAATTGGAGCCTTATACCACCATAGTCCGCAGCGTTCTAAGATAACATG
GTAATCAATAGATACATGCTGTCTGGCTAGTACCTTTTTGGCGAAGGATTGTAA
GGAAGGAAACAT
Malawi Lil-20/1 (1983) MGF 360 10L (AY261361.1:23735-24802)
SEQ ID No. 74
TTACACTGTGTCATGCCCATCATAGTCAAAATATACCATCATATTTTTTGATTTATAGTTTTTTAATAGATGATATATTTTTT
TAGGATCTGCTTCTTTTAACGTTAATAGCGAGGAGTCTGGACTATAAATATCTATTGATAAACGATGATACATCAAAAAGTAA
TTCCGCTGGTCTGCCAGGGCTTTTGCCTCTTCAAAGGCATCGGCTCCCAGGTCTATACAAAAGAACAAGTTATCCATATTATA
GAATTGTACGCAGGCAAGCATAGCCTGATTAATATTAGCTCCTAAGAGAAAACAGTAATATATGGTTAAAAAGTTGTTATCTT
TAGTGCAGGCTATGCACATCATTTCATCCATGTCCATGCGGATCTTTTCCTTTTCATACAAATCATGTAGGTCAAACAGCTTA
TTAAAACAAAGAGCACAGATTAACCACCACGTATTCAGATGCTTAAAATGTTGGTAAAAATAAGAAATGGCCTCCCTAAGATT
ATCCTGCAATGCCAGGATAAAACAGTATATAGTCAACATATCATCATCCGACATATTACTTAATATGTCAGTGTCTTCTACTA
ACCTTCTCAGCTTCCAATATATAGACGACTTTATTTCCCTTATAATGACATAGGTTGGAAAAGGATTATTATTAAAAAGTTTA
AGACATAAGATAATATTACTACTAGTAGTGCCATGATGTATTAATTTAAAAAACATATGCATAACCTTATTTTTATCCACTTG
GTACTTGGCTCCTAATTCCCAGCAAAATTCTCGAACAGGCGGCGTATTAGCGCAAATTAATCCATAGTTGATGTCCGCGCCCC
ATTCCGTAAACAGTTTTATTAACTGATAGTTGTTTTCCTTTGTAGCCAACATTAGTGCCGTATTAAGGTCCAAGCCATCTGCA
AAGCTTGGCAGCTTTATTAACATATGTTTGCAATCAAGTGAAATTGGAGCCTTATACCACCATAGTCCGCAATGTTTTAAGAT
ATAATGATAATCAATGGATACATGATGTCTAGCTAATACCTTTTTGGCGAAGGATTGTAAAGAAGGAAACAT

In an embodiment the attenuated ASFV of the invention comprises a functional version of MGF 360 10L. Suitably the functional version of MGF 360 10L comprises the sequence of SEQ ID No. 62, 63, 64, 65, 66, 67, 68, 69, 70, 71, 72, 73 or 74. Suitably the functional version of MGF 360 10L comprises a sequence having at least 70%, at least 80%, at least 90% or at least 95% identity with SEQ ID No. 62, 63, 64, 65, 66, 67, 68, 69, 70, 71, 72, 73 or 74. Suitably the functional version of MGF 360 10L consists of the sequence of SEQ ID No. 62, 63, 64, 65, 66, 67, 68, 69, 70, 71, 72, 73 or 74.

### MGF 360 11L Gene Sequences

Georgia 2007/1 MGF 360 11L (NC_044959.1:26465-27526)
SEQ ID No. 75
TTATCTTTGTTCATAATCAAGAAAAATCCCCATATTTATTGAGTGATAATTTTTTAACATGCAATTTATTTTTTCAGGGTCCG
TAACGATCGACAACAGAGAAATAACCGGATTGTAATGCTTTAATGATAAGGCATGGGCTATCAGATAATTTTCCTTTTGTTCT
GCCAAAGCTTTGCCCTCCTCAAAGGCATCGGCACCCAGGTCTATACAAAAGAACAGGTTTCCAAGATTATAGTTTTGTATGGA
AACAAGCATGGCTTGATTGATGTTGGCTCCCATGATAAAACAGTAGTAAATGGCCGAATAGCTATAATCTTGGATGCAGGCTA
TGTGCATCATTTCATCAATATCCATGCGGACCCTTTCTATTTCGTACAGCTCGTGAAGGTCGAACACGTTGTTGTAAAAAAGG
GCGCACATGAGCCGCCACCTATGTAGACGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAACAGGTTGAC
CTTATAAATAATTTATTGCGTCGAATATGAAGCATAATGGGGCCATTATGCCACCACAGGCCACAACACTTCAGGACATGATA
TTGATCTACCGGTATACACTGCCCGGCCAGTACTTTCTTCGTGAGGGATTGCAGGGAAGGCAACAT
China/2018/AnhuiXCGQ MGF 360 11L (MK128995.1:26467-27528)
SEQ ID No. 76
TTATCTTTGTTCATAATCAAGAAAAATCCCCATATTTATTGAGTGATAATTTTTTAACATGCAATTTATTTTTTCAGGGTCCG
TAACGATCGACAACAGAGAAATAACCGGATTGTAATGCTTTAATGATAAGGCATGGGCTATCAGATAATTTTCCTTTTGTTCT
GCCAAAGCTTTGCCCTCCTCAAAGGCATCGGCACCCAGGTCTATACAAAAGAACAGGTTTCCAAGATTATAGTTTTGTATGGA
AACAAGCATGGCTTGATTGATGTTGGCTCCCATGATAAAACAGTAGTAAATGGCCGAATAGCTATAATCTTGGATGCAGGCTA
TGTGCATCATTTCATCAATATCCATGCGGACCCTTTCTATTTCGTACAGCTCGTGAAGGTCGAACACGTTGTTGTAAAAAAGG
GCGCACATGAGCCGCCACCTATGTAGACGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAACAGGTTGAC
CTTATAAATAATTTATTGCGTCGAATATGAAGCATAATGGGGCCATTATGCCACCACAGGCCACAACACTTCAGGACATGATA
TTGATCTACCGGTATACACTGCCCGGCCAGTACTTTCTTCGTGAGGGATTGCAGGGAAGGCAACAT
L60 MGF 360 11L (NC_044941.1:21052-22113)
SEQ ID No. 77
TTATCTTTGTTCATAATCAAGAAAAATCCCCATATTTATTGAGTGATAATTTTTTAACATGTAATTTATTTTTTCAGGGTCCA
TAACGTTCGACAACAGAGAAATGACTGGATTGTAATGCTTTAATGATAAGGCATGGGCTATCAGATAATTTTCCTTTTGTTCT
GCCAAAGCTTTGCCCTCCTCAAAGGCGTTGGCGCCCAGGTCTATACAAAAGAACAGGTTTCCAAGATTATAGTTTTGTATGGA
AACCAGCATGGCTTGATTGATGTTGGCTCCCATGATAAAACAGTAGTAAATGGCCGAATAGCTATAATCTTGGATGCAGGCTA
TGTGCATCATTTCATCGATATCCATGCGGACCCTTTCTATTTCATACAGCTCGTGAAGGTCGAACACGTTGTTGTAAAAAAGG
GCGCACATGAGCCGCCACCGATGTAGGCGCGGGTATTTCTGGTAAAAGTAGCGAATAGCATCTTTGAGGTCATAGTCCACCGC
TATAGCATACCAGTATTTGGTTAAAACAGTGCTAAAGCTATCATCATGGTCTAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTCGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATATGAAGCATAATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Benin 97/1 MGF 360 11L (NC_044956.1:20703-21764)
SEQ ID No. 78
TTATCTTTGTTCATAATCAAGAAAAATCCCCATATTTATTGAGTGATAATTTTTTAACATGTAATTTATTTTTTCAGGGTCCA
TAACGTTCGACAACAGAGAAATGACTGGATTGTAATGCTTTAATGATAAGGCATGGGCTATCAGATAATTTTCCTTTTGTTCT
GCCAAAGCTTTGCCCTCCTCAAAGGCGTTGGCGCCCAGGTCTATACAAAAGAACAGGTTTCCAAGATTATAGTTTTGTATGGA
AACCAGCATGGCTTGATTGATGTTGGCTCCCATGATAAAACAGTAGTAAATGGCCGAATAGCTATAATCTTGGATGCAGGCTA
TGTGCATCATTTCATCGATATCCATGCGGACCCTTTCTATTTCATACAGCTCGTGAAGGTCGAACACGTTGTTGTAAAAAAGG
GCGCACATGAGCCGCCACCGATGTAGGCGCGGGTATTTCTGGTAAAAGTAGCGAATAGCATCTTTGAGGTCATAGTCCACCGC
TATAGCATACCAGTATTTGGTTAAAACAGTGCTAAAGCTATCATCATGGTCTAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGCTACTGGTTTTATCGCGCGTCGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATATGAAGCATAATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Mkuzi 1979 MGF 360 11L (AY261362.1:28164-29225)
SEQ ID No. 79
TTATCTTTGTTCATAATCAAGAAAAATCCCCATATTTATTGAGTGATAATTTTTTAACATGTAATTTATTTTTTCAGGGTCCA
TAACGATCGACAACAGAGAAATGACCGGATTGTAATGCTTTAATGATAAGGCATGGGCTATCAGATAATTTCCCTTTTGTTCT
GCCAAAGCTTTGCCCTCCTCAAAGGCGTTGGCGCCCAGGTCTATACAAAAGAACAGGTTTCCAAGATTATAGTTTTGTATGGA
AACCAGCATAGCTTGATTGATGTTGGCTCCCATGATAAAACAGTAGTAAATGACCGAATAGCTATAATCTTGGATGCAGGCTA
TGTGCATCATTTCATCAATATCCATGCGGACCCTTTCTATTTCATACAGCTCGTGAAGGTCGAACACGTTGTTGTAAAAAAGG
GCGCACATGAGCCGCCACCTATGTAGGCGCGGGTATTTCTGGTAAAAGTAGCGAATAGCATCTTTGAGGTCATAGTCCACCGC
TATAGCATACCAGTATTTGGTTAAAACAGTGCTAAAGCTATCATCATGGTCTAGCATGAAGGTTATCTCCATGAGCCCTCTTA
ACTCCCACATGATTTCTCCCCTCAGATCCAGATTATCTATAATCCTTAAATTGGGGTTATTGGAAAACACCTCGTGGCAAAAG
ATAATATTGCTACTGGTTTTATCGCGCGTCGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATATGAAGCATAATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Warmbaths MGF 360 11L (AY261365.1:26388-27449)
SEQ ID No. 80
TTATCTTTGTTCATAATCAAGAAAAATCCCCATATTTATTGAGTGATAATTTTTTAACATGCGATTTATTTTTTCAGGGTCCG
TAACCACCGACAACAGAGAAATGACCGGATTGTAATGCTTTAATGATAAGGCATTGGCTATCAGATAATTTCCCTTTTGTTCT
GCCAAAGCTTTGCCCTCCTCAAAGGCGTTGGCACCCAGGTCTATACAAAAGAACAGGTTCCCAAGATTATAGTTTTGTATGGA
AACCAGCATGGCTTGATTGATGTTGGCTCCCATGATAAAACAGTAGTAGATGGCTGAATAGCTATAATCTTGGACGCAGGCTA
TGTGCATCATTTCATCAATGTCCATGCGGACCCTTTCTATTTCATACAGCTCGTGAAGGTCGAACACGTTGTTGTAAAAAAGG
GCGCACATGAGCCGCCACCTATGTAGCCGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGTTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATATTCTATTACGTCGAATATGAAGCATTATGGGGCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Pretorisuskop/96/4 MGF 360 11L (AY261363.1:27214-28275)
SEQ ID No. 81
TTATCTTTGTTCATAATCAAGAAAAATCCCCATATTTATTGAGTGATAATTTTTTAACATGCGATTTATTTTTTCAGGGTCCG
TAACCACCGACAACAGAGAAATGACCGGATTGTAATGCTTTAATGATAAGGCATTGGCTATCAGATAATTTCCCTTTTGTTCT
GCCAAAGCTTTGCCCTCTTCAAAGGCGTTGGCACCCAGGTCTATACAAAAGAACAGGTTCCCAAGATTATAGTTTTGTATGGA
AACCAGCATGGCTTGATTGATGTTGGCTCCCATGATAAAACAGTAGTAAATGGCTGAATAGCTATAATCTTGGACGCAGGCTA
TGTGCATCATTTCATCAATGTCCATGCGGACCCTTTCTATTTCATACAGCTCGTGAAGGTCGAACACGTTGTTGTAAAAAAGG
GCGCACATGAGCCGCCACCTATGTAGCCGCGGGTATTTCTGGTAAAAGTAGCGGATAGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGTTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATATTCTATTACGTCGAATATGAAGCATTATGGGGCCATTATGCCACCACAGGCCACAACACTTCAGGATATGATA
ATGATCTACCGACACGCACTGCCCAGCCAGTACTTTCTTCGTGAGGGATTGCAGGGAAGGCAACAT
Tengani 62 MGF 360 11L (AY261364.1:21627-22688)
SEQ ID No. 82
TTATCTTTGTTCATAATCAAGAAAAATCCCCATATTTATTGAGTGATAATTTTTTAACATGCAATTTATTTTTTTAGGGTCTG
TAACGACCGACAACAGAGAAATGACCGGATTGTAATGCTTTAATGATAAGGCATCGGCTATCAGATAATTTCCCTTTTGTTCT
GCCAAAGCTTTGCCTTCCTCAAAGGCGTTGGCTCCCAGGTCTATACAAAAGAACAGGTTCCCAAGATTATAGTTTTGTATGGA
AACCAGCATGGCTTGATTGATGTTGGCTCCCATGATAAAACAGTAGTAAATGGCCGAATAGCTATAATCTTGGACGCAGGCTA
TGTGCATCATTTCATCAATGTCCATGCGGACCCTTTCTATTTCGTACAGCTCGTGAAGGTCGAACACGTTGTTGTAAAAAAGG
GCGCACATGAGCCGCCACCTATGTAGACGCGGGTATTTCTGGTAAAAGTAGCGGATCGCATCTTTGAGGTCATAGTCCACCGC
TATCGCGTACCAGTATTTGGTTAAAACAGTGCTAAAGCTATCATCATGGTCCAGCATGAAGGTTATCTCCATGAGCCCTCTTA
ACTCCCACATGATTTCCCCCCTCAGATCCAGATTATCTATAATCCTTAAATTGGGGTTATTGGAAAACACCTCGTGGCAAAAG
ATAATATTGTTACTGGTTTTATCGCGCGTCGTATCAAAGAAAATTTTTAAAATATACTCTCTTTCTAAATATTCTTTGGCACC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATAGTTTATTACGTCGAATCTGAAGCATAATGGGTCCATTATGCCACCACAGGCCACAACACTTCAGGATATGATA
ATGATCCACCGACACGCACTGCCCGGCCAGTACTTTCTTCGTGAGGGATTGCAGGGAAGGCAACAT
Warthog MGF 360 11L (AY261366.1:23157-24218)
SEQ ID No. 83
TTATCTTTGTTCATAATCAAGAAAAATCCCCATATTTATTGAGTGATAATTTTTTAACATGCGATTTATTTTTTCAGGGTCCG
TAACCACCGACAACAGAGAAATGACCGGATTGTAATGCTTTAATGATAAGGCATTGGCTATCAGATAATTTCCCTTTTGTTCT
GCCAAAGCTTTGCCCTCCTCAAAGGCGTTGGCCCCCAGGTCTATACAAAAGAACAGGTTCCCAAGATTATAGTTTTGTATGGA
AACCAGCATGGCTTGATTGATGTTGGCTCCCATGATAAAACAGTAGTAAATGGCTGAATAGCTATAATCTTGGACGCAGGCTA
TGTGCATCATTTCATCAATGTCCATGCGGACCCTTTCTATTTCATACAGCTCGTGAAGGTCGAACACGTTGTTGTAAAAAAGA
GCGCACATGAGCCGCCACCTATGTAGCCGCGGGTATTTCTGGTAAAAGTAGCGGATAGCATCTTTGAGGTCATAGTCCACCGC
TATCGCGTACCAGTATTTGGTTAAAACAGTACTAAAGCTATCATCATGGTCCAGCATGAAGGTTATCTCCATGAGCCCTCTTA
ACTCCCACATGATTTCTCCCCTTAGATCCAGATTATCTATAATCCTTAAATTGGGGTTATTGGAAAACACCTCGTGGCAAAAG
ATAATATTGTTACTGGTTTTATCGCGCGTTGTATCAAAGAAAATTTTTAAAATATACTCTCTTTCTAAATATTCTTTGGCTCC
CAGCTCTTTGCACAGATCACGGGTATTTTCCGTGAGAGCACAAATCATTCCATAGTTAATATCTGCACCCCATTCAGTAAACA
GCTTTATCAAGTCATGATTATTCTCCTTCACGGCTTTCATCAGTCCTATGTTTAACTCGATACCTTGACTAAAGCAGGTTGAC
CTTATAAATATTCTATTACGTCGAATATGAAGCATTATGGGGCCATTATGCCACCACAGGCCACAACACTTCAGGATATGATA
ATGATCCACCGATACGCACTGCCCGGCCAGTACTTTCTTCGTGAGGGATTGCAGGGAAGGCAACAT
Malawi Lil-20/1 (1983) MGF 360 11L (AY261361.1:24831-25892)
SEQ ID No. 84
TTATCTTCGTTCATAATCAAGAAAAACCCTCATATTTATTGAGTGATAATTTTTTAACATGCAATTTATTTTTTCAGGGTCCA
TAATGACCGACAACAGAGAAATGACCGGATTGTAATGCTTTAATGATAAGGCATCGGCTATCAGATAATTTTCCTTTTGTTCT
GCCAAAGCTTTCCCCTCCTCAAAGGCGTTGGCCCCCAGATCTATACAAAAGAACATGTTCCCAAGATTATAGTTTTGTATAGA
AACAAGCATGGCTTGATTGATGTTGGCCCCCATGATAAAACAGTAATAAATAGCCGCATAGCTATAATCTTGGACGCAGGCTA
TGTGCATCATTTCATCAATATCCATGCGGACCCTTTCTATTACATACAGCTCGTGAAGGTCGAACACGTTGTTGTAAAAAAGG
GCGCACATAAGCCGCCAGTGGTGTAGATGGGTATACCTCTGATAAAAATAATGGATAGCCTTTTTGAGGCCATAGTCTACCGC
TATGGCGTACCAGTATTTGGTTAACATATTGCTAAAGGAGTTGTCATGGTCCAACATGTAGGTTATCTCCATGAGTCCCCTTA
GCTCCCACATGATTTCCTCCCTCAGATCCAAATTATCCACATTTTTCAGGTTGGGGTTATTGGAAAACATTTCGTGGCATAAG
ATAATATTACTGTCGGTTTTGTTGCGCGTCGTATCAAAGAAAATTTTTAAAATATAATCTCTATCTAAACGATCTTTGGCTCC
TAGTTCTCTGCATAGATTACGGGTGTGCTCCGTATGGGCACAAATCAGTCCATAATTAATATCGGCACCCCACTCGGCAAACA
GCCTTATCAAGTCATGGTTGTTTTCCTTCGCGGCTTTCATCAGTACGGTGTTCAATTCAATACCCTCGCTAAAACAGGTTGAC
CTTATAAACATTTTGTGACGATAAACGTAAAATGTAATAGGGCCATCATACCACCACAACCCGCAGCACTTTAAGACATGATA
TTGATCTACCGGTACACACTGTTTGGCCAGCACTTTCTTAGTTAAAGATTGTAAGGAAGGCAACAT
Kenya 1950 MGF 360 11L (AY261360.1:30655-31716)
SEQ ID No. 85
TTATCTGCGTTCATAATCGAGAAAAACCACCATATTCTTTGAGTGATAATTTTTTAACATAGAATTTATCTTTTCTGGGTCGG
TCACGGCCGACAACAGGGAAATCACCGGGTTATAATGTTTTAATGATAAGGCATCGGCTATCAAATAATTTTCCTTTTGTATT
GCTAGGGTTTTGCCTTCTTCAAAGGCGTTGGCACCCAGGTCTATACAAAAGAACATGTTCCCGAGATTATAGTTTTGTATGGA
AACAAACATGGCTTGATTGATGTTGGCCCCCATGATAAAACAGTAGTAGATAGCTGAATAGCTATAATCTTGGATGCAGGCTA
TGTGCATCATCTCATCGATATCCATGCGGACCCTTTCTATTTCATACAGCTCGTGAAGGTCGAACACATTGTTATAAAAAAGG
GCGCACATAAGCCGCCACCGATGTAGATGGGTATACTTCTGATAAAAATAGTGGATGGCCTTTTTGAGGCCGTAGTCTACCGC
TATGGCGTACCAGTATTTGGTTAACATATTGCTGAAGGAGTTGTCATGGTCCAACATGTAGGTTATCTCCATGAGTCCCCTTA
GCTCCCACATGATTTCCTCCCTCAGATCCAAATCATCCACATGTTTCAGATTGGGGTTATTGGAAAACATTTCGTGGCATAAG
ATAATATTACTGTCGGTTTTGTTGCGCGTCGTATCAAAGAAAATTTTTAAAATATACTCTCTATCTAAACGGTCTTTGGCTCC
AAGTTCTCTGCATAGATTGCGGGTGTGCTCCGAATGAGCACAAACCAGTCCATAATTAATATCGGCACCCCACTCAACAAACA
GCCTTATCAAGTCATGATTGTTTTCCTTCGCGGCTTTCATCAGTACGGTGTTCAATTCAATACCCTCGCTAAAACAGGTTGAC
TTTATAAACATTTTGTTACGATACGTATAAAATGTAATAGGGCCATCATACCACCACAACCCGCAGCATTTCAGGATATGATA
TTGATCTACCGGTATACACTGTTTGGCCAGCACTTTCTTGGATAGGGATTGCAAGGAAGGCAACAT
Ken05/Tk1 MGF 360 11L (NC_044945.1:28407-29468)
SEQ ID No. 86
TTATCTGCGTTCATAATCGAGAAAAACCACCATATTCTTTGAGTGATAATTTTTTAACATAGAATTTATCTTTTCTGGGTCGG
TCACGGCCGACAACAGGGAAATCACCGGGTTATAATGTTTTAATGATAAGGCATCGGCTATCAAATAATTTTCCTTTTGTATT
GCTAGGGCTTTGCCTTCTTCAAAGGCGTTGGCACCCAGGTCTATACAAAAGAACATGTTCCCGAGATTATAGTTTTGTATGGA
AACAAACATGGCTTGATTGATGTTGGCCCCCATGATAAAACAGTAGTAAATAGCTGAATAGCTATAATCTTGGATGCAGGCTA
TGTGCATCATCTCATCGATATCCATGCGGACCCTTTCTATTTCATACAGCTCGTGAAGGTCGAACACATTGTTATAAAAAAGG
GCGCACATAAGCCGCCACCGATGTAGATGGGTATACTTCTGATAAAAATAGTGGATGGCCTTTTTGAGGCCGTAGTCTACCGC
TATGGCGTACCAGTATTTGGTTAACATATTGCTGAAGGAGTTGTCATGGTCCAACATGTAGGTTATTTCCATGAGTCCCCTTA
GCTCCCACATGATTTCCTCCCTCAGATCCAAATCATCCACATGTTTCAGATTGGCGTTATTGGAAAACATTTCGTGGCATAAG
ATAATATTACTGTCGGTTTTGTTGCGCGTCGTATCAAAGAAAATTTTTAAAATATACTCTCTATCTAAACGGTCTTTGGCTCC
AAGTTCTCTACATAGATTGCGGGTGTGCTCCGAATGAGCACAAACCAGTCCATAATTAATATCGGCACCCCACTCAACAAACA
GCCTTATCAAGTCATGATTGTTTTCCTTCGCGGCTTTCATCAGTACGGTGTTCAATTCAATACCCTCGCTAAAACAGGTTGAC
TTTATAAACATTTTGTTACGATACGTATAAAATGTAATAGGGCCATCATACCACCACAACCCGCAGCATTTCAGGATATGATA
TTGATCTACCGGTATACACTGTTTGGCCAGCACTTTCTTGGATAGGGATTGCAAGGAAGGCAACAT
Ken06.Bus MGF 360 11L (NC_044946.1:24534-25595)
SEQ ID No. 87
TTATCTGCGTTCATAATCGAGAAAAACCACCATATTCTTTGAGTGATAATTTTTTAACATAGAATTTATCTTTTCCGGGTCGG
TCACGGCCGACAACAGGGAAATCACCGGGTTATAATGTTTTAATGATAAGGCATCGGCTATCAAATAATTTTCCTTTTGTATT
GCTAGGGCTTTGCCTTCTTCAAAGGCGTTGGCACCCAGGTCTATACAAAAGAACATGTTCCCGAGATTATAGTTTTGTATGGA
AACAAACATGGCTTGATTGATGTTGGCACCCATGATAAAACAGTAGTAAATAGTTGAATAGCTATAATCTTGGATGCAGGCTA
TGTGCATCATCTCATCGATATCCATGCGGATCCTTTCTATTTCATACAGCTCGTGAAGGTCGAACACATTGTTATAAAAAAGG
GCGCACATGAGCCGCCACCGATGTAGATGGGTATACTTCTGATAAAAATAGTGGATGGCCTTTTTGAGGCCGTAGTCTACCGC
TATGGCGTACCAGTATTTGGTTAACATGTTGCTGAAGGAGTTGTCATGGTCCAACATGTAGGTTATCTCCATGAGTCCCCTTA
GCTCCCACATGATTTCCTCCCTCAGGTCCAAATCATCCACATGTTTCAGATTGGGGTTATTGGAAAACATTTCGTGGCATAAG
ATAATATTACTGTCGGTTTTGTTGCGCGTCGTATCAAAGAAAATTTTTAAAATATACTCTTTATCTAAACGGTCTTTGGCCCC
TAGTTCTCTGCATAGATCGCGGGTGTGCTCCGAATGAGCACAAACCAGTCCATAATTAATATCGGCACCCCACTCAGTAAATA
ATTTTATTAGGTCATGATTGTTTTCCTTCGCGGCTTTCATCAGTACGGTGTTCAATTCAATACCCTCGCTAAAACAGGTTGAC
TTTATAAACATTTTGTTACGATACGTATAAAATGTAATAGGGCCATCATACCACCACAACCCGCAACTTTTCAGGATATGATA
TTGATCTACCGGTATACACTGTTTGGCCAGCACTTTTTTGGATAGGGATTGCAAGGAAGGCAACAT

In an embodiment the attenuated ASFV of the invention comprises a functional version of MGF 360 11L. Suitably the functional version of MGF 360 11L comprises the sequence of SEQ ID No. 75, 76, 77, 78, 79, 80, 81, 82, 83, 84, 85, 86 or 87. Suitably the functional version of MGF 360 11L comprises a sequence having at least 70%, at least 80%, at least 90% or at least 95% identity with SEQ ID No. 75, 76, 77, 78, 79, 80, 81, 82, 83, 84, 85, 86 or 87. Suitably the functional version of MGF 360 11L consists of the sequence of SEQ ID No. 75, 76, 77, 78, 79, 80, 81, 82, 83, 84, 85, 86 or 87.

### MGF 360 12L Gene Sequences

Georgia 2007/1 MGF 360 12L (LR743116.1:30355-31407)
SEQ ID No. 88
TCATCTTAAATCATAGGTAAGGAAGATCATCATATTTTTTGAAACGTAATTTTTTAACGCATGATCTATGATTTCAGGGTCCG
TGCTTTTAGGCAACGGGGTGGTGGCCGGACTATAAATCTTTAGGGATAAAATGTTCTTTATAAGCTCATACCCTTCCCCTAAA
GCTGTAGTACCCTCTTCGAAAACATCAGCCCCCAGATCTATACAAAAGAACATGTTTTCTATATTATAGTACTGTATTGAGCT
AAGCATGGCTTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTACTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTTTCCTTGGCACCTAG
CTCTCGACAGAGGTCCCAGGTGTGCTCCGTGTTGACAGATACCAGCCCGTAGTTGATGTCCGCCCCCCACTCTGCAAACAGTT
TTATAAGGTTGTAGTTGTTTTCCCTTACAGCCTTCACTAACGCCGTATTTAGGTTTAAGCCCTCTTTAATACCTGCTGATTTT
ATGAGCCTTAGGTTATGATCAAACGTGATCGGAGCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
China/2018/AnhuiXCGQ MGF 360 12L (MK128995.1:29384-30436)
SEQ ID No. 89
TCATCTTAAATCATAGGTAAGGAAGATCATCATATTTTTTGAAACGTAATTTTTTAACGCATGATCTATGATTTCAGGGTCCG
TGCTTTTAGGCAACGGGGTGGTGGCCGGACTATAAATCTTTAGGGATAAAATGTTCTTTATAAGCTCATACCCTTCCCCTAAA
GCTGTAGTACCCTCTTCGAAAACATCAGCCCCCAGATCTATACAAAAGAACATGTTTTCTATATTATAGTACTGTATTGAGCT
AAGCATGGCTTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTACTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTTTCCTTGGCACCTAG
CTCTCGACAGAGGTCCCAGGTGTGCTCCGTGTTGACAGATACCAGCCCGTAGTTGATGTCCGCCCCCCACTCTGCAAACAGTT
TTATAAGGTTGTAGTTGTTTTCCCTTACAGCCTTCACTAACGCCGTATTTAGGTTTAAGCCCTCTTTAATACCTGCTGATTTT
ATGAGCCTTAGGTTATGATCAAACGTGATCGGAGCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Tengani 62 MGF 360 12L (AY261364.1:24540-25592)
SEQ ID No. 90
TCATCTTAAATCATAGGTAAGGAAGATCATCATATTTTTTGAAACGTAATTTTTTAACGCATGATCTATGATTTCAGGGTCCA
TGCTTTTAGGCAACGGGGTGGTGGCCGGACTATAAATCTTTAGGGATAAAATGTTCTTTATAAGCTCATACCCTTCCCCTAAA
GCTGTAGTACCCTCTTCGAAAACATCAGCCCCCAGATCTATACAAAAGAACATGTTTTCTATATTATAGTACTGTATTGAGCT
AAGCATGGCCTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCAGGTGTGCTCCGTGTTGACAGATACCAGCCCGTAGTTGATGTCCGCCCCCCACTCTGCAAACAGTT
TTATAAGGTTGTAGTTGTTTTCCCTTACAGCCTTTACTAACGCCGTATTTAGGTTTAAGCCCTCTTTAATACCTGCTGATTTT
ATGAGCCTTAGGTTATGATCAAACGTGATCGGAGCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Warthog MGF 360 12L (AY261366.1:26036-27088)
SEQ ID No. 91
TCATCTTAAATCGTAGGTAAGGAAGATCATCATATTTTTTGAAACGTAATTTTTTAACGCATGATCTATAATTTCAGGGTCCG
TGCTTTTAGGCAACGGGGTGGTGGCCGGACTATAAATCTTTAGGGATAAAATGTTCTTTATAAGCTTATACCCTTCCCCTAAA
GCTATAGTGCCCTCTTCGAAAGCATCAGCCCCCAGATCCATACAAAAGAACATGTTTTCTATATTATAGTACTGTATTGAGGT
AAGCATGGCCTGATTGATGTTGGCGCCCAGGACATAGCAGTAGTACATGGTTGAAAGGTTGTGGTCTTTGATGCAGGCGATCC
GCATCATCTCTTCTATGTCCATATGGATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTGAGTAGTAATGCACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCGGGTGTGCTCCGTGTTGACAGATACCAGCCCATAGTTGATGTTCGCCCCCCACTCTGCAAACAGTT
TTATAAGGTTGTAATTGTTTTCCCTTACAGCCTTCACTAACGCCGTATTTAGGTCTAAGCCCTCTTTAATGCCTGCTGATTTT
ATGAGCTTTAGGTTATGATCAAACGTGATCGGACCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Warmbaths MGF 360 12L (AY261365.1:29294-30346)
SEQ ID No. 92
TCATCTTAAATCGTAGGTAAGGAAGATCATCATATTTTTTGAAACGTAATTTTTTACCGCATGATCTATGATTTCAGGGTCCG
TGCTTTTAGGCAACGGGGTGGTGGCCGGACTATAAATCTTTAGGGATAAAATGTTCTTTATAAGCTTATACCCTTCCCCTAAA
GCTATAGTACCCTCTTCGAAAGCATCAGCCCCCAGATCCATACAAAAGAACATGTTTTCTACATTATAGTACTGTATTGAGGT
AAGCATGGCCTGATTGATGTTGGCGCCCAGAACATAGCAGTAGTACATGGTTGAAAGGTTGTGGTCTTTGATGCAGGCGATCC
GCATCATCTCTTCTATGTCCATATAGATCTTGTCCTTTTCATACGCCTCATGAAGGTCAAACACATTATTAAAACAAAGAGCA
CATGTTAACCGCCATGTATTCAGGTGTGTATATTTTTGGTAAAAATACTGTATGGCCTCTTTCAGGTTATAGCGTATGGCTAT
AGCGTACCAGTATTTGAGTAGTAATGTACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCGGGTGTGCTCCGTGTTGACAGATACCAGCCCATAGTTGATGTCCGCCCCCCACTCTGCAAACAGTT
TTATAAGGTTGTAGTTGTTTTCCCTTACAGCCTTCACTAACGCCGTATTTAGGTCTAAGCCCTCTTTAATGCCTGCTGATTTT
ATGAGCTTTAGGTTATGATCAAACGTGATCGGACCATCATGCCACCATAGGTCATAACACTTTAAAAGATAATGTTGGTTCGT
GGGCACGCATTGTCCAGCCAACACCTTTTTGGTCAGAGATTGCAGGGAAGGCAACAT
Pretorisuskop/96/4 MGF 360 12L (AY261363.1:30098-31150)
SEQ ID No. 93
TCATCTTAAATCGTAGGTAAGGAAGATCATCATATTTTTTGAAACGTAATTTTTTAACGCATGATCTATAATTTCAGGGTCCG
TGCTTTTAGGCAACGGGGTGGTGGCCGGACTATAAATCTTTAGGGATAAAATGTTCTTTATAAGCTTATACCCTTCCCCTAAA
GCTATAGTGCCCTCTTCGAAAGCATCAGCCCCCAGATCCATACAAAAGAACATGTTTTCTATATTATAGTACTGTATTGAGGT
AAGCATGGCCTGATTGATGTTGCCCCCCAGGACATAGCAGTAGTACATGGTTGAAAGGTTGTGGTCTTTGATGCAGGCGATCC
GCATCATCTCTTCTATGTCCATATGAATCTTGTCCTTTTCATACGCCTCATGAAGGTCAAACACATTATTAAAACAAAGAGCA
CATGTTAACCGCCACGTATTCAGGTGTGTATATTTTTGGTAAAAATACTGTATGGCCTCTTTCAGGTTATAGCGTATGGCTAT
AGCGTACCAGTATTTAAGTAGTAATGTACTGAGCGAAAACTCATTATTTAGCAGATCGGTTTTTTCTATTAACTCCCTTAACT
CCCAGAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTCTTCATTCAAGGTCTCCTTGGCACCTAG
CTCTCGACAGAGGTCCCAGGTGTGCTCCGTGTTGACAGATACCAGCCCATAGTTGATGTTCGCCCCCCACTCTGCAAACAGTT
TTATAAGGTTGTAGTTGTTTTCCCTTACAGCCTTCACTAACGCCGTATTTAGGTCTAAGCCCTCTTTAATGCCTGCTGATTTT
ATGAGCTTTAGGTTATGATCAAACGTGATCGGACCATCATGCCACCATAGGTCGTAACACTTTAAAAGATAATGTTGGTTTGT
GGGCACGCATTGTCCAGCCAACACCTTTTTGGTCAGAGATTGCAGGGAAGGCAACAT
Mkuzi 1979 MGF 360 12L (AY261362.1:31073-32125)
SEQ ID No. 94
TCATCTTAAATCATAGGAAAGGAAGATCATCATATTTTTTGAAAAGTAATTTTTTAACGCATGATCTATGATTTCAGGGTCCG
TGCTTTTAGGCAACGGGATGGTGGTCGGACTATAAATCTTTAGAGATAAAATGTTTTTTATAAGCTCATACCCTTCCCCTAAA
GCTGTAGTACCCTCTTCGAAAACATCAGCCCCCAGATCCATACAAAAGAACATGTTTTCTATATTATAGTACTGTATTGAGGT
AAGCATGGCCTGATTGATGTTGGCGCCCAACATATAGCAGTAGTACATGGTTGAAAGGTTGTGGTCTTTAATGCAGGCGATCC
GCATCATCTCTTCTATGTCCATATAGATCTTGTCCTTTTCATACGCCTCATGGAGGTCAAACACATTATTAAAACAAAGAGCA
CATGTTAACCGCCACGTATTCAGGTGTGTATATTTTTGGTAAAAATACTGTATGGCTTCTTTCAGGTTATAGCGTATGGCTAT
AGCGTACCAGTATTTGAGTAGCAATGTACTGAGCGAAAACTCATTATTTAGCAGATCGGTTTTTTCTATTAACTCCCTTAACT
CCCAAAAAATTTCTATCCTCATTTTTATATTATTTACTTTTTGTAATATCGGATTGTTGGAAAACACCTCATGGCATAAAATA
ATGTTACTACTAGTTTTATGAAACTTTAGATCTATAAAAATTTGTAAAATTTCTTTTTCATTCAAGGTCTCCTTGGCACCTAG
CTCTCGACAGAGGTCCCAGGTGTGCTCCGAGCTGACAGATACCAGCCCATAGTTGATGTCCGCCCCCCACTCTGCAAACAATT
TTATAAGGTTGTAGTTGTTTTCCTTTACAGCCTTCACCAACGCCGTATTTAGGTCTAAGCCCTCTTTAATGCCTGCTGATTTT
ATGAGCCTTAGGTTATGATCAAACGTGATCGGAGCATCATGCCACCATAGGTCATAATATTTTAAAAGATAATGTTGGTTCGT
GGGCACGCATTGTCCGGCCAACACCTTTTTGGTCAAAGATTGCAGGGAAGGCAACAT
L60 MGF 360 12L (NC_044941.1:23965-25017)
SEQ ID No. 95
TCATCTTAAATCATAGGAAAGGAAGATCATCATATTTTTTGAAAAGTAATTTTTTAACGCATGATCTATGATTTCAGGGTCCG
TGCTTTTAGGCAACGGGATGGTGGTCGGACTATAAATCTTTAGAGATAAAATGTTTTTTATAAGCTCATACCCTTCCCCTAAA
GCTGTAGTACCCTCTTCGAAAACATCAGCCCCCAGATCCATACAAAAGAACATGTTTTCTATATTATAGTACTGTATTGAGGT
AAGCATGGCCTGATTGATGTTGGCGCCCAACATATAGCAGTAGTACATGGTTGAAAGGTTGTGGTCTTTGATGCAGGCAATCC
GCATCATCTCTTCTATGTCCATATGGATCTTGTCCTTTTCATACGCCTCATGGAGGTCAAACACATTATTAAAACAAAGAGCA
CATGTTAACCGCCACGTATTCAGGTGTGTATATTTTTGGTAAAAATACTGTATGGCCTCTTTCAGGCTATAGCGTACGGCTAT
AGCGTACCAGTATTTGAGTAGTAATGTACTGAGCAAAAACTCATTATTTAGCAGATCGGTTTTTTCTATTAACTCCCTTAACT
CCCAGAAAATTTCTATCCTCAATTTCAGATTATTTACTTTTTGTAATATCGGATTGTTGGAAAACACCTCATGGCATAAAATA
ATGTTACTACTAGTTTTATGAAACTTTAGATCTATGAAAATTTGTAAAATTTCCTCTTCATTCAAGATCTCCTTGGCACCTAA
CTCTTGACAGAGGTCCCGGGTATGCTCCGTGTTGACAGATACCAGCCCATAGTTGATGTCCGCCCCCCACTCTGTAAATAGTT
TTATAAGGCTGTAGTTGTTTTCCCTTACAGCCTTTACTAACGCCGTATTTAGGTCTAAGCCCTCTTTAATGCCTGATGATTTT
ATGAGCCTTAGGTTATGATCAAACGTGATCGGAGCATTATACCACCATAGATCATAATATTTTAAAAGATAATGTTGGTTCGT
GGGCACGCATTGTCCAGCCAACACCTTTTTGGTCAAAGATTGCAGGGAAGGCAACAT
Benin 97/1 MGF 360 12L (NC_044956.1:23616-24668)
SEQ ID No. 96
TCATCTTAAATCATAGGAAAGGAAGATCATCATATTTTTTGAAAAGTAATTTTTTAACGCATGATCTATGATTTCAGGGTCCG
TGCTTTTAGGCAACGGGATGGTGGTCGGACTATAAATCTTTAGAGATAAAATGTTTTTTATAAGCTCATACCCTTCCCCTAAA
GCTGTAGTACCCTCTTCGAAAACATCAGCCCCCAGATCCATACAAAAGAACATGTTTTCTATATTATAGTACTGTATTGAGGT
AAGCATGGCCTGATTGATGTTGGCGCCCAACATATAGCAGTAGTACATGGTTGAAAGGTTGTGGTCTTTGATGCAGGCAATCC
GCATCATCTCTTCTATGTCCATATGGATCTTGTCCTTTTCATACGCCTCATGGAGGTCAAACACATTATTAAAACAAAGAGCA
CATGTTAACCGCCACGTATTCATGTGTGTATATTTTTGGTAAAAATACTGTATGGCCTCTTTCAGGCTATAGCGTACGGCTAT
AGCGTACCAGTATTTGAGTAGTAATGTACTGAGCAAAAACTCATTATTTAGCAGATCGGTTTTTTCTATTAACTCCCTTAACT
CCCAGAAAATTTCTATCCTCAATTTCAGATTATTTACTTTTTGTAATATCGGATTGTTGGAAAACACCTCATGGCATAAAATA
ATGTTACTACTAGTTTTATGAAACTTTAGATCTATGAAAATTTGTAAAATTTCCTCTTCATTCAAGATCTCCTTGGCACCTAA
CTCTTGACAGAGGTCCCGGGTATGCTCCGTGTTGACAGATACCAGCCCATAGTTGATGTCCGCCCCCCACTCTGTAAATAGTT
TTATAAGGCTGTAGTTGTTTTCCCTTACAGCCTTTACTAACGCCGTATTTAGGTCTAAGCCCTCTTTAATGCCTGATGATTTT
ATGAGCCTTAGGTTATGATCAAACGTGATCGGAGCATTATACCACCATAGATCATAATATTTTAAAAGATAATGTTGGTTCGT
GGGCACGCATTGTCCAGCCAACACCTTTTTGGTCAAAGATTGCAGGGAAGGCAACAT
Malawi Lil-20/1 (1983) MGF 360 12L (AY261361.1:27682-28731)
SEQ ID No. 97
TCATCTTAAATCGTAGGAAAGGAAGGTCATCATATTTTTTGATTTATAATTTTTTAACACATGATCTATTATTTCAGGGTCCG
TGTTTTTAGGTAACGGGGCAGTGGACGGACTATAGATCTTTAGGGATAAAATGTTCCTTATAAGCTCATACCCTTCCCCTACA
GGCATATTCTCTTCGAAAACATCAGCCCCCAGATCCATACAAAAGAACATGTTTTCTATATTATAGTACTGTATTGAGGCAAG
CATGGCCTGATTAATGTTGGCGCCCAGGACATAGCAGTAGTATATCGTTGAAAGGTTGTGGTCTTTGATGCAGGCTACTCGCA
TCATCTCTTCTAAGTCCATATAGATCTTGTCCTTTTCATACGCCTCATGAAGGTCAAACACATTATTAAAACAAAGAGCACAT
GTTAACCGCCACGTATTCAGGTGTGTATATTTTTGATAAAAATACTGTATGGCCTCTTTCAGGTTATAGCGTACGGCTATAGC
GTACCAGTATTTGAGTAATAATGTATTGAGTGAAAACTCATTATTTAACAGATCGGTTTTTTTTATTAACTCCCTTAACTCCC
AGAAAATTTCTATCCTCATTTTCAGATTATTTACTTTTTGTAATAACGGATTGTTGGAAAACACCTCATGGCATAAAATAATG
TTACTACTAGTTTTATAAAACTTTAGATCGATGAAAATTCGTAAAATTTCCTCTTCATTTAAGGTCTCCTTGGCGCCTAGCTC
TCTGCAGAGGTCCCGGGTGTGCTCCGTGTTGACAGATACCAGCCCGTAGTTGATGTCCGCCCCCCACTCTGTAAAAAGCTTTA
TAAGGTTGTAGTTGTTTTCCCTTACAGCCTTTACTAACGCCGTATTTAGGTCTAAGCCATCTTTAATGCCTGCTGATTTTATC
AGCCCTAGGTTATGATCAAACGTGATCGGAGAATCATACCACCATAGGTCGTAACACTTTAAAAGATAATATTGGTCCGTCGG
CAGGCATTGTCCAGCCAGTACCTTTTTGGTCAGAGATTGCAGGGAAGGCAACAT
Kenya 1950 MGF 360 12L (AY261360.1:33549-34604)
SEQ ID No. 98
TCATCTTAGATCATAAGTAAGGAAGACCATCATATTTTTTGAAAAGTAATTTTTTAACTCATGATCTATAATTTCAGGGTCCG
TGCTTTTAGGCAACGGGGTGGTGGCCGGGCTATAAATCTTTAGGGATAAAATATTCCTTATAGGCTCATACCCTTCCTGTCCC
ACTAGAGCCATACCCTCTTCAAAAGCATCGGCCCCCAGATCCATACAAAAGAATATGTTTTCTATATTATAGTACTGTATTGA
GGTAAGCATGGCCTGATTGATGTTGGCGCCCAGCATATAGCAGTAGTATATCGTTGAAAGGCTGTGGTCTTTGATGCAGGCTA
TCCGCATCATCTCTTCTAAATCCATACAGATCTTGTCCTTTTCATACGCCTCATGAAGGTCAAACACGTTATTAAAACAAAGA
GCACATGTTAATCGCCACGTATTCAGGTGCGTATATTTTTGGTAAAAATACTGTATGGCCTCTTTCAAGTTGTAGCGTACGGC
TATAGCGTACCAGTATTTGAGTAATAGTGTATTGAGCGAAAACTCATTATTTAGCAGATCGGTTTTTTCTATTATCTCCCTTA
ACTCCCAAAAAATTTCTATCCTCATTTTCAGGTTATTTACCTTTTTTAAAAGTAGATTATCGGAAAACACCTCATGACATAAG
ATAATGTTACTACTGGTTTTATGAAACTTTAAATCTATGAAAATTTGTAAAATATCCCCTTCATTTAAGGTCTTCTTGGCGCC
TAGCTCTCGACAGAGGTCCCAGGTGTGCTCCGTGTTGACAGATATCAGCCCGTAGTTGATGTTCGCTCCCCACTCCGTAAATA
GTTTTATAAGACTGTAGTTGTTTTCCTTTACAGCCTTCACTAATGCCATGTTTAGGTCTAAGCCTTCTTGAAGGCCTGCTGAT
TTTATCAGCCTTAGGTTATGATCAAACGTGATCGGAGCATTATTCCACCATAGATCGTAACACTTTAAAAGATAATGTTGGTC
CTCGGGCAGACATTGTCCAGCCAGCACCTTTTTGGTCAGAGATTGCAGGGAAGGCAACAT
Ken06.Bus MGF 360 12L (NC_044946.1:27428-28483)
SEQ ID No. 99
TCATCTTAGATCATAAGTAAGGAAGACCATCATATTTTTTGAAAAGTAATTTTTTAACGCATGATCTATAATTTCAGGGTCCG
TGCTTTTAGGCAATGGGGTAGTGGCCGGGCTATAAATCTTTAGGGATAAAATATTCCTTATAGGCTCATACCCTTCCTGTCCC
ACTAGAGCCATACCCTCTTCAAAAGCATCGGCCCCCAGATCCATACAAAAGAATATGTTTTCTATATTATAGTACTGTATTGA
GGTAAGCATGGCCTGATTGATGTTGGCGCCCAGCATATAGCAGTAGTATATTGTTGAAAGGTTGTGGTCTTTGATGCAGGCTA
TCCGCATCATCTCTTCTAAATCCATACAGATCTTGTCCTTTTCATACGCCTCATGAAGGTCAAACACGTTATTAAAACAAAGA
GCACATGTTAACCGCCACGTATTCAGGTGTGTATATTTTTGGTAAAAATACTGTATGGCCTCTTTCAAGTTGTAGCGTACGGC
TATAGCGTACCAGTATTTGAGTAATAGTGTATTGAGCGAAAACTCATTATTTAGCAGATCGGTTTTTTCTATTATCTCCCTTA
ACTCCCAAAAAATTTCTATCCTCATTTTCAGGTTATTTACCCTTTTTAAAAGTAAATTATCGGAAAACACCTCATGACATAAG
ATAATGTTACTACTGGTTTTATAAAATTTTAAATCTATGAAAATTTGTAAAATATCCCCTTCATTTAAGGTCTTCTTGGCGCC
TAGCTCTCGACAGAGGTCCCAGGTGTGCTCCGTGTTGACAGATATCAGCCCGTAGTTAATGTTCGCCCCCCACTCCGTAAATA
GTTTTATAAGACTGTAGTTGTTTTCCTTTACAGCCTTCACTAATGCCATGTTTAGGTCTAAGCCTTCTTGAATGCCTGCTAAT
TTTATCAGCCTTAGGTTATGATCAAACGTGATCGGAGCATTATTCCACCATAGATCGTAACACTTTAAAAGATAATGTTGGTC
CTCGGGCAGACATTGTCTAGCCAGCACCTTTTTGGTCAAAGATTGCAGGGAAGGCAACAT

In an embodiment the attenuated ASFV of the invention comprises a functional version of MGF 360 12L. Suitably the functional version of MGF 360 12L comprises the sequence of SEQ ID No. 88, 89, 90, 91, 92, 93, 94 95, 96, 97, 98 or 99. Suitably the functional version of MGF 360 12L comprises a sequence having at least 70%, at least 80%, at least 90% or at least 95% identity with SEQ ID No. 88, 89, 90, 91, 92, 93, 94 95, 96, 97, 98 or 99. Suitably the functional version of MGF 360 12L consists of the sequence of SEQ ID No. 88, 89, 90, 91, 92, 93, 94 95, 96, 97, 98 or 99.

### MGF 360 13L Gene Sequences

Georgia 2007/1 MGF 360 13L (LR743116.1:31568-32629)
SEQ ID No. 100
CTATAGTATATTATGAGAATATTCCCAAATGATGGATAAGTTTTTTGATTTATAATCTTTTAATAAACTGCTTATTTCTTCGG
GGTCCTTTAAGTTTAGTGGCAAGGAAGCATCTGAGCTGTAAATATCCAAAGCCAAACTATGGCTCAGAAAATTATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGCGCGTAACAGTAATATATTGTTAATGGATTGTTATCCTTGGTAG
AAGCCAGACATATCATGTCATGGACGTCTATTTGGATGTTTTCCTTGTGGTACATCTCATGAAGCTCATATATTTTGTTATAA
TACAGGAGACATTTTAATCGCCATTCATTAAGATCCGTATATTTCTCATCTAGAAAACAAATGGCGTCCTTACAATCGTATTG
TACTGCTTTGGCGTACCAATACTTCACTAGTAAACCATTTAACTCGTCCGTTTCTTTTATTTCTATGAGCCCCCATAGTCTTT
TATAAATTAAGCCCCTTAATTGTATAACAAATTTGTTTTCTAAAATAGGATTATTCATAAAAATTTCATGGCACAAAATAATA
CTGCCGCTGGTTTTATTGTGCATTATCCTGGTAAAAATACGGAAAATATCGTTGTCCTCTAGAGTTTCTTTGGCGCCTAGCTG
TCTACACAACTCTCGGATGTGCTTCGTATTGATAGAAAGCAAACCATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAATATTTCAAAATGCAGTGTTCATC
TATTGACAAACACTGGCTGGCTATCGTCTTTTTGACGAGGGTCTGCAGAGAGAGCGGCAACGACAT
China/2018/AnhuiXCGQ MGF 360 13L (MK128995.1:30597-31658)
SEQ ID No. 101
CTATAGTATATTATGAGAATATTCCCAAATGATGGATAAGTTTTTTGATTTATAATCTTTTAATAAACTGCTTATTTCTTCGG
GGTCCTTTAAGTTTAGTGGCAAGGAAGCATCTGAGCTGTAAATATCCAAAGCCAAACTATGGCTCAGAAAATTATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGCGCGTAACAGTAATATATTGTTAATGGATTGTTATCCTTGGTAG
AAGCCAGACATATCATGTCATGGACGTCTATTTGGATGTTTTCCTTGTGGTACATCTCATGAAGCTCATATATTTTGTTATAA
TACAGGAGACATTTTAATCGCCATTCATTAAGATCCGTATATTTCTCATCTAGAAAACAAATGGCGTCCTTACAATCGTATTG
TACTGCTTTGGCGTACCAATACTTCACTAGTAAACCATTTAACTCGTCCGTTTCTTTTATTTCTATGAGCCCCCATAGTCTTT
TATAAATTAAGCCCCTTAATTGTATAACAAATTTGTTTTCTAAAATAGGATTATTCATAAAAATTTCATGGCACAAAATAATA
CTGCCGCTGGTTTTATTGTGCATTATCCTGGTAAAAATACGGAAAATATCGTTGTCCTCTAGAGTTTCTTTGGCGCCTAGCTG
TCTACACAACTCTCGGATGTGCTTCGTATTGATAGAAAGCAAACCATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAATATTTCAAAATGCAGTGTTCATC
TATTGACAAACACTGGCTGGCTATCGTCTTTTTGACGAGGGTCTGCAGAGAGAGCGGCAACGACAT
Tengani 62 MGF 360 13L (AY261364.1:25753-26814)
SEQ ID No. 102
CTATAGTATATTATGAGAATATTCCCAAATGATGGATAAGTTTTTTGATTTATAATCTTTTAATAAACTGCTTATTTCTTCGG
GGTCCTTTAAGTTTAGTGGCAAGGAAGCATCTGAGCTGTAAATATCCAAAGCCAAACTATGGCTCAGAAAATTATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGCGCGTAACAGTAATATATTGTTAATGGGTTGTTATCCTTGGTAG
AAGCCAGACATATCATGTCATGGACGTCTATTTGGATGTTTTCCTTGTGGTACATCTCATGAAGCTCATATATTTTGTTATAA
TACAGGAGACATTTTAATCGCCATTCATTAAGATCCGTATATTTCTCATCTAGAAAGCAAATGGCGTCCTTACACTCGTATTG
TACTGCTTTGGCGTACCAATACTTCACTAGTAAACCGTTTAACTCATCCGTTTCTTTTATTTCTATGAGCCCCCATAGTCTTT
TATAAATTAAGCCCCTTAATTGTATAGTAAATTTATCTTCTATATTAGGATTATTCATAAATATATCATGGCACAAAATAATA
CTGCCGCTGGTTTTATTGTGCATTATCTTGGTGAAAATACGGAAAATATCGTCGTCTTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCTATATGGAGGTCTATATCTTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TATCGACAAAGACTGGTTGGCTACCGTCTTTTTGACGAGGGTCTGCAGAGAGAGCGGTAATGACAT
Warthog MGF 360 13L (AY261366.1:27277-28338)
SEQ ID No. 103
CTATAGTATATTATGAGAATATTCCCAAATGATGGATAAGTTTTTTGATTTATAATCCTTTAATAAACTGCTTATTTCTTCGG
GGTCCTTTAAGTTTAGTGGCAAGGAAGCATCTGAGCTGTAAATATCCAAAGTCAAACTATGGCTTAGAAAATGATAACCTTTT
TGTTCCGCTATAGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGGGCGTAACAATAATATATTGTTAATGGATTGTTATCCTTGGTAG
AAGCCAGACATATCATGTCATGGACGTCTATTTGGACTTTTTCCTTGTGGTACATCTCATGAAGCTCATATATTTTGTTATAA
TACAGGAGACATTTTAATCGCCATTCATTAAGATCCGTATATTTCTCATCTAGAAAGCAAATGGCGTCCTTACACTCGTATTG
TACTGCTTTGGCGTACCAATACTTCACTAGTAAATCGTTTAACTCATCCGTTTCTTTTATTTCTATGAGCCCCCATAGTCTTT
TACAAATTAAGCCCCTTAATTGTATAGTAAATTTGTTTTCTATATTAGGATTATTCATAAATATCTCATGGCACAAAATAATA
CGGCCGCTGGTTTTATTGTGCATTATCTTGGTGAAAATACGGAAAATATCGTTGTCCTCTAGAGTTTCTTTGGCACCTAGCTG
TCTACACAACTCTCGGATGTGCTCCGTATTGATAGAAAGCAAACTATAGTTGATATGTGCGCCCCATTCTGTAAAGAGCTTTA
TCAGACTATAGTTGTTTTCCTTAACAGCTATTATTAATGCTATATGGAGGTCTATATCTTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TGTCGACAAACACTGGCTGGCTACCGTCTTTTTGACGAGGGTCTGCAGAGAGAGCGGCAACGACAT
Warmbaths MGF 360 13L (AY261365.1:30498-31559)
SEQ ID No. 104
CTATAGTATATTATGAGAATATTCCCAAATGATGGATAAGTTTTTTGATTTATAACCCTTTAATAAACTGCTTATTTCTTCGG
GGTCCTTTAAGTTTAGTGGCAAGGAAGCATCTGAGCTGTAAATATCCAAAGCCAAACTATGGCTTAGAAAATGATAACCTTTT
TGTTCCGCTATGGCACGACCCTCTTCAAAGGCATTACCACCCAAATCTATACAGAAAAATATATTACCGATGTTATAATATTG
TACTGAAGTAAGCATAGCTTGGTTGATGTTGCCCCCCAGGGCGTAACAGTAATATATTGTTAATGGATTGTTATCCTTGGTAG
AAGCCAGACATATCATGTCATGGACGTCTATTTGGACTTTATCCTTGTGGTACATCTCATGAAGCTCATATATTTTGTTATAA
TACAGGAGACATTTTAATCGCCATTCATTAAGACCCGTATATTTCTCATCTAGAAAGCAAATGGCGTCCTTACACTCGTATTG
TACTGCTTTGGCGTACCAATACTTCACTAGTAAATCGTTTAACTCATCCGTTTCTTTTATTTCTATGAGCCTCCATAGTCTTT
TACAAATTAAGCCCCTTAATTGTATAGTAAATTTGTTTTCTATATTAGGATTATTCATAAATATCTCATGGCACAAAATAATA
CGGCCGCTGGTTTTATTGTGCATTATCTTGGTGAAAATACGGAAAATATCGTTGTCCTCTAGAGTTTTTTTGGCACCTAGCTG
TCTACACAACTCTCGGATGTGTTCCGTATTGATAGAAAGCAAACTATAGTTGATATGTGCGCCCCATTCTGTAAAGAGCTTTA
TCAGACTATAGTTGTTTTCCTTAACAGCTATTATTAATGCTACATGGAGATCTATATCTTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TGTCGACAAACACTGGCTGGCTACCGTTTTTTTGACGAGGGTCTGCAGAGAGAGCGGCAACGACAT
Pretorisuskop/96/4 MGF 360 13L (AY261363.1:31307-32368)
SEQ ID No. 105
CTATAGTATATTATGAGAATATTCCCAAATTATGGATAAGTTTTTTGATTTATAATCCTTTAAAAAACTGCTTATTTCTTCGG
GGTCCTTTAAGTTTAGTGGCAAGGAAGCATCTGAGCTGAAAATATCCAAAGCCAAACTATGGCTTAGAAAATTATAACCTTTT
TGTTCCGCTATGGCACGACCCTCTTCAAAGGCATTACCACCCAAATCTATACAGAAAAATATATTACCGATGTTATAATATTG
TATTGAAGTAAGCATAGCTTGGTTGATGTTGCCCCCCAGGGCGTAACAGTAATATATTGTTAATGGATTGTTATCCTTGGTAG
AAGCCAGACATATCATGTCATGGACGTCTATTTGGACTTTTTCCTTGTGGTACATCTCATGAAGCTCATATATTTTGTTAAAA
TACAGGAGACATTTTAATCGCCATTCATTAAGATCCGTATATTTCTCTTCTAGAAAGCAAATGGCGTCCTTACACTCGTATTG
TACTGCTTTGGCGTACCAATACTTCACTAGTAAATCGTTTAACTCATCCGTTTCTTTTATTTCTATGAGCCCCCATAGTCTTT
TACAAATTAAGCCCCTTAATTGTATAGTAAATTTGTTTTCTATATTAGGATTATTCATAAATATATCATGGCACAAAATAATA
CGGCCGCTGGTTTTATTGTGCATTATCTTGGTGAAAATACGGAAAACATCGTCGTCTTCTAGAGTTTCTTTGGCGCCTAGCTG
TCTACACAACTCTCGGATGTGCTCCGTATTAATAGAAAGCAAACTATAGTTGATATGTGCGCCCCATTCTGTAAAGAGCTTTA
TCAGACTATAGTTGTTTTCCTTAACAGCTATTATTAATGCTATATGGAGGTCTATATCCTCTCCTAAAAATCCTGATTTTATT
TGTATTTGGCCACGATCCATACAAAGCTTGAGGGGGACATCATGCCACCATAGGCCACAGTGTTTCAGAATGCAGTGTTCATC
TATCGACAAACACTGGCTGGCTACCGTCTTTTTGACGAGGGTCTGCAGAGAGAGCGGCAACGACAT
Mkuzi 1979 MGF 360 13L (AY261362.1:32286-33347)
SEQ ID No. 106
CTATAATATATTATGAGAATATTCCCAAATGATGGATAAGTTTTTTGATTTATAATCTTTTAATAAACTGCTTATTTCTTCGG
GGTCCTTTAAGTTTAATGGCAAGGAAGCATCTGAACTGTAAATATCCAAAGTCAAGCTATGGCTTAAAAAATTATAACCTTTT
TGTTCCGCTATGGCACGACCCTCTTCAAAGGCATTACCCCCCAAATCTATACAGAAAAATATATTACCGATGTTATAATATTG
TACTGAAGTGAGCATAGCTTGGTTGATGTTGCTCCCCAGGGCGTAACAGTAATATATTGTTAATAGATTGTTATCCTTGGCAC
AAGCCAGACATATCATGTCATGGACGTCTATTTGGACTTTTTTCTTGTGGTACATCTCATGAAGCTCATATATTTTGTTATAA
TACAGGAGACATTTTAATCGCCATTCGTTAAGATCCGTATATTTCTCATCTAGAAAGCAAATGGCGTTCTTACAAACGTATTG
TACTGCTTTGGCATACCAATACTTCACTAGTAAATCATTTAACTTGTCCGTTTCTTTTATTTCTATGAGCCCCCATAGTCTTT
TATAAATTAAGCCCCTTAATTGTATAACAAATTTGTTTTCTAAAATAGGATTATTCATAAAAATTTCATGGCACAAAATAATA
CTGCCGCTGGTTTTATTGTGCATTATCCTGGTGAAAATACGGAAAATATCGTTGTCCTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAGTATTTCAAAATGCAGTGTTCATC
TATTGACAAACACTGGCTGGCTACCGTCTTTTTGACGAGGGTCTGCAGAGAGAGCGGCAACGACAT
L60 MGF 360 13L (NC_044941.1:25189-26250)
SEQ ID No. 107
CTATAATATATTATGAGAATATTCCCAAATGATGGATAAGTTTTTTGATTTATAATCTTTTAATAAACTGCTTATTTTTTCGG
GGTCCTTTAAGTTTAATGGCAAGGAAGCATCTGAACTGTAAATATCCAAAGTCAAGCTATGGCTTAAAAAATTATAACCTTTT
TGTTCCGCTATGGCACGACCCTCTTCAAAGGCATTACCACCCAAATCTATACAGAAAAATATATTACCGACGTTATAATATTG
TACTGAAGTGAGCATAGCTTGGTTGATGTTGCCCCCCAGGGCGTAACAGTAATATATTGTTAATAGATTGTTATCCTTGGCAC
AAGCCAGACATATCATGTCATGGACGTCTATTTGGACTTTTTTCTTGTGGTACATCTCATGAAGCTCATATATTTTGTTATAA
TACAGGAGACATTTTAATCGCCACTCGTTAAGATCCGTATATTTCTCATCTAGAAAGCAAATGGCGTTCTTACAAACGTATTG
TACTGCTTTGGCATACCAATACTTCACTAGTAAATCATTTAACTCGTCCGTTTCTTTTATTTCTATGAGCCCCCATAGTCTTT
TATAAATTAAGCCCCTTAATTGTATAACAAATTTGTTTTCTAACATAGGATTATTCATAAAAATTTCATGGCACAAAATAATA
CTGCCGCTGGTTTTATTGTGCATTATCCTGGTGAAAATACGGAAAATATCGTTGTCCTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAGTATTTCAAAATGCAGTGTTCATC
TATTGACAAACACTGGCTGGCTACCGTCTTTTTGACGAGGGTCTGCAGAGAGAGCGGCAACGACAT
Benin 97/1 MGF 360 13L (NC_044956.1:24840-25901)
SEQ ID No. 108
CTATAATATATTATGAGAATATTCCCAAATGATGGATAAGTTTTTTGATTTATAATCTTTTAATAAACTGCTTATTTTTTCGG
GGTCCTTTAAGTTTAATGGCAAGGAAGCATCTGAACTGTAAATATCCAAAGTCAAGCTATGGCTTAAAAAATTATAACCTTTT
TGTTCCGCTATGGCACGACCCTCTTCAAAGGCATTACCACCCAAATCTATACAGAAAAATATATTACCGACGTTATAATATTG
TACTGAAGTGAGCATAGCTTGGTTGATGTTGCCCCCCAGGGCGTAACAGTAATATATTGTTAATAGATTGTTATCCTTGGCAC
AAGCCAGACATATCATGTCATGGACGTCTATTTGGACTTTTTTCTTGTGGTACATCTCATGAAGCTCATATATTTTGTTATAA
TACAGGAGACATTTTAATCGCCACTCGTTAAGATCCGTATATTTCTCATCTAGAAAGCAAATGGCGTTCTTACAAACGTATTG
TACTGCTTTGGCATACCAATACTTCACTAGTAAATCATTTAACTCGTCCGTTTCTTTTATTTCTATGAGCCCCCATAGTCTTT
TATAAATTAAGCCCCTTAATTGTATAACAAATTTGTTTTCTAACATAGGATTATTCATAAAAATTTCATGGCACAAAATAATA
CTGCCGCTGGTTTTATTGTGCATTATCCTGGTGAAAATACGGAAAATATCGTTGTCCTCTAGAGTTTCTTTGGCGCCTAGCTG
TCTACACAACTCTCGGATGTGCTTCGTATTGATAGAAAGCAAACTATAGTTGATATTTGCGCCCCACTCTGTAAAGAGCTTTA
TCAGACTATAGTTGTTTTCCTTAACAGCTATTATTAATGCCACACGAAGGTCTATATCTTCTCCTAAAAATCCTGATTTTATT
TGTATTCGGCCACGATCCATACAAAGCTTGAGAGGAGCATCATGCCACCATAGGCCACAATATTTCAAAATGCAGTGTTCATC
TATTGACAAACACTGGCTGGCTACCGTCTTTTTGACGAGGGTCTGCAGAGAGAGCGGCAACGACAT
Malawi Lil-20/1 (1983) MGF 360 13L (AY261361.1:28920-29980)
SEQ ID No. 109
TTAGAGTATATTATGAGAGTAATCCAAAATGATGGATAAATTTTTTGATTTATAATTTTTTAACAAACTGCTTATTTCTTCGG
GGTCCTTTAAGTTTAATGGCAAGGAAGCATCCGAGCTATAAATATTCAAAACCAAACTATGGCTTAGAAAATTATAACCTTTT
TGTCTCGCTATGGCACTACCCTCTTCAAAGGCATTACCACCCAAATCTATACAGAAATATATATTACCGATGTTATAATATTG
CACTGAAGTAAGCATAGCTTGGTTGATGTTGCCCCCCAGGGCATAACAGTAATATATTGTTAATGGATTGTTATCCCTGATAC
AAGCCAGGGATAACATTTCATTAACGTCTATTTGGATTTTTTCCTTGTGGTATATATCATGAAGCTCATATATTTTGTTATAA
TATAGGTAACATTTTAATCGCCATTCATTAAGATCCGTATATTTCTCATCCAGAAAGCAAATGGCATCTTTATAATCGTATTG
TACTGCTTTGGCGTACCAATATTTCACTAGTAATTCACTTAACTCGTCCGTTTCTTTTATTTCTATAAGCCCGCATAGTCTTT
TATAAATTAAGCGCCTTAATTGTGCAGCAAATTTGTTTTCTAAATTAGGATTATTTATAAATATCTCATGGCACAAAATAACA
CTGCCGCTGGTTTTATTATGCATTATCTTGTTGAAAATACAGAAAATATCGTAGTCCTCCAGAGTTTCTTTGGCGCCTAGCTG
TCTACACAAATCTCGGACGTGCTTCGTATTGATAGAAAGCAGACTATAGTTGATATTTGCGCCCAACTCTGTAAAAAGCTTTA
TTAGATTATAGTTGTTTTCCTTAACAGCTATTATTAATGCCACACAAAGGTCTATATCTTCTCCTAAAAATCCCGATTTTATT
TGTATTCGGCCACGATCCATATAAAGCTTGAGAGGGGCATCATGCCACCATAAGTCACAGTATTTCAAAATGTGTTCATCTAT
CGACAAACATGATGTACTGGCTACCGTCTTTTTGACGAGGGTTTGTAGAGAGAGCGGCGCCGACAT
Kenya 1950 MGF 360 13L (AY261360.1:34752-35812)
SEQ ID No. 110
TTAGAGTATTTTATGAGAGTAATCCAGAATGATGGATAAATTTTTTGATTTATAATTTTTTAATAAACTGCTTATTTCTTCGG
GAACCTTTAAGTTTAATGGCAAGGAAGCATCTGAGCTATAAATATTCAAAATTAAACTATGGCATAAAAAATTATAACCATTT
TGTCTCGCTATGGCACTACCCTCTTCAAAGGCATTACCACCCAAATCTATACAGAAATATATATTACCGATGTTATAATATTG
TACTGAAGTAAGCATAGCTTGGTTGATGTTGCCCCCCAGGGCATAACAATAATATATTGTTAATAGATTGTTATCCCTGATAC
AAGCCAGAGATAACATTTCATTGACGTCTATTTGGATTTTTTTCTTGTGGTATATATCATGAAGCTCATATATTTTGTTATAA
CATAGGTAACATTTTAATCGCCATTCATCAAGATCCGTATATTTTTCATCCAGAAAGCAAATGGCATCTTTATGATCGTATTG
TACTGCATTGGCGTACCAATATTTCACTAGTAATTCGCTTAACTCGTCCGTTTCTTTTATTTCTATAAGCCCGCATAATCTTT
TATAAATTAAGCGCCTTAATTGTGCAGCAAAGTTGTTTTCTAATTTAGGATTATTTATAAATATCTCATGGCACAAAATAATA
CTGCCGCTAGTTTTATTATGCATTATTTTATTGAAAATACAGAAAATATCGTAGTCGTCTAGAGTTTCTTTGGCGCCTAGCTG
TCTACACAAATCTCGGGCGTGCTTCGTATTGATAGAAAGCAGACTATAGTTGATATATGCGCCCCACTCTGTAAATAACTTTA
TCAGACTATAGTTATTTTCCTTAACAGCTATTATTAATGCCACACAAAGGTCTATATCTTCTCCTAGGAATCCCGATTTTATG
TATATTCGTCCACGATCTATGTAAAGCTTGAGGGGAGCATCATGCCACCATAAGCCACAGTATTTCAAAATGTGTTCATCTAT
CGACAAACATGATGTACTGGCTACCGTCTTTTTGACGAGGGTCTGCAGAGAGAGCGGCGACGACAT
Ken06.Bus MGF 360 13L (NC_044946.1:28644-29704)
SEQ ID No. 111
TTAGAGTATTTTATGAGAGTAATCCAGAATGATGGATAAATTTTTTGATTTATAATTTTTTAATAAACTGCTTATTTCTTCGG
GATCCTTTAAGTTTAATGGCAAGGAAGCATCTGAGCTATAAATATTCAAAACCAAACTATGGCTTAAAAAATTATAACCATTT
TGTCTCGCTATGGCACTACCCTCTTCAAAGGCATTACCACCCAAATCTATACAGAAATATATATTACCGATGTTATAATATTG
TACTGAAGTAAGCATAGCTTGATTGATGTTGCCCCCCAGGGCATAACAATAATATATTGTTAATAGATTGTTATCCCTGATAC
AAGCCAGAGATAACATTTCATTGACGTCTATTTGGATTTTTTCCTTGTGGTATATATCATGAAGCTCATATATTTTGTTATAA
CATAGGTAACATTTTAATCACCATTCATCAAGATCCGTATATTTTTCATCCAGAAAGCAAATGGCATCTTTATGGTCGTATTG
TACTGCATTGGCGTACCAATATTTCACTAGCAATTCGCTTAACTCGTCCGTTTCTTTTATTTCTATAAGCCCGCATAGTCTTT
TATAAATTAAGCGCCTTAATTGTGCAGCAAAGTTGTTTTCTAAATTAGGATTATTTATAAATATCTCATGGCACAAAATAATA
CTGCCGCTAGTTTTATTATGCATTATCTTGTTGAAAATACAGAAAATATCGTAGTCGTCTAGAGTTTCTTTGGCGCCTAGCTG
TCTACACAAATCTCGGGCGTGCTTCGTATTGATAGAAAGCAGACTATAGTTGATATATGCGCCCCACTCTGTAAATAACTTTA
TCATACTATAGTTGTTTTCCTTAACAGCTATTATTAATGCCACACAAAGGTCTATATCTTCTCCTAGGAATCCCGATTTTATG
TATATTCGCCCACGATCTATGTAAAGCTTGAGGGGAGCATCATGCCACCATAAGCCACAGTATTTCAAAATGTGTTCATCTAT
CGACAAACATGATGTACCGGCTACCGTCTTTTTGACGAGGGTCTGCAGAGAGAGCGGCGACGACAT

In an embodiment the attenuated ASFV of the invention comprises a functional version of MGF 360 13L. Suitably the functional version of MGF 360 13L comprises the sequence of SEQ ID No. 100, 101, 102, 103, 104, 105, 106, 107, 108, 109, 110 or 111. Suitably the functional version of MGF 360 13L comprises a sequence having at least 70%, at least 80%, at least 90% or at least 95% identity with SEQ ID No. 100, 101, 102, 103, 104, 105, 106, 107, 108, 109, 110 or 111. Suitably the functional version of MGF 360 13L consists of the sequence of SEQ ID No. 100, 101, 102, 103, 104, 105, 106, 107, 108, 109, 110 or 111.

### MGF 360 14L Gene Sequences

Georgia 2007/1 MGF 360 14L (LR743116.1:32814-33887)
SEQ ID No. 112
TTAGTCTATAACTATAATTTCTGGATGGGCTGTAAGATACTCTTCGGCTCGTTTCAGATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCAGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTGATGCAAATCTCTTTTTCACGACACAACTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGCAGAACTCCTGCACAGGATTTTAGCCTGGCCACTTCTTTTAAAATTTCCAGAAGACGGGGTTCGGATACAGGCGTTAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCGAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
China/2018/AnhuiXCGQ MGF 360 14L (MK128995.1:31843-32916)
SEQ ID No. 113
TTAGTCTATAACTATAATTTCTGGATGGGCTGTAAGATACTCTTCGGCTCGTTTCAGATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCAGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTGATGCAAATCTCTTTTTCACGACACAACTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGCAGAACTCCTGCACAGGATTTTAGCCTGGCCACTTCTTTTAAAATTTCCAGAAGACGGGGTTCGGATACAGGCGTTAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCGAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
Tengani 62 MGF 360 14L (AY261364.1:26983-28056)
SEQ ID No. 114
TTAGTCTATAACTATAATTTCTGGATGGGCTGTAAGATACTCTTCAGCTCGTTTCAGATTTTTTGAAGTATATGACTTTAGCA
TATCATATATTTCCTGGGGTTCGGTTACATCTAATGCCAAGGTCACATCACGGCTGAAAAGCTGCTTTACTAAGAAAATGTTG
CTCAAGTTATACATATAAGCTTTGTGCGCAATGCGTTGTGCCCTATCAAAATCAGTAGCCCCCAAATCGATGCAGAAAAACAT
GTTTAAAGTATTATTGTTATAGATAGAAAGATTCATGCCATAATCGAGACTAGCCCCTAACCTATAACAGTAATAAATGGCCG
CATAATTTTTTTTCTGCAAGCAAGCAAATTTCATCATCAGATTAGGGCTGATGCAAATCTCTTTTTCACGACACAACTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGCAGAACTCCTGCACAGGATTTTAGCCTGGCCACTTCTTTTAAAATTTCCAGAAGACGGGATTCGGATACAGGCGTTAA
GCCACCCAGTTCCGTGCACAGCCGCTTTAGATGCACGGCAGGAACACGTATAAGTCCATATTCAGGATTTGCGCCCCAATCCA
CAAATAAACGTATAAGCTCAAGATTATCGCTCTTCACGGCCTTTACTAGCGCCGCTTCAAGACAAAGATCATCCTCAGAAAAA
CACTGTACATGTTTATACGAAAAAATTTGCTTACAATTATTACATAGGTGAATAGGACCTAAATCCCACCACAAACCAAAACG
CTGCAACGTATAATCATAGTCACTTGAAAGATAATTGCATGCCACAACCTTTTTGGCCAATGTTTGTAAAGACAACAT
Warthog MGF 360 14L (AY261366. 1:28495-29568)
SEQ ID No. 115
TTAGTCTATAACTATAATTTCTGGATGGGCTGTAAGATACTCTTCGGCTCGTTTCAGATTTTTTGAAGTATATGACTTTAGCA
TATCATATATTTCCTGGGGTTCGGTTACATCTAATGCCAAGGTCACATCACGGCTGAAAAGCTGCTTTACTAAAAAAATGTTG
CTCAAGTTATACATATAAGTTTTGTGCGCAATGCGTTGTGCCCTATCAAAATCAGTAGCCCCCAAATCGATGCAGAAAAACAT
GTTTAAAGTATTATTGTTATAGATAGAAAGATTCATGCCATAATCGAGACTAGCCCCTAACCTATAACAGTAATAAATGGCCG
CATAATTTTTTTTCCGCAAGCAAGCAAATTTCATCATCAGATTAGGGCTGATACAAATCTCTTTTTCACGACACAGCTCGTGT
ATGTCAAAAATGTTATTAAAATAAAGGCTACAAGCTACCCGCCAATAGAGGTGATTTTTATGCCTTTTATAAAAATAGTGAAT
AGCCTTTGTAAAATTATGTCGTAATGCCAGGGCAAACCAAAACTTTGTTAATAGGTGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGCTTGGCCACTTCTTTTAAAATTTCCAGAAGACGGGGTTCGGATACAGGCGTTAA
GCCTCCCAGTTCCGTGCACAGCCGCTTTAGATGCACGGCAGGAACACGTATAAGTCCATATTCAGGATTTGCGCCCCAATCCA
CAAATAAACGTATAAGCTCAAGATTATCGCTCTTCACGGCCTTTACTAGCGCCGCTTCAAGACAAAGATCATCCTCAGAAAAA
CACTGTAAATGTTTATACGAAAAAATTTGCTTACAATTGTTACATAGGTGAATAGGACCTAAATCCCACCACAAACCAAAACG
CTGCAACATATAATCATAGTCACTTGAAAGATAATTGCATGCCACAACCTTTTTGGCCAACGTTTGTAAAGACAACAT
Warmbaths MGF 360 14L (AY261365.1:31747-32820)
SEQ ID No. 116
TTAGTCTATAACTATAATTTCTGGATGGGCTGTAAGATACTCTTCGGCTCGTTTCATATTTTTTGAAGTATATGCCTTTAGCC
TATCATATATTTCCTGGGGTTCGGTTACATCTAATGCCAAGGTCACATCACGGCTGAAAAGCTGCTTTACTAAAAGAATGTTG
CTCAAGTTATACATATAAGCTTTGTGCGCAATGCGTTGTGCCCTATCAAAATCAGTAGCCCCCAAATCGATGCAGAAAAACAG
GTTTAAAGAATTATTGTTATAGATAGAAAGATTCATGCCATAATCGAGACTAGCTCCCAACCTATAACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGCGCAAACCAAAACTTTGTTAATAGGTGGTGCGCCGTATCCCCCATCAACGGAA
TTTTTGAACAGCTGTACATAACTGTGTCTAAAGTGGTTCTAGTTATGGTTTCCAAGAGTGGGTTATAACAAAACATGTCATAA
CCCAGTAGAACTCCTGCACAGGATTTTAGGTTGGCCACTTCTTTTAAAATTTCCAGAAGACGGGGTTCGGATACAGGTGTTAA
GCCTCCCAGTTCTGTGCACAGCCGCTTTAGATGCACGGTAGGAACACATATAAGTCCATATTCAGGATTTGCACCCCAATCCA
CAAATAAACGTATAAGCTCAAGATTATCCCTCTTCACGGCCTTTACTAGCGCCGCTTCAAGACAGAGATCATCCTCAGAAAAA
CACTGTAAATGTTTATACGAAAAAATTTGCTTACAATTGTTACACAGGTGAATAGGACCTAAATCCCACCACAAACCAAAACG
CTGCAACATATAATCATAGTCACTTGAAAGATAATTGCATGCCACAACCTTTTTGGCCAACGTTTGTAAAGACAACAT
Pretorisuskop/96/4 MGF 360 14L (AY261363.1:32525-33598)
SEQ ID No. 117
TTAGTCTATAACTATAATTTCTGGATGGGCTGTAAGATACTCTTCGGCTCGTTTCAGATTTTTTGAAGTATATGACTTTAGCC
TATCATATATTTCCTGGGGTTCGGTTACATCTAATGCCAAGGTCACATCACGGCTGAAAAGCTGCTTTACTAAGAAAATGTTG
CTCAAGTTATACATATAAGCTTTGTGCGCAATGTGTTGTGCCCTATCAAAATCAGTAGCCCCCAAATCGATGCAGAAAAACAT
GTTTAAAGTATTATTGTTATAGATAGAAAGATTCATGCCATAATCGAGACTAGCCCCTAACCTATAACAGTAATAAATGGCCG
CATAATTTTTTTTCTGCAAGCAAGCAAATTTCATCATCAGATTAGGGCTGATGCAAATCTCTTTTTCACGACACAGCTCGTGT
ATGTCAAAAATGTTATTAAAATAAAGGCTACAAGCTACCCGCCAATAGAGGTGATTTTTATGCCTTTTATAGAAATAGTGAAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGCTTGGCCACTTCTTTTAAAATTTCCAGAAGACGGGGTTCGGTTACAGGCGTTAA
GCCTCCCAGTTCCGTGCACAGCCGCTTTAGATGCACGGCAGGAACACGTATAAGTCCATATTCAGGATTTGCGCCCCAATCCA
CAAATAAACGTATAAGCTCAAGATTATCGCTCTTCACGGCCTTTACTAGCGCCGCTTCAAGACAAAGATCATCCTCAGAAAAA
CACTGTAAATGTTTATACGAAAAAATTTGCTTACAATTGTTACACAGGTGAATAGGACCTAAATCCCACCACAAACCAAAACG
CTGCAACGTATAATCATAGTCACTTGAAAGATAATTGCATGCCACAACCTTTTTGGCCAACGTTTGTAAAGACAACAT
Mkuzi 1979 MGF 360 14L (AY261362.1:33547-34620)
SEQ ID No. 118
TTAGTCTATAACTATAATTTCTGGATGGGCTGTAAGATACTCTTCGGCCCGTTTCATATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCAGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGCTGATTTTTATGCCTTTTATAAAAATAGTGGAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGGTTGGCCACTTCTTTTAAAATTTCCAGAAGACGGGGTTCGGATACAGGCGTTAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCAAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
L60 MGF 360 14L (NC_044941.1:26422-27495)
SEQ ID No. 119
TTAGTCTATAACTATAATTTCTGGATGGGCTGTAAGATACTCTTCGGCTCGTTTCATATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCGGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGCTGATTTTTATGCCTTTTATAAAAATAGTGGAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGGTTGGCCACTTCTTTTAAAATTTCCAGAAGACGGGGTTCGGATACAGGCGTCAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCAAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
Benin 97/1 MGF 360 14L (NC_044956.1:26073-27146)
SEQ ID No. 120
TTAGTCTATAACTATAATTTCTGGATGGGCTGTAAGATACTCTTCGGCTCGTTTCATATTTTTTGAAGTATATGTCTTTAGCA
TATCATATATTTCCTGGGGTTCGGTTACATCTAATACCAAGGTCACATCACGGCTGAAAAGCTGCTTTACTAAGAAAATGTTG
CTCAAGTTATACATATAAGCTTTGTGCGCAATGAGTTGTGCCCTATCAAAATCGGCGGCCCCCAAATCAATACAGAAAAACAT
GTTTAAAGTATTATTGTTATAGATAGAAAGATTCATGCCATAATCGAGACTAGCCCCCAACCTATGACAGTAATAAATGGCCG
CGTAATTTTTTTCCCGCAAGCAAGCAAATTTCATCATCAGATTAGGGCTAATGCAAATCTCTTTTTCACGACACAGCTCGTGT
ATGTCAAAAATGTTATTAAAATAAAGGCTACAAGCTACCCGCCAATAGAGCTGATTTTTATGCCTTTTATAAAAATAGTGGAT
AGCCTTTGTAAAATTATGTCGTAATGCCAGGGCAAACCAAAACTTTGTTAATAGGAGGTGCGCCGTATCCCCCGTCAACGGAA
TGTTTGAACAGGTGTACGTAACTGTGTCTAAAGTGGTTCTAGTTACGGTTTCCAAGAGTGGATTATGACAAAACATGTCATAA
CCCAGTAGAACTCCTGCACAGGATTTTAGGTTGGCCACTTCTTTTAAAATTTCCAGAAGACGGGGTTCGGATACAGGCGTCAA
GCCTCCCAGTTCCGCACACAGCCGCTTTAGATACACGGCAGGAACACGTATAAGCCCATATTCAGGATTTGCGCCCCAATCCA
CAAATAAACGTATAAGTTCAAGATTATCGCTCTTCACGGCCTTTACTAGCGCCGCTTCAAGACAAAGATCATCCTCAGAAAAA
CACTGTAAATGTTTATACGAAAAAACTTGCTTACAATTGTTACATAGGTGAATAGGACCTAAATCCCACCACAAACCAAAACG
CTGCAACGTATAATCATAGTCACTTGAAAGATAATTGCATGCCACAACTTTTTTGGCCAACGTTTGTAAAGACAACAT
Malawi Lil-20/1 (1983) MGF 360 14L (AY261361.1:30193-31266)
SEQ ID No. 121
TTAGTCTATAACTACAATTTCTGGATGGGCTGTAAAATACTCTTCGGCTTGTTTTAGATTTTTTGACGTATATGTATTTAGCA
TATCATATATTGCCTGGGGCTCAGTTAGATCTAATATCAAGCTCACATCACGGCTGAAAAGCTGTTTTACTAAAAAAAGGTTG
CTCAAGTTATACATATAAACTCTACGCGCAATGAGTCGCGCCCTATCAAAGTTAGTAGCCCCCAAATCAATGCAGAAAAATAG
GTTTAAAGTATTATTGTTATAGATAGATAGATTCATGCCATAATCAAGACTAGCCCCTAACATATGACAGTAATAAATGGCTG
CATAATTTTCTTCCCGCAAGCAAGCAAATTTCATCATCAGATTAGGGCTAATGCAAATCTCTTTTTTACAACACAGCTCATGC
AAGTCAAAAATGTTGTTAAAATAAAGGCTACAAGTCAGTCGCCAATAGAGCTGATTTTTATGCTTTTCGTAGAAATAGTGAAT
AGCTTTTGTAAAATTATGTCGTAATGCCAGGGCAAACCAAAACTTTGATAATAGGAGGTGCGCCGTATCCCCCGTCAACGGAA
TTTTTGAACGGGTGTACATAACTGTGTCTAAGGTGGTTCTAGTCACGGTTTCCAAGAGTGGATTATGACAAAACATGTCATAA
CCCAGTAGAACTCCTGCACAGGATTTCAGATCGGCCACTTCTTTTAAAATCTCCAGAAGACGAGATTCGGATACAGGTGTTAA
GCCTCCTAGTTCCATGCACAGCCGCTTTAGATGCACGGCAGGAACACGTATAAGTCCATATTCAGGATTTGCGCCCCAATCCA
CAAACAATCGTATAAGCTCAAGATTATCGCTCTTCACGGCCTTTACTAGCGCCGCTTCAAGACAAAGATCATCCTCAGAAAAA
CACTGTAAATGTTTATATGAAAAAATTTGCTTACATGTATTACATAAGTGAATAGGACCTAAATCCCACCACAAACCAAAACG
CTGCAGCGTATAATCATAATCACTTGAAAGATAATTGCATGCCACAACTTTTTTGGCCAGCGTTTGTAAAGACAACAT
Kenya 1950 MGF 360 14L (AY261360.1:36121-37194)
SEQ ID No. 122
TTAGTCTATAACTACAATTTCTGGATGGGCTGTAAAATACTCTTCGGCTCGTTTTAGATTTTTTGACGTATATGTCTTTAGCA
TATCATATATTGCCTGGGGTTCGGTTATATTTAATACCAAGCTCGCATCACGGCTGAAAAGCTGTTTTACTAAAAAAATGTTG
CTCAAGTTATACATATAAACTCTATGCGCAATGAGTCGCGCTCTATCAAAGTTAGTAGCCCCCAAATCAATACAGAAAAATAG
GTTTAAAGTATTATTGTTATAGATAGATAGATTCATGCCATAATCGGGACTAGCCCCCAACATATGACAGTAATAAATGGCTG
CATAATTTTCTTCCCGCAGGCAAGCAAATTTCATCATTAGATTAGGGCTAATGCAAATCTCTTTTTTAGAACACAGCTCATGC
AAGTCAAAAATATTATTAAAATAAAGGCTACAAGCCAGCCGCCAATAGAGCTGATTTTTATGCTTTTCATAGAAATAGTGAAT
CGCTTTTGTAAAATTATGTCGTAATGCCAGGGCAAACCAAAACTTCGTTAATAGGAGGTGCGCCGTATCCCCCGTCAACGGAA
TTTTTGAACGGGTGTACATAACGGTGTTTAAAGTGGTTCTAGTCACGGTTTCCAAGAGTGGATTATGACAAAACATGTCATAA
CCCAGTAGAACTCCTGCACAGGATTTCAGATCGGCCACTTCTTTTAAAATTTCCAGAAGACGAGATTCGGAGACAGGTGTTAA
GCCTCCTAGTTCTGTGCACAGCCGTTTTAGATCCACGGCAGGAACACGTATAAGTCCATATTCAGGATTTGCGCCCCAATCCA
CAAACAATCGTATAAGCTCAAGATTATCGCTCTTCACGGCCTTTACTAGCGCCGCGTCAAGACAAAGATCATCCTCAGAAAAA
CACTGCAAATGTTGATACGAAAAAATTTGCTTACATGTATTACATAAGTGAATAGGACCTAAATCCCACCACAAACCAAAACG
CTGCAGCGTATAATCATAGTCACTTGAAAGATAATTGCATGCCACAACCTTTTTGGCCAACGTTTGTAAAGACAACAT
Ken06.Bus MGF 360 14L (NC_044946.1:29917-30990)
SEQ ID No. 123
TTAGTCTATAACGACAATTTCTGGATGGGCTGTAAAATACTCTTCGGCTCGTTTTAGATTTTTTGACGTATATGTCTTTAGCA
TATCATATATTGCCTGGGGTTCGGTTATATTTAATACCAAGCTCGCATCACGGCTGAAAAGCTGTTTTACTAAAAAAATGTTG
CTCAAGTTATACATATAAACTCTATGCGCAATGAGTCGCGCTCTATCAAAGTTAGTAGCCCCCAAATCAATGCAGAAAAATAG
GTTTAAAGTATTATTGTTATAGATAGATAGATTCATGCCATAATCGGGACTAGCCCCCAACATATGACAGTAATAAATGGCTG
CATAATTTTCTTCCCGCAGGCAAGCAAATTTCATCATTAGATTAGGGCTAATGCAAATCTCTTTTTTAGAACACAGCTCATGC
AAGTCAAAAATGTTATTAAAATAAAGGCTACAAGTTAGCCGCCAATACAGCTGATTTTTATGTTTTTCGTAGAAATAGTGAAT
CGCTTTTGTAAAATTATGCCGTAATGCCAGGGCAAACCAAAACTTCGTTAATAGGAGGTGCGCCGTATCCCCCGTCAACGGAA
TTTTTGAACGGGTATACATAACTGTGTTTAAAGTGGTTCTAGTCACGGTTTCCATAAGTGGATTATGACAAAACATGTCATAA
CCCAGTAGAACTCCTGCACAGGATTTCAGATCGGCCACTTCTTTTAAAATTTCCAGAAGACGAGATTCGGAGACAGGTGTTAA
GCCTCCTAGTTCCGTGCACAGCCGTTTTAGATGCACGGCAGGAACACGTATAAGTCCATATTCAGGATTTGCGCCCCAATCCA
CAAACAATCGTATAAGCTCAAGATTATCGCTCTTCACGGCCTTTACTAGCGCCGCGTCAAGACAAAGATCATCCACAGAAAAA
CACTGCAAATGTTGATACGAAAAAATTTGCTTACATGTATTACATAAGTGAATAGGACCTAAATCCCACCACAAACCAAAACG
CTGCAGCGTATAATCATAGTCACTTGAAAGATAATTGCACGCCACAACTTTTTTGGCCAACGTTTGTAAAGACAACAT

In an embodiment the attenuated ASFV of the invention comprises a functional version of MGF 360 14L. Suitably the functional version of MGF 360 14L comprises the sequence of SEQ ID No. 112, 113, 114, 115, 116, 117, 118, 119, 120, 121, 122, or 123. Suitably the functional version of MGF 360 14L comprises a sequence having at least 70%, at least 80%, at least 90% or at least 95% identity with SEQ ID No. 112, 113, 114, 115, 116, 117, 118, 119, 120, 121, 122, or 123. Suitably the functional version of MGF 360 14L consists of the sequence of SEQ ID No. 112, 113, 114, 115, 116, 117, 118, 119, 120, 121, 122, or 123.

### MGF 360 21R Gene Sequences

Georgia 2007/1 MGF 360 14L
SEQ ID No. 124
ATGTCTACTCCACTTTCTCTACAGACTCTTGTTAAAAAAGTGCTGGCCACACAGCACATATCTAAAGAACACTACTTTATTTT
GAAATATTGTGGTTTATGGTGGCATGAAGCGCCGATTACGATTTGCATTGATGAGGATAGCCAAATATTGATAAAATCGGCAA
GCTTCAAAGAAGGCTTATCTTTAGATATCGCATTAATGAAAGTCGTGCAAGAAAATAACCATGATTTAATAGAGTTGTTTACC
AAGTGGGGTGCAGATATCAACTCTAGCTTAGTTACTGTTAATACGGAGTATACCCGGAACCTTTGTCAGAAATTAGGCGCAAA
GGAAGCTTTGAATGAAAGGGATATTTTACAAATATTTTATAAAACACGTCATCTTAAAACTAGCAGTAATATTATTTTATATA
ATGAATTGTTTTCTAATAATCTCCTTTTCCAAAATATAGAGAGATTGAGTTTAATAGTTTATAGGGGCTTGAAAAACTTATCA
ATCAACTTTATATTGGATGATATTTCATTTAGCGAAATGTTAACTAGATACTGGTATAGTATGGCGATATTATATAACCTTAC
TGAAGCCATCCAATATTTTTATCAACGATATAGGCATTTTAAAGATTGGCGGCTTATATGTGGGCTTTCTTTTAACAATTTGT
CTGACCTTCATGAAGTATATAACTTAGAGAAGACGGATATAGACATTGATGAAATGATGAAGTTGACCTGTAGTACGTATGAT
GGTAATTATTCGACTATTTATTATTGTTTTATGTTGGGGGCTGACATCAATCGGGCAATGTTAACCTCGGTAATAAACTTTCA
TATTGGTAACTTGTTCCTTTGTATAGATTTAGGAGCTGATGCTTTCGAAGACAGCATGGAACTAGCAAAACAAAAGAATAATA
ATATATTAGTAGAAATATTATCATTTAAAAATTATTATAGTTCAAATACCTCTCTTTTATCAATAAAAACGACAGATCCGGAA
AAAATTAATGCCTTATTAGATGAAGAAAAGTATGAGTCAAAAAATATGTTAATGTATGAAGAATTATCTCATTGA
China/2018/AnhuiXCGQ MGF 360 14L
SEQ ID No. 125
ATGTCTACTCCACTTTCTCTACAGACTCTTGTTAAAAAAGTGCTGGCCACACAGCACATATCTAAAGAACACTACTTTATTTT
GAAATATTGTGGTTTATGGTGGCATGAAGCGCCGATTACGATTTGCATTGATGAGGATAGCCAAATATTGATAAAATCGGCAA
GCTTCAAAGAAGGCTTATCTTTAGATATCGCATTAATGAAAGTCGTGCAAGAAAATAACCATGATTTAATAGAGTTGTTTACC
AAGTGGGGTGCAGATATCAACTCTAGCTTAGTTACTGTTAATACGGAGTATACCCGGAACCTTTGTCAGAAATTAGGCGCAAA
GGAAGCTTTGAATGAAAGGGATATTTTACAAATATTTTATAAAACACGTCATCTTAAAACTAGCAGTAATATTATTTTATATA
ATGAATTGTTTTCTAATAATCTCCTTTTCCAAAATATAGAGAGATTGAGTTTAATAGTTTATAGGGGCTTGAAAAACTTATCA
ATCAACTTTATATTGGATGATATTTCATTTAGCGAAATGTTAACTAGATACTGGTATAGTATGGCGATATTATATAACCTTAC
TGAAGCCATCCAATATTTTTATCAACGATATAGGCATTTTAAAGATTGGCGGCTTATATGTGGGCTTTCTTTTAACAATTTGT
CTGACCTTCATGAAGTATATAACTTAGAGAAGACGGATATAGACATTGATGAAATGATGAAGTTGACCTGTAGTACGTATGAT
GGTAATTATTCGACTATTTATTATTGTTTTATGTTGGGGGCTGACATCAATCGGGCAATGTTAACCTCGGTAATAAACTTTCA
TATTGGTAACTTGTTCCTTTGTATAGATTTAGGAGCTGATGCTTTCGAAGACAGCATGGAACTAGCAAAACAAAAGAATAATA
ATATATTAGTAGAAATATTATCATTTAAAAATTATTATAGTTCAAATACCTCTCTTTTATCAATAAAAACGACAGATCCGGAA
AAAATTAATGCCTTATTAGATGAAGAAAAGTATGAGTCAAAAAATATGTTAATGTATGAAGAATTATCTCATTGA
Tengani 62 MGF 360 14L
SEQ ID No. 126
ATGTCTACTCCACTTTCTCTACAGACTCTTGTTAAAAAAGTGCTGGCCACACAGCACGTATCTAAAGATCACTACTTTATTTT
AAAATATTGTGGTTTGTGGTGGCATAAGGCCCCGATTACGATTTGCATTGATGAGGATAGCCAAATATTGATAAAATCGGCAA
GCTTTAAAGAAGGCTTACCTTTAGATATGGCATTAATGAAAGCTGTACAGGAAAACAACTATGAAATGATCATGTTGTTTACT
GAGTGGGGTGCAGACATTAACTCTAGCTTAGTCACTGTTAATACGGAGCGTACCCGGAACCTTTGTCGAGAATTAGGCGCGAA
GGAAATTTTGAATGAAAAAGAAATTTTAGAAATATTTTATAAAACATGTCATATTAAAACTAGCAGTAATTTTATTCTATGCC
ATGAATTGTTATCCAATAATCCCCTTTTCCAAAATATAGAGAGTTTGAGTTTAATAGTTTATAGGGGCTTGAAAAACTTATCA
ATTAACTTTATATTGGATGATATTTCATTTAGCGAAATGTTAACTAGATACTGGTATAGCTGGGCGATATTATATAACCTTAC
TGAAGCTATTCAATATTTCTATCAAACATACAAGCATTTTAAAGATTGGCGGCTAATATGTGGACTTGCTTATAATAACGTGT
TTGATCTTCATGAAATTTATAACATAGAAAAGACTGATTTAGATATTGATGAAATGATGCAGTTGGCTTGTATGTATGATAGT
AATTATTCAACTATTTATTATTGTTTTATGTTGGGGGCTAATATCAATCAGGCAATGTTAACCTCGGTAATAAACCATTATAT
TAATAACTTGTTCCTTTGTATAGATTTGGGAGCTGACGCCGTCAAAGACAGCATGGAACTAGCAAAACAAAAGAATTATAATA
TATTAGTAAAAATATTAACATTTAAAAATTATAGTCCAGACTCTTCTCTTTTATCATTAAAAACGACAGATCCGGAAAAAATT
AATGCCTTACTAGATGAAGAAAAGTATGAGTCGAAAAATATGTTAATGTATGAAGAATCATCTCGTTGA
Pretorisuskop/96/4 MGF 360 14L
SEQ ID No. 127
ATGCCTTCTCCACATTCTCTACAGACTCTTGCTAAAAAAATACTGGCTACACAGCAGATATCTACAGATCACTACTTTATTTT
AAAATATTGTGGTTTATGGTGGCATGGAGCTCCAATTATGCTTTCTACTAATGAGGATAATCAATTGATGATAAAATCGGCAA
GCTTCAAAGAAGGCTTATCTTTAGATTTGGCATTAATGAAAGTCGTGCAAGAAAACAATCATGATTTAATAAAGCTGTTTACC
GAGTGGGGTGCAGACATCAACTCTAGCTTCGTCACTGTTAATATGGAGTGTACCCGAAACCTCTGTCGGGAATTAGGCGCGAA
GGAAGCTTTGAATGAAAGGGATATTTTACAAATATTTTATAAAACACGTGATATTAAAACTAGCAGTCATGTTATTTTATGCC
ATGAATTATTATCCAATAACCCCCTTTTCCAGAATATAGAGAGAATGCGTTCAATAATTTATAGGAGTCTGGAAAAACTATCA
ATCAACTTTATATTGGATGATATTTCATTTAGTGAAATGTTAACTAGACACTGGTATGGGTTGGCGATATTATATAACCTAAC
TGAAGCTATCCAATATTTCTATGAAAAATACAAGCATTTTAAAAATTGGCGGCTAATATGTGGACTTTCTTTTAACAATCTGT
CTGATCTTTATGAAATATATAATTTAGAGAAGGTTGATATGAACATTGATGAAATGATGTATCTGGCCTGTAGTATATATGAT
GGTAATTATTCAACTATTTATTATTGTTTTGTATTGGGAGCTGATATCAATCAGGCAATGTTAACCTCAGTAATAAACCATTG
TATTGGTAACTTGTTCCTTTGTATAGATTTAGGGGCTGATGCTTTCGAAGACAGCATGGAACTAGCAAAGCAAAAAAATGATA
ATATTTTTATTAGTATATTATCATTTAAAAATTACAGTCCAGACTCTTCTCTTTTGTCATTAAAAATGACAGATCCGGAAAAA
ATTAATGCCTTACTAGATGAAGAAAAGTATGAGTCAAAAAATATGTTAATGTTTGA
Mkuzi 1979 MGF 360 14L
SEQ ID No. 128
ATGCCTACTCCACTTTCTCTACAGACTCTTGCTAAAAAAGTACTGGCCACACAGTATATATCCATAGATTATTACTTTATTTT
GAAATATTGTGGTTTATGGTGGCATGGAGCCCCTATTATGATTTCCATTGATAAGAATAAAAGCCAAATACTGATAAAATCGG
CAAGCTTCAAAGAAGGCTTATCTTTAGATATCGCATTAATGAAAGCTGTGCAGGAAAACAATCATGATTTAATAAAGCTGTTT
ACCGAGTGGGGTGCAGACATCAACTCTAGCTTAGTCACTGTTAATACGGAGTGTACCCGAAACCTCTGTCGGGAATTAGGCGC
GAAGGAAGCTTTGAATGAAAGGGATATTTTACAAATATTTTATAAAACACGTGATATTAAAACTAGCAGTAATGTTATACTAT
GCCATGAATTGTTATCCAATAACCCCCTTTTCCAAAATATAGAGAGAATGCGTTCAATAATTTATAGGAGTCTGGAAAAACTA
TCAATTAACTTTATATTGGATGATATTTCATTTAGCGAAATGTTAACTAGACACTGGTATAGGTTGGCGATATTATATAACCT
AACTGAAGCTATCCAATATTTCTATGAAAAATACAAGCATTTTAAAGATTGGCGGCTAATATGTGGGCTTTCTTTTAACAATC
TGTCTGACCTTCATGAAATATATAACTTAGAGAAGGTTGATATGGACATTGATATAATGATGTATCTGGCCTGTAGTACGTAT
GATGGTAATTATTCAACTATTTATTATTGTTTTGTATTGGGGGCTGATATCAATCAGGCAATGTTAACCTCGGTAATAAACCA
TCGTATTGGTAACTTGTTCCTTTGTATAGATTTAGGGGCTGATGCCTTCGAAGACAGTATGAAACTAGCAAAACAAAAGAATC
ATAATATATTAGTACATATATTATCGTTTAAAAACTATAGTCCAGACTTCTCCCTTTTATCAATAAAAACGACAGATCCAGAG
AAAATTAATGCCTTACTAGATGAAGAAAAGTATGAGTCAAAAAATATGTTAATGTATGATGAATTTGACGCCTGTACTAATAA
TGTTTAA
Warmbaths MGF 360 14L
SEQ ID No. 129
ATGCCTACTCCACTTTCTCTACAGACTCTTGCTAAAAAAGTACTGGCCACACAGTATATATCCATAGATTATTACTTTATTTT
GAAATATTGTGGTTTATGGTGGCATGGAGCCCCTATTATGATTTCCATTGATAAGAATAAAAGCCAAATACTGATAAAATCGG
CAAGCTTCAAAGAAGGCTTATCTTTAGATATCGCATTAATGAAAGCTGTGCAGGAAAACAATCATGATTTAATAAAGCTGTTT
ACCGAGTGGGGTGCAGACATCAACTCTAGCTTAGTCACTGTTAATACGGAGTGTACCCGAAACCTCTGTCGGGAATTAGGCGC
GAAGGAAGCTTTGAATGAAAGGGATATTTTACAAATATTTTATAAAACACGTGATATTAAAACTAGCAGTAATGTTATACTAT
GCCATGAATTGTTATCCAATAACCCCCTTTTCCAAAATATAGAGAGAATGCGTTCAATAATTTATAGGAGTCTGGAAAAACTA
TCAATTAACTTTATATTGGATGATATTTCATTTAGCGAAATGTTAACTAGACACTGGTATAGGTTGGCGATATTATATAACCT
AACTGAAGCTATCCAATATTTCTATGAAAAATACAAGCATTTTAAAGATTGGCGGCTAATATGTGGGCTTTCTTTTAACAATC
TGTCTGACCTTCATGAAATATATAACTTAGAGAAGGTTGATATGGACATTGATATAATGATGTATCTGGCCTGTAGTACGTAT
GATGGTAATTATTCAACTATTTATTATTGTTTTGTATTGGGGGCTGATATCAATCAGGCAATGTTAACCTCGGTAATAAACCA
TCGTATTGGTAACTTGTTCCTTTGTATAGATTTAGGGGCTGATGCCTTCGAAGACAGTATGAAACTAGCAAAACAAAAGAATC
ATAATATATTAGTACATATATTATCGTTTAAAAACTATAGTCCAGACTTCTCCCTTTTATCAATAAAAACGACAGATCCAGAG
AAAATTAATGCCTTACTAGATGAAGAAAAGTATGAGTCAAAAAATATGTTAATGTATGATGAATTTGACGCCTGTACTAATAA
TGTTTAA
Ken05/Tk1 MGF 360 14L
SEQ ID No. 130
ATGCCTACACCGTCTTCTTTACAAGTCCTTGTCAAAAGGGTGTTGGACTACCAGCATCATGTATCTGAAGATGATTATTGTAT
TTTACAGCATTGTGGGTTGTGGTGGCATGGAAGTCCAATTATGTTTTTTACTAATGAAAATCATCAAAGGATGATAAAATCAG
CAAGCTTTAAAGATGGTTTAGAAATAAATCTCGCATTAATGAAGGCCGTACAGGAAAACAATTGTATCTTAATAGAGCTGTTT
ACCGAATGGGGTGCAGACATTAATTATGGATTGATTACTGTCAATACGGAATATACCCGAAACCTATGTCGAAATCTAGGTGC
CAAGGAGGCGTTAAATACACGGAAAATTTTAGATGTATTTTTAAATTTAAAGGATTTTAAAAGTAGTAATCATATTATTCTAT
GCCATGAATTGTTATCTAATAATCCTCTTTTACTAAGTGAAAATAATGATTATTTGAGAAAAATAATTAATTGTAATCTAAGG
AGAATATCAATTAACTTTATATTGGATGAAATTTCATTTAATGAAAAGCTAACCAGATTCTGGTATAAACAGGCAGTACTAAA
TAATCTTACTGAAGCTATCCAATATTTCTATCAAAAATATAAGCAGTTTAAAGACTGGCGACTAATATGTGGACTTGCTTTAA
ATAATGTATTTGATCTTCATGAAATATATAACAAAGAGAAGGCTGATATAGATATTAATCAAATGATAGAGATAACCTGTACA
TATATGTGTAGTTATCCCACCATTTACTATTGTTTTGTAATGGGGGCTGACATTAATCGGGCAATGATTACCTCAGTTACAAA
ATCTTATACTGGTAACTTGTTCTTTTGTATAGATTTAGGAGCTACTGCCTTCGAAGAGTGCTTAGAAATAGCAAAACAACAGA
ATGATAATGAATTGGTAAAGGTATTATCATTGAAAAACTATTACAGTCCAGATAGCTCCCTTTTATCATTAAAAATAACAGAT
CCGGAAAAAATTAATATCTTATTAGATGATGAAACATATGAGTCAAAAAATGAGTTAATATATGAAGAATTAATATAA
Kenya 1950 MGF 360 14L
SEQ ID No. 131
ATGTCCACACCATCTTCTTTACAAGTCCTTGTCAAAAGGGTGCTGGACTTCCAGCATGTGTCTGAAGATGATTACTGTATTTT
AAAATGTTGTGGGTTATGGTGGCATGGAGGCCCAATCATGCTTTCTACGAATGAGGATAATCAAATGATGATAAAATCAGCAA
GTTTTAAAGATGGTTTAGAGATAAATCTCGCATTAATGATGGCCGTACAAGAAAACAATTGTAGCCTAATAGAGCTGTTTACC
GAATGGGGTGCAGACATTAATTCTGGATTGGTTACTGTCAACACGGAATACACCCGAAACCTATGTCGAAATCTAGGTGCTAA
GGAGACATTAAATAAACGGGAAATTTTAGATGTATTTTTAAAATTAAAGAATTTTAAAAGTAGTAATAATATTATTCTATCCC
ATGAATTATTATCTAATAATCCTCTTTTCCTAAGTGAAGATAATGATTATTTTAGAAGAATAATTAATTGTAATCTAAGGAGA
ATATCAATTAACTTTATATTGGATGAAATTTCATTTAATGAAAAGCTAACCAGATTCTGGTATAGTCAGGCGGTACTATATAA
TCTTACTGAAGCTATCCAATATTTCTATCAAAAATATGAACATCTCAATGAGTGGCGTTTAATTTGTGCGCTTTCTTTTAATA
ATGTGTTTGATCTTCATGAAATATATAACAAAGAAAAGGTTGGTATGGATATTAATCAAATGATAGAGATAACCTGTGCATAT
ATGTGTAGTTATTCCACCATTTACTATTGTTTTGTAATGGGAGCTGACATTAATCGGGCAATGATTATCTCGGTTACAAAATC
TTATACTTATAACTTGTTCTTTTGTATAGATTTAGGAGCTACTGCCTTCGAAGAGTGTTTAGAAATAGCAAAACAACAGAATA
ATAATGAATTAGTAAAAATATTATCATTGAAAAACTATTATAGTCCAGATAGCTCCCTTATATCATTAAAAATAACAGATCCG
GAGAAAATTAATATCTTATTAGATGAGGAAAATTATGAGTCAAAAAATGAGTTAATATATGAAGAATCTAATATAAATAATAG
CGATGATATATTTTGA
Ken06.Bus MGF 360 14L
SEQ ID No. 132
ATGCCATCTACTTTACAAGCACTTGCTAAAAAAGTACTGGCCACACCGCATATATCTAAAAAATATTGTCAGTCTAAGGAGTA
CTGTCATATATTAAAATGTTGTGGTTTATGGTGGCATGATGCTCCAATTACAATTTATCCTTGTATAAATAAAATATTGATAA
AAACAGCAAGCTTTAAACATGGTTTAGAGTTGAATGTCGCATTCATGAAAGCTGTACAGGAAAATAATTATGATCTAATAAGG
CTGTTTATTGAGTGGGGTGCGGATATCAACTTTGGGTTGGCCACTGTTAATACAGACCGTACCCGGGACCTGTGCCGGGAACT
AGGTGCGAAGGAAGCTTTAAGTGAAAAAGAAATTTTAGAAATATTTGATAAAATACAGTATATTAAAAGTAGCAGTAATATTA
TTGTATCCCATGAATTATTATCTAATAACCCCCTTTTCTTAAATAATGATCAATTGAAATTAAGAATGTTCGATGAACTACAT
AAACTATCAATCAACTTTGTATTACATGAGATATCATTTAATGAAATGCTAACTAGGTATTGGTATAGTATGGCGATACTATA
TAACCTCACCGCAGCCATTCAATATTTTTATCAATCCTACAAGCATTTTAAAGATTGGCGGTTAATATGTGGGCTTGCTTATA
ACAACGTGTTTGATCTTCATGAAATATATAACAAAGAGAAGGTTGATATGGATATTAATCAAATGATGCAGTTGGCCTGTATG
TATGATGGTAATTATACAACCATTTATTATTGTTTTATGTTGGGAGCTGACATTAATCGGGCAATGATTACCTCGGTTACAAA
ATCTTGTGATGGTAACTTATTCCTTTGTATAGATTTGGGGGCTGATGTATTTGAAGAGAGTATGAAAATGGCAATAGAAGATC
ATAATGACGTATTAGAAAGTATCTTATCATTTAAAAATTATTATAGTTCAGACGTTTCTCTGTTATCATTAAAGACGACAGAT
CCAGAAAAAATTAATGCCTTGTTAGAAGAAGAAAGTTACAAATCAAAAAATAGGTTGATATATAAAAGTTATTGA
Malawi Lil-20/1 (1983) MGF 360 14L
SEQ ID No. 133
ATGATGCCATCTACTTTACAAACACTTGCTAAAAAAACATTGGCCACACAGCATATATCTAAAAAATATTGGCCGTCTGAGGA
GTATTGTTATATATTAAAATGTTGTGGTTTATGGTGGCATGATTCTCCAATCACGATTTTTACTTGTATAAAACAAATATTAA
TAAAAACAGCAAACTTTAAACATGGTTTAGACTTAAACCTTGCATTAATGAAAGCTGTACAAGAAAATAACTATGAATTGATC
ATGTTGTTTACTGAGTGGGGTGCGGATATCAACTTGGGGTTGATCACTGTTAATACAGAGTGCACCCGAGATTTGTGCCAAAA
GTTAGGTGCAAAGGAAGCTTTGAGTGCAAAGGAAATTTTAGAAATATTTTATAAAATACAGTATATTAAGAGTAGCAATAATA
TTATTATATCTCATGAATTAATATCTAATCATCCCCTTTTCCTAAATAATGATCAATTGAAATTAAGAATTGTTGGTGAACTA
AATGCTATATCAATCAACTTTATATTGGATGAGATATCATTCAATGAAATGCTAACTAGGTATTGGTATAGTATGGCGATACT
ATATAAACTCCCTGCAGCCATTCAATATTTTTATCAATCATACAAGTATTTTAAAGATTGGCGGTTAATATGTAGCCTTGCTT
ATAACAACGTGTTTGACCTTCATGAAATTTATAATAAAGAGAAGACTGATATAAACATTGATGAAATGATGCGGTTGGCCTGT
AGGTATGATGGTAATTATACAACTATTTATTATTGTTTTATGTTGGGGGCTGACATTAATCAGGCAATGATTACCTCGGTCAT
GAATCTTTGGGACGGTAACTTATTTCTTTGTATAGATTTGGGGGCTGATGTATTTGAAGAGTGTATGAAAATAGCAATAGAAG
ATCATAATGGTGTATTAGAAAGTATCTTATCATTTAAAAATTATTATAGTCCAGACGTTTCTCTGTTATCATTAAAAACGACA
GATCCGGAAAAAATTAATGACTTGCTAGATGAAGATATCTATAAATCAAAAAATAGGTTGATATATAAAAGTTGTTGA

In an embodiment the attenuated ASFV of the invention comprises a functional version of MGF 360 21R. Suitably the functional version of MGF 360 21R comprises the sequence of SEQ ID No. 124, 125, 126, 127, 128, 129, 130, 131, 132 or 133. Suitably the functional version of MGF 360 21R comprises a sequence having at least 70%, at least 80%, at least 90% or at least 95% identity with SEQ ID No. 124, 125, 126, 127, 128, 129, 130, 131, 132 or 133. Suitably the functional version of MGF 360 21R consists of the sequence of SEQ ID No. 124, 125, 126, 127, 128, 129, 130, 131, 132 or 133.

### MGF 505 1R Gene Sequences

Georgia 2007/1 MGF 505 1R (LR743116.1:28707-30302)
SEQ ID No. 134
ATGTTCTCTCTCCAGAACTTATGTCGAAAAACATTACCTAACCGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATACCCTCATTC
CCGTAAATGAAGCCCTGAGAACAGCAGCATCTGAAGAAAATTATGAGATCGTGAGCCTTTTATTAGCGTGGGAGGGGAACCTT
TACTATGCTATTATAGGGGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATCATGCGGCAATGCTTTTTTGATTGTATTTTATATC
AAGCTGTAAAATATAGTAAGTTTCGCGTTCTTCTTTACTTTAAACATAGATTAGAGGATGATTTGCCCTTCACTCATTTACTT
ATTGAAAAGGCATGTAAAGATCATAATTATGAAGTTATTAAATGGATATATGAAAACCTACATATCTACAATATGATAGATAC
CTTTGAATGTGCTATTGCCCATAAGGATCTACATCTATATTGTTTGGGGTATAGATTTATATATAACAGAATCGTACCCGATA
AGTATCATCATTTAGATATTCGCATGCTTTCAAGCCTACAACTCCTACATAAGGTGGCAGCCAAAGGATACTTAGATTTTATC
CTAGAAACCTTAAAGTATGATCATAATAAAGATAATATAAATATTATTCTAACACAAGCTGCAACCTATAACCATAGAAAAAT
TTTAATCTATTTCATTCCTCAATCAACCCACGCACAGATAGAACAATGTTTACTAGTGGCGATAAAAGCAAAATCTTCCAGGA
AAACCTTGAACTTACTACTGTCTCACCTAAACCTTTCCATCAACCTCATCAAAAAAATAAGCCATTATGTTGCCACTTACAAT
TCAACAAATATAATAGGCATTCTGAGTATGCGGCGGAAAAAGAAGATATATTTAGATATCATATTGACAAAATTTGTAAAAAA
AGCTATTTTTAATAAGTTTGTCGTTCGATGTATGGATACATTTTCTATAAACCCGGAAAGAATCCTTAAAATAGCCGCGCGAA
TAAATAGGATGATGTTAGTGAAAAAAATATCTGAACATGTTTGGAAAAATCATGCGGTTAGACTTAAATACCTTAAACATGCG
GTACACACGATGAAGCATAAAGATGGGAAAAATAGACTCATGAACTTTATCTATGATCGCTGTTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACGATACGATTCAAAAGCCTTCTTTTAGATTGTTCACATATCATAGGTAAAAACGCTCATGATGCTACCAATATCAACATC
GTGAACAAGTATATCGGCAACCTGTTTGTTATGGGAGTTCTTAGCAAAAAAGAAATCTTACAGGACTATCCATCCATTTATTC
TAAACAATACATGCCTTAG
China/2018/AnhuiXCGQ MGF 505 1R (MK128995.1:27736-29331)
SEQ ID No. 135
ATGTTCTCTCTCCAGAACTTATGTCGAAAAACATTACCTAACCGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATACCCTCATTC
CCGTAAATGAAGCCCTGAGAACAGCAGCATCTGAAGAAAATTATGAGATCGTGAGCCTTTTATTAGCGTGGGAGGGGAACCTT
TACTATGCTATTATAGGGGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATCATGCGGCAATGCTTTTTTGATTGTATTTTATATC
AAGCTGTAAAATATAGTAAGTTTCGCGTTCTTCTTTACTTTAAACATAGATTAGAGGATGATTTGCCCTTCACTCATTTACTT
ATTGAAAAGGCATGTAAAGATCATAATTATGAAGTTATTAAATGGATATATGAAAACCTACATATCTACAATATGATAGATAC
CTTTGAATGTGCTATTGCCCATAAGGATCTACATCTATATTGTTTGGGGTATAGATTTATATATAACAGAATCGTACCCGATA
AGTATCATCATTTAGATATTCGCATGCTTTCAAGCCTACAACTCCTACATAAGGTGGCAGCCAAAGGATACTTAGATTTTATC
CTAGAAACCTTAAAGTATGATCATAATAAAGATAATATAAATATTATTCTAACACAAGCTGCAACCTATAACCATAGAAAAAT
TTTAATCTATTTCATTCCTCAATCAACCCACGCACAGATAGAACAATGTTTACTAGTGGCGATAAAAGCAAAATCTTCCAGGA
AAACCTTGAACTTACTACTGTCTCACCTAAACCTTTCCATCAACCTCATCAAAAAAATAAGCCATTATGTTGCCACTTACAAT
TCAACAAATATAATAGGCATTCTGAGTATGCGGCGGAAAAAGAAGATATATTTAGATATCATATTGACAAAATTTGTAAAAAA
AGCTATTTTTAATAAGTTTGTCGTTCGATGTATGGATACATTTTCTATAAACCCGGAAAGAATCCTTAAAATAGCCGCGCGAA
TAAATAGGATGATGTTAGTGAAAAAAATATCTGAACATGTTTGGAAAAATCATGCGGTTAGACTTAAATACCTTAAACATGCG
GTACACACGATGAAGCATAAAGATGGGAAAAATAGACTCATGAACTTTATCTATGATCGCTGTTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACGATACGATTCAAAAGCCTTCTTTTAGATTGTTCACATATCATAGGTAAAAACGCTCATGATGCTACCAATATCAACATC
GTGAACAAGTATATCGGCAACCTGTTTGTTATGGGAGTTCTTAGCAAAAAAGAAATCTTACAGGACTATCCATCCATTTATTC
TAAACAATACATGCCTTAG
Tengani 62 MGF 505 1R (AY261364.1:22891-24486)
SEQ ID No. 136
ATGTTCTCTCTTCAGAACCTATGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATAACCTCATTC
CCGTAAATGAAGCCCTAAGAATAGCAGCATCTGAAGAAAATTATGAGATCGTGAGTCTTTTATTAGCATGGGAGGGGAACCTT
TACTATGCTATTATAGGGGCTCTAGAGGGCAACCGCCACGACTTAATTCGTAAGTATGATGACCAAATCAAGGACCATCATGA
AATTCTGCCATTCATTGACGATCCAGTAATATTTCACAAATGCCATATCATGCGGCAATGCTTTTTTGATTGTATTTTATATC
AAGCTGTAAAATATAGTAAGTTTCGCGTTCTTCTTTCTTTTAAACATAGATTAAGGGATGATTTGCCCTTCACTCATTTACTT
ATTGAAAAGGCATGTAAAGATCATAATTATGAAGTAATTAAATGGATATATGAAAACCTACATATCTACAATATGATAGATAC
CTTTGAATGTGCTATTGCCCATAAGGATCTACGTCTATATTGTTTGGGGTATAGATTTATATATAACAGGATCGTACCCGATA
AGTATCATCATTTAGATATTCGCATGCTTTCAAGCCTACAACTCCTGCATAAGGTGGCAGCCAAAGGATACTTAGATTTTATC
CTAGAAACCTTAAAGTATGATCATAATAAAGATAATATAAATATTATTCTAACACAAGCTGCAACCTATAACCATAGAAAAAT
TTTAATCTATTTCATTCCTCAATCAACCCACGCACAGATAGAACAATGTTTACTAGTGGCGATAAAAACAAAATCTTCCAAGA
AAACCTTGAACTTACTACTGTCTCACCTAAACCTTTCCATCAATCTCATCAAAAAAATAAGCCATTATGTTGCCACTTACAAT
TCAACAAATATAATAGGCATTCTGAGTATGCGGCGGAAAAAGAAGATATATTTAGATATCATATTGACAAAATTTGTAAAAAA
AGCTATTTTTAATAAATTTGTCGTTCGATGTATGGATACATTTTCTATAAACCCGGAAAGAATCCTTAAAATAGCCGCGCGAA
TAAATAGGACGATGTTAGTGAAAAAAATATCTGAACATGTTTGGAAAAATCATGCGGTTAGACTTAAATACCTTAAACATGCG
GTACACACGATGAAGCATAAAGATGGGAAAAATAGACTCATGAACTTTATCTATGATCGCTGTTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACGATACGATTCAAAAGCCTTCTTTTAGATTGTTCACATATCATAGGTAAAAACGCTCATGATGCTACCAATATCAACATC
GTGAACAAGTATATCGGCAACCTGTTTGTTATGGGAGTTCTTAGCAAAAAAGAAATCTTACAGGACTATCCATCCATCTATTC
TAAACATTACATGCCTTAG
Warmbaths MGF 505 1R (AY261365.1:27651-29246)
SEQ ID No. 137
ATGTTCTCTCTCCAGAACCTATGTCGAAAAACTTTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACGTACCCTCATTC
CCGTAAATGAAGCCCTGAGAATAGCAGCATCTGAAGAAAATTATGAGATCGTGGGCCTTTTATTAGCGTGGGAGGGGAACCTT
TACTATGCTATTATAGGAGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATAATCCGACGATGCTTTTTTAATTGTATTTTATATC
AAGCTGTAAAATATAGTAAGTTTCGCGTTCTTCTTTACTTTAAACATAGATTAGAGGATGATTTGCCCCTCACTCATTTACTT
ATTGAAAAGGCATGTGAAGATCATAATTATGAAGTTATTAAATGGATATATGAAAACCTACATACCTACGATATAATGGATAC
CTTTGAATGTGCTATTGCCCATAAGGATCTACGTCTATATTGTTTGGGGTATACATTTATATATAACAGGATCGTACCCTATG
AGTATCATCATTTAGATATTCTCATTCTTTCAAGCCTACAACTCCTGCATAAGGTGGCAGCCAAAGGATACTTAGATTTTATC
CTAGAAACCTTAAAGTATGATCATAACAATGATAATTTAGATATTATTCTAACACAAGCTGCAACATATAACCATAGAAAAAT
TTTAACCTATTTCATTCCTCAATTAACCTACGCACAAATAGAACAATGTTTGTTCATGGCGATAAAAAGAAAATCTTCCAAGA
AAACCTTGAACTTACTACTGTCTCACCTAACCCTTTCCATCGAACTCATCAAAAAAATAAGCCAATATGTTGTCACTTACAAT
TCAACAAATATAATAGGCATTCTGAGTATGAAGCGGAAAAAGAAGATATATTTAGATATCATGTTGACAAAATATGTAAAATA
CGCTATTTTTAATAAATATGTCGTTCGATGTATGGATAGATTTTCCATAAACCCGGAAAGAATCATCAAAATGGCCGCGCGAA
TAAATAGGATGATGTTAGTGAAAAAAATATCTGAACATGTTTGGAAAAATCATGCGGCTAGACTTAAACACCTTAAGCATGCG
GTACACACGATGAAGCATAAAGATGGGAAAAATAGACTCATGAACTTTATCTATGATCGCTGCTATTACCATATGCAAGGGGA
AGAAATCTTTAGCCTCGCAAGATTTTATGCAATCCATCATGCACCAAAGTTGTTTGACGTTTTTTATGATTGTTGTATCCTAG
ATACTATACAATTCAAAAGCCTTCTTTTAGATTGTTCACATATCATAGGTAAAAACGCTCATGATGCTACCAATATCAACATC
GTGAACAAGTATATTGGCGACCTGTTTGCTATGGGAGTTCTTAGCAAAAAAGAAATCTTACAGGACTATCCATCCATCTATTC
TAAACATTACATGCTTTAG
Mkuzi 1979 MGF 505 1R (AY261362.1:29425-31020)
SEQ ID No. 138
ATGTTCTCTCTCCAGAACTTATGTCGAAAAACATTACCTGATTGTAAACTTCCTGAATTTTTTGACGATTATATATTACAACT
GCTGGGATTATACTGGGAAAACCATGGAACTATTCAACGGGCAGGAAACAACTGTGTGCTTATACAGCAACATACCCTTATTC
CCATAAATGAAGCCCTGAGAACAGCAGCATCTGAAGAAAATTATGAGATCGTGAGCCTTTTATTAGCATGGGAGGGGAATCTT
TACTATGCTATTATAGGGGCTCTAGAGGGCAACCGCCACGACTTAATTCGTAAATATGATGACCAAATCAAGGACCATCATGA
AATTCTGCCATTCATTGACGATCCAGTAATATTTCACAAATGCCATATCATGCGGCGATGCTTTTTTGATTGTATTTTATATC
AAGCTGTAAAATATAGTAAGTTTCGCGTTCTTCTTTATTTTAAATATAGGTTAGAGGATGATTTACCCCTCACTCATTTACTT
ATTGAAAAGGCATGTGAAAATCATAATTATGAAGTTATTAGATGGATATATGAAAACCTACATATCTACAATATGATAGATAC
CTTTGAATGTGCTATTGCCCATAAGGATCTACGTCTATATTGTTTGGGGTATACATTTATATATAACAGGATCGTACCCAATA
AGTATCATCATATAGATATTCTCATTCTTTCAAGCCTACAACTCCTGCATAAGGTGGCAGCCAAAGGATACTTAGACTTTATC
CTAGAAACCTTAAAGTATGATCATAACAATGATAATTTAGATATTATTCTAACACAAGCTGCAACATATAACCATAGAAAAAT
TTTAACCTATTTTATTCCTCAATCAACCTACGCACAAATAGAACAATGTTTGATGGTGGCGATAAAAACAAAATCTTCCAAGA
AAACCTTGAATTTACTATTGTCCCACCTAAACCTTTCCATCAAACTCATCAAAAAAATAAGCCAATATGTTGTCACTTACAAT
TCAACAAATATAATAGGCATTCTGAGTATGAAGCGAAAAAAGAAGATATATTTAGATATCTTGTTGACAAAATTTGTAAAAAA
TGCTATTTTTAATAAATTTGTCGTTCGATATATGGATACGTTTTCTATAAACCCGGAAAAAATCGTCAAAATGGCCGCGCGAA
TAAATAAGATGATGTTAGTGAAAAAAATATCTGAACATATTTGGAAAAATCATGCGGCTAGACTTGAACACCTTAAACATGCG
GTACACACGATGAAGCATAAAGATGGGAAAAATAGACTCATGAACTTTATCTATGAGTACTGCTATTACCATATGCAAGGGGA
AGAAATTTTTAGCCTCGCAAGATTTTATGCAATCCATCATGCACCAAAGTTGTTCGACGTTTTTTATAATTGTTGTATCCTAG
ATACGATACGATTCAAAAGCCTTCTTTTAGATTGTTCACATATCATAGGTAAAAACGCTCATGATGCTACTAATATCAACATC
GTGAACAAGTATATTGACAACCTGTTTGCTATGGGAGTTCTTAGCAAAAAAGAAATCTTACAGGACTATCCATCCATTTATTC
TAAACATTATATGCCTTAG
Warthog MGF 505 1R (AY261366.1:24387-25982)
SEQ ID No. 139
ATGTTTTCTCTCCAGAACTTGTGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATACAGCAACATACCCTCATTC
CCGTAAACGAAGCCCTGAGAATAGCAGCATCTGAAGAAAATTATGAGATCGTGAGCCTTTTATTAGCGTGGGAGGGAAACCTT
TACTATGCTATTATAGGAGCTCTAGAGGGCAACCGCCACGACTTAATTCGTAAATATGATGACCAAATCAAGGACCATCATGA
AATTCTGCCATTCATTGACGATCCAGTCATATTTCACAAATGCCATATCATGCGGCGATGCTTTTTTGATTGTATTTTATATC
AAGCTGTAAAATATAGTAAGTTTCGCGTTCTTCTTTATTTTAAATATAGGTTAGAGAATGATTTGCCCCTCGCTCATTTACTT
ATTAAAAAGGCATGTGAAGATCATAATTATGAAGTTATTAAATGGATATATGAAAACCTACATATCTACAATATAATGGATAC
CTTTGGATGTGCTATTGCCCATAAGGATCTACGTCTATATCGTTTGGGGTATACATTTATATATAACAGGATCGTACCCTATA
AGTATCATTATTTAGATGTTCTCATTCTTTCAGGCCTACATCTCCTGTATAAGGTGGCAGCCAAAGGATACTTAGATTTTATC
CTAGAAACCTTAAAGTATGATCATAACAATGATAATTTAGATATTATTCTAACACAAGCTGCAACATATAACCATAGAAAAAT
TTTAACCTATTACATTCCTCAATTAACCTATGCACAAATAGAACAATGTTTGTTCATGGCGATTAAAAAAAAATCTTCCAAGA
AAACCTTGAACTTACTACTGTCTCACCTAAAGCTTTCCATCAAACTCATCAAAAAAATAAGCCAATATGTTGCCACTTACAAT
TCAACAAATATAATAGGCATTCTGAATATGCGGCGGAAAAAGAAGATATATTTAGATATCATATTGACAAAATTTGTAAAAAA
AGCTATTTTTAATAAATTTGTCGTTCGATGTATGGATACATTTTCCATAAACCCGGAAAGAATCATCAAAATGGCCGCGCGAA
TAAATAAGATGTTGTTAGTGAAAAAAATATCTGAACATGCTTGGAAAAATCATGCGGCTAGACTTAAACACCTTAAGCATGCG
GTATACACGATGAAACATAAAGATGGGAAAAATAGACTCATGAACTTGATCTATGATCACTACTATTACCATATGCAAGGGGA
AGAAATCTTTAGCCTCGCAAGATTTTATGCAATCCATCATGCACCAAAGTTGTTTGACGTTTTTTATGATTGTTGTCTCCTAG
ATACTATACGATTTAAAAACCTTCTTTTAGATTGTTCACACATCATAGGTAAAAACGCTCATGATGCTACTAATATCACTATC
GTGAACAAGTATATTGGCAACCTGTTTGCTATGGGAGTTCTTAGCAAAAAAGAAATCTTACAGGACTATCCATCCATCTATTC
TAAACATTACATGCCTTAG
L60 MGF 505 1R (NC_044941.1:22320-23915)
SEQ ID No. 140
ATGTTCTCTCTCCAGAACTTATGTCGAAAAACATTACCTGACTGTAAACTTCCTGAATTTTTTGACGATTATATATTACAACT
GCTGGGATTATACTGGGAAAACCATGGAACTATTCAACGGGCAGGAAACAACTGTGTGCTTATACAACAACATACCCTCATTC
CCGTAAATGAAGCCCTAAGAATAGCAGCATCTGAAGAAAATTATGAGATCGTGGGCCTTTTATTAGCGTGGGAGGGGAACCTT
TACTATGCTATTATAGGGGCTCTAGAGGGTAACCGCTACAACTTAATTCGTAAATATGATGATCAAATCAAGGACCATCATGA
CATTCTGCCATTCATTGATGATCCAATCATATTTCACAAATGCCATATCATGCGGCGATGCTTTTTTGATTGTATTTTATATC
AAGCTGTAAAATATAGTAAGTTTCGTGTTCTTCTTTATTTTAAATATACATTAGAGGATGATTTGCCCCTCGTTCATTTACTT
ATTGAAAAGGCATGTGAAGATCATAATTATGAAGTTATTAAATGGATATATGAAAACCTACATGTCTGCCATATAATAGATAC
CTTTGACTGTGCTATTGCCCATAAAGATCTACGTTTATATTGTTTGGGGTATACATTTATATACAACAGGATTGTACCCTATA
AGTATCATCATTTAGATATTCTCATACTTTCAAGCCTACAACTCCTACATAAGGTGGCGGCCAAAGGATACTTAGATTTTATC
CTAGAAACCTTAAAGTATGATCATAATATAGATAATTTAGATGTTATTCTAACACAAGCTGCAACATATAACCATAGAAAAAT
TTTAACCTATTTTATTCCTCAATCAACCTACGCACAAATAGAACAATGTTTGTTCGTGGCGATAAAAACAAAATCTTCCAAGA
AAACCTTGAACTTACTACTGTCTCACCTAAACCTTTCCATCAAACTCATCCAAAAAATCAGCCAATATGTTGCCACTTTCAAT
TCAACAAATATAATAGGCATTCTGAGTATGAAGCGGAAAAAGAAGATATATTTGGATATCATATTGACAAAATTTGTAAAAAA
TGCTATTTTTAATAAATTTGTCGTTCGATGTATGGAGAGATTTTCTATAAACCCGGAAAGAATCGTCAAAATGGCTGCGCGTA
TAAATAAGATGATGTTAGTGAAAAAAATATCTGAACATGTTTGGAAAAATCATGCGGCTAGACTTAAACACCTTAAACATGCG
GTACACACGATGAAGCATAAAGATGGGAAAAATAGACTCATGAACTTTATCTATGAACACTGCTATTACCATATGCAAGGGGA
AGAAATTTTTAGCCTCGCAAGATTTTATGCAATCCATCATGCACCAAAGTTGTTCGACGTTTTTTATAATTGTTGTATCCTAG
ATACGATACGATTCAAAAGCCTTCTTTTAGATTGTTCACATATCATAGGTAAAAACGCTCATGATGCTACTAATATCAACATC
GTGAACAAGTATATTGGCAACCTGTTTGCTATGGGAGTTCTTAGCAAAAAAGAAATCTTACAGGACTATCCATCCATCTATTC
TAAACATTATATGCCTTAG
Benin 97/1 MGF 505 1R (NC_044956.1:21971-23566)
SEQ ID No. 141
ATGTTCTCTCTCCAGAACTTATGTCGAAAAACATTACCTGACTGTAAACTTCCTGAATTTTTTGACGATTATATATTACAACT
GCTGGGATTATACTGGGAAAACCATGGAACTATTCAACGGGCAGGAAACAACTGTGTGCTTATACAACAACATACCCTCATTC
CCGTAAATGAAGCCCTAAGAATAGCAGCATCTGAAGAAAATTATGAGATCGTGGGCCTTTTATTAGCGTGGGAGGGGAACCTT
TACTATGCTATTATAGGGGCTCTAGAGGGTAACCGCTACAACTTAATTCGTAAATATGATGATCAAATCAAGGACCATCATGA
CATTCTGCCATTCATTGATGATCCAATCATATTTCACAAATGCCATATCATGCGGCGATGCTTTTTTGATTGTATTTTATATC
AAGCTGTAAAATATAGTAAGTTTCGTGTTCTTCTTTATTTTAAATATACATTAGAGGATGATTTGCCCCTCGTTCATTTACTT
ATTGAAAAGGCATGTGAAGATCATAATTATGAAGTTATTAAATGGATATATGAAAACCTACATGTCTGCCATATAATAGATAC
CTTTGACTGTGCTATTGCCCATAAAGATCTACGTTTATATTGTTTGGGGTATACATTTATATACAACAGGATTGTACCCTATA
AGTATCATCATTTAGATATTCTCATACTTTCAAGCCTACAACTCCTACATAAGGTGGCGGCCAAAGGATACTTAGATTTTATC
CTAGAAACCTTAAAGTATGATCATAATATAGATAATTTAGATGTTATTCTAACACAAGCTGCAACATATAACCATAGAAAAAT
TTTAACCTATTTTATTCCTCAATCAACCTACGCACAAATAGAACAATGTTTGTTCGTGGCGATAAAAACAAAATCTTCCAAGA
AAACCTTGAACTTACTACTGTCTCACCTAAACCTTTCCATCAAACTCATCCAAAAAATCAGCCAATATGTTGCCACTTTCAAT
TCAACAAATATAATAGGCATTCTGAGTATGAAGCGGAAAAAGAAGATATATTTGGATATCATATTGACAAAATTTGTAAAAAA
TGCTATTTTTAATAAATTTGTCGTTCGATGTATGGAGAGATTTTCTATAAACCCGGAAAGAATCGTCAAAATGGCTGCGCGTA
TAAATAAGATGATGTTAGTGAAAAAAATATCTGAACATGTTTGGAAAAATCATGCGGCTAGACTTAAACACCTTAAACATGCG
GTACACACGATGAAGCATAAAGATGGGAAAAATAGACTCATGAACTTTATCTATGAACACTGCTATTACCATATGCAAGGGGA
AGAAATTTTTAGCCTCGCAAGATTTTATGCAATCCATCATGCACCAAAGTTGTTCGACGTTTTTTATAATTGTTGTATCCTAG
ATACGATACGATTCAAAAGCCTTCTTTTAGATTGTTCACATATCATAGGTAAAAACGCTCATGATGCTACTAATATCAACATC
GTGAACAAGTATATTGGCAACCTGTTTGCTATGGGAGTTCTTAGCAAAAAAGAAATCTTACAGGACTATCCATCCATCTATTC
TAAACATTATATGCCTTAG
Pretorisuskop/96/4 MGF 505 1R (AY261363.1:28449-30044)
SEQ ID No. 142
ATGTTCTCTCTCCAGAACTTATGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTGTACAACAACATACCCTCATTC
CCGTAAACGAAGCCCTGAGAATAGCAGCGTCTGAAGAAAATTATGAGATCGTGAGCCTTTTATTAGCGTGGGAGGGAAACCTT
TACTATGCTATTATAGGAGCTCTAGAGGGCAACCGCCACGACTTAATTCGTAAATATGATGACCAAATCAAGGACCATCATGA
AATTCTGCCATTCATTGACAATCCAGTCATATTTCACAAATGCCATATAATGCGGCGATGCTTTTTTGATTGTATTTTATATC
AAGCTGTAAAATATAGTAAGTTTCGCGTTCTTCTTTATTTTAAATATAGGTTAGAGAATGATTTGCCCCTCGCTCATTTACTT
GTTGAAAAGGCATGTGAAGATCATAATTATGAAGTTATTAAATGGTTATATGAAAACCTACATATCTACAATATAATGGAGAC
CTTTGAATGTGCTATTGCCCATAAGGATCTACGTCTATATCGTTTGGGGTATACATTTATATATAACAGGATCGTACCCTATA
AGTATCATTATTTAGATGTTCTCATTCTTTCAGGCCTACATCTCCTGTATAAGGTGGCAGCCAAAGGATACTTAGATTTTATC
CTAGAAACCTTAAAGTATGATCATAACAATGATAATTTAGATATTATTCTAACACAAGCTGTAACATATAACCATAGAAAAAT
TTTAACCTATTACATTCCTCAATTAACCTATGCACAAATAGAACAATGTTTGTTCATGGCGATTAAAAAAAAATCTTCCAAGA
AAACCTTGAACTTACTACTGTCTCACCTAAAGCTTTCCATCAAACTCATCAAAAAAATAAGCCAATATGTTGCCACTTACAAT
TCAACAAATATAATAGGCATTCTGAATATGAAGCGGAAAAAGAAGATATATTTAGATATCATATTGACAAAATTTGTAAAATA
CGCTATTTTTAATAAATATGTCGTTCGTTGTATGGATACATTTTCCATAAACCCGGAAAGAATCATCAAAATGGCCGCGCGAA
TAAATAAGATGTTGTTAGTGAAAAAAATATCTCAACATGCTTGGAAAAATCATGCGGCTAGACTTAAACACCTTAAGCATGCG
GTATACACGATGAAACATAAAGATGGGAAAAATAGACTCATGAACTTGATCTATGATCACTACTATTACCATATGCAAGGGGA
AGAAATCTTTAGCCTCGCAAGATTTTATGCAATCCATCATGCACCAAAGTTGTTTGACGTTTTTTATGATTGTTGTCTCCTAG
ATACTATACGATTTAAAAGCCTTCTTTTAGATTGTTCACACATCATAGGTAAAAACGCTCATGATGCTACTAATATCACTATC
GTGAACAAGTATATTGGCAACCTGTTTGCTATGGGAGTTCTTAGCAAAAAAGAAATCTTACAGGACTATCCATCCATCTATTC
TAAACATTACATGCCTTAG
Kenya 1950 MGF 505 1R (AY261360.1:31904-33496)
SEQ ID No. 143
ATGTTTTCTCTCCAGAACTTATGTCGAAAAACCTTACCTGACCGTAAACTTCCTGAATTTTTTGACGATTATGTATTACAACT
GCTGGGATTATACTGGGAAAACCATGGAACGATTCAACGAGCAGGAAACAACTGTGTACTTATCCAACAACATAACCTCATTC
CCGTAAATGAAGCCCTAAGAATAGCAGCATCTGAAGAAAATTATGAGATCGTGAGCCTTTTATTAGCTTGGGAGGGGAACCTT
TACTATGCTATTATAGGGGCTCTAGAGGGTAATCGCCACAACTTAATTCGTAAATATGATGACCAAATCAAGGACCATCATGA
AATTCTGCCATTTATTGACGATCCAGTCATATTTCACAAATGTCATATGATGCGGCGATGCTTTTTTAATTGTATTTTGTATC
AAGCTGTAAAATATAGTAAGTTTAGCGTTCTTCTATATTTTAAATATATATTAAAGGAGAATTTGCCCCTCGTTCATTCACTT
ATTGAAAAGGCGTGTGAAGATCATAATTATGAAGTTATTAAATGGATCTATGAAAACCTACATATCTATGAAATAATGGATAC
CTTTAAATGTGCTATTGCCCATAAAGATCTACATTTATATAGTTTAGGATATACATTTATATATAATAGGATCGTACCCTATA
AGTATCATCATTTAGATATTCGCATACTTTCAAGGCTACAACTTCTACATAAGGTGACAGCCAAAGGATACTTGGACTTTATC
CTAGAAACCTTAAAGTATGATCATAATAAAGATAATATAAATATTATTCTGACACAAGCTGCAACCTATAACCATAGAAACAT
TTTAACCTATTTCATTCCTCAATCAACCTACGCGCAGATAGAACAATGCTTATTCGTGGCAATAAAAACAAACGCTTCCAAGA
AAACCTTGAATTTACTATTATCTCACCTAAACCTTTCTATCAAACTCGTCAAAAAATTAAGCCAATATGTTGTTGCTTACAAG
TCAACAAATATCATAAGCATTCTGAGTATGCAGCAAAAAAAGAAGATATATTTAGATATCATTTTGACAAAGGTTGTAAAAAA
CGCTATTTTTATTAAATTTGTCATTGGATGTATGGTTACATTTTCCATAAACCCAGAAAGAATTGTCAAAATGGCCGCGCGAA
TAAAAAAGATGAAGTTAGTGAAAAATATATCTGAACATGTTTGGAAAAATCATGCGGCTAAACTTAAACACCTTAAGCATGCG
GTACACACGATGAAGCATCAAGAAGGAAAAAATAGATTAATGAACTTTATCTATGATCACTGTTATTACCACATGCAAGGAGA
AGAGATCTTTAGTCTTGCAAGATTTTATGCAATCCATCATGCGCCAAAATTGTTCGACGTTTTTTATGATTGTTGTATCCTAG
ATACGATACGATTTAAAAGCCTGCTTTTAGATTGTTCATATATCATAGCTAAAAACGCTCATGATGCTAGTATTAACATCGTG
AATAAGTATATTGGCAATCTATTTGCTATGGGGGTTCTTAGCAAAAAAGAAATCTTACAAGACTATCCATCCATCTATTCTAA
GGATTACATGCTTTAG
Ken06.Bus MGF 505 1R (NC_044946.1:25784-27376)
SEQ ID No. 144
ATGTTCTCTCTCCAGAACTTATGTCGAAAAACCTTACCTGACTGTAAACTTCCTGAGTTTTTTGACGATTATGTATTACAACT
GCTGGGCTTATACTGGGAAAATCATGGTACGATTCAACGAGCAGGAAACAACTGTGTACTTATCCAACAACATAACCTCATTC
CCGTAAATGAAGCCCTAAGAATAGCAGCATCTGAGGAAAATTATGAGATCGTGAGCCTTTTATTAGCGTGGGAGGGGAACCTT
TACTATGCTATTATAGGGGCTCTAGAGGGGAACCACCACAACTTAATTCGTAAATACGATGACCAAATCAAGGACCATCATGA
AATTCTGCCATTTATTGACGATCCAGTCATATTTCACAAATGTCATACAATGCGGCGATGCTTTTTTAATTGTATTTTATATC
AAGCTGTAAAATATAGTAAGTTCAGCGTTCTTCTATACTTTAAATATATATTAAAGGAGAATTTGCCCCTCGTCCACTCACTT
ATTGAAAAGGCGTATAAATATCATAATTATGAGGTTATTAAATGGATCTATGAAAACCTACATATCTATGATATCATAAATAC
TTTTAAATGTGCTATTGCCCATAAAGATCTACGTTTATATTGTTTAGGGTATACATTTGTATATAATAGAATCGTACCCTATA
AGTATTATCATTTAGATATTCGCATCCTTTTAAGGCTACAACTTCTACATAAGGTGACAGCCAAAGGATACTTGGATTTTATC
CTAGAAACCTTAAAGTATGATCATAATACAAACAATATAGATATTATCCTAACACAGGCTGCAACCTATAACCATAGAAACAT
TTTAACCTATTTCATTCCTCAATCAACCTACGCACAAATAGAACAATGTTTATTCGTGGCGATAAAAACAAATGCTTCCAAGA
AAACCTTGAATTTACTATTATCTCACCTAAATCTTTCTATCAAACTCGTCAAAAAATTAAGCCAATATGTTGTCGCTTACAAG
TCAACAAATATCATAAGCATTCTGAGTAGGCAGCAAAAAAAGAAGATATATTTAGATATCATTTTGACAAAGGTTGTAAAAAA
TGCTGTTTTTAATAAATTTGTCATTGGATGTATGGTTACATTTTCCATAAACCCAGAAAGAATTGTCAAAATGGCCGCGCGAA
TAAAAAAGATGAAGTTAGTAAAAAATATATCTGAACATGTTTGGAAAAATCATGCGGTTAAACTTAAATATCTTAAGCATGCG
GTACACACGATGAAGCATCAAGAAGGAAAAAATAGATTAATGAACTTTATCTATGATCACTGTTATTACCACATGCAAGAAGA
AGAGATCTTTAGCCTCGCAAGATTTTATGCAATCCATCATGCGCCAAAATTGTTCGACGTTTTTTATGATTGTTGTATCCTAG
ATACGATACGATTTAAAAGCCTGCTTTTAGATTGTTCACATATCATAGTTAAAAACGCTCATGATGCTAGTATTAACATCGTG
AACAAGTATATTGGCAATCTATTTGCTATGGGAGTTCTTAGCAAAAAAGAAATCTTACAAGACTATCCATCTATCTATTCTAA
GGATTACATGCTTTAG
Ken05/Tk1 MGF 505 1R (NC_044945.1:29657-31249)
SEQ ID No. 145
ATGTTCTCTCTCCAAAACTTATGTCGAAAAACCTTACCTGACTGTAAACTTCCTGAATTTTTTGACGATTATGTATTACAACT
GCTGGGATTATACTGGGAAAACCATGGAACGATTCAACGAGCAGGAAACAACTGTGTACTTATCCAACAACATAACCTCATTC
CCGTAAATGAAGCCCTAAGAATAGCAGCATCTGAAGAAAATTATGAGATCGTGAGCCTTTTATTAGCGTGGGAGGGGAACCTT
TACTATGCTATTATAGGGGCTCTAGAGGGTAACCGCCACAACTTAATTCGTAAATATGATGACCAAATCAAGGACCATCATGA
AATTCTGCCATTTATTGACGATCCAGTTATATTTCACAAATGTCATATAATGCGGCGATGCTTTTTTAATTGTATGTTGTATC
AAGCTGTAAAATATAGTAAGTTTAGCGTTCTTCTATATTTTAAATATATATTAAAGGAAAATTTGCCCCTCGTCCACTCACTC
ATTGAAAAAGCGTATAAATATCATAATTATGAGGTTATTAAATGGATCTATGAAAATCTACATATCTATGATATCATAAATAC
TTTTAAATATGCTATTGCCCATAAAGATCTACGTTTATATTGTTTAGGGTATACATTTGTATATAATAGGATCGTACCCTATA
AGTATTATCATTTAGATATTCGCATCCTTTTAAGGCTACAACTTCTACATAAGGTGACAGCCAAAGGATACTTGGATTTTATC
CTAGAAACCTTAAAGTATGATCATAATACAAACAATATAGATATTATCCTAACACAGGCTGCAACCTATAACCATAGAAACAT
TTTAACCTATTTCATTCCTCAATCAACCTACGCACAAATAGAACAATGCTTATTCGTGGCAATAAAAACAAACGCTTCCAAAA
AAACCTTGAATTTACTATTATCTCACCTAAACCTTTCTATCAAACTCGTCAAAAAATTAAGCCAATATGTTGTCGCTTACAAG
TCAACAAATATCATAAGCATTCTGAGTATGCAGCAAAAAAAGAAGATATATTTAGATATCATTTTGACAAAGGTTGTAAAAAA
CGCTGTTTTTATTAAATTTGTCATTGGATGTATGGTTACATTTTCCATAAACCCAGAAAGAATTGTCAAAATGGCCGCGCGAA
TAAAAAAGATGAAGTTAGTAAAAAATATATCTGAACATGTTTGGAAAAATCATGCGGCTAAACTTAAACATCTTAAGCATGCG
GTACACACGATGAAGCATCAAGAAGGAAAAAATAGACTCATGAACTTTATCTATGATCACTGCTATTACCACATGCAAGGAGA
AGAGATCTTTAGTCTTGCAAGATTTTATGCAATCCATCATGCGCCAAAATTGTTCGACGTTTTTTATGATTGTTGTATCCTAG
ATACGATACGATTTAAAAGCCTGCTTTTAGATTGTTCACATATTATAGCTAAAAACGCTCATGATGCTAGTATTAACATCGTG
AATAAGTATATTGGCAATCTATTTGCTATGGGAGTTCTTAGCAAAAAAGAAATCTTACAAGACTATCCATCCATCTATTCTAA
ATATGACATACTTTAG
Malawi Lil-20/1 (1983) MGF 505 1R (AY261361.1:26041-27633)
SEQ ID No. 146
ATGTTCTCTCTCCAGAACTTATGTCGAAAAACATTACCTGACTGTAAACTTCCTGAATTTTTTGACGAATATATATTACAACT
GCTGGGATTATACTGGGAAAACCATGGAACTATTCAACGAGCAGGAAACAACTGTGTGCTTATCCAGCAACATAACCTTATTC
CCGTAAATGAAGCCCTAAGAATAGCAGCATCTGAAGAAAATTATGAGATCGTGAGCCTTTTATTAGCGTGGGAGGGAAACCTT
TACTATGCTATTATAGGGGCTCTAGAGGGCAACCGCCCCGACTTAATTCGTAAATATGATGACCAAATCAAGGACCATCATGA
AATTTTGCCATTCATCGACGATCCAATCATATTTCACAAATGTCACATAATGCGGCGATGCTTTTTTAATTGTATTTTATATC
AAGCTGTAAAATATAGTAAGTTTCGTGTTCTTCTTTATTTTAAACATAGGTTAGGGGATGATTTGCCCCTCACTCATTTACTT
ATTGAAAAGGCATGTGAAGATCATAATTATGAAGTTATTAAATGGATCTATGAAAACCTACATAGCTACAATATAATGGATAC
CTTTGAATGTGCTATTGCCCATAAGGATCTACGTCTATATTGTTTGGGGTATACATTTATATATAATAGGATCGTACCCTATA
AGTATCATCATTTAGATATTTGCATACTTTCAAGCCTACAACTCCTGCATAAGGTGGCAGCCAAAGGATACTTGGATTTTATC
CTAGAAACCTTAAAGTATGATCATAACATAAATAATATAGATATTATTCTAACACAAGCTGCAACCTATAACCATAGAAAAAT
TTTAACCTATTTCATTCCTCAATTAACCTACGCACAGATAGAACAATGTTTACTAGTGGCGATAAAAACAAAAGCTTCCAAGA
AAACCTTGAACTTACTACTGTCTCACCTAAACCTTTCCATCAAACTCATCAAAAAAATAAGCCAATATGTTGTCACTTACAAT
TCAACAAATATCATAAGCATTCTGAGTATGCGGCGGAAAAAGAAGATATATTTAGATATCATTTTGACAGAGTTTGTAAAAAA
CGCTATTTTTAATAAATTTGTCGTTCGATGTATGGATACATTTTCCATAAACCCGGAAAGAATTGTCAAAATGGCCGCACGAA
TAAATAGGATGATGTTAGTGAAAAATATATCTGAACGTGTTTGGAAAAATCATGCGGTTAAACTTAAACACCTTAAGCATGCG
GTACATACGATGAAGCATCAAGAAGGAAAAAATAGACTCATGAACTTTATCTATGATCACTGCTATTACCACATGCAAGGGGA
AGAGATCTTTGGCCTCGCAAGATTTTATGCAATCCATCATGCACCCAAGTTGTTTGACGTTTTTTATGATTGCTGCATGCTAG
ATGCTACACGATTTAAAAGCCTGCTTTTAGATTGTCCACATATCATAGGTAAAAACGCTTATGATGCTGGTATCAACCTCGTG
AACAAATATATTGGCAACCTATTTGCTATGGGGGTTCTTAGCAAAAAAGAAATCTTACAGGACTATCCATCTATCTATTCTAA
ACATGATATGTTTTAG

In an embodiment the attenuated ASFV of the invention comprises a functional version of MGF 505 1R. Suitably the functional version of MGF 505 1R comprises the sequence of SEQ ID No. 134, 135, 136, 137, 138, 139, 140, 141, 142, 143, 144, 145 or 146. Suitably the functional version of MGF 505 1R comprises a sequence having at least 70%, at least 80%, at least 90% or at least 95% identity with SEQ ID No. 134, 135, 136, 137, 138, 139, 140, 141, 142, 143, 144, 145 or 146. Suitably the functional version of MGF 505 1R consists of the sequence of SEQ ID No. 134, 135, 136, 137, 138, 139, 140, 141, 142, 143, 144, 145 or 146.

### MGF 505 2R Gene Sequences

Georgia 2007/1 MGF 505 2R (NC_044959.1:33119-34699)
SEQ ID No. 147
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAAACCATACATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCAAGAAGACAGCGGCCAAAGGGCTCCTCCACTTTGTGCTAGAAACGTTAAAATACGGCGGCAAC
ATAGATACCGTCCTGACCCAAGCCGTAAAGTACAATCATAGAAAACTTTTAGATTATTTTCTGCGTCAACTACCTCGTAAACA
TATTGAAAAACTTTTGTTGCTGGCCGTGCAGGAAAAGGCTTCTAAAAAAACATTGAACTTACTGTTGTCACATTTAAACTACT
CCGTGAAACGCATCAAAAAACTACCGCGCTATGTGATAGAGTACGAGTCCACCTTGGTGATAAAGATTTTATTAAAAAAAAGA
GTGAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTTATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TATTAGACTGTTTAGAAATTATTACTAAAAAATCTTGCTATAGTATCCTGGAAATCTTAGAAAAACATATTATTTCCCTGTTT
ACTATGAAAGTTATGACTGAAGAAGAAAAAAACCTATGTTTAGAAATATTATATAAAGTAATTCATTATAAAACAATACAATG
TTAA
China/2018/AnhuiXCGQ MGF 505 2R
SEQ ID No. 148
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAAACCATACATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCAAGAAGACAGCGGCCAAAGGGCTCCTCCACTTTGTGCTAGAAACGTTAAAATACGGCGGCAAC
ATAGATACCGTCCTGACCCAAGCCGTAAAGTACAATCATAGAAAACTTTTAGATTATTTTCTGCGTCAACTACCTCGTAAACA
TATTGAAAAACTTTTGTTGCTGGCCGTGCAGGAAAAGGCTTCTAAAAAAACATTGAACTTACTGTTGTCACATTTAAACTACT
CCGTGAAACGCATCAAAAAACTACCGCGCTATGTGATAGAGTACGAGTCCACCTTGGTGATAAAGATTTTATTAAAAAAAAGA
GTGAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTTATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TATTAGACTGTTTAGAAATTATTACTAAAAAATCTTGCTATAGTATCCTGGAAATCTTAGAAAAACATATTATTTCCCTGTTT
ACTATGAAAGTTATGACTGAAGAAGAAAAAAACCTATGTTTAGAAATATTATATAAAGTAATTCATTATAAAACAATACAATG
TTAA
Mkuzi 1979 MGF 505 2R (AY261362.1:34826-36406)
SEQ ID No. 149
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAGACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCGAGAAGACAGCGGCCAAAGGGCTCCTCCACTTTGTGCTAGAAACGTTAAAATACGGCGGCAAC
ATAGATACCGTCCTGACCCAAGCCGTAAAGTACAATCATAGAAAACTTTTAGATTATTTTCTGCGTCAACTACCTCGTAAACA
TATTGAAAAACTTTTGTTGCTGGCTGTTCAGGAAAAGGCTTCTAAAAAAACATTGAACTTACTGTTGTCACATTTAAACTACT
CCGTGAAACGCATCAAAAAACTGCTGCGCTATGTGATAGAGTATGAGTCCACCTTGGTGATAAAGATTTTGTTAAAAAAAAGA
GTAAACCTGATAGATGCCATGTTGGAAAAGATGGTAAGATATTTTTCTGCGACAAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTTTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTCATTTAGCCAAGCTCTATGTTGCTCAAAA
TGCGGCCACCCAATTCAGAGACATTTGTAAGGACTGTTACAAACTGGATGTGGCACGGTTTAAACCGCGGTTTAAGCAACTAA
TATTAGACTGTTTAGAAATTGTTACTAAAAAATCTTGCTATAGTATCCTGGAAATCTTAGAAAAACATATTATTTCCCTATTT
ACTATGAAAGTTATGACTGAAGAAGAAAAAAACCTATGTTTAGAAATATTATATAAAGTAATTCATTATAAAACAATACAATG
TTAA
L60 MGF 505 2R (NC_044941.1:27701-29281)
SEQ ID No. 150
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCCTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAGACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCGAGAAGACAGCGGCCAAAGGGCTCCTCCACTTTGTGCTAGAAACGTTAAAATACGGCGGCAAC
ATAGATACCGTCCTGACCCAAGCCGTAAAGTACAACCATAGAAAACTTTTAGATTATTTTCTGCGTCAACTACCTCGTAAACA
TATTGAAAAGCTTTTGTTGCTGGCTGTTCAGGAAAAGGCTTCTAAAAAAACATTGAACTTACTGTTGTCACATTTAAACTACT
CCGTGAAACGCATCAAAAAACTGCTGCGCTATGTGATAGAGTATGAGTCCACCTTGGTGATAAAGATTTTGTTAAAAAAAAGA
GTAAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTCATTTAGCCAAGCTCTATGTTGCTCAAAA
TGCGGCCACCCAATTCAGAGACATTTGTAAGGACTGTTACAAACTGGATGTGGCACGGTTTAAACCGCGGTTTAAGCAACTAA
TATTAGACTGTTTAGAAATTGTTACTAAAAAATCTTGCTATAGTATCCTGGAAATCTTAGAAAAACATATTATTTCCCTATTT
ACTATGAAAGTTATGACTGAAGAAGAAAAAAACCTATGTTTAGAAATATTATATAAAGTAATTCATTATAAAACAATACAATG
TTAA
Benin 97/1 MGF 505 2R (NC_044956.1:27352-28932)
SEQ ID No. 151
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCATAGGAGACGACCACATACTCATACGACGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCCTGCAGGGTGATCAATATGACCTGATCCATAAGTATGAAAACCAAATCGGCGACTTTCATTTTATCTT
ACCATTGATTCAAGACGCGAATACGTTTGAAAAATGCCACGCTTTAGAACGTTTTTGTGGTGTTTCATGTCTGCTAAAACATG
CTACAAAATACAACATGCTCCCTATTCTCCAAAAATACCAAGAAGAGCTGTCTATGAGAGCGTATCTTCACGAAACCCTATTT
GAACTAGCATGCCTATGGCAGAGGTATGATGTCCTTAAATGGATAGAGCAGACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAACATCTCGAGAAGACAGCGGCCAAAGGGCTCCTCCACTTTGTGCTAGAAACGTTAAAATACGGCGGCAAC
ATAGATACCGTCCTGACCCAAGCCGTAAAGTACAACCATAGAAAACTTTTAGATTATTTTCTGCGTCAACTACCTCGTAAACA
TATTGAAAAGCTTTTGTTGCTGGCTGTTCAGGAAAAGGCTTCTAAAAAAACATTGAACTTACTGTTGTCACATTTAAACTACT
CCGTGAAACGCATCAAAAAACTGCTGCGCTATGTGATAGAGTATGAGTCCACCTTGGTGATAAAGATTTTGTTAAAAAAAAGA
GTAAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACGGATATCGTAATCCATACTTCTTATGTTTGGGAGG
ATGATCTAGAACGTCTTACTCGTCTTAAAAATATGGTATACACCATAAAGTACGAACATGGGAAAAAAATGTTAATTAAAGTC
ATGCACGGCATATACAAAAACTTATTATACGGCGAAAGGGAAAAAGTCATGTTTCATTTAGCCAAGCTCTATGTTGCTCAAAA
TGCGGCCACCCAATTCAGAGACATTTGTAAGGACTGTTACAAACTGGATGTGGCACGGTTTAAACCGCGGTTTAAGCAACTAA
TATTAGACTGTTTAGAAATTGTTACTAAAAAATCTTGCTATAGTATCCTGGAAATCTTAGAAAAACATATTATTTCCCTATTT
ACTATGAAAGTTATGACTGAAGAAGAAAAAAACCTATGTTTAGAAATATTATATAAAGTAATTCATTATAAAACAATACAATG
TTAA
Tengani 62 MGF 505 2R (AY261364.1:28261-29830 )
SEQ ID No. 152
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCGTAGGAGACGACCACATACTCATACGGCGGGATCTCATCCTTTCCACCA
ACGAGGCCTTAAGAATGGCGGGAGAGGAAGGAAACAATGAAGTAGTAAAGCTCTTGTTACTGTGGAAGGGAAATCTTCATTAC
GCCGTCATAGGAGCCTTGCAGGGTGATCAATATGACCTGATCCATAAGTATGAAAACCAAATCGGCGACTTTCATCTTATCTT
ACCATTGATTCAAGACGCGAAAACGTTTGAAAAATGCCATGCTTTAGAACGTTTTTGTGGTGTTTCATGTCTGCTAAAACATG
CTACAAAATACAACATGCTCCCTGTTCTCCAAAAATACCAAGAAGAGCTGTCCATGAGAGCATATCTTTGCGAAACCCTATTT
GAACTAGCATGCCTATGGCAGAGGTATGATGTCCTTAAATGGATAGAGCAAACCATGCATGTTTACGACCTAAAGATTATGTT
TAATATTGCCATCTCCAAGAGGGATCTGACTATGTACTCCTTAGGATATATTTTCCTTTTTGATAGAGGGAACACCGAAGCTA
CGTTGCTAACGCAATATCTCGAGAAGACAGCGGCCAAAGGGCTCCTCCACTTTGTGCTAGAAACGTTAAAATACGGCGGTAAC
ATAGATATTGTCCTGACCCAAGCCGTAAAGTACAATCATAGAAAACTTTTAGATTATTTTCTGCGTCAACTACCTCGTAAACA
TATTGAAAAGCTTTTGTTGCTGGCCGTGCAGGAAAAGGCTTCTAAAAAAACATTGAACTTACTGTTGTCACATTTAAACTACT
CCGTGAAACGCATCAAAAAACTGCTGCGCTATGTGATAGAGTACGAGTCCACCTTGGTGATAAAGATTTTATTAAAAAAAAGA
GTAAACCTGATAGATGCCATGTTGGAAAAGATGGTAAGATATTTTTCTGCGACGAAAGTGAGGACGATCATGGATGAGCTTTT
GATTAGTCCGGAAAGAGTCATTAAGATGGCTATACAGAAAATGAGAACGGATATCGTAATCCATACTTCTTATGTATGGGAGG
ATGATCTAGAACGACTTACTCGTCTTAAAGATATGGTATACACCGTAAAGTACGAACATGGGAAAAAAATGTTAATTAAAGTC
ATACACGGCATATACAAAAACTTATTATACGGCGAAAAAGAAAAGGTCATGTTTCATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TGTTAGACTGTTTAGAAATTGTTACTAAAAAATCTTGCTATAGTATTCTGGAAATCTTAGAAAAACATATGATTTCCCTATTT
ACTATGAAAGTTATGACTGAAGAAGAAAAAAACCTATGTTTAGAAATATTATATAAGTAA
Pretorisuskop/96/4 MGF 505 2R (AY261363.1:33795-35374)
SEQ ID No. 153
ATGTTTTCCCTTCAAGACCTTTGCCGAAAGCATCTTTTTATTCTTCCCGATGTTTTTGGCGAGCATGTACTACAACGATTAGG
ACTGTATTGGAGATGTCACGGCTCCCTTCAACGCGTAGGAGACGACCACATCCTCATACGGCGGGATCTCATCCTTTCCACCA
ACGAGGCCTTAAGAATGGCGGGAGAAGAAGGAAACAATGAAGTAGTAAAGCTCTTGTTACTGTGGAAGGGAAATCTTCATTAC
GCCATCATAGGAGCCTTGCAGGGTGATCAATATGACCTGATCCATAAGTATGAAAACCAAATCGGCGACTTTCATCTTATCTT
ACCATTGATTCAAGATGCGAAAACGTTTGAAAAATGCCACGCTTTAGAACGTTTTTGTGGTGTTTCATGTCTGCTAGAACATG
CTACAAAATACAACATGCTCCCTATTCTCCAAACATACCAAGAAGAGCTGTCTATGAGAGCATATCTTCGCGAAACCCTATTT
GAACTAGCGTGCCTGTGGCAGAGGTATGATGTCCTTAAATGGATAGAGCAAACCATGCATGTTTACGACCTAAAGATTATGTT
TAATATTGCCATCTCCAAGAGGGATCTGACGATGTATTCCTTAGGATATATTCTTCTTTTTGATAGAGAGAACACCGAAGCTA
CGTTGTTAACACAACATCTCGAGAAGACAGCGGCCAAAGGGCTCCTCTACTTTGTGCTAGAAACGTTAAAATACGGCGGTAAC
ATAGATATCGTCCTGACTCAAGCCGTAAAGTACAATCATAGAAAACTTTTAGATTATTTTCTGCGTCAACTACCTCGTAAACA
TATTGAAAAGCTTTTGTTGCTGGCCGTGCAGGAAAAGGCTTCTAAAAAAACATTGAACTTACTGTTGTCCCATCTAAACTACT
CCGTGAAACGTATCAAAAAACTGCTGCGCTATGTGATAGAGTACGAGTCCACCTTGGTGATAAAGATTTTATTAAAAAAAAGA
GTAAACCTGATAGATGCCATGTTGGAAAAGATGGTAAGATATTTTTCTGCGACGAAAGTGAGGACGATCATGGATGAGCTTTT
GATTAGTCCGGAAAGAGTCATTAAGATGGCTATACAGAAAATGAGAACGGATATCGTAATCCATACTTCTTATGTTTGGGAGG
ATGATCTAGAACGACTTACTCGTCTTAAAAATATGGTATACACCATAAAGTACGAACATGGGAAAAAAATGTTAATTAAAGTC
ATGCACGGCATATACAAAAACTTATTATACGACGAAAGAGAAAAGGTCATGTTTCATTTAGCCAAGCTCTATGTTGCTCAAAA
CGCGGCCACCCAATTCAGAGACATTTGTAAGGACTGTTACAAACTGGATGTGGCACGGTTTAAACCGCGGTTTAAGCAACTAA
TGTTAGACTGTTTAGAAATTGTTACTAAAAAATCTTGCTATAGTATCCTGGAAATCTTAGAAAAACATATTATTTCCCTATTT
ACTATGAAAGTTATGACTGAAGAGGAAAAAAACCTATGTTTAGAAATATTATATAAGTAA
Warmbaths MGF 505 2R (AY261365.1:33029-34597)
SEQ ID No. 154
ATGTTTTCCCTTCAAGACCTTTGCCGAAAGCATCTTTTTATTCTTCCCGATGTTTTTGGCGAGCATGTATTACAACGATTAGG
ACTGTATTGGAGATGTCACGGCTCCCTTCAACGCGTAGGAGACGACCACATACTCATACGGCGGGATCTCATCCTTTCCACCA
ACGAGGCCTTAAGAATGGCGGGAGAAGAAGGAAACAATGAAGTAGTAAAGCTCTTGTTACTGTGGAAGGGAAATCTTCATTAC
GCCATCATAGGAGCCTTGCAGGGTGATCAATATGACCTAATCCATAAGTATGAAAACCAAATCGGCGACTTTCATCTTATCTT
ACCATTGATTCAAGATGCGAAAACGTTTGAAAAATGCCACGCTTTAGAACGTTTTTGTGGTGTTTCATGTCTGCTAAAACATG
CTACAAAATACAACATGCTCCCTATTCTCCAAACATACCAAGAAGAGCTGTCTATGAGAGTATATCTTCGCGAAACCCTATTT
GAACTAGCATGCCTATGGCAGAGGTATGATGTCCTTAAATGGATAGAGCAAACCATGCATGTTTACGACCTAAAGGTTATGTT
TAATATTGCCATCTCCAAGAGAGATCTGACTATGTATTCCTTAGGATATATTCTTCTTTTTGATAGAGAGAACACCGAAGCTA
CGTTGCTAACGCAACATCTCGAGAAAACAGCGGCCAAGGGGCTCCTCCACTTTGTGCTAGAAACGTTAAAATACGGCGGTAAC
ATAGATATCGTCCTGTCCCAAGCCGTAAAGTACAATCATAGAAAACTTTTAGATTATTTTCTGCGTCAACTACCTCGTAAAAA
TATTGAAAAACTTTTGTTGCTGGCTGTTCAGGAAAAGGCTTCTAAAAAAACATTGAACTTACTGTTGTCCCATTTAAACTACT
CCGTGAAACGCATCAAAAAACTGCTGCGCTATGTGATAGAGTACGAGTCCACCTTGGTGATAAAGATTTTATTAAAAAAAAGA
GTAAACCTGATAGATGCCATGTTGGAAAAGATGGTAAGATATTTTTCTGCGACGAAAGTGAGGACGATCATGGATGAGCTTTC
GATTAGTCCGGAAAGAGTCATTAAGATGGCTATACAGAAAATGAGAACAGATATCGTAATCCATACTTCTTATGTTTGGGAGG
ATGATCTAGAACGACTTACTCGTCTTAAAAATATGGTATACGCCATAAAGTATGAACATGGGAAAAAAATGTTAATGAAAGTC
ATGCACGGCATATACAAAAACTTATTATACGGCGAAAGAGAAAAGGTCATGTTTCATTTAGCCAAGCTCTATGTTGCTCAAAA
CGCGGCCACCCAATTCAGAGACATTTGTAAGGACTGTTACAAACTGGATGTGGCACGGTTTAAGCAACTAACGTTAAACTGTT
TAGAAATTATTACTAAAAAATCTTGCTATAGTATCCTGGAAATCCTAGAAAAACATATTATTTCCCTATTTACTATGAAAGTT
ATGACTGAAGAAGAAAAAAACCTATGTTTAGAAATATTATATAAAGTAATTCATTATAATACAATACAATGTTAA
Malawi Lil-20/1 (1983) MGF 505 2R (AY261361.1:31541-33121)
SEQ ID No. 155
ATGTTTTCCCTTCAAGATCTTTGCCGAAAGCATCTTTTTATTCTTCCCGATGTTTTTGGCGAGCATGTACTACAACAGTTAGG
ACTGTACTGGAAACGTCACGGCTCTCTTCAACGCATAGGAGACGACCACATACTCATACGGCGGGACCTCATCCTTTCCACCA
ACGAGGCCTTAAAAATGGCGGGAGAGGAAGGAAACAATGAAGTAGTAAAGCTCTTGTTACTGTGGGAGGGAAACCTTCATTAT
GCCATCATAGGGGCCTTACAGGGTGATCAATATGATCTGATCCATAAGTATGAAAACCAAATCGAAGACTATCATCATATCTT
GCCATTGATTCAAGATGCGAAAACGTTTGAAAAATGCCACGCCTTAGAACGTTTTTGTGATGTTCCATGTCTGCTAGAACATG
CTACAAAACACAACATGCTCCCTATTCTCCAAAAATACCAAGAAGAGCTGTCTATAAGAGTGTATCTTCGCGAAACCCTATTC
GAACTAGCATGCCTATGGCAGAGGTATGATGTTCTTAAATGGATAGAGCAAACCATGCATGTTTACGACCTAAAGATTATGTT
TAATATTGCCATCTCCAAGAGGGATCTAAGCATGTACTCCTTAGGATATGTTCTCCTTTTTGATAGAGGAAACATCGAAGCTA
CGTTCCTAACGCAACATCTCGAGAAGACAGCGGCCAAAGGGCTCCTCCACTTTGTGCTAGAAACGTTAAAATACGGCGGTAAC
TTAAATATCGTCCTGTCCCAAGCCGTAAAATACAATCATAGAAAACTTTTGGATTATTTTCTGCGTCAACTACCTCGTAAAAA
TATTGAAAAACTTTTGTTGCTGGCCGTGCAGGAAAAGGCTTCTAAAAAAACATTGAACCTACTGTTGTCACACTTAAACTACT
CCGTGAAACACATCAAAAAACTGCTGCGCTATGTGATAGAGTACGAGTCTACCTTGGTGATAAAACTTTTATTGAAAAAAAGA
GTAAACCTGATAGACGCCGTGTTGGAAAAGAATGTAAGATATTTTTCTGCGATTAAAGTGAGGACTATTATGGATGAGCTTTC
GATTAGTCCGGAAAGAGTCATTAAGATGGCCATACAGAAAATGAGAACGGATATTGTGATTCAGACTTCTTATATTTGGGAGG
ATGATCTAGAAAGACTTATTCGTCTTAAAAATATGGTATACACCATAAAGTATGAACATGGGAAAAAAATGTTAATTAAAGTT
ATTCACGGCATATACAAAAACTTATTATACGGCGAAAAAGAAAAAGTCATGTTTCATTTAGCCAAACTCTATGTTGCTCAAAA
CGCGGCCACCCAATTCAGGGACATTTGTAAGGACTGTTGCAAACTGGATGTGGCGCGGTTTAAACCGCGGTTTAAACAACTAA
TTTTAGACTGTTTAGAAATGGTTACTAAAAAATCTTGCTTTAGTATTATAGAAATCTTAGAAAACTATATTATTTCCCTATTT
GTGATGAAAGTCATCACTGAAGAAGAAAAAAACCTATGTTTAGAACTATTATATAAAGTAATTAGTTACAAAACGATATAATG
TTAA
Ken05/Tk1 MGF 505 2R (NC_044945.1:35094-36674)
SEQ ID No. 156
ATGTTTTCCCTTCAAGACCATTGCCGAAAGCATCTTTTTATTCTTCCCGATGTTTTTGGCGAGCATGTACTACAACGGTTAGG
ACTGTACTGGGGACGTCACGGCTCTCTTCAACGAATCGGGGACGATCACATACTCATACGGCGGGACCTCATCCTTTCCACCA
ACGAGGCCTTAAAAATGGCGGGAGAAGAAGGAAACAATGAAGTAGTAAAGCTCTTGTTACTGTGGGAGGGAAATCTTCATTAT
GCTATCATAGGGGCTTTACAGGGTGATCAATATGACCTAATCCATAAGTATGAAAACCAAATCGAAGACTATCATCATATCTT
ACCATTGATTCAAGATGCGGAAACGTTTGAAAAATGCCACGCCTTAGAACGTTTTTGTGATGTTCCATGTCTGCTAGAACATG
CTACAAAACACAACATGCTCCCTATTCTCCAAAAATATCAAGAAGAGTTGTCTATAAGAGTGTATCTACGCGAAACCCTATTC
GAACTAGCATGCCTATGGCAGAGGTATGATATTCTTAAATGGATAGAGCAAACCATGCATGTTTACGATCTAAAAATTATATT
TAATATTGCCATCTCCAAGAGGGATCTAAGCATGTACTCCTTAGGATATGTTCTCCTTTTTGATAGAGGGAACACCGAAGCTA
CCTTGTTAACGCAACACCTCGAGAAGACAGCGGCCAAGGGGCTCCTCCACTTTGTGCTAGAAACGTTAAAATACGGCGGTAAC
TTAAATATCGTCCTGTTCCAAGCCGTAAAATACAATCATAGAAAACTTTTAGATTATTTTCTGCGTCAACTACCCCGTAAAAA
TATTGAAAAACTTTTGTTGCTGGCTGTGCAGGAAAAAGCTTCTAAGAAAACATTGAACTTACTGTTGTCACATTTAAACTACT
CCGTGAAACGCATCAAAAAACTGCTGCGCTATGTGATAGAGTACGAGTCTACCTTGGTGATAAAGATTTTATTAAAAAAAAGA
GTAAACCTAATAGACGCCGTGTTGGAAAAGACTGTAAGATATTTTTCTGAGACGAAAGTAAAGACTATTATGGATGAGCTTTC
GATTAATCCGGAAAAAGTCATTAAGATGGCCATACAGAAAATGAGAACGGATATTGTGATCCAAACTTCTTATATTTGGGAGG
ATGATCTAGAAAGACTTATTCGTCTTAAAAATATGGTATACACCATAAAGTATGAACATGGGAAAAAAATGTTAATGAAAGTT
ATTCACGGCATATACAAAAACTTATTACACGATGAAAAAGAAAAAGTCATGTTTCATTTAGCCAAGTTCTATATTGCTCAAAA
CGCGGCCACCCAATTCAGAGACATTTGTAAAGACTGTTGCAAACTGGATGTGGCGCGGTTTAAACCGCGGTTTAAACAACTAA
TTTTAGACTGTTTAGAAATTGTTACTAAAAAATCTTGCTTTAGTATTATAGAAATTTTAGAAAACCATATTATTTCCCTATTT
ATGATGAAAGTTATCACTGAAGATGAAAAAAACCTAGGTTTAGAATTATTATATAAAGTAATTAGTTACAAAATGATATCATA
TTAA
Kenya 1950 MGF 505 2R (AY261360.1:37419-38999)
SEQ ID No. 157
ATGTTTTCCCTTCAAGACCTTTGCCGAAAGCATCTTTTTATTCTTCCCGATGTTTTTGGCGAGCACGTACTACAACAATTGGG
GCTGTACTGGGAAAAACACGGCTCTCTTCAACGAATAGGGAACGATCACATACTCATACGGGGGATCTCATCCTTTCTATCA
ACGAGGCCTTAAAAATAGCGGCAGAGGAAGGAAACAATGAAGTAGTAAAGCTTTTGTTACTATGGAAGGGAAATCTTCATTAT
GCCATCATAGGAGCTTTGGAGGGTGACCAATATGACCTGATCTATACGTATGAAAACCAAATTGAAGACTATCATCATATCTT
GCCATTGATTCAAGATGCGAAAACGTTTGAAAAATGCCACGCCTTGGAACGTTTTTGTGATGTTCCATGCCTGCTAGAGCATG
CTATAAAACACAACATGCTCCCTATTCTCCAAAAATATCAAGAAGAACTGTTTATAAGAGTGTATCTCCGCGAAACCCTATTT
GAACTAGCATGCCTATGGCAGAGGTATGATATTCTTAAATGGATAGAGCAAACCATGCATGTTTACGATCTAAAAATTATATT
TAATATTGCCATCTCCAAGAGGGATCTAAGCATGTACTCCTTAGGATATGTTCTCCTTTTTGATAGAGGGAACACCGAAGCTA
CCTTGTTAACGCAACACCTCGAGAAGACAGCGGCCAAGGGGCTCCTCCACTTTGTGCTAGAAACGTTAAAATACGGCGGTAAC
ATAAACATCGTCCTATCCCAAGCCGTAAAATACAATCATAGGAAACTTTTAGATTATTTTCTGCGTCAACTACCTCGTAAAAA
TATTGAAAAACTTTTGTTGTTAGCCGTACAGGAAAAGGCTTCTAAGAAAACATTGAACTTACTGTTGTCTTATTTAAACTACT
CCGTGAAACGCATCAAAAAACTGCTGCGCTATGTGATAGAGTACGAGTCCACACTGGTGATAAGGATTTTATTAAAAAAAAGA
ATAAACCTAATAGACGCCGTGTTGGAAAAGACTGTAAGATATTTTTCTGAGACGAAAGTAAAGACTATTATGGATGAGCTTTC
GATTAATCCGGAAAAAGTTATTAAGATGGCCATACAGAAAATGAGAACGGATATTGTGATCCAAACTTCTTATATTTGGGAGG
ATGATCTAGAAAGACTTATTCGTCTTAAAAATATGGTATACACCATAAAGTATGAACATGGGAAAAAAATGTTAATGAAAGTT
ATTCACGGCATATACAAAAACTTATTACACGATGAAAAAGAAAAAGTCATGTTTCATTTAGCCAAGTTCTATATTGCTCAAAA
CGCGGCCACCCAATTCAGAGACATTTGTAAAGACTGTTGCAAACTGGATGTGGCGCGGTTTAAACCGCGGTTTAAACAACTAA
TTTTAGACTGTTTAGATATTATTACTAAAAAAACGTGCCTCAATATTATGGAAATCTTAGAAAATCATATTATTTCGCTATTT
GCTATGAAAATGATGACTGAAGATGAAAAAAACCTAGGTTTAGAATTATTATATAAAGTAATTAGTTACAAAATGATATCATA
TTAA
Ken06.Bus MGF 505 2R (NC_044946.1:31225-32799)
SEQ ID No. 158
ATGTTTTCCCTTCAAGACCTTTGCCGAAAGCATCTTTTTATTCTTCCCGATGTTTTTGGCGAGCATGTACTACAACAATTGGG
GCTGTACTGGGAAAAACACGGCTCTCTTCAACGAATAGGGAACGATCACATACTCATACGGCGGGATCTCATCCTTTCTATCA
ACGAGGCCTTAAAAATAGCGGCAGAGGAAGGAAACAATGAAGTAGTAAAGCTCTTGTTACTGTGGAAGGGAAATCTTCATTAT
GCCATCATAGGAGCCTTGCAGGGTGACCAATATGACCTCATCCATACGTACGAAAACCAAATCGAAGACTATCATCATATCTT
GCCATTGATTCAAGATGCGAAAACGTTTGAAAAATGCCACGCCTTGGAACGTTTTTGTGATGTTCCATGCCTACTAGAACATG
CTACAAAACACAACATGCTCCCTATTCTCCAAAAATACCAAGAAGAGCTGTTTATAAGAGTGTATCTCCGCGAAACCCTATTT
GAACTAGCATGCCTATGGCAGAGGTATGATATCCTTAAATGGATAGAGCAAACCATGCATGTTTACGACCTAAAGATTATGTT
TAATATTGCCATCTCCAAGAGGGATCTAAGCATGTACTCTTTAGAATATATTCTCTTTTTTAATAGAGGGAACACCGATGTTG
CGTTAGCAACGTTGCTAACGCAACATCTCGAGAAGACAGCGGCCAAGGGGCTCCTCCACTTTGTGCTAGAAACGTTAAAATAC
GGCGGTAACATAAACATCGTCCTCTCCCAAGCCGTAAAATACAATCATAGAAAACTTTTAGATTATTTTCTGCGTCAACTACC
TCGTAAAAATATTGAAAAACTTTTGTTGCTGGCCGTACAGGAAAGGGCTTCTAAGAAAACATTGAACTTACTGTTGTCTTATT
TAAACTACTCCGTGAAACGCATCAAAAAACTGCTGCGCTATGTGATAGAGCATGAGTCCACACTGGTGATAAGGATTTTATTA
AAAAAAAGAATAAACCTAATAGACGCCGTGTTGGAAAAGACTGTAAGATATTTTTCTATGACTAAAGTGAGGACGATCATGGA
TGAGCTTTCGATTAATCCGGAAAAAGTCATTAAAATGGCCGTGCAGAAAATGAGAACGGATATCGTGATCCATACTTCTTATG
TTTGGGAGGATGATCTAGAAAGACTTATTCGTCTTAAAAATATGCTATACACTATAAAGTATGAGCATGGAAAAAAAATGCTA
ATTAAAGTCATTCACGGCATATACAAAAACTTATACGGCGAAAAAGAAAAAGTCATGTTTAATTTGGCCAAGTTCTATGCTGC
TCAAAACGCGGCCACCCAATTCAAAGACACTTGTAAAGACTGTTGCAAACTGGATGTGGCGCGGTTTAAACAACTAATTTTAG
ACTGTTTAGACATTATTACTAAAAAAACGTGCCTCAGTATCATGGAAATCTTAGAAAATCATATTATTTCGCTATTTGCTATG
AAAATGATGACGGAGGATGAAAAAAACTTAGGTTTAGAAATATTATATAAAGTAATTAGTTACAAAATGATATCATATTAA

In an embodiment the attenuated ASFV of the invention comprises a functional version of MGF 505 2R. Suitably the functional version of MGF 505 2R comprises the sequence of SEQ ID No. 147, 148, 149, 150, 151, 152, 153, 154, 155, 156, 157 or 158. Suitably the functional version of MGF 505 2R comprises a sequence having at least 70%, at least 80%, at least 90% or at least 95% identity with SEQ ID No. 147, 148, 149, 150, 151, 152, 153, 154, 155, 156, 157 or 158. Suitably the functional version of MGF 505 2R consists of the sequence of SEQ ID No. 147, 148, 149, 150, 151, 152, 153, 154, 155, 156, 157 or 158.

### MGF 505 6R Gene Sequences

Benin 97/1 MGF 505 6R
SEQ ID No. 257
ATGTTCTCCCTACAGGACCTCTGTCGGAAGAACACTTTCTTCCTTCCAAATGATTTTAGCAAGCATACTCTACAACGGCTGGG
GTTGTATTGGAAAGAGCATGGATCCGTCCATCGAATAGAAAAGGACAGCATAATGATACAGAATGAATTGGTTCTTTCTATCA
ATGATGCTTTACAGCTTGCAGGAGAGGAGGGGGACACAGATGTGGTACAGCTCTTGTTACTATGGGAGGGAAATCTGCATTAT
GCCATCATAGGAGCTTTGAAGACTGAGAATTATAATCTAGTATGTGAGTACCATAGCCAAATTCAGGACTGGCATATTCTCCT
ACCCTTGATTCAAGATCCAGAAACATTCGAAAAATGTCATGATTTAAGCCTTGGATGTGACCTTATATGCCTTCTCCAACATG
CTGTAAAATGTGATATGCTTTCTATTCTTGTTAAATATAAGGAGGATTTACTAAATGTAAGGATTAGGCATCGTACCCAATCC
CTGTTTGTTTTGGCATGCGAAAATCGGAGATTTGAGATTATTGAATGGATAGGTCAAAATCTGTCAATTCCTGAACCTGAGGC
CATTTTTAGCATTGCTATTGTTACAAAAGATGTAGAACTGTTTTCCTTAGGATATAAAATTATTTTTGATTACATGCAAAGAC
AAGGAATTTTTCAATTAACCAATGTAGTTCGCATGCTTCTGCTAAATCGTCATATTGGTATGGCAATAGAAAAAGGACTTTTA
CCTTTTATTCTGGAAACTTTAAAATATGGTGGTAGTGTAAAAAGAGCTTTATCTTATGCAGTAATAGATAATAAAAGAAAAAT
TATAGACTATCTTGTACGCCATGAAAATATACCCCGAGGAACTATTGAAAGACTTTTGCATCTAGCTGTGAAAAAACAATCTT
CCAGGAAAACTTTGAACTTGTTGCTATCTTACATAAATTACAAGGTGAAAAATGTTAAAAAGCTGGTAGAGCATGTAGTAGAT
CACAAATCCACTCTTGTGTTAAAAATTTTATTGGAAAAAAAGGAAAATCTAGTGGATGCTGTTTTAACAAGACTTGTAAAACA
TTCTACATATTTCCAGGTGAGAGAATTTATCCAGGAGTTTTCCATCAGCCCAGAAAAATTCATTAAAATAGCTGTGCGGGAAA
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGGCGGTTTTTGATAGACATCATTCACAGCATTTACCAAAGTTACTCACTAAAACACGA
AGATATTCTTAAACTGGCAACATTTTATGTCAAACACAATGCAATCACCCATTTTAAAGATCTCTGCAAATATCTTTGGCTGA
ACAGAGGAACAGAAAGTAAGAAACTGTTTTTAGAGTGCTTGGAAATTGCTGATGAGAAGGAGTTTCCTGATATTAAAAATATT
GTGAGTGAATATATTAACTACTTGTTTACTGCAGGAGCTATTACCAAGGAAGAAATCATGCAAGCTTATGCTTTAGAGTATGA
CATGTATTAA
L60 MGF 505 6R
SEQ ID No. 258
ATGTTCTCCCTACAGGACCTCTGTCGGAAGAACACTTTCTTCCTTCCAAATGATTTTAGCAAGCATACTCTACAACGGCTGGG
GTTGTATTGGAAAGAGCATGGATCCGTCCATCGAATAGAAAAGGACAGCATAATGATACAGAATGAATTGGTTCTTTCTATCA
ATGATGCTTTACAGCTTGCAGGAGAGGAGGGGGACACAGATGTGGTACAGCTCTTGTTACTATGGGAGGGAAATCTGCATTAT
GCCATCATAGGAGCTTTGAAGACTGAGAATTATAATCTAGTATGTGAGTACCATAGCCAAATTCAGGACTGGCATATTCTCCT
ACCCTTGATTCAAGATCCAGAAACATTCGAAAAATGTCATGATTTAAGCCTTGGATGTGACCTTATATGCCTTCTCCAACATG
CTGTAAAATGTGATATGCTTTCTATTCTTGTTAAATATAAGGAGGATTTACTAAATGTAAGGATTAGGCATCGTACCCAATCC
CTGTTTGTTTTGGCATGCGAAAATCGGAGATTTGAGATTATTGAATGGATAGGTCAAAATCTGTCAATTCCTGAACCTGAGGC
CATTTTTAGCATTGCTATTGTTACAAAAGATGTAGAACTGTTTTCCTTAGGATATAAAATTATTTTTGATTACATGCAAAGAC
AAGGAATTTTTCAATTAACCAATGTAGTTCGCATGCTTCTGCTAAATCGTCATATTGGTATGGCAATAGAAAAAGGACTTTTA
CCTTTTATTCTGGAAACTTTAAAATATGGTGGTAGTGTAAAAAGAGCTTTATCTTATGCAGTAATAGATAATAAAAGAAAAAT
TATAGACTATCTTGTACGCCATGAAAATATACCCCGAGGAACTATTGAAAGACTTTTGCATCTAGCTGTGAAAAAACAATCTT
CCAGGAAAACTTTGAACTTGTTGCTATCTTACATAAATTACAAGGTGAAAAATGTTAAAAAGCTGGTAGAGCATGTAGTAGAT
CACAAATCCACTCTTGTGTTAAAAATTTTATTGGAAAAAAAGGAAAATCTAGTGGATGCTGTTTTAACAAGACTTGTAAAACA
TTCTACATATTTCCAGGTGAGAGAATTTATCCAGGAGTTTTCCATCAGCCCAGAAAAATTCATTAAAATAGCTGTGCGGGAAA
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGGCGGTTTTTGATAGACATCATTCACAGCATTTACCAAAGTTACTCACTAAAACACGA
AGATATTCTTAAACTGGCAACATTTTATGTCAAACACAATGCAATCACCCATTTTAAAGATCTCTGCAAATATCTTTGGCTGA
ACAGAGGAACAGAAAGTAAGAAACTGTTTTTAGAGTGCTTGGAAATTGCTGATGAGAAGGAGTTTCCTGATATTAAAAGTATT
GTGAGTGAATATATTAACTACTTGTTTACTGCAGGAGCTATTACCAAGGAAGAAATCATGCAAGCTTATGCTTTAGAGTATGA
CATGTATTAA
China/2018/AnhuiXCGQ MGF 505 6R
SEQ ID No. 259
ATGTTCTCCCTACAGGACCTCTGTCGGAAGAACATTTTCTTCCTTCCAAATGATTTTAGCAAGCATACCCTACAATGGCTGGG
ATTATATTGGAAAGAGCATGGATCCGTCCATCGAGCAGAAAAAGACAGCATAATGATACAGAATGAATTGGTTCTTTCTATCA
ATGATGCTTTACAGCTTGCAGGAGAGGAGGGGGACACAGATGTAGTACAGCTCTTGTTATTATGGGAGGGAAATCTGCATTAT
GCCATCATAGGAGCCTTGAAGACTGAAAAATATAACCTAATATGTGAGTATCATAGCCAAATTCAGGACTGGCATATTCTCCT
ACCCATGATTCAAGATCCAGAAACATTCGAAAAATGTCATGATTTAAGCCTTGGATGTGACTTTATTTGCCTTCTCCAACATG
CTGTAAAATACAACATGCTTTCTATTCTTGTCAAATATAAGGAGGATCTACTAAATGCAAGGATTAGGCATCGTATCCAATCC
CTGTTTGTTTTGGCATGCGAAAATCGGAGAATTGAAATTATTGATTGGATAGGCCAAAATCTGCCAATTCCTGAACCTGATGC
CATTTTTAGCATTGCTGTTGCTACAAGAGATTTAGAACTGTTTTCCTTAGGGTACAAGATTATTTTTGATTACATGCAAAGAC
AGGGAATCATTCAATTAACCAATGGAGTTCGCATGGTTGTGCTAAATCGTCACATTAGCATGGCAATAGATAATGGTCTTTTA
CCTTTTGTTCTGGAAACTTTAAAACATGGTGGGAATATACATAGAGCCTTATCTTATGCAGTAACACACAATAGAAGAAAAAT
TCTGGATTATCTTATTCGCCAGAAAAATATAGCCCCTAATACAATTGAAAGACTTTTATATCTGGCCGTGAAAAATCAATCTT
CCAGGAAAACTTTGAACTTGTTGCTATCTTACATAAATTACAAGGTGAAAAATGTTAAAAAGCTGGTAGAGCATGTAGTAAAT
GAGAAATCCACTCTTGTGTTAAAAATTTTATTAGAAAAAAAGGAAAATCTAGTGGATGCTGTTTTAACAAGACTTGTAAAACA
TTCTACATATTTCCAGGTGAGAGAATTTATCCAGGAGTTTTCCATCAGCCCAGAAAAATTCATTAAAATAGCTGTGCGGGAAA
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGGCGATTTTTGGTAGACATCATTCACAGCATTTACCAAAGTTACTCACTAAAACACGA
AGATATTCTTAAACTGGCAACATTTTATGTCAAACACAATGCAATCACCCATTTTAAAGACCTCTGCAAATATCTTTGGCTGA
ACAGAGGAACAGAAAGTAAGAAACTGTTTTTAGAGTGTTTAGAAATTGCTGATGAGAAGGAGTTTCCTGATATTAAAAGTATT
GTGAGTGAATATATTAACTACTTGTTTACTGCAGGAGCTATTACCAAGGAAGAAATCATGCAAGCCTATGATGCTTTAGAGTA
G
Georgia 2007/1 MGF 505 6R
SEQ ID No. 260
ATGTTCTCCCTACAGGACCTCTGTCGGAAGAACATTTTCTTCCTTCCAAATGATTTTAGCAAGCATACCCTACAATGGCTGGG
ATTATATTGGAAAGAGCATGGATCCGTCCATCGAGCAGAAAAAGACAGCATAATGATACAGAATGAATTGGTTCTTTCTATCA
ATGATGCTTTACAGCTTGCAGGAGAGGAGGGGGACACAGATGTAGTACAGCTCTTGTTATTATGGGAGGGAAATCTGCATTAT
GCCATCATAGGAGCCTTGAAGACTGAAAAATATAACCTAATATGTGAGTATCATAGCCAAATTCAGGACTGGCATATTCTCCT
ACCCATGATTCAAGATCCAGAAACATTCGAAAAATGTCATGATTTAAGCCTTGGATGTGACTTTATTTGCCTTCTCCAACATG
CTGTAAAATACAACATGCTTTCTATTCTTGTCAAATATAAGGAGGATCTACTAAATGCAAGGATTAGGCATCGTATCCAATCC
CTGTTTGTTTTGGCATGCGAAAATCGGAGAATTGAAATTATTGATTGGATAGGCCAAAATCTGCCAATTCCTGAACCTGATGC
CATTTTTAGCATTGCTGTTGCTACAAGAGATTTAGAACTGTTTTCCTTAGGGTACAAGATTATTTTTGATTACATGCAAAGAC
AGGGAATCATTCAATTAACCAATGGAGTTCGCATGGTTGTGCTAAATCGTCACATTAGCATGGCAATAGATAATGGTCTTTTA
CCTTTTGTTCTGGAAACTTTAAAACATGGTGGGAATATACATAGAGCCTTATCTTATGCAGTAACACACAATAGAAGAAAAAT
TCTGGATTATCTTATTCGCCAGAAAAATATAGCCCCTAATACAATTGAAAGACTTTTATATCTGGCCGTGAAAAATCAATCTT
CCAGGAAAACTTTGAACTTGTTGCTATCTTACATAAATTACAAGGTGAAAAATGTTAAAAAGCTGGTAGAGCATGTAGTAAAT
GAGAAATCCACTCTTGTGTTAAAAATTTTATTAGAAAAAAAGGAAAATCTAGTGGATGCTGTTTTAACAAGACTTGTAAAACA
TTCTACATATTTCCAGGTGAGAGAATTTATCCAGGAGTTTTCCATCAGCCCAGAAAAATTCATTAAAATAGCTGTGCGGGAAA
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGGCGATTTTTGGTAGACATCATTCACAGCATTTACCAAAGTTACTCACTAAAACACGA
AGATATTCTTAAACTGGCAACATTTTATGTCAAACACAATGCAATCACCCATTTTAAAGACCTCTGCAAATATCTTTGGCTGA
ACAGAGGAACAGAAAGTAAGAAACTGTTTTTAGAGTGTTTAGAAATTGCTGATGAGAAGGAGTTTCCTGATATTAAAAGTATT
GTGAGTGAATATATTAACTACTTGTTTACTGCAGGAGCTATTACCAAGGAAGAAATCATGCAAGCCTATGATGCTTTAGAGTA
G
Mkuzi 1979 MGF 505 6R
SEQ ID No. 261
ATGTTCTCCCTACAGGACCTCTGTCGGAAGAACACTTTCTTCCTTCCAAATGATTTTAGCAATCATACCCTACAACGGCTGGG
GTTATATTGGAAAGAGCATGGATCCGTCCATCGAATAGAAAAGGACAGCATAATGATACAGAATGAATTGGTTCTTTCTATCA
ATGATGCTTTACATCTTGCAGGAGAGGAGGGGAACACAGATGTAGTACAGCTCTTGTTATTATGGGAGGGAAATCTGCATTAT
GCCATCATAGGAGCCTTGAAGACTGAGAAATATAACCTAATATGTGAGTACCATAGCCAAATTCAGAACTGGCATGTTCTCCT
CCCCTTGATTCAAGATCCAGAAACATTCGAAAAATGTCATGATTTAAGCCTTGGATGTGACTTTATTTGCCTTCTCCAACATG
CTGTAAAATGTGACATGCTTTCTATTCTTGTTAAATATAAGGAGGATCTACTAAATGCAAGGATTAGGCATCGTATCCAATCC
CTGTTTGTTTTGGCATGCGAAAATCGGAGATTTGAGATTATTGAATGGATAGGTCAAAATCTGCCAATTCCTGAACCTGAGGC
CATTTTTAGCATTGCTATTGTTACAAAAGATATAGAACTGTTTTCCTTAGCGTACAAGCTTATTTTTGATTACATGCAAAGAC
AAGGAACTTTTCAATTAACCAATATGGTCCGCATGCTTCTGCTAAATCGGTACATTGGTATGGCAATAGAAAAAGGACTTTTA
CCCTTTATTCTGGAAACTTTAAAATATGGTGGTAGTGTAAATAGAGCTTTATCTTATGCAGTAATAGATAATAAAAGAAAAAT
TATAGACTATCTTGTACGCCATGAAAATATACCCCGTGGAACTATTGAAAGACTTTTGCATCTAGCTGTGAAAAAACAATCTT
CCAGAAAAACTTTGAACTTGTTGCTATCTTACATAAATTACAAGGTGAAAAATGTTAAAAAGCTGGTAGAGCATGTAGTAGAT
CACAACTCTACTCTTGTGTTAAAAATTTTATTGGAAAAAAAGGAAAATCTAGTGGATGCTGTTTTAACAAGACTTGTAAAACA
TTCTACATATTTCCGGGTAAGAGAATTTATCCAGGAGTTTTCCATCAGCCCAGAAAAATTCATTAAAATAGCTGTGCGGGAAA
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGGCGGTTTTTGATAGACATCATTCACAGCATTTACCAAAGTTATTCACTAAAACATGA
AGATATTTTTAAACTGGCAACATTTTATGTCAAACACAATGCAATCACCCATTTTAAAGATCTCTGCAAATATCTTTGGCTGA
ACAGAGGAACAGAAAGTAAGAAACTGTTTTTAGAGTGCTTGGAAATTGCTGATGAGAAGGAGTTTCCTGATATTAAAAGCATT
GTGAGTGAATATATTAACTACTTGTTTACTGCAGGAGCTATTACCAAGGAAGAAATCATGCAAGCCTATAATGCTTTAGAGTA
G
Pretorisuskop/96/4 MGF 505 6R
SEQ ID No. 262
ATGTTCTCCCTTCAGGACCTCTGTCGGAAGAACATTTTCTTCCTTCCAAACGATTTTAGCAAGCATACCCTACAATGGCTGGG
GTTATATTGGAAAGAGCATGGATCCGTCCATCGAGCAGAAAAAGACAGCATAATGATACAGAATGAATTGGTTCTTTCTATCA
ATGATGCTTTACTGCTTGCAGGAGAGGAGGGGGACACAGATGTAGTACAGCTCTTGTTGCTATGGGAAGGAAATCTGCATTAT
GCCATCATAGGAGCCTTGAAGACTGAGAAATATAGCCTAGTATGTGAGTACCATAGCCAAATTCAGGACTGGCATGTTCTCCT
CCCCTTGATTCAAGATCCAGAAACATTCGAAAAATGTCATGATTTAAGCCTTGAATGTGACTTTATATGCCTTCTACAACATG
CTGTAAAATGTGACATGCTTTCTATTCTTGTTAAATATAAAGAGGATCTACTAAATGTAAGGATTAGGCATCGTATCCAATCC
CTGTTTGTTTTGGCATGCGAAAATCGGAGATTTGAGATTATTGAATGGATAGGTCAAAATCTGCCAATTCCTGAACCTGAGGC
CATTTTTAGCATTGCTATTGTTACAAAAGATATAGAACTGTTTTCCTTAGGATACAAGCTTATTTTTGATTACATGCAAAGAC
AAGGAACTTTTCAATTAACCAATATGGTTCGCATGCTTCTGCTAAATCGTTACATTGGTATGGCAATAGAAAAAGGCCTTTTA
CCCTTTATCGTGGAAACTTTAAAATATGGTGGTAGTGTAAATAGAGCTTTATCTTATGCAGTCATAGATAATAAAAGAAAAAT
TATAGACTATCTTGTACGTCATGAAAATATACCCCGTGGAACTATTGAAAGACTTTTGCATCTAGCTGTGAAAAAACAATCTT
CCAGAAAAACTTTGAACTTGTTGCTATCTTACATAAACTACAAGGTGAAAAATGTTAAAAAGCTGGTAGAGCATGTAGTAGAT
CACAACTCTACCCTTGTGTTAAAAATTTTATTGGAAAAAAAGGAAAATTTAGTGGATGCTGTTTTAACAAGACTTGTAAAACA
TTCTACATATTTCCGGGTGAGAGAATTTATCCAGGAGTTTTCCATCAGCCCAGAAAAATTCATTAAAATAGCTGTGCGGGAAC
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGGCGGTTTTTGATAGACATCATTCACAGCATTTACCAAAGTTACTCACTAAAACACGA
AGATATTCTTAAACTGGCAATATTTTATGTCAAATACAATGCAATCACCCATTTTAAAGATCTCTGCAAATATCTTTGGCTGA
ACAGAGGAACAGAAAGTAAGAAACTGTTTTTAGAGTGCTTGGAAATTGCTGATGAGAAGGAGTTTCCTGATATTAAAAGCATT
GTGAGTGAATATATTAACTACTTGTTTACTGCAGGAGCTATTACTAAGGAAGAAATCATATAA
Tengani 62 MGF 505 6R
SEQ ID No. 263
ATGTTCTCCCTTCAGGACCTCTGTCGGAAGAACACTTTCTTCCTTCCAAATGATTTTAGCAAGCATACCCTACAACGGCTGGG
GTTATATTGGAAAGAGCATGGATCCGTCCATCGAGCAGAAAAAGACAGCATAATGATACAGAATGAATTGGTTCTTTCTATCA
ATGATGCTTTACAGCTTGCAGGAGAGGAGGGGGACACAGATGTAGTACAGCTCTTATTATTATGGGAAGGAAATCTTCATTAT
GCCATCATAGGAGCCTTGAAGACTGAGAAATATAACCTAATATATGAGTACCATAGCCAAATTCAGGACTGGCATGTTCTCTT
ACCCATGATTCAAGATCCAGAAACATTCGAAAAATGTCATGATTTAAGCCTTGCATGTGACTTTATATGCCTTCTCCAACATG
CTATAAAATATAACATGCTTTCTATCCTTGTCAAATATAAGGAAGACTTACTAAATGTAAGGATTAGGTATCGTATCCAATCC
CTGTTTGTTTTGGCATGTGAAAACCGGAGAATTGAGATTATTGAATGGATAGGTCAAAATCTGCCAATTCCTGAACCTGATGC
CATTTTTAGCATTGCTGTTGCTACAAGAGATTTAGAACTGTTTTCCTTAGGGTACAAGATTATTTTTGATTACATGCAAAGAC
AGGGAATCTTTCAATTAACCAATGGAGTTCGCATGGTTGTGCTAAATCGTCACATTAGCATGGCAATAGATAATGGTCTTTTA
CCCTTTGTTCTGGAAACTTTAAAACATGGTGGGAATATACATAGAGCCTTATCTTATGCAGTAACACACAATAAAAGAAAAAT
TCTGGATTATCTTATTCGCCAGAAAAATATAGCCCCTAATACAATTGAAAGACTTTTATATCTGGCCGTGAAAAATCAATCAT
CCAGAAAAACTTTGAACTTGTTACTATCTTACATAAATTACAAGGTGAAAAATGTTAAAAAGCTGGTAGAGCATGTAGTAAAT
GAGAAATCCACTCTTGTGTTAAAAATTTTATTAGAAAAAAAGGAAAATCTAGTGGATGCTGTTTTAACAAGACTTGTAAAACA
TTCTACATATTTCCAGGTGAGAGAATTTATCCAGGAGTTTTCCATCAGCCCAGAAAAATTCATTAAAATAGCTGTGCGGGAAA
AGAAAAATGTGTTGATCGAGGCTATTTCTGAAGATATTTGGGAAAATCCCACAGAAAGAATTACTTATCTCAAACAGATAGTG
CACACCATAAAATATGAAAGTGGAAGGCGATTTTTGGTAGACATCATTCACAGCATTTACCAAAGTTACTCACTAAAACACGA
AGATATTCTTAAACTGGCAACATTTTATGTCAAACACAATGCAACCACCCATTTTAAAGATCTCTGCAAATATCTTTGGCTGA
ACAGAGGAACAGAAAGTAAAAAACTCTTTTTAGAGTGTTTAGAAATTGCTGATGAGAAGGAGTTTCCTGATATTAAAAGCATT
GTGAGTGAATATATTAACTACTTGTTTACTGCAGGAGCTATTACCAAGGAAGAAGCCTATGGATGCTTTAGAGTAGCCATGTA
TTAA
Warmbaths MGF 505 6R
SEQ ID No. 264
ATGTTCTCCCTACAGGACCTCTGTCGGAAGAACATTTTCTTCCTTCCAAACGATTTTAGCAAGCATACCCTACAATGGCTGGG
GTTATATTGGAAAGAGCATGGATCCGTCCATCGAGCAGAAAAAGACAGCATAATGATACAGAATGAATTGGTTCTTTCTATCA
ATGATGCTTTACTGCTTGCAGGAGAGGAGGGGGACATAGATGTAGTACAGCTCTTGTTATTATGGGAGGGTAATCTGCATTAT
GCCATCATAGGAGCCTTGAAGACTGAGAAATATAACCTAATATGTGAGTACCATAGCCAAATTCAGGACTGGCATATTCTCCT
ACCCATGATTCAAGATCCAGAAACATTCGAAAAATGTCATGATTTAAGCCTTGCATGTGACTTTATATGCCTTCTCCAACATG
CTGTAAAATATAACATGCTTTCTATCCTTGTCAAATATAAGGAAGACTTACTAAATGTAAGGATTAGGTATCGTATCCAATCC
CTGTTTGTTTTGGCATGCGAAAACCGGAGAATTGAGATTATTCATTGGATAGGCCAAAATCTGCCAATTCCTGAACCTGATGC
CATTTTTAGCATTGCTGTTGCTACAAGAGATTTAGAACTGTTTTCCTTAGGGTACAAGATTATTTTTGATTACATGCAAAGAC
AGGGAATCTTTCAATTATCCAATGGAGTTCGCATGGTTGTGCTAAATCGTCACATTAGCATGGCAATAGATAATGGACTTTTA
CCCTTTGTTCTGGAAACTTTAAAACATGGTGGGAATATACATAGAGCCTTATCTTATGCAGTAACACACAATAGAAGAAAAAT
TCTGGATTATCTTATTCGCCAGAAAAATATAGCCCCTAATACAATTGAAAGACTTTTATATCTGGCCGTGAAAAATCAATCTT
CCAGGAAAACTTTGAACTTGTTGCTATCTTACATAAATTACAAGGTGAAAAATGTTAAAAAGCTGGTAGAGCATGTAGTAAAT
GAGAAATCCACTCTTGTGTTAAAAATTTTATTGGAAAAAAAGGAAAATCTAGTGGATGCTGTTTTAACAAGACTTGTAAAACA
TTCTACATATTTCCAGGTGAGAGAATTTATCCAGGAGTTTTCCATCAGCCCAGAAAAATTCATTAAAATAGCTGTGCGGGAAA
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGACGATTTTTGATAGAGATCATTCACAGCATTTACCAAAGTTACTCACTAAAACACGA
AGATATTCTTAAACTGGCAACATTTTATGTCAAACACAATGCAACCACCCATTTTAAAGATCTCTGCAAATATCTTTGGCTGA
ACAGAGGAACAGAAAGTAAGAAACTGTTTTTAGAGTGTTTAGAAATTGCTGATGAGAAGGAGTTTCCTGATATTAAAAGTATT
GTGAGTGAATATATTAACTACTTGTTTACTGCAGGAACTATTACCAAGGAAGAAATCATGCAAGCCTATGATGCTTTAGAGTA
G
Warthog MGF 505 6R
SEQ ID No. 265
ATGTTCTCCCTTCAGGACCTCTGTCGGAAGAACATTTTCTTCCTTCCAAATGATTTTAGCAAGCATACCCTACAATGGCTGGG
GTTATATTGGAAAGAGCATGGATCCGTCCATCGAGCAGAAAAAAACAGCATAGTGATACAGAATGAATTGGTTCTTTCTATCA
ATGATGCTTTACAGCTTGCAGGGGAAGAGGGGGACACAGATGTAGTACAGCTCTTGTTATTATGGGAGGGAAATCTGCATTAT
GCCATCATAGGAGCCTTGAAGACTGAGAAATATAATCTAATATGTGAGTACCATAGCCAAATTCAGGACTGGCATGTTCTCCT
CCCCTTGATTCAAGATCCAGAAACATTCGAAAAATGTCATGATTTAAGCCTTGCATGTGACTTTATATGCCTTCTACAACATG
CTGTAAAATATAACATGCTTTCTATCCTTGTCAAATATAAGGAAGACTTACTAAATGTAAGGATTAGGTATCGTATCCAATCC
CTGTTTGTTTTGGCATGTGAAAACCGGAGAATTGAGATTATTGATTGGATAGGCCAAAATCTGCCAATTCCTGAACCTGATGC
CATTTTTAGCATTGCTGTTGCTACAAGAGATTTAGAACTGTTTTCCTTAGGGTACAAGATTATTTTTGATTACATGCAAAGAC
AGGGAATCTTTCAATTATCCAATGGAGTTCGCATGGTTGTGCTAAATCGTCACATTAGCATGGCAATAGATAATGGACTTTTA
CCCTTTGTTCTGGAAACTTTAAAACATGGTGGGAATATACATAGAGCCTTATCTTATGCAGTAACACACAATAGAAGAAAAAT
TCTGGATTATCTTATTCGCCAGAAAAATATAGCCCCTAATACAATTGAAAGACTTTTATATCTGGCCGTGAAAAATCAATCTT
CCAGGAAAACTTTGAACTTGTTGCTATCTTACATAAATTACAAGGTGAAAAATGTTAAAAAGCTAGTAGAGCATGTAGTAAAT
GAGAAATCCACTCTTGTGTTAAAAATTTTATTGGAAAAAAAGGAAAATCTAGTGGATGCTGTTTTAACAAGACTTGTAAAACA
TTCTACATATTTCCAGGTGAGAGAATTTATCCAGGAGTTTTCCATCAGCCCAGAAAAATTCATTAAAATAGCTGTGCGGGAAA
AGAAAAATGTGTTAATCGAGGCTATTTCTGAAGATATTTGGGAAAATCCCACAGAAAGAATTACTTATCTCAAACAGATAGTG
CACACCATAAAATATGAAAGTGGAAGACGATTTTTGATAGACATCATTCACAGCATTTACCAAAGTTACTCACTAAAACACGA
AGATATTCTTAAACTGGCAACATTTTATGTCAAATACAATGCAATCACCCATTTTAAAGATCTCTGCAAATATCTTTGGCTGA
ACAGAGGAACAGAAAGTAAGAAACTGTTTTTAGAGTGTTTAGAAATTGCTGATGAGAAGGAGTTTCCTGATATTAAAAGCATT
GTGAGTGAATATATTAACTACTTGTTTACTGCAGGAGCTATTACCAAGGAAGAAATCATGCAAGCCTATGATGCTTTAGAGTA
G

In an embodiment the attenuated ASFV of the invention comprises a functional version of MGF 505 6R. Suitably the functional version of MGF 505 6R comprises the sequence of SEQ ID No. 257, 258, 259, 260, 261, 262, 263, 264 or 265. Suitably the functional version of MGF 505 2R comprises a sequence having at least 70%, at least 80%, at least 90% or at least 95% identity with SEQ ID No. 257, 258, 259, 260, 261, 262, 263, 264 or 265. Suitably the functional version of MGF 505 6R consists of the sequence of SEQ ID No. 257, 258, 259, 260, 261, 262, 263, 264 or 265.

In an embodiment the invention provides an ASFV which comprises one or more, such as all, of the following sequences from the Benin 97/1 strain:
SEQ ID No. 55 (MGF 360 6L), SEQ ID No. 68 (MGF 360 10L), SEQ ID No. 78 (MGF 360 11L), SEQ ID No. 96 (MGF 360 12L), SEQ ID No. 108 (MGF 360 13L) and SEQ ID No. 120 (MGF 360 14L), and SEQ ID No. 141 (MGF 505 1R) and SEQ ID No. 151 (MGF 505 2R).

In an embodiment the invention provides an ASFV which comprises one or more, such as all, of the following sequences from the China/2018/AnhuiXCGQ strain:
SEQ ID No. 52 (MGF 360 6L), SEQ ID No. 63 (MGF 360 10L), SEQ ID No. 76 (MGF 360 11L), SEQ ID No. 89 (MGF 360 12L), SEQ ID No. 101 (MGF 360 13L), SEQ ID No. 113 (MGF 360 14L) and SEQ ID No. 125 (MGF 360 21R), and
SEQ ID No. 135 (MGF 505 1R) and SEQ ID No. 148 (MGF 505 2R).

In an embodiment the invention provides an ASFV which comprises one or more, such as all, of the following sequences from the Georgia 2007/1 strain:
SEQ ID No. 51 (MGF 360 6L), SEQ ID No. 62 (MGF 360 10L), SEQ ID No. 75 (MGF 360 11L), SEQ ID No. 88 (MGF 360 12L), SEQ ID No. 100 (MGF 360 13L), SEQ ID No. 112 (MGF 360 14L) and SEQ ID No. 124 (MGF 360 21R), and
SEQ ID No. 134 (MGF 505 1R) and SEQ ID No. 147 (MGF 505 2R).

In an embodiment the invention provides an ASFV which comprises one or more, such as all, of the following sequences from the Ken05/Tk1 strain: SEQ ID No. 35 (MGF 110 6L), SEQ ID No. 44 (MGF 110 8L), and
SEQ ID No. 58 (MGF 360 6L), SEQ ID No. 65 (MGF 360 10L), SEQ ID No. 86 (MGF 360 11L) and SEQ ID No. 130 (MGF 360 21R), and
SEQ ID No. 145 (MGF 505 1R) and SEQ ID No. 156 (MGF 505 2R).

In an embodiment the invention provides an ASFV which comprises one or more, such as all, of the following sequences from the Ken06.Bus strain:
SEQ ID No. 36 (MGF 110 6L), and
SEQ ID No. 61 (MGF 360 6L), SEQ ID No. 64 (MGF 360 10L), SEQ ID No. 87 (MGF 360 11L), SEQ ID No. 99 (MGF 360 12L), SEQ ID No. 111 (MGF 360 13L), SEQ ID No. 123 (MGF 360 14L) and SEQ ID No. 132 (MGF 360 21R), and
SEQ ID No. 144 (MGF 505 1R) and SEQ ID No. 158 (MGF 505 2R).

In an embodiment the invention provides an ASFV which comprises one or more, such as all, of the following sequences from the Kenya 1950 strain:
SEQ ID No. 37 (MGF 110 6L), SEQ ID No. 45 (MGF 110 8L), and
SEQ ID No. 60 (MGF 360 6L), SEQ ID No. 66 (MGF 360 10L), SEQ ID No. 85 (MGF 360 11L), SEQ ID No. 98 (MGF 360 12L), SEQ ID No. 110 (MGF 360 13L), SEQ ID No. 122 (MGF 360 14L) and SEQ ID No. 131 (MGF 360 21R), and
SEQ ID No. 143 (MGF 505 1R) and SEQ ID No. 157 (MGF 505 2R).

In an embodiment the invention provides an ASFV which comprises one or more, such as all, of the following sequences from the L60 strain:
SEQ ID No. 54 (MGF 360 6L), SEQ ID No. 67 (MGF 360 10L), SEQ ID No. 77 (MGF 360 11L), SEQ ID No. 95 (MGF 360 12L), SEQ ID No. 107 (MGF 360 13L) and SEQ ID No. 119 (MGF 360 14L), and
SEQ ID No. 140 (MGF 505 1R) and SEQ ID No. 150 (MGF 505 2R).

In an embodiment the invention provides an ASFV which comprises one or more, such as all, of the following sequences from the Malawi Lil-20/1 (1983) strain:
SEQ ID No. 38 (MGF 110 6L), SEQ ID No. 46 (MGF 110 8L), and
SEQ ID No. 59 (MGF 360 6L), SEQ ID No. 74 (MGF 360 10L), SEQ ID No. 84 (MGF 360 11L), SEQ ID No. 97 (MGF 360 12L), SEQ ID No. 109 (MGF 360 13L), SEQ ID No. 121 (MGF 360 14L) and SEQ ID No. 133 (MGF 360 21R), and
SEQ ID No. 146 (MGF 505 1R) and SEQ ID No. 155 (MGF 505 2R).

In an embodiment the invention provides an ASFV which comprises one or more, such as all, of the following sequences from the Mkuzi 1979 strain:
SEQ ID No. 39 (MGF 110 6L), SEQ ID No. 47 (MGF 110 8L), and
SEQ ID No. 57 (MGF 360 6L), SEQ ID No. 69 (MGF 360 10L), SEQ ID No. 79 (MGF 360 11L), SEQ ID No. 94 (MGF 360 12L), SEQ ID No. 106 (MGF 360 13L), SEQ ID No. 118 (MGF 360 14L) and SEQ ID No. 128 (MGF 360 21R), and
SEQ ID No. 138 (MGF 505 1R) and SEQ ID No. 149 (MGF 505 2R).

In an embodiment the invention provides an ASFV which comprises one or more, such as all, of the following sequences from the Pretorisuskop/96/4 strain:
SEQ ID No. 40 (MGF 110 6L), SEQ ID No. 48 (MGF 110 8L), and
SEQ ID No. 56 (MGF 360 6L), SEQ ID No. 70 (MGF 360 10L), SEQ ID No. 81 (MGF 360 11L), SEQ ID No. 93 (MGF 360 12L), SEQ ID No. 105 (MGF 360 13L), SEQ ID No. 117 (MGF 360 14L) and SEQ ID No. 127 (MGF 360 21R), and
SEQ ID No. 142 (MGF 505 1R) and SEQ ID No. 153 (MGF 505 2R).

In an embodiment the invention provides an ASFV which comprises one or more, such as all, of the following sequences from the Tengani 62 strain:
SEQ ID No. 41 (MGF 110 6L), and
SEQ ID No. 71 (MGF 360 10L), SEQ ID No. 82 (MGF 360 11L), SEQ ID No. 90 (MGF 360 12L), SEQ ID No. 102 (MGF 360 13L), SEQ ID No. 114 (MGF 360 14L) and SEQ ID No. 126 (MGF 360 21R), and
SEQ ID No. 136 (MGF 505 1R) and SEQ ID No. 152 (MGF 505 2R).

In an embodiment the invention provides an ASFV which comprises one or more, such as all, of the following sequences from the Warmbaths strain:
SEQ ID No. 42 (MGF 110 6L), SEQ ID No. 49 (MGF 110 8L), and
SEQ ID No. 53 (MGF 360 6L), SEQ ID No. 73 (MGF 360 10L), SEQ ID No. 80 (MGF 360 11L), SEQ ID No. 92 (MGF 360 12L), SEQ ID No. 104 (MGF 360 13L), SEQ ID No. 116 (MGF 360 14L) and SEQ ID No. 129 (MGF 360 21R), and
SEQ ID No. 137 (MGF 505 1R) and SEQ ID No. 154 (MGF 505 2R).

In an embodiment the invention provides an ASFV which comprises one or more, such as all, of the following sequences from the Warthog strain:
SEQ ID No. 43 (MGF 110 6L), SEQ ID No. 50 (MGF 110 8L), and
SEQ ID No. 72 (MGF 360 10L), SEQ ID No. 83 (MGF 360 11L), SEQ ID No. 91 (MGF 360 12L), SEQ ID No. 103 (MGF 360 13L), SEQ ID No. 115 (MGF 360 14L), and
SEQ ID No. 139 (MGF 505 1R).

The translation products (i.e. protein sequences) of these genes are given below:

### Benin 97/1 MGF Gene Protein Sequences

translation of Benin 97/1 MGF 110-11L (A9JKR9)
SEQ ID No. 1
MKYSWKNGGGDYWPIIIRHCCFYLVFSIAFVGYIVFAYYKNLHLNTAMKLLALLCILIWLSQPGLNRPLSIFYMKQNLPRTYT
PPIRELEYWCTYGKHCDFCWECRNGICKNKVWDDMSSVQEHSYPMEHCMIHRQCKYIRDGPIFQVECTMQTSDATHLINA
translation of Benin 97/1 MGF 110-12L (A9JKS1)
SEQ ID No. 2
MKVFLGLLLGYSTILILTYQSPTTQWCFYEISLKILNHHSMEKWRDKNWSIIIRYYCFYLVFSFAFAGCVAFAICKNLRLCTT
MKLLMLLNILVLLSQPILNN
translation of Benin 97/1 MGF 360-6L (A9JKS8)
SEQ ID No. 3
MNSLQVLTKKVLIENKAFSEYHEDDIFILQQLGLWWHNGPIGFCKQCKMVTSGSMSCSDVDSYELDRALVRAVKKNQTDLIKL
FVLWGANINYGIICAKTERTKDLCIELGANPEFLDVGLYNMFVDLIKQQKVLLAIDIYYDNISILDSFDSHDFYVLIDFIYNC
FILNLDEKEKMIKNTYVLKFWFKIAIEFNLIKPIRFLSKKFPHLDYWRLKTAVYLGNVDEIHHAYFQENIRLDPNDMMSLACM
YPQNKLGIYYCFALGANINTALETLIGFINHEVNREITFFSNYGIWSNVHFCISLGANPYTKKIQETLLRQEKNVIMKLLFKK
GLLSPHSILHKKILEPSEVRKIISTYEYTETFHSFSLLRDNLR
translation of Benin 97/1 MGF 360-10L
SEQ ID No. 4
MVPSLQSFAKKVLASQHVSIDYHVILERCGLWWYKAPISLDCKHMLIKLPNFADGLDLNTALMLATKENNYQLIKMFTDWGAD
INYGLICANTPPIREFCWELGAKYQVDKKKIMHIFFKLIHPNTTSNNIILCLKFENDNPFSAYVIIREIKSCIHWKLKNLAED
TNVLSNISDGDMLTIYCFIVALQDNLREAISYVYQHFKYLNTWWLTCALCYNKLFDLHNLYEKEKIRMDMDEMMRIACTKDNN
FLTIYYCFILGANINLAMIASIRFYNMDNLFFCIDLGADAFEEAKALAEQQNYYLISHRLSLDIYSPDSSLLTLKEADPNKIY
RLLKNYKSKSMLAYLNYDINDTSL
translation of Benin 97/1 MGF 360-11L (A9JKU4)
SEQ ID No. 5
MLPSLQSLTKKVLAGQCVSVDHYHILKCCGLWWHNGPIMLHIRRNKLFIRSTCFSQGIELNIGLMKAVKENNHDLIKLFTEWG
ADINYGMICALTENTRDLCKELGAKEYLEREYILKIFFDTTRDKTSSNIIFCHEVFSNNPNLRIIDNLDLRGEIMWELRGLME
ITFMLDHDDSFSTVLTKYWYAIAVDYDLKDAIRYFYQKYPRLHRWRLMCALFYNNVEDLHELYEIERVRMDIDEMMHIACIQD
YSYSAIYYCFIMGANINQAMLVSIQNYNLGNLFFCIDLGANAFEEGKALAEQKENYLIAHALSLKHYNPVISLLSNVMDPEKI
NYMLKNYHSINMGIFLDYEQR
translation of Benin 97/1 MGF 360-12L (A9JKU9)
SEQ ID No. 6
MLPSLQSLTKKVLAGQCVPTNQHYLLKYYDLWWYNAPITFDHNLRLIKSSGIKEGLDLNTALVKAVRENNYSLIKLFTEWGAD
INYGLVSVNTEHTRDLCQELGAKEILNEEEILQIFIDLKFHKTSSNIILCHEVFSNNPILQKVNNLKLRIEIFWELRELIEKT
DLLNNEFLLSTLLLKYWYAIAVRYSLKEAIQYFYQKYTHMNTWRLTCALCENNVFDLHEAYEKDKIHMDIEEMMRIACIKDHN
LSTMYYCYMLGANINQAMLTSIQYYNIENMFFCMDLGADVFEEGTTALGEGYELIKNILSLKIYSPTTIPLPKSTDPEIIDHA
LKNYFSKNMMIFLSYDLR
translation of Benin 97/1 MGF 360-13L (A9JKU7)
SEQ ID No. 7
MSLPLSLQTLVKKTVASQCLSIDEHCILKYCGLWWHDAPLKLCMDRGRIQIKSGFLGEDIDLRVALIIAVKENNYSLIKLFTE
WGANINYSLLSINTKHIRELCRQLGAKETLEDNDIFRIFTRIMHNKTSGSIILCHEIFMNNPMLENKFVIQLRGLIYKRLWGL
IEIKETDELNDLLVKYWYAKAVQYVCKNAICFLDEKYTDLNEWRLKCLLYYNKIYELHEMYHKKKVQIDVHDMICLACAKDNN
LLTIYYCYALGGNINQAMLTSVQYYNVGNIFFCIDLGGNAFEEGRAIAEQKGYNFLSHSLTLDIYSSDASLPLNLKDPEKISS
LLKDYKSKNLSIIWEYSHNIL
translation of Benin 97/1 MGF 360-14L (A9JKV1)
SEQ ID No.
MLSLQTLAKKVVACNYLSSDYDYTLQRFGLWWDLGPIHLCNNCKQVFSYKHLQCFSEDDLCLEAALVKAVKSDNLELIRLFVD
WGANPEYGLIRVPAVYLKRLCAELGGLTPVSEPRLLEILKEVANLKSCAGVLLGYDMFCHNPLLETVTRTTLDTVTYTCSNIP
LTGDTAHLLLTKFWFALALRHNFTKAIHYFYKRHKNQLYWRVACSLYENNIFDIHELCREKEICISPNLMMKFACLREKNYAA
IYYCHRLGASLDYGMNLSIYNNNTLNMFFCIDLGAADFDRAQLIAHKAYMYNLSNIFLVKQLFSRDVTLVLDVTEPQEIYDML
KTYTSKNMKRAEEYLTAHPEIIVID
translation of Benin 97/1 MGF 505-1R (A9JKU5)
SEQ ID No. 9
MFSLQNLCRKTLPDCKLPEFFDDYILQLLGLYWENHGTIQRAGNNCVLIQQHTLIPVNEALRIAASEENYEIVGLLLAWEGNL
YYAIIGALEGNRYNLIRKYDDQIKDHHDILPFIDDPIIFHKCHIMRRCFFDCILYQAVKYSKFRVLLYFKYTLEDDLPLVHLL
IEKACEDHNYEVIKWIYENLHVCHIIDTFDCAIAHKDLRLYCLGYTFIYNRIVPYKYHHLDILILSSLQLLHKVAAKGYLDFI
LETLKYDHNIDNLDVILTQAATYNHRKILTYFIPQSTYAQIEQCLFVAIKTKSSKKTLNLLLSHLNLSIKLIQKISQYVATEN
STNIIGILSMKRKKKIYLDIILTKFVKNAIFNKFVVRCMERFSINPERIVKMAARINKMMLVKKISEHVWKNHAARLKHLKHA
VHTMKHKDGKNRLMNFIYEHCYYHMQGEEIFSLARFYAIHHAPKLFDVFYNCCILDTIRFKSLLLDCSHIIGKNAHDATNINI
VNKYIGNLFAMGVLSKKEILQDYPSIYSKHYMP
translation of Benin 97/1 MGF 505-2R (A9JKV3)
SEQ ID No. 10
MFSLQDLCRKHLFILPDVFGEHVLQRLGLYWRCHGSLQRIGDDHILIRRDLILSTNEALRMAGEEGNNEVVKLLLLWKGNLHY
AVIGALQGDQYDLIHKYENQIGDFHFILPLIQDANTFEKCHALERFCGVSCLLKHATKYNMLPILQKYQEELSMRAYLHETLF
ELACLWQRYDVLKWIEQTMHVYDLKIMFNIAISKRDLTMYSLGYIFLFDRGNTEATLLTQHLEKTAAKGLLHFVLETLKYGGN
IDTVLTQAVKYNHRKLLDYFLRQLPRKHIEKLLLLAVQEKASKKTLNLLLSHLNYSVKRIKKLLRYVIEYESTLVIKILLKKR
VNLIDAMLEKMVRYFSATKVRTIMDELSISPERVIKMAIQKMRTDIVIHTSYVWEDDLERLTRLKNMVYTIKYEHGKKMLIKV
MHGIYKNLLYGEREKVMFHLAKLYVAQNAATQFRDICKDCYKLDVARFKPRFKQLILDCLEIVTKKSCYSILEILEKHIISLF
TMKVMTEEEKNLCLEILYKVIHYKTIQC

### MGF 110 5L Protein Sequences

Ken05/Tk1 MGF 110 5L protein
SEQ ID No. 306
MLVIFLGLLGLLANQVLGLPNQPAGQLHPTDNPPKEELGYWCTYTESCKFCWNCQNGLCEGKLENTTILENEYVQSCIVSRWL
NKCMYDLGQGIDHVMACSEPKPWNPYKILKREWKKNVSQN
Ken06.Bus MGF 110 5L protein
SEQ ID No. 307
MLVIFLGLLGLLANQVLGLPNQPAGQLHPTDNPPKEELGYWCTYTESCKFCWNCQNGLCEGKLENTTILENEYVQSCIVSRWL
NKCMYDLGQGIHHVMPCSEPKPWNPYKILKKEWKENVSQN
Malawi Lil-20/1/1983 MGF 110 5L protein
SEQ ID No. 308
MLVIFLGILGLLANQVSSQLVGQLHPTENPSENELEYWCTYMECCQFCWDCQDGLCVNKLGNTTILENEYVHPCIVSRWLNKC
MYDLGQGIDHVMVCSQPKYWNPYKILKKEWKENNSQN
Mkuzi 1979 MGF 110 5L protein
SEQ ID No. 309
MLVIFLGILGLLANQVSSQLVGQLHPTENPSENELEYWCTYMECCQFCWDCQDGLCVNKLGNTTILENEYVHPCIVSRWLNKC
MYDLGQGIDHVMVCSQPKYWNPYKILKKEWKENNSQN
Pretorisuskop/96/4 MGF 110 5L protein
SEQ ID No. 310
MLVIFLGILGLLANQVSSQLVGQLHPTENPSENELEYWCTYMECCQFCWDCQNGLCVNKLGNTTILENEYVHPCIVSRWLNKC
MYDLGQGIDHVMVCSQPKYWNPYKILKKEWKENNSQNK
Tengani62 MGF 110 5L protein
SEQ ID No. 311
MLVIFLGILGLLVNQVSSQPVGQLHPTENPSENELEYWCTYMECCQFCWDCQNGLCVNKLGNTTILENEYVHPCIVSRWLNKC
MYDLGQGIDHVMVCSQPKYWNPYKILKKEWKENNS
Warmbaths MGF 110 5L protein
SEQ ID No. 312
MLVIFLGILGLLANQVSSQLVGQLHPTENPSENELEYWCTYMECCQFCWDCQNGLCVNKLGNTTILENEYVHPCIVSRWLNKC
MYDLGQGIDHVMVCSQPKYWNPYKILKKEWKENNSQNK
Warthog MGF 110 5L protein
SEQ ID No. 313
MLVIFLGILGLLANQVSSQLVGQLHPTENPSENELEYWCTYMECCQFCWDCQNGLCVNKLGNTTILENEYVHPCIVSRWLNKC
MYDLGQGIDHVMVCSQPKYWNPYKILKKEWKENNSQNK
China/2018/AnhuiXCGQ MGF 110 5L/6L fusion protein
SEQ ID No. 314
MLVIFLGILGLLASQVSSQLVGQLRPTEEPPEEELEYWCAYMESCQFCWDCQDGTCINKIDGSVIYKNEYVKSCLVSRWLDKC
MYDLDKGIYHTMNCNQVLGLPNQPAGQLHPTDNPPQEELEYWCTYTENCKFCWNCQNGLCEGKLENTTILENEYVQSCIVSRW
LNKCMYDLGQGIHHVMACSEPKPWNPYKILKREWKENNS
Georgia 2007/1 MGF110 5L/6L fusion protein
SEQ ID No. 315
MLVIFLGILGLLASQVSSQLVGQLRPTEEPPEEELEYWCAYMESCQFCWDCQDGTCINKIDGSVIYKNEYVKSCLVSRWLDKC
MYDLDKGIYHTMNCNQVLGLPNQPAGQLHPTDNPPQEELEYWCTYTENCKFCWNCQNGLCEGKLENTTILENEYVQSCIVSRW
LNKCMYDLGQGIHHVMACSEPKPWNPYKILKREWKENNS

### MGF 110 12L Protein Sequences

Benin 97/1 MGF 110 12L protein
SEQ ID No. 316
MKVFLGLLLGYSTILILTYQSPTTQWCFYEISLKILNHHSMEKWRDKNWSIIIRYYCFYLVESFAFAGCVAFAICKNLRLCTT
MKLLMLLNILVLLSQPILNN
China/2018/AnhuiXCGQ MGF 110 12L protein
SEQ ID No. 317
MKVFLGLLLGYSTILILTYQSPTTPWCFYEISLKIPNHHSMKCCSYPRLYEHEMFMEKWRDKNWPIIIRYYCFYLVFSFVFAG
CVAFAICKNLRLSTTMKLLMLLSILVLLLSQPILNN
Georgia 2007/1 MGF 110 12L protein
SEQ ID No. 31
MKVFLGLLLGYSTILILTYQSPTTPWCFYEISLKIPNHHSMKCCSYPRLYEHEMFMEKWRDKNWPIIIRYYCFYLVFSFVFAG
CVAFAICKNLRLSTTMKLLMLLSILVLLLSQPILNN
Ken05/Tk1 MGF 110 12L protein
SEQ ID No. 319
MKVFLGLLLGFSIILILTYQSPTTQHPPKEELAYWCTYAKSCDFCWDCQNDTCINKVINESISITSIVNCRVTRDSQSCFYEI
SVKIPNHHSMECSYPRLYEHEMFMEKWRDEYWPIIIKQCCFYLVFSIAFAGCVAFAICKNLRLRTTIKLLILLSILVWLSQPV
LN
Ken06.Bus MGF 110 12L protein
SEQ ID No. 320
MKVFLALLLGYLTILILTYQTPTTQHPPKEELPYWCTYVKNCDLCWDCQDSIYWNKVISESISINSIINCRVTCDSQSQSCFY
EILLKIPNHHSMECSYPGSYENEMFMEKWRDENWSIIIKHYCFYLVFSFAFAGCVAFAICKNLRLSTTMKLLMLLSILVCLSQ
PILNN
Kenya 1950 MGF 110 12L protein
SEQ ID No. 321
MKVFLGLLLGFSIILILTYQSPTTQHPPKEELAYWCTYAKSCDFCWDCQNDTCINKVINESISITSIVNCRVTRDSQSCFYDI
SVKIPNHHSMECSYPRLYEHEMFMEKWRDEYWPIIIKQCCFYLVFSFAFAGCVAFAICKNLRLRTTIKLLILLSILVWLSQPI
LNN
L60 MGF 110 12L protein
SEQ ID No. 322
MKVFLGLLLGYSTILILTYQSPTTQWCFYEISLKILNHHSMEKWRDKNWSIIIRYYCFYLVFSFAFAGCVAFAICKNLRLCTT
MKLLMLLNILVLLSQPILNN
Malawi Lil-20/1/1983 MGF 110 12L protein
SEQ ID No. 323
MKVFLGLLLGYSTILILTYQSPTTQHPPKEELEYWCTYAKTCDFCWDCQNDTCINKVINESISMNSIVNCRVTRDSQSQSCFY
EISLKIPNYHSMECSYPRLYKHFMSMEKWRDENWPILIRHYCFYLVFSFAFAGCVAFAICKNLRLRTTMKLLMLLSILVLLSQ
PILNN
Mkuzi 1979 MGF 110 12L protein
SEQ ID No. 324
MNATMKVFLGLLLGYSTILILTYQSPTTQWCFYEISLKILNHHSMEKWRDKNWSIIIRYYCFYLVFSFAFAGCVAFAICKNLR
LCTTMKLLMLLGILVLLSQPILNN
Warmbaths MGF 110 12L protein
SEQ ID No. 325
MKVFLGLLLGYSTILILTYQSPATQWCFYEISLKIPNHHSMECSYPRLYKHFIFMEKWRDKNWSIIIRYYCFYLVFSFAFAGC
IAFAICKNLRLCTTMKLLMLLSILVLLSQPILNN
Warthog MGF 110 12L protein
SEQ ID No. 326
MKVFLGLLLGYSTILILTYQSPATQWCFYEISLKIPNHHSMECSYPRLYKHFMFMKKWRDKNWSIIIRYYCFYLVFSFAFAGC
IAFAICKNLRLCTTMKLLMLLSILVLLSQPILNN

### MGF 360 12L Protein Sequences

Georgia 2007/1 MGF 360 12L protein
SEQ ID No. 159
MLPSLQSLTKKVLAGQCVPTNQHYLLKCYDLWWHDAPITFDHNLRLIKSAGIKEGLNLNTALVKAVRENNYNLIKLFAEWGAD
INYGLVSVNTEHTWDLCRELGAKETLNEEEILQIFIDLKFHKTSSNIILCHEVFSNNPILQKVNNIKMRIEIFWELRELIVKT
DLLNNEFSLSTLLLKYWYAIAIRYNLKEAIQYFYQKYTHLNTWRLTCALCENNVFDLHEAYEKDKIHMDIEEMMRIACIKDHN
LSTMYYCYVLGANINQAMLSSIQYYNIENMFFCIDLGADVFEEGTTALGEGYELIKNILSLKIYSPATTPLPKSTDPEIIDHA
LKNYVSKNMMIFLTYDLR*
China/2018/AnhuiXCGQ MGF 360 12L protein
SEQ ID No. 160
MLPSLQSLTKKVLAGQCVPTNQHYLLKCYDLWWHDAPITFDHNLRLIKSAGIKEGLNLNTALVKAVRENNYNLIKLFAEWGAD
INYGLVSVNTEHTWDLCRELGAKETLNEEEILQIFIDLKFHKTSSNIILCHEVFSNNPILQKVNNIKMRIEIFWELRELIVKT
DLLNNEFSLSTLLLKYWYAIAIRYNLKEAIQYFYQKYTHLNTWRLTCALCENNVFDLHEAYEKDKIHMDIEEMMRIACIKDHN
LSTMYYCYVLGANINQAMLSSIQYYNIENMFFCIDLGADVFEEGTTALGEGYELIKNILSLKIYSPATTPLPKSTDPEIIDHA
LKNYVSKNMMIFLTYDLR*
Tengani 62 MGF 360 12L protein
SEQ ID No. 161
MLPSLQSLTKKVLAGQCVPTNQHYLLKCYDLWWHDAPITFDHNLRLIKSAGIKEGLNLNTALVKAVRENNYNLIKLFAEWGAD
INYGLVSVNTEHTWDLCRELGAKETLNEEEILQIFIDLKFHKTSSNIILCHEVFSNNPILQKVNNIKMRIEIFWELRELIEKT
DLLNNEFSLSTLLLKYWYAIAIRYNLKEAIQYFYQKYTHLNTWRLTCALCFNNVFDLHEAYEKDKIHMDIEEMMRIACIKDHN
LSTMYYCYVLGANINQAMLSSIQYYNIENMFFCIDLGADVFEEGTTALGEGYELIKNILSLKIYSPATTPLPKSMDPEIIDHA
LKNYVSKNMMIFLTYDLR*
Warthog MGF 360 12L protein
SEQ ID No. 162
MLPSLQSLTKKVLAGQCVPTNQHYLLKCYDLWWHDGPITFDHNLKLIKSAGIKEGLDLNTALVKAVRENNYNLIKLFAEWGAN
INYGLVSVNTEHTRDLCRELGAKETLNEEEILQIFIDLKFHKTSSNIILCHEVFSNNPILQKVNNIKMRIEIFWELRELIEKT
DLLNNEFSLSALLLKYWYAIAIRYNLKEAIQYFYQKYTHLNTWRLTCALCENNVFDLHEAYEKDKIHMDIEEMMRIACIKDHN
LSTMYYCYVLGANINQAMLTSIQYYNIENMFFCMDLGADAFEEGTIALGEGYKLIKNILSLKIYSPATTPLPKSTDPEIIDHA
LKNYVSKNMMIFLTYDLR*
Warmbaths MGF 360 12L protein
SEQ ID No. 163
MLPSLQSLTKKVLAGQCVPTNQHYLLKCYDLWWHDGPITFDHNLKLIKSAGIKEGLDLNTALVKAVRENNYNLIKLFAEWGAD
INYGLVSVNTEHTRDLCRELGAKETLNEEEILQIFIDLKFHKTSSNIILCHEVFSNNPILQKVNNIKMRIEIFWELRELIEKT
DLLNNEFSLSTLLLKYWYAIAIRYNLKEAIQYFYQKYTHLNTWRLTCALCENNVFDLHEAYEKDKIYMDIEEMMRIACIKDHN
LSTMYYCYVLGANINQAMLTSIQYYNVENMFFCMDLGADAFEEGTIALGEGYKLIKNILSLKIYSPATTPLPKSTDPEIIDHA
VKNYVSKNMMIFLTYDLR*
Pretorisuskop/96/4 MGF 360 12L protein
SEQ ID No. 164
MLPSLQSLTKKVLAGQCVPTNQHYLLKCYDLWWHDGPITFDHNLKLIKSAGIKEGLDLNTALVKAVRENNYNLIKLFAEWGAN
INYGLVSVNTEHTWDLCRELGAKETLNEEEILQIFIDLKFHKTSSNIILCHEVFSNNPILQKVNNIKMRIEIFWELRELIEKT
DLLNNEFSLSTLLLKYWYAIAIRYNLKEAIQYFYQKYTHLNTWRLTCALCFNNVFDLHEAYEKDKIHMDIEEMMRIACIKDHN
LSTMYYCYVLGGNINQAMLTSIQYYNIENMFFCMDLGADAFEEGTIALGEGYKLIKNILSLKIYSPATTPLPKSTDPEIIDHA
LKNYVSKNMMIFLTYDLR*
Mkuzi 1979 MGF 360 12L protein
SEQ ID No. 165
MLPSLQSLTKKVLAGQCVPTNQHYLLKYYDLWWHDAPITFDHNLRLIKSAGIKEGLDLNTALVKAVKENNYNLIKLFAEWGAD
INYGLVSVSSEHTWDLCRELGAKETLNEKEILQIFIDLKFHKTSSNIILCHEVFSNNPILQKVNNIKMRIEIFWELRELIEKT
DLLNNEFSLSTLLLKYWYAIAIRYNLKEAIQYFYQKYTHLNTWRLTCALCFNNVFDLHEAYEKDKIYMDIEEMMRIACIKDHN
LSTMYYCYMLGANINQAMLTSIQYYNIENMFFCMDLGADVFEEGTTALGEGYELIKNILSLKIYSPTTIPLPKSTDPEIIDHA
LKNYFSKNMMIFLSYDLR*
L60 MGF 360 12L protein
SEQ ID No. 166
MLPSLQSLTKKVLAGQCVPTNQHYLLKYYDLWWYNAPITFDHNLRLIKSSGIKEGLDLNTALVKAVRENNYSLIKLFTEWGAD
INYGLVSVNTEHTRDLCQELGAKEILNEEEILQIFIDLKFHKTSSNIILCHEVFSNNPILQKVNNLKLRIEIFWELRELIEKT
DLLNNEFLLSTLLLKYWYAIAVRYSLKEAIQYFYQKYTHLNTWRLTCALCFNNVFDLHEAYEKDKIHMDIEEMMRIACIKDHN
LSTMYYCYMLGANINQAMLTSIQYYNIENMFFCMDLGADVFEEGTTALGEGYELIKNILSLKIYSPTTIPLPKSTDPEIIDHA
LKNYFSKNMMIFLSYDLR*
Benin 97/1 MGF 360 12L protein
SEQ ID No. 6
MLPSLQSLTKKVLAGQCVPTNQHYLLKYYDLWWYNAPITFDHNLRLIKSSGIKEGLDLNTALVKAVRENNYSLIKLFTEWGAD
INYGLVSVNTEHTRDLCQELGAKEILNEEEILQIFIDLKFHKTSSNIILCHEVFSNNPILQKVNNLKLRIEIFWELRELIEKT
DLLNNEFLLSTLLLKYWYAIAVRYSLKEAIQYFYQKYTHMNTWRLTCALCENNVFDLHEAYEKDKIHMDIEEMMRIACIKDHN
LSTMYYCYMLGANINQAMLTSIQYYNIENMFFCMDLGADVFEEGTTALGEGYELIKNILSLKIYSPTTIPLPKSTDPEIIDHA
LKNYFSKNMMIFLSYDLR*
Malawi Lil-20/1 MGF 360 12L protein
SEQ ID No. 167
MLPSLQSLTKKVLAGQCLPTDQYYLLKCYDLWWYDSPITFDHNLGLIKSAGIKDGLDLNTALVKAVRENNYNLIKLFTEWGAD
INYGLVSVNTEHTRDLCRELGAKETLNEEEILRIFIDLKFYKTSSNIILCHEVFSNNPLLQKVNNLKMRIEIFWELRELIKKT
DLLNNEFSLNTLLLKYWYAIAVRYNLKEAIQYFYQKYTHLNTWRLTCALCENNVFDLHEAYEKDKIYMDLEEMMRVACIKDHN
LSTIYYCYVLGANINQAMLASIQYYNIENMFFCMDLGADVFEENMPVGEGYELIRNILSLKIYSPSTAPLPKNTDPEIIDHVL
KNYKSKNMMTFLSYDLR*
Kenya 1950 MGF 360 12L protein
SEQ ID No. 168
MLPSLQSLTKKVLAGQCLPEDQHYLLKCYDLWWNNAPITFDHNLRLIKSAGLQEGLDLNMALVKAVKENNYSLIKLFTEWGAN
INYGLISVNTEHTWDLCRELGAKKTLNEGDILQIFIDLKFHKTSSNIILCHEVFSDNLLLKKVNNLKMRIEIFWELREIIEKT
DLLNNEFSLNTLLLKYWYAIAVRYNLKEAIQYFYQKYTHLNTWRLTCALCENNVFDLHEAYEKDKICMDLEEMMRIACIKDHS
LSTIYYCYMLGANINQAMLTSIQYYNIENIFFCMDLGADAFEEGMALVGQEGYEPIRNILSLKIYSPATTPLPKSTDPEIIDH
ELKNYFSKNMMVFLTYDLR*
Ken06.Bus MGF 360 12L protein
SEQ ID No. 169
MLPSLQSLTKKVLARQCLPEDQHYLLKCYDLWWNNAPITFDHNLRLIKLAGIQEGLDLNMALVKAVKENNYSLIKLFTEWGAN
INYGLISVNTEHTWDLCRELGAKKTLNEGDILQIFIDLKFYKTSSNIILCHEVFSDNLLLKRVNNLKMRIEIFWELREIIEKT
DLLNNEFSLNTLLLKYWYAIAVRYNLKEAIQYFYQKYTHLNTWRLTCALCENNVEDLHEAYEKDKICMDLEEMMRIACIKDHN
LSTIYYCYMLGANINQAMLTSIQYYNIENIFFCMDLGADAFEEGMALVGQEGYEPIRNILSLKIYSPATTPLPKSTDPEIIDH
ALKNYFSKNMMVFLTYDLR*

### MGF 360 13L Protein Sequences

Georgia 2007/1 MGF 360 13L protein
SEQ ID No. 170
MSLPLSLQTLVKKTIASQCLSIDEHCILKYCGLWWHDAPLKLCMDRGRIQIKSGFLGEDIDLRVALIIAVKENNYSLIKLFTE
WGANINYGLLSINTKHIRELCRQLGAKETLEDNDIFRIFTRIMHNKTSGSIILCHEIFMNNPILENKFVIQLRGLIYKRLWGL
IEIKETDELNGLLVKYWYAKAVQYDCKDAICFLDEKYTDLNEWRLKCLLYYNKIYELHEMYHKENIQIDVHDMICLASTKDNN
PLTIYYCYALGGNINQAMLTSVQYYNIGNIFFCIDLGGNAFEEGRAIAEQKGYNFLSHSLALDIYSSDASLPLNLKDPEEISS
LLKDYKSKNLSIIWEYSHNIL*
China/2018/AnhuiXCGQ MGF 360 13L protein
SEQ ID No. 171
MSLPLSLQTLVKKTIASQCLSIDEHCILKYCGLWWHDAPLKLCMDRGRIQIKSGFLGEDIDLRVALIIAVKENNYSLIKLFTE
WGANINYGLLSINTKHIRELCRQLGAKETLEDNDIFRIFTRIMHNKTSGSIILCHEIFMNNPILENKFVIQLRGLIYKRLWGL
IEIKETDELNGLLVKYWYAKAVQYDCKDAICFLDEKYTDLNEWRLKCLLYYNKIYELHEMYHKENIQIDVHDMICLASTKDNN
PLTIYYCYALGGNINQAMLTSVQYYNIGNIFFCIDLGGNAFEEGRAIAEQKGYNFLSHSLALDIYSSDASLPLNLKDPEEISS
LLKDYKSKNLSIIWEYSHNIL*
Tengani 62 MGF 360 13L protein
SEQ ID No. 172
MSLPLSLQTLVKKTVANQSLSIDEHCILKHCGLWWHDVPLKLCMDRGQIQIKSGFLGEDIDLHIALIIAVKENNYSLIKLFTE
WGANINYSLLSINTKHIRELCRQLGAKETLEDDDIFRIFTKIMHNKTSGSIILCHDIFMNNPNIEDKFTIQLRGLIYKRLWGL
IEIKETDELNGLLVKYWYAKAVQYECKDAICFLDEKYTDLNEWRLKCLLYYNKIYELHEMYHKENIQIDVHDMICLASTKDNN
PLTIYYCYALGGNINQAMLTSVQYYNIGNIFFCIDLGGNAFEEGRAIAEQKGYNFLSHSLALDIYSSDASLPLNLKDPEEISS
LLKDYKSKNLSIIWEYSHNIL*
Warthog MGF 360 13L protein
SEQ ID No. 173
MSLPLSLQTLVKKTVASQCLSTDEHCILKHCGLWWHDVPLKLCMDRGQIQIKSGFLGEDIDLHIALIIAVKENNYSLIKLFTE
WGAHINYSLLSINTEHIRELCRQLGAKETLEDNDIFRIFTKIMHNKTSGRIILCHEIFMNNPNIENKFTIQLRGLICKRLWGL
IEIKETDELNDLLVKYWYAKAVQYECKDAICFLDEKYTDLNEWRLKCLLYYNKIYELHEMYHKEKVQIDVHDMICLASTKDNN
PLTIYYCYALGGNINQAMLTSVQYYNIGNIFFCIDLGGNAFEEGRAIAEQKGYHFLSHSLTLDIYSSDASLPLNLKDPEEISS
LLKDYKSKNLSIIWEYSHNIL*
Warmbaths MGF 360 13L protein
SEQ ID No. 174
MSLPLSLQTLVKKTVASQCLSTDEHCILKHCGLWWHDVPLKLCMDRGQIQIKSGFLGEDIDLHVALIIAVKENNYSLIKLFTE
WGAHINYSLLSINTEHIRELCRQLGAKKTLEDNDIFRIFTKIMHNKTSGRIILCHEIFMNNPNIENKFTIQLRGLICKRLWRL
IEIKETDELNDLLVKYWYAKAVQYECKDAICFLDEKYTGLNEWRLKCLLYYNKIYELHEMYHKDKVQIDVHDMICLASTKDNN
PLTIYYCYALGGNINQAMLTSVQYYNIGNIFFCIDLGGNAFEEGRAIAEQKGYHFLSHSLALDIYSSDASLPLNLKDPEEISS
LLKGYKSKNLSIIWEYSHNIL*
Pretorisuskop/96/4 MGF 360 13L protein
SEQ ID No. 175
MSLPLSLQTLVKKTVASQCLSIDEHCILKHCGLWWHDVPLKLCMDRGQIQIKSGFLGEDIDLHIALIIAVKENNYSLIKLFTE
WGAHINYSLLSINTEHIRELCRQLGAKETLEDDDVFRIFTKIMHNKTSGRIILCHDIFMNNPNIENKFTIQLRGLICKRLWGL
IEIKETDELNDLLVKYWYAKAVQYECKDAICFLEEKYTDLNEWRLKCLLYENKIYELHEMYHKEKVQIDVHDMICLASTKDNN
PLTIYYCYALGGNINQAMLTSIQYYNIGNIFFCIDLGGNAFEEGRAIAEQKGYNFLSHSLALDIFSSDASLPLNLKDPEEISS
FLKDYKSKNLSIIWEYSHNIL*
Mkuzi 1979 MGF 360 13L protein
SEQ ID No. 176
MSLPLSLQTLVKKTVASQCLSIDEHCILKYCGLWWHDAPLKLCMDRGRIQIKSGFLGEDIDLRVALIIAVKENNYSLIKLFTE
WGANINYSLLSINTKHIRELCRQLGAKETLEDNDIFRIFTRIMHNKTSGSIILCHEIFMNNPILENKFVIQLRGLIYKRLWGL
IEIKETDKLNDLLVKYWYAKAVQYVCKNAICFLDEKYTDLNEWRLKCLLYYNKIYELHEMYHKKKVQIDVHDMICLACAKDNN
LLTIYYCYALGSNINQAMLTSVQYYNIGNIFFCIDLGGNAFEEGRAIAEQKGYNFLSHSLTLDIYSSDASLPLNLKDPEEISS
LLKDYKSKNLSIIWEYSHNIL*
L60 MGF 360 13L protein
SEQ ID No. 177
MSLPLSLQTLVKKTVASQCLSIDEHCILKYCGLWWHDAPLKLCMDRGRIQIKSGFLGEDIDLRVALIIAVKENNYSLIKLFTE
WGANINYSLLSINTKHIRELCRQLGAKETLEDNDIFRIFTRIMHNKTSGSIILCHEIFMNNPMLENKFVIQLRGLIYKRLWGL
IEIKETDELNDLLVKYWYAKAVQYVCKNAICFLDEKYTDLNEWRLKCLLYYNKIYELHEMYHKKKVQIDVHDMICLACAKDNN
LLTIYYCYALGGNINQAMLTSVQYYNVGNIFFCIDLGGNAFEEGRAIAEQKGYNFLSHSLTLDIYSSDASLPLNLKDPEKISS
LLKDYKSKNLSIIWEYSHNIL*
Benin 97/1 MGF 360 13L protein
SEQ ID No. 7
MSLPLSLQTLVKKTVASQCLSIDEHCILKYCGLWWHDAPLKLCMDRGRIQIKSGFLGEDIDLRVALIIAVKENNYSLIKLFTE
WGANINYSLLSINTKHIRELCRQLGAKETLEDNDIFRIFTRIMHNKTSGSIILCHEIFMNNPMLENKEVIQLRGLIYKRLWGL
IEIKETDELNDLLVKYWYAKAVQYVCKNAICFLDEKYTDLNEWRLKCLLYYNKIYELHEMYHKKKVQIDVHDMICLACAKDNN
LLTIYYCYALGGNINQAMLTSVQYYNVGNIFFCIDLGGNAFEEGRAIAEQKGYNFLSHSLTLDIYSSDASLPLNLKDPEKISS
LLKDYKSKNLSIIWEYSHNIL*
Malawi Lil-20/1 (1983) MGF 360 13L protein
SEQ ID No. 178
MSAPLSLQTLVKKTVASTSCLSIDEHILKYCDLWWHDAPLKLYMDRGRIQIKSGFLGEDIDLCVALIIAVKENNYNLIKLFTE
LGANINYSLLSINTKHVRDLCRQLGAKETLEDYDIFCIFNKIMHNKTSGSVILCHEIFINNPNLENKFAAQLRRLIYKRLCGL
IEIKETDELSELLVKYWYAKAVQYDYKDAICFLDEKYTDLNEWRLKCYLYYNKIYELHDIYHKEKIQIDVNEMLSLACIRDNN
PLTIYYCYALGGNINQAMLTSVQYYNIGNIYFCIDLGGNAFEEGSAIARQKGYNFLSHSLVLNIYSSDASLPLNLKDPEEISS
LLKNYKSKNLSIILDYSHNIL*
Kenya 1950 MGF 360 13L protein
SEQ ID No. 179
MSSPLSLQTLVKKTVASTSCLSIDEHILKYCGLWWHDAPLKLYIDRGRIYIKSGFLGEDIDLCVALIIAVKENNYSLIKLFTE
WGAYINYSLLSINTKHARDLCRQLGAKETLDDYDIFCIFNKIMHNKTSGSIILCHEIFINNPKLENNFAAQLRRLIYKRLCGL
IEIKETDELSELLVKYWYANAVQYDHKDAICFLDEKYTDLDEWRLKCYLCYNKIYELHDIYHKKKIQIDVNEMLSLACIRDNN
LLTIYYCYALGGNINQAMLTSVQYYNIGNIYFCIDLGGNAFEEGSAIARQNGYNFLCHSLILNIYSSDASLPLNLKVPEEISS
LLKNYKSKNLSIILDYSHKIL*
Ken06.Bus MGF 360 13L protein
SEQ ID No. 180
MSSPLSLQTLVKKTVAGTSCLSIDEHILKYCGLWWHDAPLKLYIDRGRIYIKSGFLGEDIDLCVALIIAVKENNYSMIKLFTE
WGAYINYSLLSINTKHARDLCRQLGAKETLDDYDIFCIFNKIMHNKTSGSIILCHEIFINNPNLENNFAAQLRRLIYKRLCGL
IEIKETDELSELLVKYWYANAVQYDHKDAICFLDEKYTDLDEW*LKCYLCYNKIYELHDIYHKEKIQIDVNEMLSLACIRDNN
LLTIYYCYALGGNINQAMLTSVQYYNIGNIYFCIDLGGNAFEEGSAIARQNGYNFLSHSLVLNIYSSDASLPLNLKDPEEISS
LLKNYKSKNLSIILDYSHKIL*

### MGF 360 14L Protein Sequences

Georgia 2007/1 MGF 360 14L protein
SEQ ID No. 181
MLSLQTLAKKVVACNYLSSDYDYTLQRFGLWWDLGPIHLCNNCKQVFSYKHLQCFSEDDLCLEAALVKAVKSDNLELIRLFVD
WGANPEYGLIRVPAVYLKRLCAELGGLTPVSEPRLLEILKEVARLKSCAGVLLGYDMFCHNPLLETVTRTTLDTVTYTCSNIP
LTGDTAHHLLTKFWFALALRHNFTKAIHYFYKRHKNHLYWRVACSLYENNIFDIHELCREKEICISPNLMMKFACLREKNYAA
IYYCHRLGASLDYGMNLSIYNNNTLNMFFCIDLGAADFDRAQLIAHKAYMYNLSNIFLVKQLFSRDVTLVLDVTEPQEIYDML
KTYTSKNLKRAEEYLTAHPEIIVID*
China/2018/AnhuiXCGQ MGF 360 14L protein
SEQ ID No. 182
MLSLQTLAKKVVACNYLSSDYDYTLQRFGLWWDLGPIHLCNNCKQVFSYKHLQCFSEDDLCLEAALVKAVKSDNLELIRLFVD
WGANPEYGLIRVPAVYLKRLCAELGGLTPVSEPRLLEILKEVARLKSCAGVLLGYDMFCHNPLLETVTRTTLDTVTYTCSNIP
LTGDTAHHLLTKFWFALALRHNFTKAIHYFYKRHKNHLYWRVACSLYFNNIFDIHELCREKEICISPNLMMKFACLREKNYAA
IYYCHRLGASLDYGMNLSIYNNNTLNMFFCIDLGAADFDRAQLIAHKAYMYNLSNIFLVKQLFSRDVTLVLDVTEPQEIYDML
KTYTSKNLKRAEEYLTAHPEIIVID*
Tengani 62 MGF 360 14L protein
SEQ ID No. 183
MLSLQTLAKKVVACNYLSSDYDYTLQRFGLWWDLGPIHLCNNCKQIFSYKHVQCFSEDDLCLEAALVKAVKSDNLELIRLFVD
WGANPEYGLIRVPAVHLKRLCTELGGLTPVSESRLLEILKEVARLKSCAGVLLGYDMFCHNPLLETVTRTTLDTVTYTCSNIP
LTGDTAHHLLTKFWFALALRHNFTKAIHYFYKRHKNHLYWRVACSLYFNNIFDIHELCREKEICISPNLMMKFACLQKKNYAA
IYYCYRLGASLDYGMNLSIYNNNTLNMFFCIDLGATDFDRAQRIAHKAYMYNLSNIFLVKQLFSRDVTLALDVTEPQEIYDML
KSYTSKNLKRAEEYLTAHPEIIVID*
Warthog MGF 360 14L protein
SEQ ID No. 184
MLSLQTLAKKVVACNYLSSDYDYMLQRFGLWWDLGPIHLCNNCKQIFSYKHLQCFSEDDLCLEAALVKAVKSDNLELIRLFVD
WGANPEYGLIRVPAVHLKRLCTELGGLTPVSEPRLLEILKEVAKLKSCAGVLLGYDMFCHNPLLETVTRTTLDTVTYTCSNIP
LTGDTAHHLLTKFWFALALRHNFTKAIHYFYKRHKNHLYWRVACSLYENNIFDIHELCREKEICISPNLMMKFACLRKKNYAA
IYYCYRLGASLDYGMNLSIYNNNTLNMFFCIDLGATDFDRAQRIAHKTYMYNLSNIFLVKQLFSRDVTLALDVTEPQEIYDML
KSYTSKNLKRAEEYLTAHPEIIVID*
Warmbaths MGF 360 14L protein
SEQ ID No. 185
MLSLQTLAKKVVACNYLSSDYDYMLQRFGLWWDLGPIHLCNNCKQIFSYKHLQCFSEDDLCLEAALVKAVKRDNLELIRLFVD
WGANPEYGLICVPTVHLKRLCTELGGLTPVSEPRLLEILKEVANLKSCAGVLLGYDMFCYNPLLETITRTTLDTVMYSCSKIP
LMGDTAHHLLTKFWFALALRHNFTKAIHYFYKRHKNHLYWRVACSLYFNNIFDIHELCREKEICISPNLMMKFACLREKNYAA
IYYCYRLGASLDYGMNLSIYNNNSLNLFFCIDLGATDFDRAQRIAHKAYMYNLSNILLVKQLFSRDVTLALDVTEPQEIYDRL
KAYTSKNMKRAEEYLTAHPEIIVID*
Pretorisuskop/96/4 MGF 360 14L protein
SEQ ID No. 186
MLSLQTLAKKVVACNYLSSDYDYTLQRFGLWWDLGPIHLCNNCKQIFSYKHLQCFSEDDLCLEAALVKAVKSDNLELIRLFVD
WGANPEYGLIRVPAVHLKRLCTELGGLTPVTEPRLLEILKEVAKLKSCAGVLLGYDMFCHNPLLETVTRTTLDTVTYTCSNIP
LTGDTAHLLLTKFWFALALRHNFTKAIHYFYKRHKNHLYWRVACSLYENNIFDIHELCREKEICISPNLMMKFACLQKKNYAA
IYYCYRLGASLDYGMNLSIYNNNTLNMFFCIDLGATDFDRAQHIAHKAYMYNLSNIFLVKQLFSRDVTLALDVTEPQEIYDRL
KSYTSKNLKRAEEYLTAHPEIIVID*
Mkuzi 1979 MGF 360 14L protein
SEQ ID No. 187
MLSLQTLAKKVVACNYLSSDYDYTLQRFGLWWDLGPIHLCNNCKQVFSYKHLQCFSEDDLCLEAALVKAVKSDNLELIRLFVD
WGANPEYGLIRVPAVYLKRLCAELGGLTPVSEPRLLEILKEVANLKSCAGVLLGYDMFCHNPLLETVTRTTLDTVTYTCSNIP
LTGDTAHLLLTKFWFALALRHNFTKAIHYFYKRHKNQLYWRVACSLYFNNIFDIHELCREKEICISPNLMMKFACLREKNYAA
IYYCHRLGASLDYGMNLSIYNNNTLNMFFCIDLGAADFDRAQLIAHKAYMYNLSNIFLVKQLFSRDVTLVLDVTEPQEIYDML
KTYTSKNMKRAEEYLTAHPEIIVID*
L60 MGF 360 14L protein
SEQ ID No. 188
MLSLQTLAKKVVACNYLSSDYDYTLQRFGLWWDLGPIHLCNNCKQVFSYKHLQCFSEDDLCLEAALVKAVKSDNLELIRLFVD
WGANPEYGLIRVPAVYLKRLCAELGGLTPVSEPRLLEILKEVANLKSCAGVLLGYDMFCHNPLLETVTRTTLDTVTYTCSNIP
LTGDTAHLLLTKFWFALALRHNFTKAIHYFYKRHKNQLYWRVACSLYFNNIFDIHELCREKEICISPNLMMKFACLREKNYAA
IYYCHRLGASLDYGMNLSIYNNNTLNMFFCIDLGAADFDRAQLIAHKAYMYNLSNIFLVKQLFSRDVTLVLDVTEPQEIYDML
KTYTSKNMKRAEEYLTAHPEIIVID*
Benin 97/1 MGF 360 14L protein
SEQ ID No. 8
MLSLQTLAKKVVACNYLSSDYDYTLQRFGLWWDLGPIHLCNNCKQVFSYKHLQCFSEDDLCLEAALVKAVKSDNLELIRLFVD
WGANPEYGLIRVPAVYLKRLCAELGGLTPVSEPRLLEILKEVANLKSCAGVLLGYDMFCHNPLLETVTRTTLDTVTYTCSNIP
LTGDTAHLLLTKFWFALALRHNFTKAIHYFYKRHKNQLYWRVACSLYFNNIFDIHELCREKEICISPNLMMKFACLREKNYAA
IYYCHRLGASLDYGMNLSIYNNNTLNMFFCIDLGAADFDRAQLIAHKAYMYNLSNIFLVKQLFSRDVTLVLDVTEPQEIYDML
KTYTSKNMKRAEEYLTAHPEIIVID*
Malawi Lil-20/1 MGF 360 14L protein
SEQ ID No. 189
MLSLQTLAKKVVACNYLSSDYDYTLQRFGLWWDLGPIHLCNTCKQIFSYKHLQCFSEDDLCLEAALVKAVKSDNLELIRLFVD
WGANPEYGLIRVPAVHLKRLCMELGGLTPVSESRLLEILKEVADLKSCAGVLLGYDMFCHNPLLETVTRTTLDTVMYTRSKIP
LTGDTAHLLLSKFWFALALRHNFTKAIHYFYEKHKNQLYWRLTCSLYENNIFDLHELCCKKEICISPNLMMKFACLREENYAA
IYYCHMLGASLDYGMNLSIYNNNTLNLFFCIDLGATNFDRARLIARRVYMYNLSNLFLVKQLFSRDVSLILDLTEPQAIYDML
NTYTSKNLKQAEEYFTAHPEIVVID*
Kenya 1950 MGF 360 14L protein
SEQ ID No. 190
MLSLQTLAKKVVACNYLSSDYDYTLQRFGLWWDLGPIHLCNTCKQIFSYQHLQCFSEDDLCLDAALVKAVKSDNLELIRLFVD
WGANPEYGLIRVPAVDLKRLCTELGGLTPVSESRLLEILKEVADLKSCAGVLLGYDMFCHNPLLETVTRTTLNTVMYTRSKIP
LTGDTAHLLLTKFWFALALRHNFTKAIHYFYEKHKNQLYWRLACSLYFNNIFDLHELCSKKEICISPNLMMKFACLREENYAA
IYYCHMLGASPDYGMNLSIYNNNTLNLFFCIDLGATNFDRARLIAHRVYMYNLSNIFLVKQLFSRDASLVLNITEPQAIYDML
KTYTSKNLKRAEEYFTAHPEIVVID*
Ken06.Bus MGF 360 14L protein
SEQ ID No. 191
MLSLQTLAKKVVACNYLSSDYDYTLQRFGLWWDLGPIHLCNTCKQIFSYQHLQCFSVDDLCLDAALVKAVKSDNLELIRLFVD
WGANPEYGLIRVPAVHLKRLCTELGGLTPVSESRLLEILKEVADLKSCAGVLLGYDMFCHNPLMETVTRTTLNTVMYTRSKIP
LTGDTAHLLLTKFWFALALRHNFTKAIHYFYEKHKNQLYWRLTCSLYFNNIFDLHELCSKKEICISPNLMMKFACLREENYAA
IYYCHMLGASPDYGMNLSIYNNNTLNLFFCIDLGATNFDRARLIAHRVYMYNLSNIFLVKQLFSRDASLVLNITEPQAIYDML
KTYTSKNLKRAEEYFTAHPEIVVID*

### MGF 505 1R Protein Sequences

Georgia 2007/1 MGF 505 1R protein
SEQ ID No. 192
MFSLQNLCRKTLPNRKLPEFFDEYILQLLGLYWENHGTIQRAGNNCVLIQQHTLIPVNEALRTAASEENYEIVSLLLAWEGNL
YYAIIGALEGNRHDLIRKYDDQIKDHHEILPFIDDPVIFHKCHIMRQCFFDCILYQAVKYSKFRVLLYFKHRLEDDLPFTHLL
IEKACKDHNYEVIKWIYENLHIYNMIDTFECAIAHKDLHLYCLGYRFIYNRIVPDKYHHLDIRMLSSLQLLHKVAAKGYLDFI
LETLKYDHNKDNINIILTQAATYNHRKILIYFIPQSTHAQIEQCLLVAIKAKSSRKTLNLLLSHLNLSINLIKKISHYVATYN
STNIIGILSMRRKKKIYLDIILTKFVKKAIFNKFVVRCMDTFSINPERILKIAARINRMMLVKKISEHVWKNHAVRLKYLKHA
VHTMKHKDGKNRLMNFIYDRCYYHMQGEEIFSLARFYAIHHAPKLFDVFYDCCILDTIRFKSLLLDCSHIIGKNAHDATNINI
VNKYIGNLFVMGVLSKKEILQDYPSIYSKQYMP*
China/2018/AnhuiXCGQ MGF 505 1R protein
SEQ ID No. 193
MFSLQNLCRKTLPNRKLPEFFDEYILQLLGLYWENHGTIQRAGNNCVLIQQHTLIPVNEALRTAASEENYEIVSLLLAWEGNL
YYAIIGALEGNRHDLIRKYDDQIKDHHEILPFIDDPVIFHKCHIMRQCFFDCILYQAVKYSKFRVLLYFKHRLEDDLPFTHLL
IEKACKDHNYEVIKWIYENLHIYNMIDTFECAIAHKDLHLYCLGYRFIYNRIVPDKYHHLDIRMLSSLQLLHKVAAKGYLDFI
LETLKYDHNKDNINIILTQAATYNHRKILIYFIPQSTHAQIEQCLLVAIKAKSSRKTLNLLLSHLNLSINLIKKISHYVATYN
STNIIGILSMRRKKKIYLDIILTKFVKKAIFNKFVVRCMDTFSINPERILKIAARINRMMLVKKISEHVWKNHAVRLKYLKHA
VHTMKHKDGKNRLMNFIYDRCYYHMQGEEIFSLARFYAIHHAPKLFDVFYDCCILDTIRFKSLLLDCSHIIGKNAHDATNINI
VNKYIGNLFVMGVLSKKEILQDYPSIYSKQYMP*
Tengani 62 MGF 505 1R protein
SEQ ID No. 194
MFSLQNLCRKTLPDCKLPEFFDEYILQLLGLYWENHGTIQRAGNNCVLIQQHNLIPVNEALRIAASEENYEIVSLLLAWEGNL
YYAIIGALEGNRHDLIRKYDDQIKDHHEILPFIDDPVIFHKCHIMRQCFFDCILYQAVKYSKFRVLLSFKHRLRDDLPFTHLL
IEKACKDHNYEVIKWIYENLHIYNMIDTFECAIAHKDLRLYCLGYRFIYNRIVPDKYHHLDIRMLSSLQLLHKVAAKGYLDFI
LETLKYDHNKDNINIILTQAATYNHRKILIYFIPQSTHAQIEQCLLVAIKTKSSKKTLNLLLSHLNLSINLIKKISHYVATYN
STNIIGILSMRRKKKIYLDIILTKFVKKAIFNKFVVRCMDTFSINPERILKIAARINRTMLVKKISEHVWKNHAVRLKYLKHA
VHTMKHKDGKNRLMNFIYDRCYYHMQGEEIFSLARFYAIHHAPKLFDVFYDCCILDTIRFKSLLLDCSHIIGKNAHDATNINI
VNKYIGNLFVMGVLSKKEILQDYPSIYSKHYMP*
Warmbaths MGF 505 1R protein
SEQ ID No. 195
MFSLQNLCRKTLPDCKLPEFFDEYILQLLGLYWENHGTIQRAGNNCVLIQQRTLIPVNEALRIAASEENYEIVGLLLAWEGNL
YYAIIGALEGNRHDLIRKYDDQIKDHHEILPFIDDPVIFHKCHIIRRCFFNCILYQAVKYSKFRVLLYFKHRLEDDLPLTHLL
IEKACEDHNYEVIKWIYENLHTYDIMDTFECAIAHKDLRLYCLGYTFIYNRIVPYEYHHLDILILSSLQLLHKVAAKGYLDFI
LETLKYDHNNDNLDIILTQAATYNHRKILTYFIPQLTYAQIEQCLFMAIKRKSSKKTLNLLLSHLTLSIELIKKISQYVVTYN
STNIIGILSMKRKKKIYLDIMLTKYVKYAIFNKYVVRCMDRFSINPERIIKMAARINRMMLVKKISEHVWKNHAARLKHLKHA
VHTMKHKDGKNRLMNFIYDRCYYHMQGEEIFSLARFYAIHHAPKLFDVFYDCCILDTIQFKSLLLDCSHIIGKNAHDATNINI
VNKYIGDLFAMGVLSKKEILQDYPSIYSKHYML*
Mkuzi1979 MGF 505 1R protein
SEQ ID No. 196
MFSLQNLCRKTLPDCKLPEFFDDYILQLLGLYWENHGTIQRAGNNCVLIQQHTLIPINEALRTAASEENYEIVSLLLAWEGNL
YYAIIGALEGNRHDLIRKYDDQIKDHHEILPFIDDPVIFHKCHIMRRCFFDCILYQAVKYSKFRVLLYFKYRLEDDLPLTHLL
IEKACENHNYEVIRWIYENLHIYNMIDTFECAIAHKDLRLYCLGYTFIYNRIVPNKYHHIDILILSSLQLLHKVAAKGYLDFI
LETLKYDHNNDNLDIILTQAATYNHRKILTYFIPQSTYAQIEQCLMVAIKTKSSKKTLNLLLSHLNLSIKLIKKISQYVVTYN
STNIIGILSMKRKKKIYLDILLTKFVKNAIFNKFVVRYMDTFSINPEKIVKMAARINKMMLVKKISEHIWKNHAARLEHLKHA
VHTMKHKDGKNRLMNFIYEYCYYHMQGEEIFSLARFYAIHHAPKLFDVFYNCCILDTIRFKSLLLDCSHIIGKNAHDATNINI
VNKYIDNLFAMGVLSKKEILQDYPSIYSKHYMP*
Warthog MGF 505 1R protein
SEQ ID No. 197
MFSLQNLCRKTLPDCKLPEFFDEYILQLLGLYWENHGTIQRAGNNCVLIQQHTLIPVNEALRIAASEENYEIVSLLLAWEGNL
YYAIIGALEGNRHDLIRKYDDQIKDHHEILPFIDDPVIFHKCHIMRRCFFDCILYQAVKYSKFRVLLYFKYRLENDLPLAHLL
IKKACEDHNYEVIKWIYENLHIYNIMDTFGCAIAHKDLRLYRLGYTFIYNRIVPYKYHYLDVLILSGLHLLYKVAAKGYLDFI
LETLKYDHNNDNLDIILTQAATYNHRKILTYYIPQLTYAQIEQCLFMAIKKKSSKKTLNLLLSHLKLSIKLIKKISQYVATYN
STNIIGILNMRRKKKIYLDIILTKFVKKAIFNKFVVRCMDTFSINPERIIKMAARINKMLLVKKISEHAWKNHAARLKHLKHA
VYTMKHKDGKNRLMNLIYDHYYYHMQGEEIFSLARFYAIHHAPKLFDVFYDCCLLDTIRFKNLLLDCSHIIGKNAHDATNITI
VNKYIGNLFAMGVLSKKEILQDYPSIYSKHYMP*
L60 MGF 505 1R protein
SEQ ID No. 198
MFSLQNLCRKTLPDCKLPEFFDDYILQLLGLYWENHGTIQRAGNNCVLIQQHTLIPVNEALRIAASEENYEIVGLLLAWEGNL
YYAIIGALEGNRYNLIRKYDDQIKDHHDILPFIDDPIIFHKCHIMRRCFFDCILYQAVKYSKFRVLLYFKYTLEDDLPLVHLL
IEKACEDHNYEVIKWIYENLHVCHIIDTFDCAIAHKDLRLYCLGYTFIYNRIVPYKYHHLDILILSSLQLLHKVAAKGYLDFI
LETLKYDHNIDNLDVILTQAATYNHRKILTYFIPQSTYAQIEQCLFVAIKTKSSKKTLNLLLSHLNLSIKLIQKISQYVATEN
STNIIGILSMKRKKKIYLDIILTKFVKNAIFNKFVVRCMERFSINPERIVKMAARINKMMLVKKISEHVWKNHAARLKHLKHA
VHTMKHKDGKNRLMNFIYEHCYYHMQGEEIFSLARFYAIHHAPKLFDVFYNCCILDTIRFKSLLLDCSHIIGKNAHDATNINI
VNKYIGNLFAMGVLSKKEILQDYPSIYSKHYMP*
Benin 97/1 MGF 505 1R protein
SEQ ID No. 9
MFSLQNLCRKTLPDCKLPEFFDDYILQLLGLYWENHGTIQRAGNNCVLIQQHTLIPVNEALRIAASEENYEIVGLLLAWEGNL
YYAIIGALEGNRYNLIRKYDDQIKDHHDILPFIDDPIIFHKCHIMRRCFFDCILYQAVKYSKFRVLLYFKYTLEDDLPLVHLL
IEKACEDHNYEVIKWIYENLHVCHIIDTFDCAIAHKDLRLYCLGYTFIYNRIVPYKYHHLDILILSSLQLLHKVAAKGYLDFI
LETLKYDHNIDNLDVILTQAATYNHRKILTYFIPQSTYAQIEQCLFVAIKTKSSKKTLNLLLSHLNLSIKLIQKISQYVATEN
STNIIGILSMKRKKKIYLDIILTKFVKNAIFNKFVVRCMERFSINPERIVKMAARINKMMLVKKISEHVWKNHAARLKHLKHA
VHTMKHKDGKNRLMNFIYEHCYYHMQGEEIFSLARFYAIHHAPKLFDVFYNCCILDTIRFKSLLLDCSHIIGKNAHDATNINI
VNKYIGNLFAMGVLSKKEILQDYPSIYSKHYMP*
Pretorisuskop/96/4 MGF 505 1R protein
SEQ ID No. 199
MFSLQNLCRKTLPDCKLPEFFDEYILQLLGLYWENHGTIQRAGNNCVLVQQHTLIPVNEALRIAASEENYEIVSLLLAWEGNL
YYAIIGALEGNRHDLIRKYDDQIKDHHEILPFIDNPVIFHKCHIMRRCFFDCILYQAVKYSKFRVLLYFKYRLENDLPLAHLL
VEKACEDHNYEVIKWLYENLHIYNIMETFECAIAHKDLRLYRLGYTFIYNRIVPYKYHYLDVLILSGLHLLYKVAAKGYLDFI
LETLKYDHNNDNLDIILTQAVTYNHRKILTYYIPQLTYAQIEQCLFMAIKKKSSKKTLNLLLSHLKLSIKLIKKISQYVATYN
STNIIGILNMKRKKKIYLDIILTKFVKYAIFNKYVVRCMDTFSINPERIIKMAARINKMLLVKKISQHAWKNHAARLKHLKHA
VYTMKHKDGKNRLMNLIYDHYYYHMQGEEIFSLARFYAIHHAPKLFDVFYDCCLLDTIRFKSLLLDCSHIIGKNAHDATNITI
VNKYIGNLFAMGVLSKKEILQDYPSIYSKHYMP*
Kenya 1950 MGF 505 1R protein
SEQ ID No. 200
MFSLQNLCRKTLPDRKLPEFFDDYVLQLLGLYWENHGTIQRAGNNCVLIQQHNLIPVNEALRIAASEENYEIVSLLLAWEGNL
YYAIIGALEGNRHNLIRKYDDQIKDHHEILPFIDDPVIFHKCHMMRRCFFNCILYQAVKYSKFSVLLYFKYILKENLPLVHSL
IEKACEDHNYEVIKWIYENLHIYEIMDTFKCAIAHKDLHLYSLGYTFIYNRIVPYKYHHLDIRILSRLQLLHKVTAKGYLDFI
LETLKYDHNKDNINIILTQAATYNHRNILTYFIPQSTYAQIEQCLFVAIKINASKKTLNLLLSHLNLSIKLVKKLSQYVVAYK
STNIISILSMQQKKKIYLDIILTKVVKNAIFIKFVIGCMVTFSINPERIVKMAARIKKMKLVKNISEHVWKNHAAKLKHLKHA
VHTMKHQEGKNRLMNFIYDHCYYHMQGEEIFSLARFYAIHHAPKLFDVFYDCCILDTIRFKSLLLDCSYIIAKNAHDASINIV
NKYIGNLFAMGVLSKKEILQDYPSIYSKDYML*
Ken06.Bus MGF 505 1R protein
SEQ ID No. 201
MFSLQNLCRKTLPDCKLPEFFDDYVLQLLGLYWENHGTIQRAGNNCVLIQQHNLIPVNEALRIAASEENYEIVSLLLAWEGNL
YYAIIGALEGNHHNLIRKYDDQIKDHHEILPFIDDPVIFHKCHTMRRCFFNCILYQAVKYSKFSVLLYFKYILKENLPLVHSL
IEKAYKYHNYEVIKWIYENLHIYDIINTFKCAIAHKDLRLYCLGYTFVYNRIVPYKYYHLDIRILLRLQLLHKVTAKGYLDFI
LETLKYDHNTNNIDIILTQAATYNHRNILTYFIPQSTYAQIEQCLFVAIKINASKKTLNLLLSHLNLSIKLVKKLSQYVVAYK
STNIISILSRQQKKKIYLDIILTKVVKNAVFNKFVIGCMVTFSINPERIVKMAARIKKMKLVKNISEHVWKNHAVKLKYLKHA
VHTMKHQEGKNRLMNFIYDHCYYHMQEEEIFSLARFYAIHHAPKLFDVFYDCCILDTIRFKSLLLDCSHIIVKNAHDASINIV
NKYIGNLFAMGVLSKKEILQDYPSIYSKDYML*
Ken05/Tk1 MGF 505 1R protein
SEQ ID No. 202
MFSLQNLCRKTLPDCKLPEFFDDYVLQLLGLYWENHGTIQRAGNNCVLIQQHNLIPVNEALRIAASEENYEIVSLLLAWEGNL
YYAIIGALEGNRHNLIRKYDDQIKDHHEILPFIDDPVIFHKCHIMRRCFFNCMLYQAVKYSKFSVLLYFKYILKENLPLVHSL
IEKAYKYHNYEVIKWIYENLHIYDIINTFKYAIAHKDLRLYCLGYTFVYNRIVPYKYYHLDIRILLRLQLLHKVTAKGYLDFI
LETLKYDHNTNNIDIILTQAATYNHRNILTYFIPQSTYAQIEQCLFVAIKINASKKTLNLLLSHLNLSIKLVKKLSQYVVAYK
STNIISILSMQQKKKIYLDIILTKVVKNAVFIKFVIGCMVTFSINPERIVKMAARIKKMKLVKNISEHVWKNHAAKLKHLKHA
VHTMKHQEGKNRLMNFIYDHCYYHMQGEEIFSLARFYAIHHAPKLFDVFYDCCILDTIRFKSLLLDCSHIIAKNAHDASINIV
NKYIGNLFAMGVLSKKEILQDYPSIYSKYDIL*
Malawi Lil-20/1 (1983) MGF 505 1R protein
SEQ ID No. 203
MFSLQNLCRKTLPDCKLPEFFDEYILQLLGLYWENHGTIQRAGNNCVLIQQHNLIPVNEALRIAASEENYEIVSLLLAWEGNL
YYAIIGALEGNRPDLIRKYDDQIKDHHEILPFIDDPIIFHKCHIMRRCFFNCILYQAVKYSKFRVLLYFKHRLGDDLPLTHLL
IEKACEDHNYEVIKWIYENLHSYNIMDTFECAIAHKDLRLYCLGYTFIYNRIVPYKYHHLDICILSSLQLLHKVAAKGYLDFI
LETLKYDHNINNIDIILTQAATYNHRKILTYFIPQLTYAQIEQCLLVAIKTKASKKTLNLLLSHLNLSIKLIKKISQYVVTYN
STNIISILSMRRKKKIYLDIILTEFVKNAIFNKFVVRCMDTFSINPERIVKMAARINRMMLVKNISERVWKNHAVKLKHLKHA
VHTMKHQEGKNRLMNFIYDHCYYHMQGEEIFGLARFYAIHHAPKLFDVFYDCCMLDATRFKSLLLDCPHIIGKNAYDAGINLV
NKYIGNLFAMGVLSKKEILQDYPSIYSKHDMF*

### DP148R

The DP148R gene is located close to the right end of the ASFV genome, at position 177915 to 178679 on the Benin 97/1 genome. The DP148R gene may also be referred to as MGF 360 18R. DP148R is expressed at early times post-infection. The amino acid sequence of the DP148R protein has no significant similarity to other proteins; the secondary structure is predicted to be predominantly helical, but no signal peptide or transmembrane domains are evident.

DP148R inhibits type I interferon. DP148R also inhibits activation of the NF-kB transcription factor (see FIG. 7). DP148R inhibits nuclear translocation of the p65 subunit of NF-kB (see FIG. 8). NF-κB controls expression of interferon and pro-inflammatory cytokines as part of the host's innate immune system response to viral infection. The inhibition of NF-κB by DP147R would results in decreased amounts of type I interferon (IFN) and pro-inflammatory cytokines and chemokines produced by cells infected with ASFV and allow ASFV to circumvent the host innate immune response, favouring virus replication and disrupting the development of adaptive responses.

The gene (i.e. nucleotide) sequences and positions in the genome of DP148R genes from different ASFV strains are presented below.

Georgia 2008/1 DP148R (MH910495.1:183350-184063)
SEQ ID No. 287
ATGTTAGAAATAGTATTGGCAACGCTGCTAGGCGACCTGCAGCGGCTCCGGGTTCTTACCCCTCAGCAGCGGGCAGTTGCCTT
CTTTCGAGCCAATACTAAGGAGCTAGAGGACTTCTTATGCTCAGATGGGCAGTCTGAGGAGGTACTGTCTGGCCCCCTTCTTA
ACCGTCTACTAGAACCCTCAGGCCCTCTTGATATTTTAACCGGATATCACCTATTTCGTCAGAATCCCAAGGCAGGTCAGTTG
CGCGGCCTTGAGGTCAAGATGCTTGAACGGTTATACGATGCTAATATTTACAATATACTGTCTCGGCTGCGGCCTGAAAAAGT
TCGCAACAAGGCTATTGAGCTATACTGGGTTTTCCGAGCTATCCATATTTGTCATGCTCCTTTAGTTTTAGATATTGTACGAT
ATGAGGAACCGGACTTTGCTGAACTGGCCTTTATTTGTGCTGCTTACTTTGGTGAACCTCAGGTAATGTATTTGCTCTACAAA
TATATGCCTCTGACCCGCGCAGTTCTTACGGATGCCATCCGGATAAGTCTTGAGAGCAACAACCAGGTAGGGATTTGCTATGC
TTACTTGATGGGAGGCAGCCTCAAGGGACTAGTCTCCGCCCCACTGCGTAAACGTCTGCGCGCCAAACTACGCTCGCAGCGCA
AAAAGAAGGACGTTCTTTCACCCCACGACTTCTTACTGCTGCTCCAGTAG
China/2018/AnhuiXCGQ DP148R (MK128995.1:183372-184085)
SEQ ID No. 288
ATGTTAGAAATAGTATTGGCAACGCTGCTAGGCGACCTGCAGCGGCTCCGGGTTCTTACCCCTCAGCAGCGGGCAGTTGCCTT
CTTTCGAGCCAATACTAAGGAGCTAGAGGACTTCTTATGCTCAGATGGGCAGTCTGAGGAGGTACTGTCTGGCCCCCTTCTTA
ACCGTCTACTAGAACCCTCAGGCCCTCTTGATATTTTAACCGGATATCACCTATTTCGTCAGAATCCCAAGGCAGGTCAGTTG
CGCGGCCTTGAGGTCAAGATGCTTGAACGGTTATACGATGCTAATATTTACAATATACTGTCTCGGCTGCGGCCTGAAAAAGT
TCGCAACAAGGCTATTGAGCTATACTGGGTTTTCCGAGCTATCCATATTTGTCATGCTCCTTTAGTTTTAGATATTGTACGAT
ATGAGGAACCGGACTTTGCTGAACTGGCCTTTATTTGTGCTGCTTACTTTGGTGAACCTCAGGTAATGTATTTGCTCTACAAA
TATATGCCTCTGACCCGCGCAGTTCTTACGGATGCCATCCGGATAAGTCTTGAGAGCAACAACCAGGTAGGGATTTGCTATGC
TTACTTGATGGGAGGCAGCCTCAAGGGACTAGTCTCCGCCCCACTGCGTAAACGTCTGCGCGCCAAACTACGCTCGCAGCGCA
AAAAGAAGGACGTTCTTTCACCCCACGACTTCTTACTGCTGCTCCAGTAG
OURT 88/3 DP148R (NC_044957.1:168827-169045)
SEQ ID No. 289
ATGCAAAATAAAATCCCAAATTTTAACCTTTTCTTTTTTTTTCTATACAGGATGTTAGAAATAGTATTGGCAACGCTGCTAGG
CGACCTGCAGCGGCTCCGGGTTCTTACCCCTCAGCAGCGGGCGGTTGCCTTCTTTCGAGCCAATACTAAGGAGCTAGAGGACT
TCTTACGCTCAGATGGGCAATCTGAGGAGATACTGTCTGGCCCCCCTCCTTAA
L60 DP148R (NC_044941.1:177973-178737)
SEQ ID No. 290
ATGCAAAATAAAATCCCAAATTTTAACCTTTTCTTTTTTTTTCTATACAGGATGTTAGAAATAGTATTGGCAACGCTGCTAGG
CGACCTGCAGCGGCTCCGGGTTCTTACCCCTCAGCAGCGGGCGGTTGCCTTCTTTCGAGCCAATACTAAGGAGCTAGAGGACT
TCTTACGCTCAGATGGGCAATCTGAGGAGATACTGTCTGGCCCCCTCCTTAACCGTCTACTAGAACCCTCATGCCCTCTTGAT
ATTTTAACCGGATATCACCTATTTCGTCAGAATCCCAAGGCAGGTCAGTTGCGCGGCCTTGAGGTCAAGATGCTTGAACGGTT
ATACGATGCTAATATTTACAATATATTGTCTCGGCTGCGACCTGAAAAAGTCCGCAACAAGGCTATTGAGCTATACTGGGTTT
TCCGAGCTATCCATATTTGTCATGCTCCTTTAGTTTTAGATATTGTACGATATGAGGAACCGGACTTTGCTGAACTGGCCTTT
ATTTGTGCTGCTTACTTTGGTGAACCTCAGGTAATGTATTTGCTCTACAAATATATGCCTCTGACCCGCGCAGTTCTTACGGA
TGCCATCCAGATAAGTCTTGAGAGCAACAACCAGGTAGGGATTTGCTATGCTTACTTGATGGGAGGCAGCCTCAAGGGACTAG
TCTCCGCCCCACTGCGTAAACGTCTGCGCGCCAAACTACGCTCGCAGCGCAAAAAGAAGGACGTTCTTTCACCCCACGACTTC
TTACTGCTGCTCCAGTAG
Benin 97/1 DP148R (NC_044956.1:177915-178679)
SEQ ID No. 291
ATGCAAAATAAAATCCCAAATTTTAACCTTTTCTTTTTTTTTCTATACAGGATGTTAGAAATAGTATTGGCAACGCTGCTAGG
CGACCTGCAGCGGCTCCGGGTTCTTACCCCTCAGCAGCGGGCGGTTGCCTTCTTTCGAGCCAATACTAAGGAGCTAGAGGACT
TCTTACGCTCAGATGGGCAATCTGAGGAGATACTGTCTGGCCCCCTCCTTAACCGTCTACTAGAACCCTCATGCCCTCTTGAT
ATTTTAACCGGATATCACCTATTTCGTCAGAATCCCAAGGCAGGTCAGTTGCGCGGCCTTGAGGTCAAGATGCTTGAACGGTT
ATACGATGCTAATATTTACAATATATTGTCTCGGCTGCGACCTAAAAAAGTCCGCAACAAGGCTATTGAGCTATACTGGGTTT
TCCGAGCTATCCATATTTGTCATGCTCCTTTAGTTTTAGATATTGTACGATATGAGGAACCGGACTTTGCTGAACTGGCCTTT
ATTTGTGCTGCTTACTTTGGTGAACCTCAGGTAATGTATTTGCTCTACAAATATATGCCTCTGACCCGCGCAGTTCTTACGGA
TGCCATCCAGATAAGTCTTGAGAGCAACAACCAGGTAGGGATTTGCTATGCTTACTTGATGGGAGGCAGCCTCAAGGGACTAG
TCTCCGCCCCACTGCGTAAACGTCTGCGCGCCAAACTACGCTCGCAGCGCAAAAAGAAGGACGTTCTTTCACCCCACGACTTC
TTACTGCTGCTCCAGTAG
Warthog DP148R (AY261366.1:180836-181548)
SEQ ID No. 292
ATGTTAGAAATAGTATTGGCAACGCTGCTAGGTGACCTGCAGCGGCTCCGGGTTCTTACCCCTCAGCAGCGGGCGGTTGCCTT
CTTTCGAGCCAATACTAAGGAGCTAGAGGACTTTTTATGCTCAGATGGGCAGTCTGAGGAGATACTGTCCGGCCCCCTCCTTA
ACCGTCTACTAGAACCCTCAGGCCCTCTTGATATTTTAACCGGATATCACTTATTTCGTCAGAATCCCAAGGCAGGTCAGGTG
CGCGGCCTTGAGGTCAAGATGCTTGAACGGTTATACGATGCTAATATTTACAATATATTGTCTCGGCTGCGACCTGAAAAAGT
TCGCAACAAGGCTGTTGAGCTATACTGGGTTTTTCGGGCTATCAATATGTGTCATGCTCCTTTAGTTTTAGATATTGTACGAT
ATGAGGAACCGGACTTTGCTGAACTGGCCTTTATTTGTGCTGCTTACTTTGGTGAACCTCAGGTAATGTATTTGCTCTACAAA
TATATGCCTCTGACCCGCGCAGTTCTTACGGATGCCATCCAGATAAGTCTTGAGAGCAACAGCCAGGTAGGGATTTGCTATGC
CTACTTGATGGGAGGCAGCCTCAAGGGCCTAGTCCGTGCCCCACTACGTAAACGTCTGCGCGCCAAACTACGCTCGCAGCGCA
AAAAGAAGGACGTTCTTCCACCCCATGACTTCTTACTGCTGCTCCAGTAA
Pretorisuskop/96/4 DP148R (AY261363.1:185149-185861)
SEQ ID No. 293
ATGTTAGAAATAGTATTGGCAACGCTGCTAGGTGACCTGCAGCGGCTCCGGGTTCTTACCCCTCAGCAGCGGGCGGTTGCCTT
CTTTCGAGCCAATACTAAGGAGGTAGAAGACTTCTTATGCTCAGATGGGCAGTCTGAGGAGGTACTGTCCGGCCCCCTCCTTA
ACCGTCTACTAGAACCCTCAGGCCCTCTTGATATTTTAACCGGATATCACTTATTTCGTCAGAATCCCAAGGCGGGTCAGGTG
CGCGGCCTTGAGGTCAAGATGCTTGAACGGTTATACGATGCTAATATTTACAATATATTGTCTCGGCTGCGACCTGAAAAAGT
TCGCAACAAGGCTGTTGAGCTATACTGGGTTTTTCGGGCTATCAATATGTGTCATGCTCCTTTAGTTTTAGATATTGTACGAT
ATGAGGAACCGGACTTTGCTGAACTGGCCTTTATTTGTGCTGCTTACTTTGGTGAACCTCAGGTAATGTATTTGCTCTACAAA
TATATGCCTCTGAGCCGCGCAGTTCTTACGGATGCCATCCAGATAAGTCTTGAGAGCAACAGCCAGGTAGGGATTTGCTATGC
CTACTTGATGGGAGGCAGCCTCAAGGGCCTAGTCCGTGCCCCACTACGTAAACGTCTGCGCGCCAAACTACGCTCGCAGCGCA
AAAAGAAGGACGTTCTTCCACCCCACGACTTCTTACTGCTGCTCCAGTAA
Tengani 62 DP148R (AY261364.1:179845-180557)
SEQ ID No. 294
ATGTTAGAAATAGTATTGGCAACGCTGCTAGGTGACCTGCAGCGGCTCCGGGTTCTTACCCCTCAGCAGCGGGCGGTTGCCTT
CTTTCGAGCCAATACTAAAGAGCTAGAGGACTTCTTATGTCCAGATGGGCAGTCTGAGGAGGTGCTGTCTGGCTCCCTCCTTA
ACCGTCTACTAGAACCCTCAGGCCCTCTTGATATTTTAACTGGATATCACCTATTTCGTCAGAATCCCAAGGCAGGTCAGTTG
CGCGGCCTTGAGGTCAAGATGCTTGAACGGTTATACGATGCTAATATTTACAACATATTGTCCCGGCTGCGACCTGAAAAAGT
TCGCAACAAGGCTGTTGAGCTATACTGGGTTTTTCGGGCTATCAATATGTGTCATGCTCCTTTAGTTTTAGATATTGTACGAA
ATGAGGAACTGGACTTTGCTGAACTGGCCTTTATTTGTGCTGCTTACTTTGGTGAGCCTCAGGTAATGTATTTGCTCTACAAA
TATATGCCTCTGACCCGCGCAGTTCTTACGGATGCCATCCAGATAAGTCTTGAGAGCAACAGCCAGGTGGGGATTTGCTATGC
CTACTTGATGGGAGGCAGCCTCAAGGGACTAGTCCGTGCCCCGCTACGTAAACGTCTGCGCGCCAAACTACGCTCGCAGCGCA
AAAAGAAGGATGTTCTTCCACCCCACGACTTCTTACTGCTGCTCCAGTAA
Ken06.Bus DP148R (NC_044946.1:181097-181870)
SEQ ID No. 295
ATGCAAAATAAAATCCCAAATTTTAACCTTTTCTTTTTTTTTCTATACAAGATGTTAGAAATAGTATTGGCAACGCTGCTGGG
TGATCTGCAGCAACTTAAGGATCTTACACCTAAGCAGCGGGCCGTAGCTTTCTTCCGAGCCAACACCAAAGAGCTAGAGGACT
TTCTCTACCCCGATGGGCAGACTGAGGAGTTACTCCCTGGATTTCTTCTTAACCATTTACTAGAACCCTCAGGCCCTATTGAA
ATTTTAACCGGATATCACCTCTTTCGTCAGAATCCAAAGGCAGGTCGCTTGCGAGGCCTTGAGGTGAAAATGCTTGAACGGTT
ATATGATGCTAATATTTACAATATGCTGGCTCGGCTACGGCCTGAGTTGGTTCGTGACAAGGCTGTTGAGCTATATTGGCTTT
TTCGAGCTATTTTAATATGTCATGGTCCCTTGGTTTTGGAGATTGTACGACATGAGACGTTGGACTTTGCAGAAACCGCCTTT
ATCTGTGCTGCTTACTTTAGTGAACCTCAGGTAATGTACGCTCTTTATAACTTTATACCACCCACTCATGCAGTCCTTGCTGA
TGCCATCCAGATGTGTCTTGAGAGCAACAGCGAGGCAGGGATTTGCTATGTCTACCTAATGGGAGGTAACCTCAAGGGCAAGG
TGCCCGGCTCGCTGCGCAAACGTCTGCGTGCCAGTCCACTTCGGCAAGAACGCAAAAAGAAAAACGTCCTTCCGCCCCACGAA
TTCCTACTCCTGCTCCACGGGATTTAA
Kenya 1950 DP148R (AY261360.1:189099-189872)
SEQ ID No. 296
ATGCAAAATAAAATCCCAAATTTTAACCTTTTCTTTTTTTTTCTATACAAGATGTTAGAAATAGTATTGGCCACGCTGCTAGG
TGATCTGCAGAAACTTAAGGATCTTACACCTCCGCAGCGGGCTGTGGCTTTCTTTCGAGCCAACACCAAAGAGCTAGAGGACT
TTCTCTACCCCGATGGGCAGTCTGAGGAGTTACTCCCTGGACTTCTCCTTAACCGTTTACTAGAACCTTCAGGTTCTATTGAC
ATTTTAACCGGTTACCACCTATTTCGTGAGAATCCAAAGGCAGGTCGCTTGCGAGGCCTTGAGGTGAAAATGCTTGAACGGTT
ATATGATGCTAATATTTACAATATGCTGGCTCGGCTACGGCCTGAGTTGGTTCGTGACAAGGCTATTGAGCTATATTGGCTCT
TTCGGGCTATTTTAATGTGTCATAGTCCCTTAGTTTTGGAGATTGTACGACATGAGACAATGGATTTTGCAGAAACCGCCTTT
ATCTGTGCCGCTTACTTTAGTGAACCTCAGGTAATGTACGCTCTTTATAAATTTATACCTATCTCTCGTGCAGTCCTTGCTGA
TGCCATTCAGATGTGTCTTGAGAGCAACAGCGAGGCGGGGATTTGCTATGCTTACCTAATGGGGGGTAGCCTCAAGGGCAAGG
TGCCTGGCTCGCTGCGCAAACGTCTGCGTGCCAGTCCACTTCGGCAAGAGCGCAAAAAGAAAAACGTCCTCCCGCCCCATGAA
TTCCTACTCATGCTCCATGGGATTTAA
Warmbaths DP148R (AY261365.1:184288-185048)
SEQ ID No. 297
ATGCAAAATAAAATCCCAAATTTTAACCTTTTCTTTTTTTTTCTATACAGGATGTTAGAAATAGTATTGGCAACGCTGCTAGG
TGACCTGCAGCGGCTCCGGGTTCTTACCCCTCAGCAGCGGGCGGTTGCCTTCTTTCGAGCCAATACTAAGGAGCTAGAGGACT
TCTTATGCTCAGATGGGCAGTCTGAGGAGATACTGTCCGGCCCCCTCCTTAACCGTCTACTAGAACCCTCAGGCCCTCTTGAT
ATTTTAACCGGATATCACTTATTTCGTCAGAATCCCAAGGCAGGTCAGTTGCGCGGTCTTGAGGTCAAGATGCTTGAACGGTT
ATACGATGCTAATATTTACAATATATTGTCTCGGCTGCGACCTGAAAAAGTTCGCAACAAGGCTATTGAGCTATACTGGGTTT
TCCGAGCTATCCATATTTGTCATGCTCCTTTAGTTTTAGATATTGTACGATATGAGGAACCGGACTTTGCTGAACTGGCCTTT
ATTTGTGCTGCTTACTTTGGTGAACCTCAGGTAATGTATTTGCTCTACAAATATATGCCTCTGACCCGCGCAGTTCTTACGGA
TGCCATCCGGATAAGTCTTGAGAGTAACAACCAGGTAGGGATTTGCTATGCTTACTTGATGGGAGGCAGCCTCAAGGGACTAG
TCTCCGCCCCACTGCGTAAACGTCTGCGCGCCAAACTACGCTCACAGCGCAAAAAGAAAGACGTTCTTTCACCCCACGACTTC
TTACTGCTGCTCCAGTAA
Mkuzi 1979 DP148R (AY261362.1:185751-186511)
SEQ ID No. 298
ATGCAAAATAAAATCCCAAATTTTAACCTTTTCTTTTTTTTTCTATACAGGATGTTAGAAATAGTATTGGCAACGCTGCTAGG
CGACCTGCAGCGGCTCCGGGTTCTTACCCCTCAGCAGCGGGCGGTTGCCTTCTTTCGAGCCAATACTAAGGAGCTAGAGGACT
TCTTATGCTCAGATGGGCAGTCTGAGGAGATACTATCTGGCCCCCTTCTTAACCGTCTACTAGAACCCTCAGGCCCTCTTGAT
ATTTTAACTGGATATCACCTATTTCGTCAGAACCCCAAGGCAGGTCAGTTACGCGGCCTTGAGGTCAAGATGCTTGAACGGTT
ATACGATGCTAATATTTACAATATATTGTCTCGGCTGCGACCTGAAAAAGTTCGCAACAAGGCTATTGAGCTTTACTGGGTTT
TTCGAGCTATCCATATTTGTCATGCTCCTTTAGTTTTAGATATTGTACGATATGAGGAACCGGACTTTGCTGAACTGGCCTTT
ATTTGTGCTGCTTACTTTGGTGAACCTCAGGTAATGTATTTGCTCTACAAATATATGCCTCTGACCCGCGCAGTTCTTACGGA
TGCCATCCGGATAAGTCTTGAGAGTAACAACCAGGTAGGGATTTGCTATGCTTACTTGATGGGAGGCAGCCTCAAGGGACTAG
TCTCCGCCCCACTGCGTAAACGTCTGTGCGCCAAACTACGCTCGCAGCGCAAAAAGAAGGACGTTCTTTCACCCCACGACTTC
TTACTGCTGCTCCAGTAA
Ken05/Tk1 DP148R (NC_044945.1:186074-186847)
SEQ ID No. 299
ATGCAAAATAAAATCCCAAATTTTAACCTTTTCTTTTTTTTTCTATACAAGATGTTAGAAATAGTATTGGCAACGCTGCTAGG
TGATCTGCAGAAACTTAAAGATCTTACACCTATGCAGCGGGCTGTGGCTTTCTTTCGAGCCAACACTAAAGAGCTAGAGGACT
TCCTCTGCCCCGATGGGCAGTCTGAGGAGTTACTCCCTGGACTTCTCCTTAACCGTTTATTAGAACCTTCAGGTCCTATTGAC
ATTTTAACCGGTTACCACCTATTTCGTGAGAATCCGAAGGCAGGTCGGCTGCGTGGCCTTGAGGTCAAGCTGCTTGAACGGTT
ATATGATGCTAACATCTACAATATATTGGCCCAGCTACGGCCTGAGTTGGTTCGCAATAAGGCTGTTGAGTTATATTGGCTCT
TTCGGGCTATTTTGATGTGTCATGGTCCCTTAGTTTTGGAGATTGTACGACATGAGACAATGGATTTTGCAGAATTAGCCTTT
ATCTGTGCTGCTTACTTTAGTGAACCTCAGGTAATGTACGCTATTTATAAATTTATACCTATCTCTTGCGCAGTCCTTGCTGA
TGCCATTCAGATGTGTCTTGAGAGTAACAGCGAGGCGGGGATTTGCTATGCTTACCTAATGGGGGGTAGCCTCAAGGGCAAGG
TGCCTGGCGCGCTGCGCAAACGTCTGCGTGCCAGTCCACTTCGGCAAGAGCGCAAAAAGAAAAACGTTCTTCCACCCCATGAA
TTCCTACTCATGCTTCATGGGATTTAA
Malawi Lil-20/1 (1983) DP148R (AY261361.1:183573-184346)
SEQ ID No. 300
ATGTTAGAAATAGTATTGGCAACGCTGCTAGGTGATCTGCAGAAGCTTAGGGATCTTACACCTATGCAGCGGGCTGTGGCTTT
CTTTCGAGTCAACACTAAAGAGCTAGAGGACTTCCTCTACCCCGATGGGCAGTCTGAGGAGTTGCTCCCTGGACTTCTCCTTA
ACCGTTTACTAGAACCTTCAGGCCCTATTGACATTTTAACCGGTTACCACCTATTTCGTGAGAACCCAAAGGCAGGTCGGCTG
CGTGGCCTTGAGGTTAAGCTGCTTGAACGGTTATATGATGCTAACATCTACAATATGTTGGCCCAAATACGACCTGAGTTGGT
TCGTATCAAGGCTATTGAGTTATATTGGCTCTTTCGGGCTATTTTAATGTGTCATAGTCCCTTAGTTTTGGAGATTGTACGAC
ATGAGACAATGGATTTTGCAGAATTAGCCTTTATCTGTGCTGCTTACTTTAGTGAACCTCAGGTAATGTACGCTCTTTATAAA
TTTATACCTATTTCTCGCGCAGTCCTTGCTGATGCCATCGAGATGTCTCTTGAGAGCAACAGCGAGACGGGGATTTGCTATGC
CTACCTAATGGGGGGTAGCCTCAAGGGCAAGGTGCCCGGCCCGCTGCGCAAACGTCTGCGTGCCAGTCCACTTCGGCAAGAGC
GCAAAAAGAAAAACGTCCTTCCGCCCCATGAATTTCTACTCATGCTCCATGGGATTTAA

The amino acid sequences of DP148R proteins from different ASFV strains is depicted below as SEQ ID Nos 11 to 19 and 301 to 305:

Benin 97/1 DP148R protein
SEQ ID No. 11
MQNKIPNFNLFFFFLYRMLEIVLATLLGDLQRLRVLTPQQRAVAFFRANTKELEDELRSDGQSEEILSGPLLNRLLEPSCPLD
ILTGYHLFRQNPKAGQLRGLEVKMLERLYDANIYNILSRLRPKKVRNKAIELYWVFRAIHICHAPLVLDIVRYEEPDFAELAF
ICAAYFGEPQVMYLLYKYMPLTRAVLTDAIQISLESNNQVGICYAYLMGGSLKGLVSAPLRKRLRAKLRSQRKKKDVLSPHDF
LLLLQ
Warthog DP148R protein: 181103 to 181549
SEQ ID No. 12
MLEIVLATLLGDLQRLRVLTPQQRAVAFFRANTKELEDFLCSDGQSEEILSGPLLNRLLEPSGPLDILTGYHLFRQNPKAGQV
RGLEVKMLERLYDANIYNILSRLRPEKVRNKAVELYWVFRAINMCHAPLVLDIVRYEEPDFAELAFICAAYFGEPQVMYLLYK
YMPLTRAVLTDAIQISLESNSQVGICYAYLMGGSLKGLVRAPLRKRLRAKLRSQRKKKDVLPPHDFLLLLQ
Kenya 1950 DP148R protein: 189417 to 189872
SEQ ID No. 13
MQNKIPNFNLFFFFLYKMLEIVLATLLGDLQKLKDLTPPQRAVAFFRANTKELEDFLYPDGQSEELLPGLLLNRLLEPSGSID
ILTGYHLFRENPKAGRLRGLEVKMLERLYDANIYNMLARLRPELVRDKAIELYWLFRAILMCHSPLVLEIVRHETMDFAETAF
ICAAYFSEPQVMYALYKFIPISRAVLADAIQMCLESNSEAGICYAYLMGGSLKGKVPGSLRKRLRASPLRQERKKKNVLPPHE
FLLMLHGI
Malawi LIL20/1 DP148R protein: 183687 to 184346
SEQ ID No. 14
MLEIVLATLLGDLQKLRDLTPMQRAVAFFRVNTKELEDFLYPDGQSEELLPGLLLNRLLEPSGPIDILTGYHLFRENPKAGRL
RGLEVKLLERLYDANIYNMLAQIRPELVRIKAIELYWLFRAILMCHSPLVLEIVRHETMDFAELAFICAAYFSEPQVMYALYK
FIPISRAVLADAIEMSLESNSETGICYAYLMGGSLKGKVPGPLRKRLRASPLRQERKKKNVLPPHEFLLMLHGI
Mkuzi DP148R protein: 185751 to 186515
SEQ ID No. 15
MQNKIPNFNLFFFFLYRMLEIVLATLLGDLQRLRVLTPQQRAVAFFRANTKELEDFLCSDGQSEEILSGPLLNRLLEPSGPLD
ILTGYHLFRQNPKAGQLRGLEVKMLERLYDANIYNILSRLRPEKVRNKAIELYWVFRAIHICHAPLVLDIVRYEEPDFAELAF
ICAAYFGEPQVMYLLYKYMPLTRAVLTDAIRISLESNNQVGICYAYLMGGSLKGLVSAPLRKRLCAKLRSQRKKKDVLSPHDF
LLLLQ
Pretorisuskop DP148R protein: 185416 to 185862
SEQ ID No. 16
MLEIVLATLLGDLQRLRVLTPQQRAVAFFRANTKEVEDFLCSDGQSEEVLSGPLLNRLLEPSGPLDILTGYHLFRQNPKAGQV
RGLEVKMLERLYDANIYNILSRLRPEKVRNKAVELYWVFRAINMCHAPLVLDIVRYEEPDFAELAFICAAYFGEPQVMYLLYK
YMPLSRAVLTDAIQISLESNSQVGICYAYLMGGSLKGLVRAPLRKRLRAKLRSQRKKKDVLPPHDFLLLLQ
Tengani62 DP148R protein: 180112 to 180558
SEQ ID No. 17
MLEIVLATLLGDLQRLRVLTPQQRAVAFFRANTKELEDFLCPDGQSEEVLSGSLLNRLLEPSGPLDILTGYHLFRQNPKAGQL
RGLEVKMLERLYDANIYNILSRLRPEKVRNKAVELYWVFRAINMCHAPLVLDIVRNEELDFAELAFICAAYFGEPQVMYLLYK
YMPLTRAVLTDAIQISLESNSQVGICYAYLMGGSLKGLVRAPLRKRLRAKLRSQRKKKDVLPPHDFLLLLQ
Warmbaths DP148R protein: 184606 to 185052
SEQ ID No. 18
MQNKIPNFNLFFFFLYRMLEIVLATLLGDLQRLRVLTPQQRAVAFFRANTKELEDFLCSDGQSEEILSGPLLNRLLEPSGPLD
ILTGYHLFRQNPKAGQLRGLEVKMLERLYDANIYNILSRLRPEKVRNKAIELYWVFRAIHICHAPLVLDIVRYEEPDFAELAF
ICAAYFGEPQVMYLLYKYMPLTRAVLTDAIRISLESNNQVGICYAYLMGGSLKGLVSAPLRKRLRAKLRSQRKKKDVLSPHDF
LLLLQ
OURT88/3 DP148R protein: 169146 to 169592
SEQ ID No. 19
MQNKIPNFNLFFFFLYRMLEIVLATLLGDLQRLRVLTPQQRAVAFFRANTKELEDFLRSDGQSEEILSGPPP
China/2018/Anhui DP148R protein
SEQ ID No. 301
MLEIVLATLLGDLQRLRVLTPQQRAVAFFRANTKELEDFLCSDGQSEEVLSGPLLNRLLEPSGPLDILTGYHLFRQNPKAGQL
RGLEVKMLERLYDANIYNILSRLRPEKVRNKAIELYWVFRAIHICHAPLVLDIVRYEEPDFAELAFICAAYFGEPQVMYLLYK
YMPLTRAVLTDAIRISLESNNQVGICYAYLMGGSLKGLVSAPLRKRLRAKLRSQRKKKDVLSPHDFLLLLQ
Georgia2007/1 DP148R protein
SEQ ID No. 302
MLEIVLATLLGDLQRLRVLTPQQRAVAFFRANTKELEDFLCSDGQSEEVLSGPLLNRLLEPSGPLDILTGYHLFRQNPKAGQL
RGLEVKMLERLYDANIYNILSRLRPEKVRNKAIELYWVFRAIHICHAPLVLDIVRYEEPDFAELAFICAAYFGEPQVMYLLYK
YMPLTRAVLTDAIRISLESNNQVGICYAYLMGGSLKGLVSAPLRKRLRAKLRSQRKKKDVLSPHDFLLLLQ
Ken05/Tk1 DP148R protein
SEQ ID No. 303
MQNKIPNFNLFFFFLYKMLEIVLATLLGDLQKLKDLTPMQRAVAFFRANTKELEDFLCPDGQSEELLPGLLLNRLLEPSGPID
ILTGYHLFRENPKAGRLRGLEVKLLERLYDANIYNILAQLRPELVRNKAVELYWLFRAILMCHGPLVLEIVRHETMDFAELAF
ICAAYFSEPQVMYAIYKFIPISCAVLADAIQMCLESNSEAGICYAYLMGGSLKGKVPGALRKRLRASPLRQERKKKNVLPPHE
FLLMLHGI
Ken06 DP148R protein
SEQ ID No. 304
MQNKIPNFNLFFFFLYKMLEIVLATLLGDLQQLKDLTPKQRAVAFFRANTKELEDFLYPDGQTEELLPGFLLNHLLEPSGPIE
ILTGYHLFRQNPKAGRLRGLEVKMLERLYDANIYNMLARLRPELVRDKAVELYWLFRAILICHGPLVLEIVRHETLDFAETAF
ICAAYFSEPQVMYALYNFIPPTHAVLADAIQMCLESNSEAGICYVYLMGGNLKGKVPGSLRKRLRASPLRQERKKKNVLPPHE
FLLLLHGI
L60 DP148R protein
SEQ ID No. 305
MQNKIPNFNLFFFFLYRMLEIVLATLLGDLQRLRVLTPQQRAVAFFRANTKELEDFLRSDGQSEEILSGPLLNRLLEPSCPLD
ILTGYHLFRQNPKAGQLRGLEVKMLERLYDANIYNILSRLRPEKVRNKAIELYWVFRAIHICHAPLVLDIVRYEEPDFAELAF
ICAAYFGEPQVMYLLYKYMPLTRAVLTDAIQISLESNNQVGICYAYLMGGSLKGLVSAPLRKRLRAKLRSQRKKKDVLSPHDF
LLLLQ

### Haemadsorption

Haemadsorption is the phenomenon whereby cells infected with ASFV adsorb erythrocytes (red blood cells) on their surface. The degree of haemadsorption induced by an ASFV may be measured using a haemadsorption (HAD) assay such as described herein (see for example Examples 1 and 3). For example, cells (such as Vero cells or porcine bone marrow cells) may be transfected with a protein or infected with an ASFV, then red blood cells added and the degree of haemadsorption detected by imaging. In this way, different proteins and viruses can be tested for their effect on haemadsorption.

EP402R and EP153R are involved in mediating haemadsorption of ASFV-infected cells.

In an embodiment the invention provides an attenuated ASF virus wherein the ability of the ASF virus to induce haemadsorption is reduced compared to a corresponding ASF virus in which expression and/or activity of the EP153R and EP402R genes is not disrupted. In an embodiment the invention provides an attenuated African Swine Fever (ASF) virus in which the expression and/or activity of the genes EP153R and EP402R is disrupted;

which comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R;
and wherein the ability of the ASF virus to induce haemadsorption is reduced compared to a corresponding ASF virus in which the expression and/or activity of the EP153R gene and/or the EP402R gene is not disrupted.

The invention also provides an attenuated ASF virus in which the expression and/or activity of the genes EP153R and EP402R is disrupted;

which comprises a functional version of one or more of the following genes:
multigene family (MGF) 110 11L and 12L,
MGF 360 6L, 10L, 11L, 12L, 13L, and 14L, and
MGF 505 1R and 2R;
and wherein the ability of the ASF virus to induce haemadsorption is reduced compared to a corresponding ASF virus in which the expression and/or activity of the EP153R gene and/or the EP402R gene is not disrupted.

The invention also provides an attenuated ASF virus in which the expression and/or activity of the genes EP153R and EP402R is disrupted;

which comprises a functional version of one or more of the following genes:
multigene family (MGF) 110 5L, 6L, 8L and 12L,
MGF 360 6L, 10L, 11L, 12L, 13L, 14L and 21R, and
MGF 505 1R and 2R;
and wherein the ability of the ASF virus to induce haemadsorption is reduced compared to a corresponding ASF virus in which the expression and/or activity of the EP153R gene and/or the EP402R gene is not disrupted.

In another aspect the invention provides an ASF virus comprising the EP402R protein of the invention and/or the polynucleotide of the invention wherein the ability of the ASF virus to induce haemadsorption is reduced compared to a corresponding ASF virus which does not comprise the EP402R protein of the invention and/or the polynucleotide of the invention.

In an embodiment of the attenuated ASF virus of the invention, the ability of the EP153R gene and/or the EP402R gene to mediate haemadsorption may be disrupted. In an embodiment, the ability of the EP153R gene to mediate haemadsorption may be disrupted. In an embodiment, the ability of the EP402R gene to mediate haemadsorption may be disrupted.

Reducing haemadsorption or disrupting the ability to mediate haemadsorption means that cells infected with the ASFV of the invention adsorb fewer red blood cells to their surface than cells infected with a wild-type ASFV or with an ASF virus corresponding to, or essentially corresponding to, the ASFV of the invention in which expression and/or activity of the EP153R and EP402R genes has not been disrupted or which does not comprise the EP402R protein of the invention and/or the polynucleotide of the invention. Reducing haemadsorption or disrupting the ability to mediate haemadsorption also means that cells transfected to express a mutant, non-functional EP153R or EP402R protein adsorb fewer red blood cells to their surface than cells transfected with a wild-type EP153R or EP402R protein. The number of red blood cells adsorbed to the surface of the infected/transfected cells may be decreased by at least 50, 60, 70, 80 or 90%. In an embodiment, haemadsorption is abolished i.e. no red blood cells adsorb to the surface of cells infected with the attenuated ASFV of the invention or transfected with a mutant, non-functional EP153R or EP402R protein.

### Gene Expression and Activity

In an embodiment the ASF virus of the present invention has disrupted expression and/or activity of the genes EP153R and EP402R. In another embodiment the ASF virus of the present invention has disrupted expression and/or activity of the genes EP153R, EP402R and K145R. These genes may be referred to herein as the “disrupted genes”.

In an embodiment the invention provides an ASFV in which expression of the genes EP153R and EP402R is disrupted. In an embodiment the invention provides an ASFV in which expression of the genes EP153R, EP402R and K145R is disrupted. Suitably expression of the EP153R gene is disrupted. Suitably expression of the EP402R gene is disrupted. Suitably expression of the K145R gene is disrupted. In an embodiment the invention provides an ASFV in which activity of the genes EP153R and EP402R is disrupted.

The term “expression” with respect to a gene refers to the ability of the ASF virus to produce the product of the gene, such as RNA and/or protein. Disruption of expression of a gene means that production of the gene product is decreased. Expression of the gene may be decreased by at least 50%, at least 60%, at least 70%, at least 80%, at least 90% and/or at least 95%. Expression of the gene may be decreased to the extent that production of the gene product, such as RNA and/or protein, is entirely abolished (i.e. the gene product is not produced at all). Disruption of gene expression decreases expression of the gene relative to the expression of the gene when it is not disrupted. For example, a mutated gene may have decreased expression in comparison to a wild-type version of the gene.

A gene the expression of which is disrupted may not be fully transcribed and translated. Transcription of the gene may be decreased by at least 50%, at least 60%, at least 70%, at least 80%, at least 90% and/or at least 95%. Transcription of the gene may be abolished (i.e. the gene may not be transcribed). Translation of the gene may be decreased by at least 50%, at least 60%, at least 70%, at least 80%, at least 90% and/or at least 95%. Translation of the gene may be abolished. The gene may be transcribed but not translated. The gene may be transcribed and translated but the protein too rapidly degraded to carry out its function. The gene may be transcribed and translated but the protein may be non-functional.

Gene expression may be measured by techniques known in the art. For example, the amount of mRNA transcribed from a gene may be quantified, such as by using quantitative polymerase chain reaction (qPCR). Alternatively or additionally, the amount of protein may be quantified, such as by using Western blotting or mass spectrometry.

The term “activity” with respect to a gene refers to the ability of the gene to carry out its functions. Different genes have different activities i.e. different functions they fulfil. A given gene may have multiple activities; disruption of gene activity means disruption of one or more of those activities. One or more activities of the gene may be disrupted whilst one or more other activities are not disrupted. Disruption of gene activity decreases the activity of the gene relative to the activity of the gene when it is not disrupted. For example, a mutated gene may have decreased activity in comparison to a wild-type version of the gene. Gene activity may be decreased to the extent that gene activity is entirely abolished.

The ASFV according to the present invention may comprise a non-functional version of the disrupted genes.

Disruption of expression of a gene may also disrupt activity of that gene as the decreased amount of gene product means the gene cannot as effectively carry out one or more of its activities.

In an embodiment the attenuated ASF virus of the invention comprises mutations that disrupt the expression and/or activity of the genes EP153R and EP402R.

Gene expression and/or activity may be disrupted by disrupting transcription of the gene into mRNA i.e. by decreasing gene transcription, such as completely abolishing gene transcription. Gene expression and/or activity may be disrupted by disrupting translation of mRNA into protein. In an embodiment the attenuated ASF virus comprises mutations that decrease transcription and/or translation of the genes. In an embodiment the attenuated ASF virus comprises mutations that cause the genes to not be transcribed and/or translated (i.e. complete abolition of transcription and/or translation).

Gene expression and/or activity may be disrupted by mutating a non-coding sequence associated with the gene, such as a promoter. In an embodiment the attenuated ASF virus comprises mutations in promoters of one or more of the disrupted genes.

Gene expression and/or activity may be disrupted by mutating a coding sequence of one or more of the disrupted genes.

### Functional Version of a Gene

The attenuated ASFV of the invention comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

In some embodiments the attenuated ASFV of the invention may comprise a functional version of one or more of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. Suitably, the attenuated ASFV comprises functional versions of two or more, such as three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one, or twenty two of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. In an embodiment the attenuated ASFV comprises functional versions of all of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. Suitably the attenuated ASF virus of the invention comprises functional versions of all ASF virus genes other than EP153R and EP402R. Suitably the attenuated ASF virus of the invention comprises functional versions of all ASF virus genes other than EP153R, EP402R and K145R.

The expression “functional version” of a gene refers to a gene the expression and activity of which have not been disrupted. A functional version of a gene may not be mutated in a manner that disrupts gene expression or gene activity. A functional version of a gene may not comprise any mutations. The coding sequence of a functional version of a gene may be complete and uninterrupted. A functional version of a gene may be fully transcribed and translated. A functional version of a gene may comprise the full coding sequence.

A functional version of a gene may correspond to the gene in a wild-type ASFV isolate. A functional version of a gene may correspond to the gene in a virulent ASFV strain. The sequence of a functional version of a gene may be identical to the sequence of the gene in a wild-type ASFV isolate or virulent ASFV strain. The sequence of a functional version of a gene may be identical to the sequence of the gene in the wild-type ASFV isolate from which the attenuated ASFV of the invention is derived. A functional version of a gene may be a natural variant of the gene in a wild-type ASFV isolate.

A functional version of a gene may comprise mutations. However, the mutations should not disrupt the expression or activity of the gene. In other words, the mutations should not affect the function of the gene. A functional version of a gene may comprise one or more synonymous mutations (i.e. mutations which do not alter the amino acid sequence of the protein the gene encodes). A functional version of a gene may comprise one or more silent mutations, which may be synonymous or non-synonymous. A functional version of a gene may comprise deletions that do not disrupt the expression or activity of the gene. A functional version of a gene may comprise one or more single nucleotide polymorphisms (SNPs) that do not disrupt the expression or activity of the gene.

### Mutations

Gene expression and/or activity are disrupted by mutating the ASFV genome i.e. by changing the nucleotide sequence of the ASFV genome. A “mutation” means a change in the nucleotide sequence of the ASFV genome relative to a known ASFV genotype. Mutations include changing one or more nucleotides to different nucleotides (i.e. substitution), adding nucleotides (i.e. insertion), removing nucleotides (i.e. deletion) and/or a combination of these. In an embodiment the ASF virus of the invention comprises one or more mutations that disrupt the expression and/or activity of the genes EP153R and EP402R. In an embodiment the ASF virus of the invention comprises one or more mutations that disrupt the expression of the K145R gene.

Mutations that disrupt gene expression and/or activity may be in non-coding sequence of the ASFV genome and/or in coding sequence of the ASFV genome. The attenuated ASF virus of the invention may comprise one or more mutations in a non-coding region that disrupt the expression and/or activity of the EP153R gene and/or one or more mutations in a non-coding region that disrupt the expression and/or activity of the EP402R gene. The ASF virus of the invention may comprise one or more mutations in a non-coding region that disrupt the expression and/or activity of the K145R gene.

The ASF virus of the invention may comprise one or more mutations in a coding region of the EP153R gene that disrupt the expression and/or activity of the EP153R gene and/or one or more mutations in a coding region of the EP402R gene that disrupt the expression and/or activity of the EP402R gene. The attenuated ASF virus of the invention may comprise one or more mutations in a coding region of the K145R gene that disrupt the expression of the K145R gene.

### Deletions

In the ASFV of the invention, expression and/or activity of genes may be disrupted by deletion. In other words, expression and/or activity of a gene may be disrupted by a mutation that is a deletion. An ASFV of the invention may be made to lack a functional version of a gene by deletion. In other words, the mutation that causes the ASFV to lack a functional version of a gene may be a deletion.

“Deletion” means removal of part of the ASFV genome nucleotide sequence. The deletion may be continuous, or may comprise deletion of a plurality of sections of sequence. Deletion may disrupt gene expression and/or activity in any of the ways described herein. Deletion may cause the ASFV to lack a functional version of the gene in any of the ways described herein.

Deletion may alter the gene product that is produced. Deletion may cause the gene to not be transcribed and/or translated. Deletion may disrupt transcription of the gene into mRNA. For example, deleting a promoter of a gene would disrupt transcription. Deletion may disrupt translation of mRNA into protein. For example, deleting a start codon would disrupt translation.

Gene expression and/or activity may be disrupted by deleting non-coding sequence associated with the gene, such as a promoter.

Gene expression and/or activity may be disrupted by deleting coding sequence of the gene. The ASFV may be made to lack a functional version of the gene by deleting coding sequence of the gene. The expression “deletion of a gene” (such as “partially deleted” or “completely deleted”) refers to deletion of a sufficient amount of coding sequence such that expression and/or activity of the gene is disrupted.

Deletion of coding sequence may be partial (i.e. part of the coding sequence is deleted). The deletion may, for example, remove at least 50, 60, 70, 80 or 90% of the coding sequence of the gene. The amount of coding sequence required to be deleted to disrupt gene expression and/or activity may be very small. For example, partial deletion of a gene may mean deletion of just the start codon (ATG) if this is sufficient to disrupt expression and/or activity of the gene. At the other extreme, the deletion may be complete, in which case 100% of the coding sequence of the gene is deleted (i.e. all of the coding sequence is absent when compared to the corresponding genome of a wild-type isolate). In other words, “completely deleted” means that all of the coding sequence of that gene has been deleted.

Partial and full deletions of a gene can be made using known techniques in the art, such as conditional targeting via Cre-LoxP and Flp-FRT systems, or by inducing a double strand break (DSB) and repair using engineered nucleases such as meganucleases, zinc finger nucleases (ZFNs), transcription-activator like effector nucleases (TALENs) and Cas in CRISPR-Cas systems. The DSB repair can be exploited to introduce a desired mutation by providing a vector comprising the desired mutated nucleotide sequence within a sequence that is homologous to the sequences flanking either side of the DSB. This results in the desired mutation being inserted at the site of the DSB. Nucleases such as those above can be engineered to induce DSB at a specific site within the genome. For example, chimeric meganucleases can be readily generated by combining known protein units to recognise a target recognition sequence within a gene or genomic region of interest. ZFNs can also be designed to target specific sequences, for example combining zinc-finger units with known specificities to bind specific regions of DNA. TALENs are artificial restriction enzymes designed by fusing a nuclease domain to DNA-binding TALE (transcription activator-like effector) domains. TALE domains are tandem arrays of amino acid repeats that recognise a single nucleotide and can be designed to target a specific region of DNA. CRISPR-Cas systems consist of a Cas (CRISPR-associated protein) nuclease and a CRISPR (clustered regularly interspaced short palindromic repeat) RNA sequence that guides the Cas protein to recognise and cleave a specific strand of DNA complementary to the CRISPR sequence. Single-stranded guide RNA (sgRNA) can therefore be designed to bind to a specific region of DNA and guide the Cas to introduce a DSB. Accordingly, provided the nucleotide (e.g. DNA or cDNA) sequence of a gene is known, a known nuclease system can be utilised to introduce a partial or full deletion to the gene.

Deletion of coding sequence may be continuous, or may comprise deletion of a plurality of sections of coding sequence. The deletion should remove a sufficient amount of coding sequence such that deletion disrupts the expression and/or activity of the gene i.e. a functional gene product, such as a protein, is no longer produced from the gene.

### Interruptions

The expression and/or activity of the genes may be disrupted by interruption of the gene. In other words, the mutation that disrupts expression and/or activity of a gene may be one that interrupts the gene.

In an embodiment of the ASFV of the present invention the genes EP153R, EP402R and K145R may each be interrupted. The EP153R gene may be interrupted. The EP402R gene may be interrupted. The K145R gene may be interrupted.

“Interruption” means the mutation alters the coding sequence of the gene such that a functional gene product, such as a protein, is no longer produced. The term “interruption” may be used herein to refer to a mutation that interrupts a gene. The mutation(s) should interrupt the coding sequence in a manner such that expression and/or activity of the gene is disrupted i.e. a functional gene product, such as a protein, is no longer produced from the gene.

Interruptions may encompass deletions (i.e. removal of one or more nucleotides) within the coding sequence of a gene, but also substitutions (i.e. replacement of one or more nucleotides with different nucleotides) and insertions (i.e. addition of one or more nucleotides) within the coding sequence of a gene.

The interruption may entirely abolish gene product production. For example, where the gene product is a protein, the interruption may render the mRNA nonsensical, causing the mRNA to be degraded and the protein to not be translated, thereby abolishing protein production. The interruption may alter the gene product that is produced. The interruption may cause the gene to not be transcribed and/or translated.

The interruption may be a point mutation (i.e. substitution, insertion or deletion of a single nucleotide). An interruption may be an insertion of one or more nucleotides. An interruption may be a deletion. A gene may comprise multiple mutations that lead to interruption of the gene.

The interruption may be a frame shift mutation, caused by insertion or deletion of nucleotides. A frame shift causes the codons downstream of the frame shift to be read as different amino acids. The protein produced may be non-functional.

The interruption may be mutation of a start codon. A start codon is typically ATG. Mutation of a start codon (e.g. point mutation of one, two or three of the nucleotides) means that translation will not start at that codon. Translation may begin at a subsequent start codon further downstream. If the subsequent start codon is in frame a version of the protein is produced that is N-terminally truncated and so may be non-functional. If the subsequent start codon is not in frame an entirely different or nonsense protein is produced, which would be non-functional. If there is no subsequent start codon, translation is entirely abolished and no protein is produced.

The interruption may be mutation of a stop codon (TAG, TAA or TGA). Mutation of a stop codon (also referred to as a nonstop mutation) causes continued translation of mRNA into a sequence that should not be translated. The resulting protein may be non-functional due to its excessive length.

### Amino Acid Changes

In embodiments of the ASF virus of the invention, the EP402R gene may comprise one or more mutations that change one or more amino acids in the ligand-binding domain of the EP402R protein. The amino acid changes in EP402R are described in detail elsewhere herein.

### Mutation Combinations in ASF Virus

The mutations that disrupt gene expression and/or activity described herein may be combined in an ASFV of the invention. In other words, the EP153R and EP402R genes in an ASFV of the invention may each be disrupted by the same type of mutation as any of the other genes or by a different type of mutation as any of the other genes. Furthermore, the K145R gene may be disrupted by the same type of mutation as any of the other genes or by a different type of mutation as any of the other genes.

For example, in an ASFV of the invention, EP153R may be disrupted by complete deletion, EP402R may be disrupted by an amino acid change in its ligand-binding domain and K145R may be disrupted by mutation of a promoter sequence. As an alternative example, EP153R may be disrupted by interruption and EP402R may be disrupted by complete deletion and K145R may be disrupted by partial deletion.

In an embodiment the invention provides an ASFV in which
the EP153R gene is completely deleted, and
the EP402R gene comprises a mutation that changes an amino acid at a position in
the EP402R protein which corresponds to Q96 of Georgia 2007/1 EP402R protein (SEQ ID No. 24) to R.

In an embodiment the invention provides an ASFV in which
the EP153R gene and the K145R gene are each completely deleted, and
the EP402R gene comprises a mutation that changes an amino acid at a position in
the EP402R protein which corresponds to Q96 of Georgia 2007/1 EP402R protein (SEQ ID No. 24) to R.

In an embodiment the invention provides an ASFV in which
the EP153R gene is completely deleted, and
the EP402R gene comprises a mutation that changes an amino acid at a position in
the EP402R protein equivalent to W99 of Georgia 2007/1 EP402R protein (SEQ ID No. 24) to D.

In an embodiment the invention provides an ASFV in which
the EP153R gene and the K145R gene are each completely deleted, and
the EP402R gene comprises a mutation that changes an amino acid at a position in
the EP402R protein equivalent to W99 of Georgia 2007/1 EP402R protein (SEQ ID No. 24) to D.

In an embodiment the invention provides an ASFV in which the EP153R gene and the K145R gene are each completely deleted, and which comprises an EP402R protein comprising the sequence of SEQ ID No. 33.

In an embodiment the invention provides an ASFV in which the EP153R gene, the EP402R gene and the K145R gene are each completely deleted.

In an embodiment the invention provides an ASFV wherein the ASFV genome is the same as that of the Georgia 2007/1 strain, except that
the EP153R gene and the K145R gene are each completely deleted, and
the EP402R gene comprises a mutation that changes Q96 of the EP402R protein to R.

In an embodiment the invention provides an ASFV in which the EP153R gene and the K145R gene are each completely deleted, and which comprises an EP402R protein comprising the sequence of SEQ ID No. 33, wherein the genome of the ASFV corresponds to that of the Georgia 2007/1 strain.

### Medical Use, Vaccine and Pharmaceutical Composition

In present invention provides an ASF virus of the invention for use in treating and/or preventing a disease in a subject. The invention also provides use of an ASF virus of the invention for manufacture of a medicament for treating and/or preventing disease in a subject. Suitably the disease is African Swine Fever.

The present invention also provides a vaccine comprising an attenuated ASF virus of the invention.

The term “vaccine” as used herein refers to a preparation which, when administered to a subject, induces or stimulates a protective immune response. In some embodiments the vaccine of the invention induces a partially protective immune response. In some embodiments the vaccine reduces severity and/or duration of ASF symptoms but does not completely abolish ASF symptoms. A vaccine can render an organism immune to a particular disease, in the present case ASF. The vaccine of the present invention thus induces an immune response in a subject which is protective against subsequent ASF virus challenge. A vaccine comprising an attenuated ASFV of the invention may be capable of inducing a cross-protective immune response against a plurality of ASF virus genotypes. In an embodiment a vaccine comprising an attenuated ASFV of the invention of a single genotype may be capable of inducing a cross-protective immune response against a plurality of ASF virus genotypes.

The vaccine may comprise a plurality of attenuated ASF viruses. The plurality of attenuated ASF viruses may correspond to a plurality of different isolates, for example, different isolates of high or unknown virulence. Such a vaccine may be capable of inducing a cross-protective immune response against a plurality of ASF virus genotypes.

The vaccine may be useful in preventing African Swine Fever. Accordingly, the invention provides a vaccine of the invention for use in treating and/or preventing African Swine Fever in a subject.

The present invention also provides a pharmaceutical composition which comprises one or more attenuated ASF virus(es) of the invention. The pharmaceutical composition may be used for treating African Swine Fever.

The vaccine or pharmaceutical composition may comprise one or more attenuated ASF virus(es) of the invention and optionally one or more adjuvants, excipients, carriers and diluents. The choice of pharmaceutical excipient, carrier or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents. The pharmaceutical compositions typically should be sterile and stable under the conditions of manufacture and storage. Formulations for parenteral administration include, but are not limited to, suspensions, solutions, emulsions in oily or aqueous vehicles, pastes, and implantable sustained-release or biodegradable formulations. Sterile injectable formulations may be prepared using a non-toxic parenterally acceptable diluent or solvent. A pharmaceutical composition of the present invention may include pharmaceutically acceptable dispersing agents, wetting agents, suspending agents, isotonic agents, coatings, antibacterial and antifungal agents, carriers, excipients, salts, or stabilizers which are nontoxic to the subjects at the dosages and concentrations employed. Preferably, such a composition can further comprise a pharmaceutically acceptable carrier or excipient for use in the treatment of disease that that is compatible with a given method and/or site of administration, for instance for parenteral (e.g. sub-cutaneous, intradermal, or intravenous injection) or intrathecal administration.

The vaccine or pharmaceutical composition may comprise one or more attenuated ASF virus(es) of the invention in an effective amount.

In an embodiment the invention provides an attenuated ASF virus of the invention which when administered to a subject induces an immune response which is protective against subsequent challenge with virulent ASF virus. In an embodiment the invention provides an attenuated ASF virus of the invention which when administered to a subject induces an immune response which is protective against subsequent challenge with virulent ASF virus of a different genotype to the attenuated ASF virus of the vaccine. In an embodiment the invention provides an attenuated ASF virus of the invention which when administered to a subject induces an immune response which is protective against subsequent challenge with virulent ASF virus of any genotype. Accordingly, the invention provides a vaccine comprising an attenuated ASF virus for use in treating and/or preventing African Swine Fever wherein the African Swine Fever is caused by an ASF virus of a different genotype to the ASF virus of the vaccine. In an embodiment the invention provides a vaccine comprising an attenuated ASF virus for use in treating and/or preventing African Swine Fever wherein the African Swine Fever is caused by an ASF virus of any genotype. In an embodiment the ASF virus of the vaccine corresponds to genotype II, such as Georgia 2007/1 strain and the vaccine is protective against infection with genotype I, II, III, IV, V, VI, VII, VIII, IX, X and/or XIV. In an embodiment the ASF virus of the vaccine corresponds to genotype II, such as Georgia 2007/1 strain and the vaccine is protective against infection with genotype I, IX, X, XIV, and/or VIII. In an embodiment the ASF virus of the vaccine corresponds to genotype II, such as Georgia 2007/1 strain and the vaccine is protective against infection with genotype I, IX, and/or X.

### Methods of Prevention/Treatment

The present invention also provides a method of preventing and/or treating ASF in a subject by administration to the subject of an effective amount of an attenuated virus, vaccine, or pharmaceutical composition of the invention.

The term “preventing” is intended to refer to averting, delaying, impeding or hindering the contraction of ASF. The vaccine may, for example, prevent or reduce the likelihood of an infectious ASFV entering a cell. The vaccine may reduce the severity and/or duration of ASF symptoms. The vaccine may completely abolish ASF symptoms.

The term “treating” is intended to refer to reducing or alleviating at least one symptom of an existing ASF infection.

The subject may be any animal which is susceptible to ASF infection. ASF susceptible animals include domestic pigs, warthogs, bush pigs and ticks.

The subject vaccinated according to the present invention may be a domestic pig.

Suitably, protective immunity as defined herein may be conferred to piglets who are fed colostrum from a vaccinated subject, such as a vaccinated mother.

### Administration

The vaccine of the invention may be administered by any convenient route, such as by intramuscular injection. Other suitable routes of administration include intranasal, oral, subcutaneous, transdermal and vaginal (e.g. during artificial insemination). In one embodiment, oral administration comprises adding the vaccine to animal feed or drinking water. In another embodiment, the vaccine may be added to bait for a wild animal, for example bait suitable for wild boar, wild pigs, bushpigs or warthogs.

The dose for pig immunisation may be from about 10³ to about 10⁶ HAD₅₀ or TCID₅₀ per pig. The dose for pig immunisation may be from about 10³ to about 10₆ TCID₅₀ per pig. The dose for pig immunisation may be less than 10⁴ HAD₅₀ or TCID₅₀ per pig. For example the dose may be between 10²-10³ HAD₅₀ or TCID₅₀. The dose may be about 10² HAD₅₀ or TCID₅₀ per pig. The dose may be determined by a veterinary practitioner within the scope of sound veterinary judgment.

The vaccine may be administered following a prime-boost regime. For example, after the first inoculation, the subjects may receive a second boosting administration some time (such as about 7, 14, 21 or 28 days) later. Typically the boosting administration is at a higher dose than the priming administration. The boosting dose may be from about 10³ to about 10⁶ HAD₅₀ or TCID₅₀ per pig. The boosting dose may be from about 10³ to about 10⁶ TCID₅₀ per pig.

### Method for Preparing a Virus

The present invention also provides a method of producing an ASF virus of the invention, the method comprising changing one or more amino acid in the ligand-binding domain of the EP402R protein wherein the amino acid change disrupts ligand-binding of the EP402R protein.

The present invention also provides a method of reducing the ability of an ASF virus to induce haemadsorption, the method comprising changing one or more amino acid changes in the ligand-binding domain of the EP402R protein wherein the amino acid changes disrupt ligand-binding of the EP402R protein.

The amino acid changes in the ligand-binding domain of EP402R may be any of the amino acid changes described herein. Such amino acid changes may be made by mutating the ASFV genome as described herein.

In an embodiment, the method comprises changing one or more amino acid in the EP402R protein at a position which corresponds to Q96 and/or W99 of the Georgia 2007/1 EP402R protein (SEQ ID No. 24).

The invention also provides a method of attenuating an ASF virus which comprises disrupting the expression and/or activity of the EP153R and EP402R genes. Suitably the method comprises disrupting the ability of the EP153R gene and/or the EP402R gene to mediate haemadsorption.

Disruption of gene expression and/or activity may be achieved by mutating the ASFV genome in any of the ways described herein.

In an embodiment the method further comprises introducing a DIVA mutation into the ASF virus. Suitably the DIVA mutation disrupts expression of the K145R gene. Suitably the K145R is at least partially deleted, preferably completely deleted. Suitably the K145R gene is interrupted. Suitably the DIVA mutation disrupts expression of the B125R gene. Suitably the B125R is at least partially deleted, preferably completely deleted. Suitably the B125R gene is interrupted.

In an embodiment of the method of producing and/or attenuating an ASFV, the EP153R gene is at least partially deleted, preferably completely deleted. In an embodiment the EP153R gene is interrupted.

In an embodiment of the method of producing and/or attenuating an ASFV, the EP402R gene is at least partially deleted, preferably completely deleted. In an embodiment the EP402R gene is interrupted. Suitably the method comprises introducing one or more mutations in the EP402R gene that reduce surface expression of the EP402R protein compared to a corresponding ASF virus that does not comprise the one or more mutations. Suitably the method comprises introducing one or more mutations in the EP402R gene that disrupt ligand binding by the EP402R protein. Suitably the method comprises introducing one or more mutations in the EP402R gene that change one or more amino acids in the ligand-binding domain of the EP402R protein. Suitably the one or more amino acids are changed to different amino acids. Suitably, changing the amino acids to different amino acids directly inhibits the interaction between EP402R and its ligand by changing the binding surface on EP402R.

In an embodiment the method comprises changing an amino acid in the EP402R protein at a position which corresponds to Q96 and/or W99 of the Georgia 2007/1 EP402R protein (SEQ ID No. 24). Suitably the amino acid in the EP402R protein at a position which corresponds to Q96 of the Georgia 2007/1 EP402R protein (SEQ ID No. 24) is changed to R or to an amino acid that is a conservative replacement of R and/or an amino acid at a position which corresponds to W99 of the Georgia 2007/1 EP402R protein (SEQ ID No. 24) is changed to D or to an amino acid that is a conservative replacement of D. Suitably the amino acid at the position which corresponds to Q96 is changed to H, K or R and/or the amino acid at the position which corresponds to W99 is changed to D, E, N or Q. Suitably the amino acid at the position which corresponds to Q96 is changed to R and/or the amino acid at the position which corresponds to W99 is changed to D.

In an embodiment the invention provides a method of attenuating an ASF virus comprising completely deleting each of the EP153R and K145R genes, changing the amino acid at the position which corresponds to Q96 of the Georgia 2007/1 EP402R protein (SEQ ID No. 24) to R and/or changing the amino acid at the position which corresponds to W99 of the Georgia 2007/1 EP402R protein (SEQ ID No. 24) to D. In an embodiment the invention provides a method of attenuating an ASF virus comprising completely deleting each of the EP153R and K145R genes, and changing the amino acid at the position which corresponds to Q96 of the Georgia 2007/1 EP402R protein (SEQ ID No. 24) to R.

In an embodiment the method of producing and/or attenuating an ASF virus of the invention may be applied to an ASF virus of any genotype (i.e. an ASF virus of any of genotypes I to XXIV). In other words, an ASF virus of any genotype may be the subject of the modifications of the method of the invention. An ASF virus of any genotype may used in the method. In an embodiment the method of producing and/or attenuating an ASF virus of the invention may be applied to an ASF virus of genotype II. In an embodiment the method of producing and/or attenuating an ASF virus of the invention may be applied to an ASF virus of the Georgia 2007/1 strain.

In an embodiment the invention provides a method of attenuating an ASF virus of the Georgia 2007/1 strain comprising completely deleting each of the EP153R and K145R genes, changing Q96 of the Georgia 2007/1 EP402R protein (SEQ ID No. 24) to R and/or changing W99 of the Georgia 2007/1 EP402R protein (SEQ ID No. 24) to D. In an embodiment the invention provides a method of attenuating an ASF virus of the Georgia 2007/1 strain comprising completely deleting each of the EP153R and K145R genes, and changing Q96 of the Georgia 2007/1 EP402R protein (SEQ ID No. 24) to R.

Methods for mutation of viral genes are known in the art. In particular, methods for deletion of viral genes are known in the art. For example, homologous recombination may be used, in which a transfer vector is created in which the relevant gene(s) are missing and used to transfect virus-infected cells. Recombinant viruses expressing the new portion of sequence may then be selected. Similar procedures may be used in order to interrupt gene expression, for example by deletion of the ATG start codon.

In some embodiments, the method of attenuating an ASF virus may comprise retaining the function of one or more of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. Suitably, the function of two or more, such as three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one, or twenty two of the following genes are retained: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R. In an embodiment the method of attenuating an ASF virus may comprise retaining the function of all of the following genes: MGF 110 3L, 6L, 7L, 8L, 10L, 11L and 12L; MGF 360 5L, 6L, 7L, 10L, 11L, 12L, 13L, 14L, 20R, 21R and 22R; and MGF 505 1R, 2R and 6R.

“Retaining the function” of a gene means that expression and activity of the gene is not affected during the attenuation process. The resultant attenuated virus should express a functional version of the gene. Suitably, the genes the function of which is to be retained are unaltered by the method of attenuation. Suitably, the sequences of the genes the function of which is to be retained are unaltered by the method of attenuation.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

### Further Aspects

The present invention also provides further aspects as defined in the following numbered paragraphs (paras).

1. An attenuated African Swine Fever (ASF) virus in which the expression and/or activity of the genes EP153R and K145R is disrupted.

2. An attenuated ASF virus according to para 1 in which the expression and/or activity of the EP402R is not disrupted.

3. An attenuated ASF virus according para 1 or 2 which comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

4. An attenuated ASF virus according to any preceding para wherein the K145R gene is at least partially deleted, preferably completely deleted.

5. An attenuated ASF virus according to para 4 wherein the K145R gene is interrupted.

6. An attenuated ASF virus according to any preceding para wherein the EP153R gene is at least partially deleted, preferably completely deleted.

7. An attenuated ASF virus according to any preceding para wherein the EP153R gene is interrupted.

8. An attenuated ASF virus according to any preceding para which comprises functional versions of all ASF virus genes other than EP153R and K145R.

9. An attenuated ASF virus according to any preceding para wherein the genome of the attenuated ASF virus corresponds to, or essentially corresponds to, genotype II.

10. An attenuated ASF virus according to para 9 wherein the genome of the attenuated ASF virus corresponds to, or essentially corresponds to, that of the Georgia 2007/1 strain.

11. An ASF virus according to any of paras 1 to 10 for use in treating and/or preventing a disease in a subject.

12. Use of an ASF virus according to any of paras 1 to 10 for manufacture of a medicament for treating and/or preventing disease in a subject.

13. A pharmaceutical composition comprising an ASF virus according to any of paras 1 to 10.

14. A pharmaceutical composition according to para 13 for use in treating and/or preventing a disease in a subject.

15. An ASF virus for use according to para 11, use of an ASF virus according to para 12, or a pharmaceutical composition for use according to para 14, wherein the disease is African Swine Fever.

16. A vaccine comprising an ASF virus according to any of paras 1 to 10.

17. A vaccine according to para 16 for use in treating and/or preventing African Swine Fever in a subject.

18. A vaccine for use according to para 17 wherein the African Swine Fever is caused by an ASF virus of a different genotype to the ASF virus of the vaccine.

19. A method for treating and/or preventing African Swine Fever in a subject which comprises the step of administering to the subject an effective amount of a pharmaceutical composition according to para 13 or a vaccine according to para 16.

20. An ASF virus for use according to para 11, use of an ASF virus according to para 12, a pharmaceutical composition for use according to para 14, a vaccine for use according to para 17, or a method according to para 19, wherein the subject is a domestic pig.

21. A vaccine for use according to any of paras 17, 18 or 20, or a method according to claim 19 or 20, in which the vaccine is administered following a prime-boost regime.

22. A method of attenuating an ASF virus which comprises disrupting the expression and/or activity of the EP153R and K145R genes.

23. A method according to para 22 wherein the K145R gene is at least partially deleted, preferably completely deleted.

24. A method according to para 23 wherein the K145R gene is interrupted.

25. A method according to any of paras 22 to 24 wherein the EP153R gene is at least partially deleted, preferably completely deleted.

26. A method according to any of para 22 to 25 wherein the EP153R gene is interrupted.

The invention will now be further described by way of Examples, which are meant to serve to assist one of ordinary skill in the art in carrying out the invention and are not intended in any way to limit the scope of the invention.

### EXAMPLES

### Example 1-Identification of EP402R/CD2v Mutants that Reduce HAD

Mutations were made in African swine fever virus (ASFV) Benin isolate EP402R protein (CD2v) and tested for their effect on haemadsorption (HAD).

A model of the extracellular, N-terminal, IgG-like, ligand-binding domain of CD2v was generated and used to predict the functional amino acid residues involved in binding of CD2v to its ligand. These residues were individually mutated to generate a set of mutant CD2v proteins.

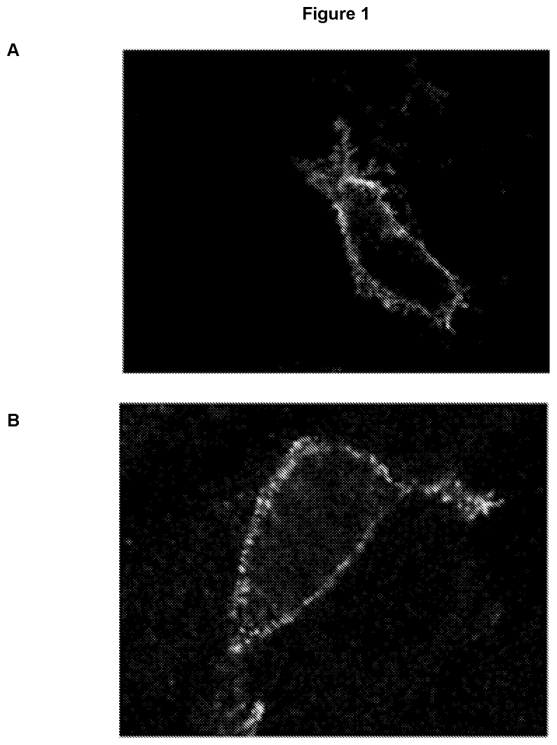

Vero cells were infected with modified vaccinia virus Ankara expressing T7RNA polymerase and transfected with plasmids (pcDNA3) expressing wild-type or mutant CD2v full-length proteins with a C-terminal HA epitope tag. Pig red blood cells were added and cells observed for attachment of red blood cells to the surface. Expression of the wild-type or mutant CD2v proteins was confirmed both by confocal microscopy using permeabilised cells and Western blotting using an antibody recognising the HA tag and a secondary antibody. Cell surface expression of the wild-type or mutant CD2v was also confirmed by staining non-permeabilised cells with sera from pigs immunised with attenuated ASFV containing a wild-type CD2v gene followed by a secondary antibody (FIG. 1).

Mutation of residues E99 or Y102 in Benin CD2v abrogated HAD. FIG. 2 shows exemplary images from the HAD assay. FIG. 2A shows cells transfected with a plasmid expressing wild-type Benin CD2v. HAD of red blood cells is observed around three cells. FIG. 2B shows cells expressing CD2v with the Y102 residue mutated to D. Partial HAD is observed around one cell. FIG. 2C shows cells expressing CD2v with residue E99 mutated to R. No HAD is observed.

Benin CD2v residue E99 is strongly conserved in ASFV, as shown by an alignment of the amino acid sequence of CD2v ligand-binding domain from different ASFV isolates of varying genotypes (FIG. 3). The equivalent residue in other isolates (highlighted in yellow in FIG. 3) is either identical (E) or has the same charge (Q).

The residue corresponding to Benin CD2v E99 in Georgia CD2v, Q96, was mutated to R and the ability of the mutant protein to induce HAD was tested using the HAD assay described above (wild type and mutant CD2v protein expressed from plasmids in Vero cells). Mutation of Q96 in Georgia CD2v abrogated HAD. FIG. 4 shows exemplary images from the HAD assay of Vero cells with pig red blood cells. FIG. 4A shows Vero cells transfected with a plasmid expressing wild-type Benin CD2v. HAD of red blood cells is observed around four cells. FIG. 4B shows Vero cells expressing wild-type Georgia CD2v. HAD is observed around two cells. FIG. 4C shows Vero cells expressing Georgia CD2v with residue Q96 mutated to R. No HAD is observed. FIG. 4D shows non-transfected Vero cells. No HAD is observed.

Using the same assay, the following mutations were determined to impact HAD in Benin: N16R, I 19R, W21D, Y 76D, E99R, Y102D. In addition, a combination of E99R+N108R was determined to impact HAD in Benin.

The following mutations were determined to impact HAD in Georgia: S15R, W19D, Q96R, N104R, and K108D. In addition, combinations of S15R+W19D and Q96R+N104R were determined to impact HAD in Georgia.

The following mutations were determined to impact HAD in N10 Genotype IX: W20D, R125D, Q112R+N121R.

### Example 2-Screening for DIVA Markers

For a gene to serve as a Differentiation of Infected from Vaccinated Animals (DIVA) marker, the protein expressed from the gene must be immunogenic. In other words, a subject infected with a virus expressing the DIVA protein must produce antibodies that specifically bind the DIVA protein. In this way, animals vaccinated with a DIVA vaccine (virus lacking the DIVA marker gene) can be differentiated from animals infected with wild type virus (which expresses the DIVA marker gene) because sera of vaccinated animals will not comprise antibodies to the DIVA marker protein, whereas sera of infected animals will comprise antibodies to the DIVA marker protein.

A selection of ASFV genes that might serve as DIVA markers were screened by expressing each gene in cells and testing whether the protein produced could be detected by sera taken from pigs that had previously been infected with ASFV. Detection by the sera would indicate that the protein, expressed by ASFV in the infected pigs, had induced an antibody response in the infected pigs. Such proteins were therefore candidates for DIVA markers.

In particular, 71 plasmids coding for individual ASFV genes (excluding known essential genes) fused to an HA or V5 epitope tag were transfected into Vero cells. The cells were fixed, permeabilised and stained with antisera from pigs that had been infected with different strains of ASFV, followed by a fluorescently labelled secondary antibody. Confocal microscopy was used to assess whether the expressed gene could be detected by the sera. In parallel the cells were stained with an antibody against the HA or V5 tag fused to the ASFV gene and a different fluorescently labelled secondary antibody to confirm expression of the protein.

The pig sera used for staining the cells were from pigs from immunisation studies that had been immunised with the following ASFV strains: BeninΔDP148R (5 pigs), BeninΔMGF (6 pigs), OURT88/3 (5 pigs) and GeorgiaΔMGF (4 pigs). For each pig, a pre-immunisation serum sample (as a control) and a post-immunisation, pre-challenge serum sample were used.

An initial screen of the genes was conducted using sera from BeninΔDP148R immunised pigs (pre-immunisation sera from day 0, post-immunisation sera from day 38 post-immunisation). ASFV genes CP204L, B646L and E183L were used as positive controls and were detected using post-immunisation sera. Pre-immunisation sera did not detect any genes.

Six ASFV genes were detected using BeninΔDP148R post-immunisation sera, as shown below in Table 7 (++ indicates strong detection, + indicates weak detection, − indicates no detection).

TABLE 7

| | BeninΔDP148R post-immunisation serum (day 38) | | | | |
|---|---|---|---|---|---|
| ASFV gene | Pig 1 | Pig 2 | Pig 3 | Pig 4 | Pig 5 |
| B125R | ++ | ++ | − | − | + |
| B175L | ++ | ++ | − | + | − |
| E184L | ++ | ++ | − | − | + |
| H339R | − | ++ | − | ++ | ++ |
| K145R | ++ | ++ | − | − | − |
| M448R | ++ | − | + | ++ | + |

The six ASFV genes detected in the initial screen were then tested with pig serum from the other three immunisation studies.

Table 8 below shows detection of ASFV genes using post-immunisation sera from 6 pigs immunised with BeninΔMGF virus (boosted on day 15, post-immunisation serum taken on day 38 post-immunisation; pre-immunisation sera were negative).

TABLE 8

| | BeninΔMGF post-immunisation serum (day 38) | | | | | |
|---|---|---|---|---|---|---|
| ASFV gene | Pig 1 | Pig 2 | Pig 3 | Pig 4 | Pig 5 | Pig 6 |
| B125R | ++ | ++ | + | ++ | ++ | ++ |
| B175L | ++ | ++ | ++ | ++ | ++ | ++ |
| E184L | ++ | ++ | ++ | ++ | ++ | + |
| H339R | − | − | + | + | ++ | ++ |
| K145R | + | − | − | ++ | ++ | ++ |
| M448R | ++ | − | ++ | − | + | ++ |

Table 9 below shows detection of ASFV genes using post-immunisation sera from 5 pigs immunised with OURT88/3 virus (post-immunisation serum taken on day 20 post-immunisation; pre-immunisation sera were negative except for pig 2).

TABLE 9

| | OURT88/3 post-immunisation serum (day 20) | | | | |
|---|---|---|---|---|---|
| ASFV gene | Pig 1 | Pig 2 | Pig 3 | Pig 4 | Pig 5 |
| B125R | ++ | ++ | + | + | − |
| B175L | ++ | ++ | + | ++ | + |
| E184L | ++ | ++ | + | ++ | ++ |
| H339R | ++ | ++ | + | ++ | + |
| K145R | ++ | ++ | + | + | + |
| M448R | ++ | ++ | + | ++ | + |

Table 10 below shows detection of ASFV genes using post-immunisation sera from 4 pigs immunised with GeorgiaΔMGF virus (post-immunisation serum taken on day 34 post-immunisation; pre-immunisation sera taken on day-3 were negative). 2 pigs (A) were immunised with 103 GeorgiaΔMGF; 2 pigs (B) were immunised with 104 GeorgiaΔMGF.

TABLE 10

| | GeorgiaΔMGF post-immunisation serum (day 34) | | | |
|---|---|---|---|---|
| ASFV gene | Pig 1 (A) | Pig 2 (A) | Pig 3 (B) | Pig 4 (B) |
| B125R | − | ++ | + | − |
| B175L | + | − | − | + |
| E184L | − | − | ++ | + |
| H339R | − | + | − | − |
| K145R | − | ++ | + | − |
| M448R | − | − | ++ | − |

K145R protein was detected by 65% of sera and B125R was detected by 75% of sera. Each of the B125R, B175L, E184L, H339R, K145R and M448R genes was individually deleted. The B175L, E184L, H339R or M448R genes could not be deleted, suggesting that that are essential for virus replication. Thus, the screen identified the K145R and B125R genes as the most promising potential DIVA markers.

FIG. 5 shows K145R and B125R expressed in cells. Vero cells were transfected with plasmids expressing K145R or B125R with a HA epitope tag fused in frame. The expressed proteins were detected in permeabilised cells using an antibody against HA and imaged using a confocal microscope. Green staining shows the expressed proteins and blue DAPI stain detects DNA. FIG. 5A shows K145R and FIG. 5B shows B125R.

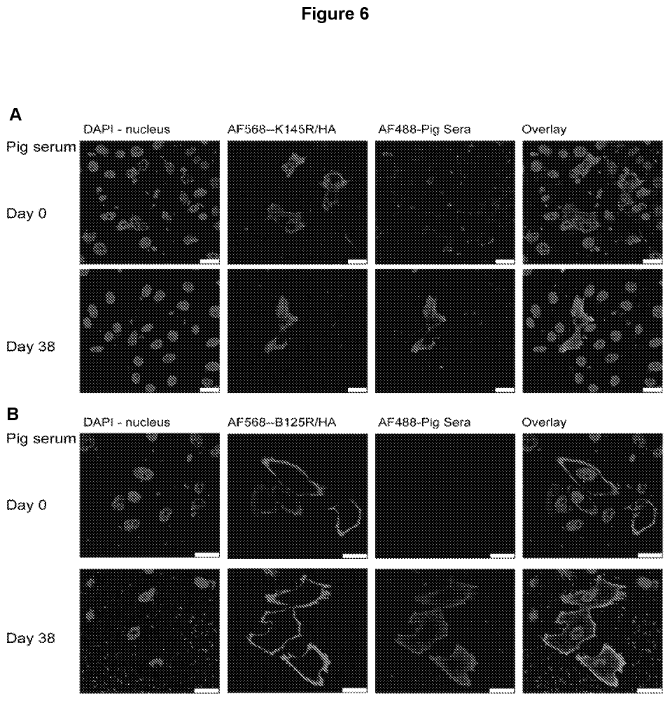

FIG. 6 shows an example from the screening process of K145R (FIG. 6A) and B125R (FIG. 6B) expressed in Vero cells and detected by antisera from pigs immunised with ASFV. Cells were fixed, permeabilised and stained with anti-HA (red) to detect the expressed proteins and with sera from pigs immunised with an attenuated genotype|Benin97/1 gene deleted ASFV strain (green). Images are shown of cells stained with sera collected before immunisation and at day 38 post-immunisation. DNA is stained in blue.

### Example 3-Generation of Non-HAD ASFV GeorgiaΔK145RΔEP153RCD2vQ96R

Based on the findings described in the Examples above, ASFV was generated in which the K145R and EP153R genes were deleted and the EP402R/CD2v protein was mutated to comprise the Q96R amino acid substitution. Georgia 2007/1 strain (a strain of ASFV genotype II) was used. The ASFV is accordingly designated GeorgiaΔK145RΔEP153RCD2vQ96R.

The ability of GeorgiaΔK145RΔEP153RCD2vQ96R to induce HAD was tested. Porcine bone marrow cells were infected with GeorgiaΔK145RΔEP153RCD2vQ96R or wild type Georgia 2007/1 as control, pig red blood cells were added and the cells observed for attachment of red blood cells to the surface. HAD was observed in cells infected with wild type Georgia 2007/1 at 1 day post-infection (FIG. 7A-red blood cells accumulate around infected cells) whilst HAD was not observed in cells infected with GeorgiaΔK145RΔEP153RCD2vQ96R at 1 day post-infection (FIG. 7B).

### Example 4-Georgia AK145RΔEP153RCD2vQ96R is Attenuated and Induces Protection Against Challenge

### Vaccination Experimental Protocol

A group of six Large White/Landrace pigs (Group K) varying in weight from 17 to 19 kg and aged 7 weeks old were immunised by the intramuscular route with 104 TCID₅₀ in 1 ml with GeorgiaΔK145RΔEP153RCD2vQ96R and boosted after 21 days by the same route with the same dose. After a further 18 days the Group K immunised pigs and a control group of 3 non-immune pigs (Group M) were challenged by the intramuscular route with 103 TCID₅₀ in 1 ml with virulent genotype II ASF virus Georgia 2007/1. After a further 20 days pigs were terminated. This experimental protocol is depicted in FIG. 8.

### Temperature and Clinical Scores

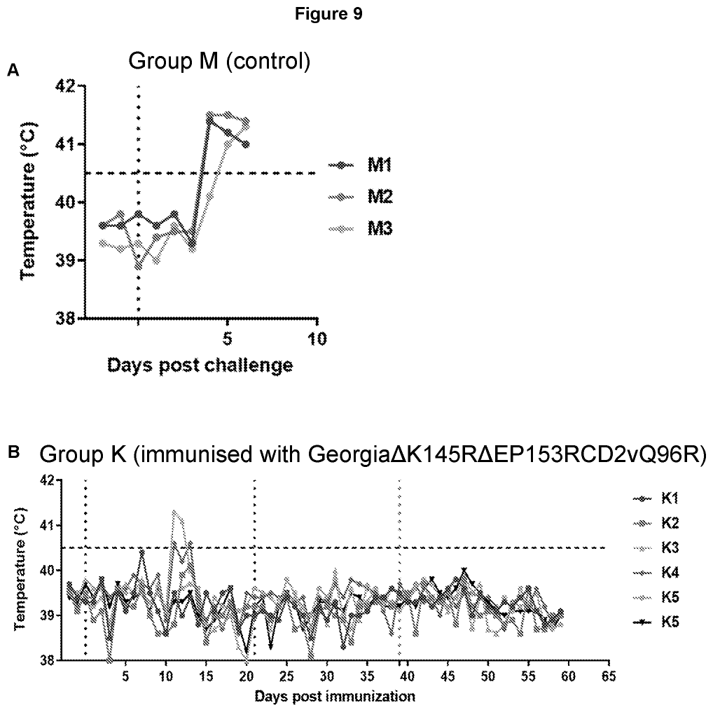

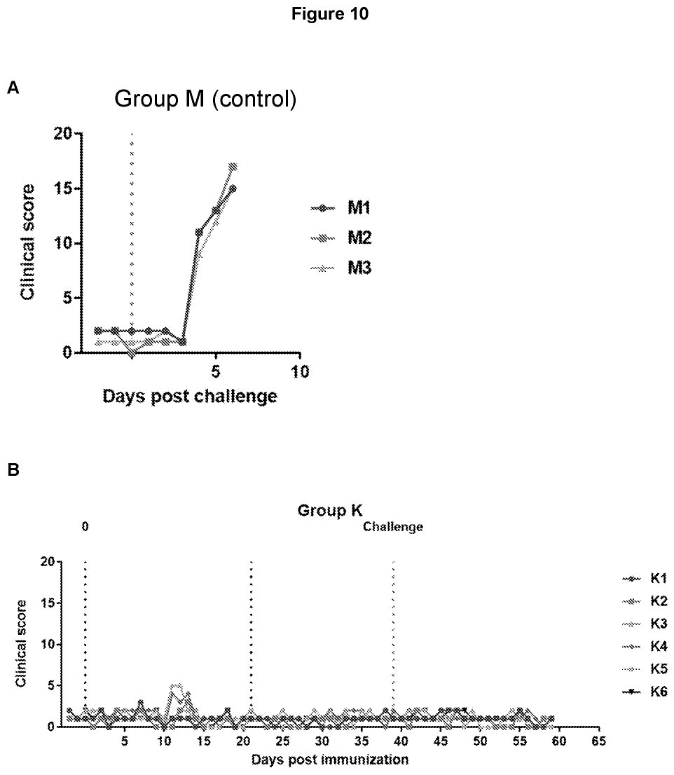

Temperatures (FIG. 9) and clinical scores (FIG. 10) of the pigs were recorded daily using a standard scoring system (King et al., 2011). The clinical scores include temperatures and other signs such as loss of appetite or lethargy.

The control group M of non-immune pigs developed an increased temperature (FIG. 9A) and other clinical signs typical of acute ASFV, including not eating and lethargy, from day 4 post-challenge and were euthanised on day 6 post-challenge at a moderate severity end point (FIG. 10A).

Two of the pigs in the immunised group K had a transient increase in temperature above 40.6 for 2 days starting at day 11 post-immunisation (FIG. 9B). These and all other pigs in Group K had no other clinical signs post-boost or after challenge (FIG. 10B).

Previous work had shown that deletion of K145R had minimal direct attenuating effects.

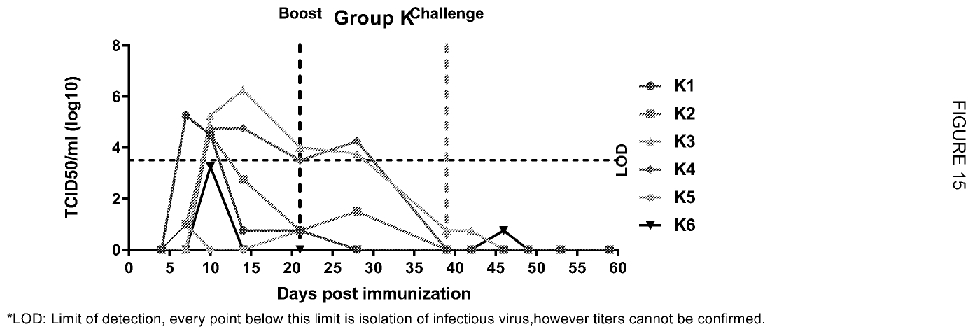

Viremia data is shown in FIG. 15.

### Scoring of Macroscopic Lesions at Post-Mortem

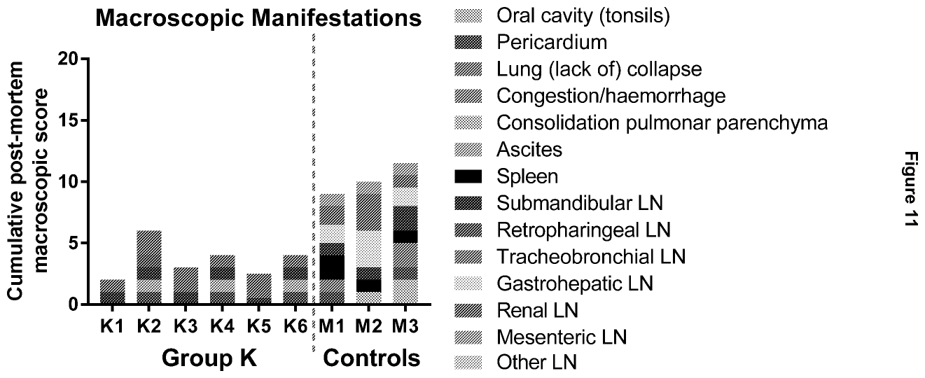

At necropsy macroscopic lesions in different organs and cavities were scored according to a standardised scoring system. In FIG. 11 scores are shown on the y axis and pig numbers shown on the x axis. Pigs from group K (immunised with GeorgiaΔK145RΔEP153RCD2vQ96R) showed few lesions, which were mainly slightly enlarged renal or submandibular lymph nodes. In contrast the control non-immune pigs in Group M had lesions typical of acute ASFV including enlarged and haemorrhagic lymph nodes and enlarged spleen.

### Antibody Response of Immunised Pigs

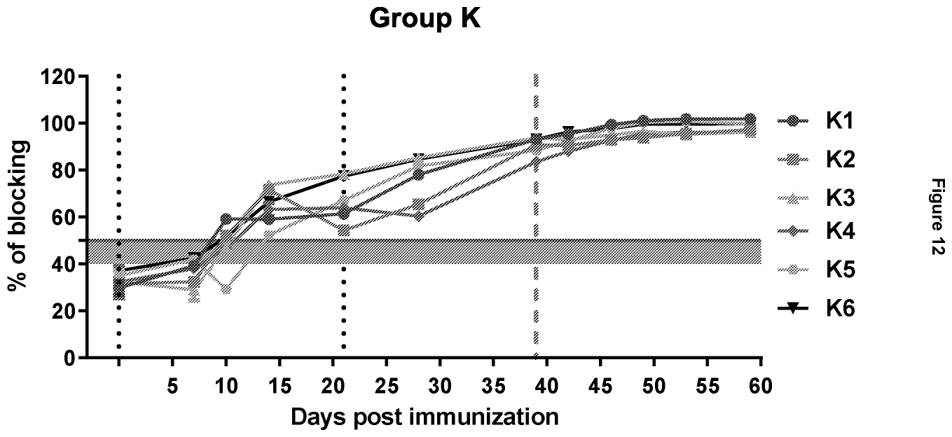

Sera collected from pigs of group K at different days before immunisation and at different days after immunisation, boost and challenge were tested for levels of antibodies against the major ASFV capsid protein VP72/B646L using a commercial competitive ELISA assay. In FIG. 12 the % of blocking is shown on the y axis and days post-immunization on the x axis. Data for different pigs is shown as indicated by different colours. The grey line indicates the cut-off value indicated by the manufacturer for detection of positive sera. The results show that by day 14 post-immunisation values were positive for all pigs and levels reached a plateau which was maintained during the experiment.

### Cell-Mediated Immune Response

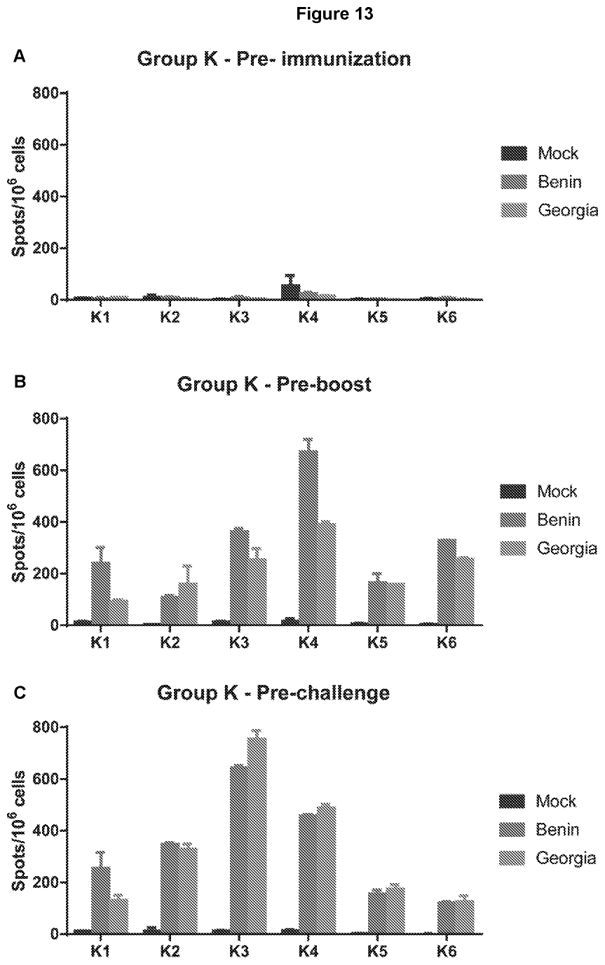

Peripheral blood mononuclear cells (PBMCs) were collected from pigs of group K before immunisation with GeorgiaΔK145RΔEP153RCD2vQ96R, before boost and before challenge with Georgia 2007/1 virus. The PBMCs were stimulated with ASFV and the number of interferon gamma producing cells measured as an indicator of the cellular immune response to ASFV (FIG. 13).

The PBMCs were mock stimulated (blue bars) or stimulated with ASFV genotype|Benin97/1 infectious virus (red bars) or ASFV genotype II Georgia 2007/1 virus (green bars). Numbers of interferon gamma producing cells were measured and are shown per 10⁶ cells on the y axis. The pig number is given on the x-axis. The results show very low or no detectable IFN gamma producing cells before immunisation, as expected (FIG. 13A). By day 21 before the boost numbers of IFN gamma producing cells had increased varying between about 100 and 750 cells depending on the ASFV virus used for stimulus (FIG. 13B). The numbers of IFN gamma producing cells following ASFV stimulus were maintained at good levels at day 39 before the challenge (FIG. 13C). IFN gamma production was stimulated by both genotype I and genotype II isolates suggesting a cross-genotype cellular response was induced. As expected, mock-stimulation did not cause a detectable response.

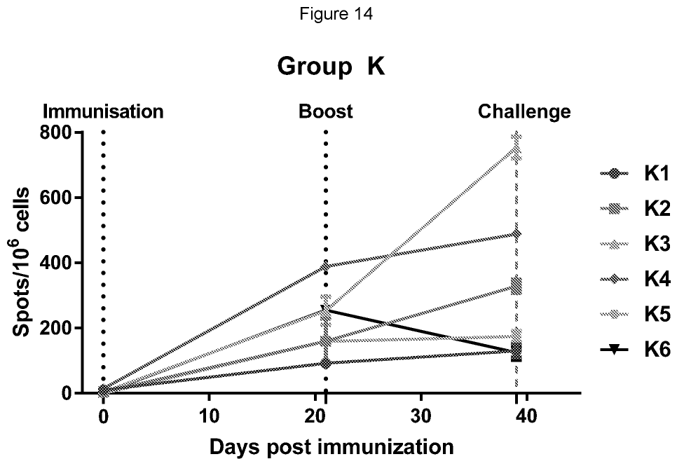

FIG. 14 shows the number of IFN gamma producing cells for different pigs following stimulation with Georgia 2007/1 isolate over time.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in virology, molecular biology or related fields are intended to be within the scope of the following claims.

1. An attenuated African Swine Fever (ASF) virus in which the expression and/or activity of the genes EP153R and EP402R is disrupted;
and which comprises a functional version of one or more of the following genes:
multigene family (MGF) 110 3L, 6L, 7L, 8L, 10L, 11L and 12L,
MGF 360 5L, 6L, 7L, 10L, 11L, 12L, 13L, 14L, 20R, 21R and 22R, and
MGF 505 1R, 2R and 6R.

2. An attenuated ASF virus according to claim 1 further comprising a Differentiation of Infected from Vaccinated Animals (DIVA) mutation.

3. An attenuated ASF virus in which the expression and/or activity of the genes EP153R and EP402R is disrupted and which comprises a DIVA mutation.

4. An attenuated ASF virus according to claim 2 or 3 wherein the DIVA mutation disrupts expression of the K145R gene.

5. An attenuated ASF virus according to claim 4 wherein the K145R gene is at least partially deleted, preferably completely deleted.

6. An attenuated ASF virus according to claim 4 wherein the K145R gene is interrupted.

7. An attenuated ASF virus according to any of claims 1 to 6 wherein the EP153R gene is at least partially deleted, preferably completely deleted.

8. An attenuated ASF virus according to any of claims 1 to 6 wherein the EP153R gene is interrupted.

9. An attenuated ASF virus according to any of claims 1 to 8 wherein the ability of the ASF virus to induce haemadsorption is reduced compared to a corresponding ASF virus in which the expression and/or activity of the EP153R gene and/or the EP402R gene is not disrupted.

10. An attenuated ASF virus according to any of claims 1 to 9 wherein surface expression of the EP402R protein is reduced compared to a corresponding ASF virus in which the expression and/or activity of the EP402R gene is not disrupted.

11. An attenuated ASF virus according to any of claims 1 to 10 wherein the EP402R gene comprises one or more mutation that disrupts ligand binding by the EP402R protein.

12. An attenuated ASF virus according to claim 10 or 11 wherein the EP402R gene comprises one or more mutation that changes one or more amino acid in the ligand-binding domain of the EP402R protein.

13. An attenuated ASF virus according to claim 12 wherein the one or more amino acid is changed to a different amino acid.

14. An attenuated ASF virus according to claim 13 wherein the change to a different amino acid directly inhibits the interaction between EP402R and its ligand by changing the binding surface on EP402R.

15. An attenuated ASF virus according to any of claims 11 to 14 wherein the one or more mutations change an amino acid at a position in the EP402R protein which corresponds to Q96 and/or W99 of Georgia 2007/1 EP402R protein (SEQ ID No. 24).

16. An attenuated ASF virus according to claim 15 wherein the amino acid at the position which corresponds to Q96 is changed to R or to an amino acid that is a conservative replacement of R and/or the amino acid at the position equivalent to W99 is changed to D or to an amino acid that is a conservative replacement of D.

17. An attenuated ASF virus according to claim 16 wherein the amino acid at the position which corresponds to Q96 is changed to H, K or R and/or the amino acid at the position which corresponds to W99 is changed to D, E, N or Q.

18. An attenuated ASF virus according to claim 17 wherein the amino acid at the position which corresponds to Q96 is changed to R and/or the amino acid at the position which corresponds to W99 is changed to D.

19. An attenuated ASF virus according to any of claims 1 to 12 wherein the EP402R gene is at least partially deleted, preferably completely deleted.

20. An attenuated ASF virus according to any of claims 1 to 12 wherein the EP402R gene is interrupted.

21. An attenuated ASF virus according to claim 1 or any of claims 7 to 20 which comprises functional versions of all ASF virus genes other than EP153R and EP402R.

22. An attenuated ASF virus according to any of claims 1 to 20 which comprises functional versions of all ASF virus genes other than EP153R, EP402R and K145R.

23. An attenuated ASF virus according to any of claims 1 to 22 wherein the genome of the attenuated ASF virus corresponds to, or essentially corresponds to, genotype II.

24. An attenuated ASF virus according to claim 23 wherein the genome of the attenuated ASF virus corresponds to, or essentially corresponds to, that of the Georgia 2007/1 strain.

25. An EP402R protein comprising one or more amino acid change in the ligand-binding domain wherein the amino acid change disrupts ligand-binding of the EP402R protein.

26. An EP402R protein comprising one or more amino acid change at a position which corresponds to Q96 and/or W99 of the Georgia 2007/1 EP402R protein (SEQ ID No. 24).

27. An EP402R protein according to claim 26 wherein the amino acid at the position which corresponds to Q96 is changed to R or to an amino acid that is a conservative replacement of R and/or the amino acid at the position which corresponds to W99 is changed to D or to an amino acid that is a conservative replacement of D.

28. An EP402R protein according to claim 27 wherein the amino acid at the position which corresponds to Q96 is changed to H, K or R and/or the amino acid at the position which corresponds to W99 is changed to D, E, N or Q.

29. An EP402R protein according to claim 28 wherein the amino acid at the position which corresponds to Q96 is changed to R and/or the amino acid at the position which corresponds to W99 is changed to D.

30. An EP402R protein according to any of claims 25 to 29 comprising an amino acid sequence having at least 70% sequence identity with any of SEQ ID Nos 21 to 30 or SEQ ID Nos 242 to 246.

31. An EP402R protein according to claim 30 comprising the amino acid sequence of any of SEQ ID Nos 31, 32, 33 or 379.

32. A polynucleotide encoding the EP402R protein of any of claims 25 to 31.

33. A polynucleotide according to claim 32 comprising a sequence having at least 70% identity with any of SEQ ID Nos 229 to 241.

34. A vector comprising the polynucleotide of any of claims 32 to 33.

35. An ASF virus comprising the EP402R protein of any of claims 25 to 31.

36. An ASF virus comprising the polynucleotide of claim 32 or 33.

37. An ASF virus according to claim 35 or 36 wherein the ability of the ASF virus to induce haemadsorption is reduced compared to a corresponding ASF virus which does not comprise the EP402R protein of any of claims 25 to 31 or the polynucleotide of claim 32 or 33.

38. An ASF virus according to any of claims 35 to 37 which is attenuated.

39. An ASF virus according to any of claims 35 to 38 further comprising a DIVA mutation.

40. An ASF virus according to claim 39 wherein the DIVA mutation disrupts expression of the K145R gene.

41. An ASF virus according to claim 40 wherein the K145R gene is at least partially deleted, preferably completely deleted.

42. An ASF virus according to claim 40 wherein the K145R gene is interrupted.

43. An ASF virus according to any of claims 35 to 42 wherein expression and/or activity of the EP153R gene is disrupted.

44. An ASF virus according to claim 43 wherein the EP153R gene is at least partially deleted, preferably completely deleted.

45. An ASF virus according to claim 43 wherein the EP153R gene is interrupted.

46. An ASF virus according to any of claims 35 to 45 wherein the ASF virus genome corresponds to, or essentially corresponds to, genotype II.

47. An ASF virus according to claim 46 wherein the ASF virus genome corresponds to, or essentially corresponds to, that of the Georgia 2007/1 strain.

48. An ASF virus according to any of claims 1 to 24 or any of claims 35 to 47 for use in treating and/or preventing a disease in a subject.

49. Use of an ASF virus according to any of claims 1 to 24 or any of claims 35 to 47 for manufacture of a medicament for treating and/or preventing disease in a subject.

50. A pharmaceutical composition comprising an ASF virus according to any of claims 1 to 24 or any of claims 35 to 47.

51. A pharmaceutical composition according to claim 50 for use in treating and/or preventing a disease in a subject.

52. An ASF virus for use according to claim 48, use of an ASF virus according to claim 49, or a pharmaceutical composition for use according to claim 51, wherein the disease is African Swine Fever.

53. A vaccine comprising an ASF virus according to any of claims 1 to 24 or any of claims 35 to 47.

54. A vaccine according to claim 53 for use in treating and/or preventing African Swine Fever in a subject.

55. A vaccine for use according to claim 54 wherein the African Swine Fever is caused by an ASF virus of a different genotype to the ASF virus of the vaccine.

56. A method for treating and/or preventing African Swine Fever in a subject which comprises the step of administering to the subject an effective amount of a pharmaceutical composition according to claim 50 or a vaccine according to claim 53.

57. An ASF virus for use according to claim 48 or 52, use of an ASF virus according to claim 49 or 52, a pharmaceutical composition for use according to claim 51 or 52, a vaccine for use according to claim 54 or 55, or a method according to claim 56, wherein the subject is a domestic pig.

58. A vaccine for use according to any of claim 54, 55 or 57, or a method according to claim 56 or 57, in which the vaccine is administered following a prime-boost regime.

59. A method of producing an ASF virus of any of claims 35 to 47, the method comprising changing one or more amino acid in the ligand-binding domain of the EP402R protein wherein the amino acid change disrupts ligand-binding of the EP402R protein.

60. A method of producing an ASF virus of any of claims 35 to 47, the method comprising changing one or more amino acid in the EP402R protein at a position which corresponds to Q96 and/or W99 of the Georgia 2007/1 EP402R protein (SEQ ID No. 24).

61. A method of reducing the ability of an ASF virus to induce haemadsorption, the method comprising changing one or more amino acid changes in the ligand-binding domain of the EP402R protein wherein the amino acid changes disrupt ligand-binding of the EP402R protein.

62. A method of reducing the ability of an ASF virus to induce haemadsorption, the method comprising changing an amino acid in the EP402R protein at a position which corresponds to Q96 and/or W99 of the Georgia 2007/1 EP402R protein (SEQ ID No. 24).

63. A method according to any of claims 59 to 62 further comprising disrupting the expression and/or activity of the EP153R gene.

64. A method of attenuating an ASF virus which comprises disrupting the expression and/or activity of the EP153R and EP402R genes.

65. A method according to claim 64 comprising disrupting the ability of the EP153R gene and/or the EP402R gene to mediate haemadsorption.

66. A method according to any of claims 59 to 65 further comprising introducing a DIVA mutation into the ASF virus.

67. A method according to claim 66 wherein the DIVA mutation disrupts expression of the K145R gene.

68. A method according to claim 67 wherein the K145R gene is at least partially deleted, preferably completely deleted.

69. A method according to claim 67 wherein the K145R gene is interrupted.

70. A method according to any of claims 63 to 69 wherein the EP153R gene is at least partially deleted, preferably completely deleted.

71. A method according to any of claims 63 to 69 wherein the EP153R gene is interrupted.

72. A method according to any of claims 64 to 71 wherein the EP402R gene is at least partially deleted, preferably completely deleted.

73. A method according to any of claims 64 to 71 wherein the EP402R gene is interrupted.

74. A method according to any of claims 64 to 73 comprising introducing one or more mutations in the EP402R gene that reduce surface expression of the EP402R protein reduced compared to a corresponding ASF virus that does not comprise the one or more mutations.

75. A method according to any of claims 64 to 74 comprising introducing one or more mutations in the EP402R gene that disrupt ligand binding by the EP402R protein.

76. A method according to claim 74 or 75 comprising introducing one or more mutations in the EP402R gene that change one or more amino acids in the ligand-binding domain of the EP402R protein.

77. A method according to claim 76 wherein the one or more amino acids are changed to different amino acids.

78. A method according to claim 77 wherein the change to different amino acids directly inhibits the interaction between EP402R and its ligand by changing the binding surface on EP402R.

79. A method according to any of claims 75 to 78 comprising changing an amino acid in the EP402R protein at a position which corresponds to Q96 and/or W99 of the Georgia 2007/1 EP402R protein (SEQ ID No. 24).

80. A method according to any of claims 59 to 79 wherein an amino acid in the EP402R protein at a position which corresponds to Q96 of the Georgia 2007/1 EP402R protein (SEQ ID No. 24) is changed to R or to an amino acid that is a conservative replacement of R and/or an amino acid at a position which corresponds to W99 of the Georgia 2007/1 EP402R protein (SEQ ID No. 24) is changed to D or to an amino acid that is a conservative replacement of D.

81. A method according to claim 80 wherein the amino acid at the position which corresponds to Q96 is changed to H, K or R and/or the amino acid at the position which corresponds to W99 is changed to D, E, N or Q.

82. A method according to claim 81 wherein the amino acid at the position which corresponds to Q96 is changed to R and/or the amino acid at the position which corresponds to W99 is changed to D.
